(19) United States
(12) Patent Application Publication
(10) Pub. No.: US 2025/0110405 A1
(43) Pub. Date: Apr. 3, 2025

(54) MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 18/885,948

(22) Filed: Sep. 16, 2024

(30) Foreign Application Priority Data
Sep. 29, 2023 (JP) 2023-169702

(51) Int. Cl. G03F 7/039; C07C 69/76; C07D 307/00; C08F 22/10; C08F 22/14; C08F 22/26; G03F 7/00; G03F 7/20

(52) U.S. Cl. CPC G03F 7/0397; C07C 69/76; C07D 307/00; C08F 22/1006; C08F 22/14; C08F 22/26; G03F 7/2006; G03F 7/70025; G03F 7/70033; C07C 2601/08; C07C 2603/20

(57) ABSTRACT

A monomer containing as a polymerizable group an acenaphthylene structure having an acid labile group of tertiary ester type attached thereto is provided as well as a polymer comprising repeat units derived from the monomer. A chemically amplified resist composition comprising the polymer has advantages including high sensitivity, high contrast, improved lithography properties, e.g., EL, LWR and profile, collapse resistance during fine pattern formation, and etch resistance after development.

### CROSS-REFERENCE TO RELATED APPLICATIONS

This non-provisional application claims priority under 35 U.S.C. § 119(a) on Patent Application No. 2023-169702 filed in Japan on Sep. 29, 2023, the entire contents of which are hereby incorporated by reference.

### TECHNICAL FIELD

This invention relates to a monomer, polymer, chemically amplified resist composition, and pattern forming process.

### BACKGROUND ART

To meet the demand for higher integration density and operating speed of LSIs, the effort to reduce the pattern rule is in rapid progress. As the use of 5G high-speed communications and artificial intelligence (A1) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LER or LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

With the advance of miniaturization, pattern collapse during fine pattern formation and resistance during etching step are important for positive resist compositions. Patent Documents 3 and 4 disclose a polymer comprising structural units using acenaphthylene of robust structure as a polymerizable group, and containing a polar group of lactone structure or the like, an acidic group of hexafluoroisopropanol structure or carboxy group, and an acid labile group of acetal or tertiary ether structure, as partial structures. Patent Document 5 discloses a negative resist composition adapted for organic solvent development, comprising a polymer containing acenaphthylene units having a chain-like tertiary ester structure as constituent units.

These resist compositions achieve performance improvements to a certain extent, but not to a satisfactory extent. To meet the demand for further miniaturization, it is desired to have an acid labile monomer having a satisfactory acid elimination reactivity relative to acid, resistance to pattern collapse after fine pattern formation, and etch resistance during etching step.

### CITATION LIST

Patent Document 1: JP-A 2006-045311 (U.S. Pat. No. 7,482,108)
Patent Document 2: JP-A 2006-178317
Patent Document 3: WO 2022/186059
Patent Document 4: WO 2023/054126
Patent Document 5: JP 5793389

### SUMMARY OF THE INVENTION

An object of the invention is to provide a monomer, a polymer obtained from the monomer, and a chemically amplified resist composition comprising the polymer, the polymer having a high solvent solubility, the chemically amplified resist composition, when processed by photolithography using high-energy radiation such as KrF excimer laser, ArF excimer laser, EB or EUV, exhibiting a high sensitivity, high contrast, and improved lithography properties (e.g., EL and LWR), and forming a pattern of satisfactory profile having etch resistance after development. A further object of the invention is to provide a pattern forming process using the resist composition.

The inventor has found that a polymer comprising repeat units derived from a monomer having an acenaphthylene structure as a polymerizable group and having an acid labile group of tertiary ester type attached thereto has a high solvent solubility, that a chemically amplified resist composition comprising the polymer exhibits a high sensitivity, high contrast, and improved lithography properties (e.g., EL and LWR), and forms a fine pattern of satisfactory profile having collapse resistance during pattern formation and etch resistance after development.

In one aspect, the invention provides a monomer having the formula (A).

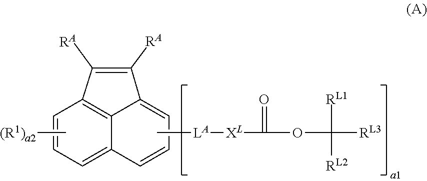

Herein a1 is 1 or 2, a2 is an integer of 0 to 4,
R^{A} is each independently hydrogen, fluorine, methyl or trifluoromethyl,
R¹ is halogen or a C₁-C₂₀ hydrocarbyl group which may contain a heteroatom, a plurality of R¹ may bond together to form a ring with the carbon atoms to which they are attached when a2 is 2 or more,
L^{A} is a single bond, ether bond, ester bond, amide bond, sulfonate ester bond, carbonate bond or carbamate bond,
X^{L} is a single bond or C₁-C₄₀ hydrocarbylene group which may contain a heteroatom,
R^{L1}, R^{L2} and R^{L3} are each independently a C₁-C₃₀ hydrocarbyl group which may contain a heteroatom, any two of R^{L1}, R^{L2} and R^{L3} may bond together to form a ring with the carbon atom to which they are attached, at least one of R^{L1}, R^{L2} and R^{L3} has at least one structure selected from a multiple bond, alicyclic and aromatic ring when they do not form a ring.

The preferred monomer has the formula (A1):

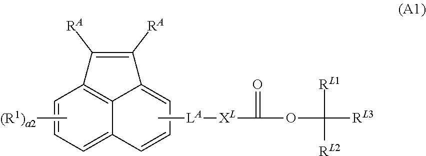

wherein a2, R^{A}, R¹, L^{A}, X^{L}, and R^{L1} to R^{L3} are as defined above.

The more preferred monomer has the formula (A2):

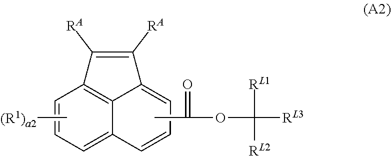

wherein a2, R^{A}, R¹, and R^{L1} to R^{L3} are as defined above.

In another aspect, the invention provides a polymer comprising repeat units derived from the monomer defined herein.

The polymer may further comprise repeat units of at least one type selected from repeat units having the formula (a1) and repeat units having the formula (a2).

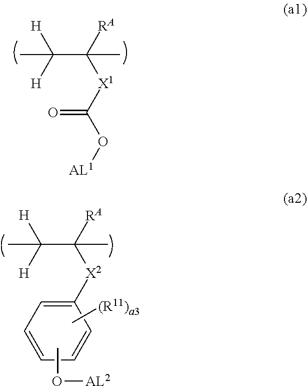

Herein R^{A} is each independently hydrogen, fluorine, methyl or trifluoromethyl,
X¹ is a single bond, phenylene, naphthylene, *—C(═O)—O—X¹¹—, or *—C(═O)—NH—X¹¹—, the phenylene or naphthylene group may be substituted with an optionally fluorinated C₁-C₁₀ alkoxy moiety or halogen, X¹¹ is a C₁-C₁₀ saturated hydrocarbylene group, phenylene, or naphthylene, the saturated hydrocarbylene group may contain a hydroxy, ether bond, ester bond or lactone ring,
X² is a single bond, *—C(═O)—O— or *—C(═O)—NH—,
* designates a point of attachment to the carbon atom in the backbone,
R¹¹ is halogen, cyano group, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL¹ and AL² are each independently an acid labile group, and
a3 is an integer of 0 to 4.

The polymer may further comprise repeat units of at least one type selected from repeat units having the formula (b1) and repeat units having the formula (b2).

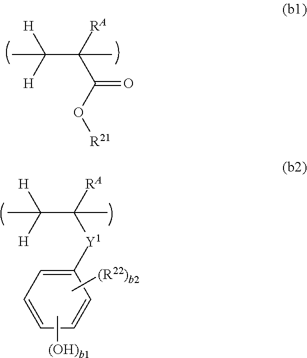

Herein R^{A} is each independently hydrogen, fluorine, methyl or trifluoromethyl,
Y¹ is a single bond or *—C(═O)—O—,*designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R²² is halogen, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
b1 is an integer of 1 to 4, b2 is an integer of 0 to 4, and b1+b2 is from 1 to 5.

The polymer may further comprise repeat units of at least one type selected from repeat units having the formulae (c1) to (c4).

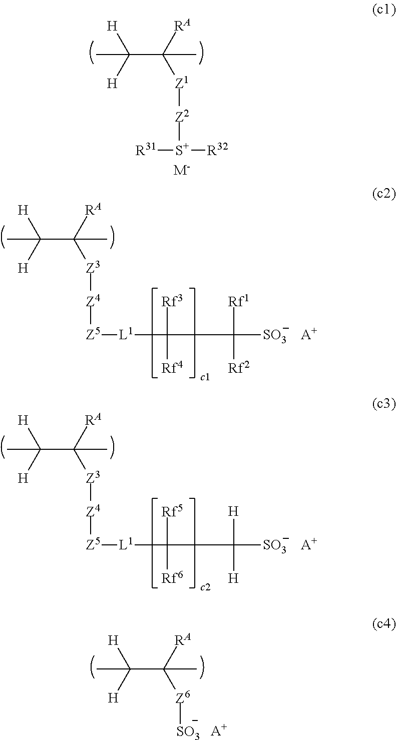

Herein R^{A} is each independently hydrogen, fluorine, methyl or trifluoromethyl,
Z¹ is a single bond or phenylene group,
Z² is **—C(═O)—O—Z²¹—, **—C(═O)—NH—Z²¹—, or **—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is each independently a single bond, phenylene, naphthylene, or *—C(═O)—O—Z³¹—, Z³¹ is a C₁-C₁₀ aliphatic hydrocarbylene group which may contain a hydroxy moiety, ether bond, ester bond or lactone ring, or phenylene or naphthylene group,
Z⁴ is each independently a single bond, ***—Z⁴¹—C(═O)—O—, ***—C(═O)—NH—Z⁴¹—, or ***—Z⁴¹—, Z⁴¹ is a C₁-C₂₀ hydrocarbylene group which may contain a heteroatom,
Z⁵ is each independently a single bond, ****—Z⁵—C(═O)—O—, ****—C(═O)—NH—Z⁵¹, or ****—O—Z⁵—, Z⁵¹ is a C₁-C₂₀ hydrocarbylene group which may contain a heteroatom,
Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹—, or *—O—Z⁶¹, Z⁶¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone, ** designates a point of attachment to Z¹, *** designates a point of attachment to Z³, **** designates a point of attachment to Z⁴,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonate ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
M⁻ is a non-nucleophilic counter ion,
A⁺ is an onium cation, and
c1 and c2 are each independently an integer of 0 to 3.

In a further aspect, the invention provides a chemically amplified resist composition comprising a base polymer containing the polymer defined herein and an organic solvent.

The chemically amplified resist composition may further comprise a quencher, a photoacid generator, and/or a surfactant.

In a still further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser radiation, ArF excimer laser radiation, EB or EUV of wavelength 3 to 15 nm.

### Advantageous Effects of Invention

When a chemically amplified resist composition comprising a polymer comprising repeat units derived from the inventive monomer is lithographically processed to form a pattern, the composition exhibits a high sensitivity, high contrast, and improved lithography properties (e.g., DOF and LWR) and forms a fine pattern of satisfactory profile having collapse resistance.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Et for ethyl, Ac for acetyl, and the broken line (---) and asterisk (*) designate a point of attachment or valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
EL: exposure latitude
CDU: critical dimension uniformity
DOF: depth of focus

### [Monomer]

One embodiment of the invention is a monomer having the formula (A).

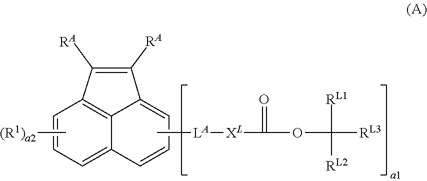

In formula (A), a1 is an integer of 1 or 2, preferably 1 for availability of the starting reactant. The subscript a2 is an integer of 0 to 4, preferably 0, 1 or 2, more preferably 0 or 1.

In formula (A), R^{A} is each independently hydrogen, fluorine, methyl or trifluoromethyl, preferably hydrogen or methyl, more preferably hydrogen.

In formula (A), R¹ is halogen or a C₁-C₂₀ hydrocarbyl group which may contain a heteroatom. Suitable halogen atoms include fluorine, chlorine, bromine and iodine. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl and 2-phenylethyl; and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—), or haloalkyl moiety.

When a2 is 2 or more, a plurality of R¹ may bond together to form a ring with the carbon atoms to which they are attached. Examples of the ring include cyclopropane, cyclobutene, cyclopentane, cyclohexane, norbornane and adamantane rings. In the ring, some or all of the hydrogen atoms may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—), or haloalkyl moiety.

In formula (A), L^{A} is a single bond, ether bond, ester bond, amide bond, sulfonate ester bond, carbonate bond or carbamate bond, preferably a single bond, ether bond or ester bond.

In formula (A), X^{L} is a single bond or C₁-C₄₀ hydrocarbylene group which may contain a heteroatom. The hydrocarbylene group may be straight, branched or cyclic. Alkanediyl groups, cyclic saturated hydrocarbylene groups, and arylene groups are exemplary. Suitable heteroatoms include oxygen, nitrogen and sulfur.

Examples of the C₁-C₄₀ hydrocarbylene group which may contain a heteroatom, represented by X^{L}, are shown below, but not limited thereto. Herein,*designates a point of attachment to the adjacent L^{A} and —C(═O)—.

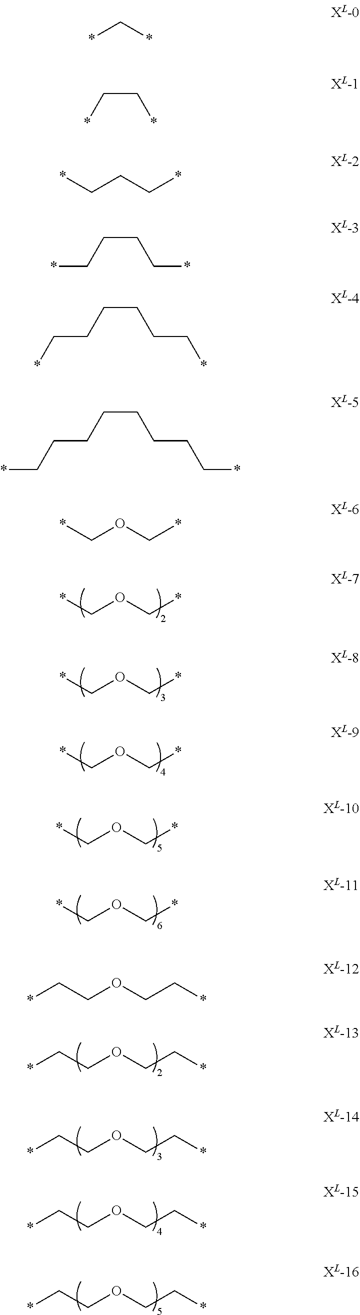

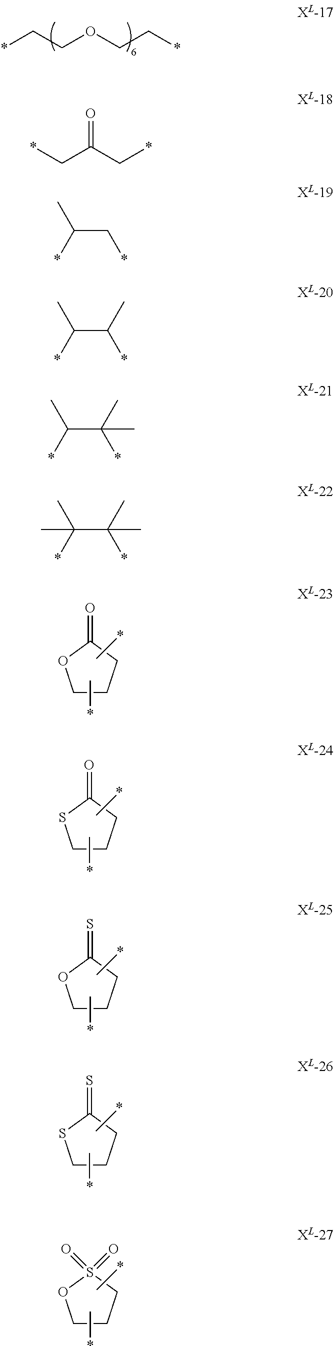

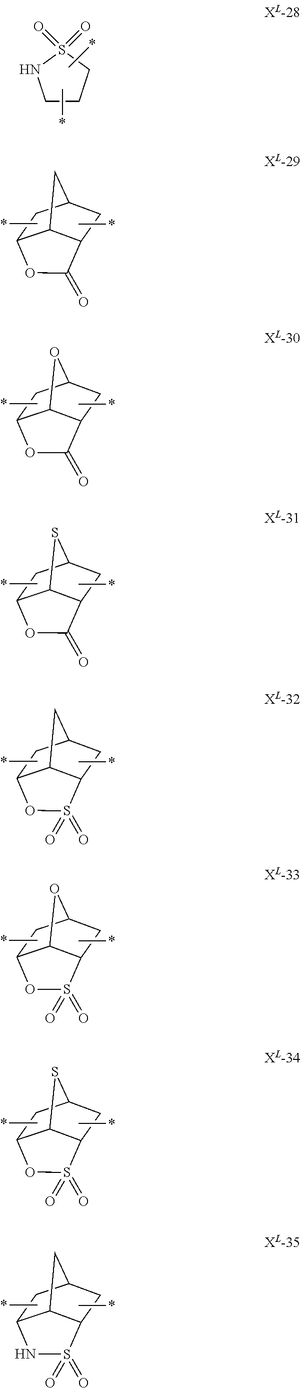

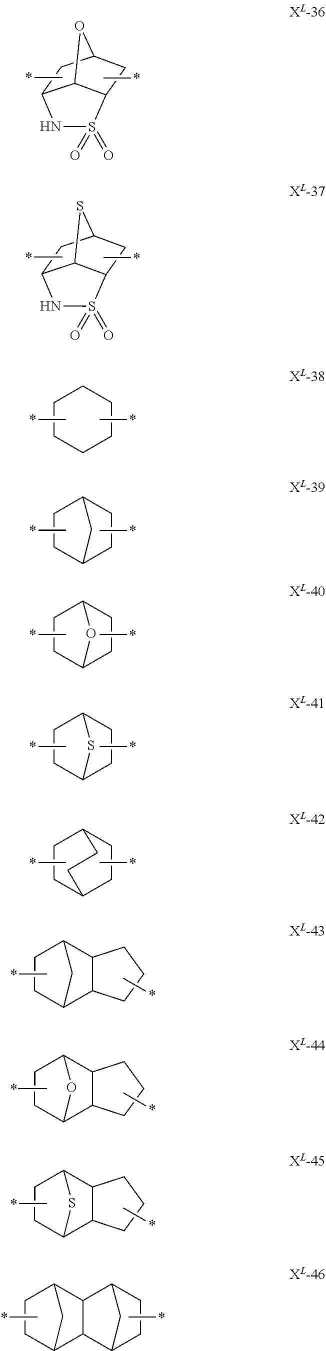

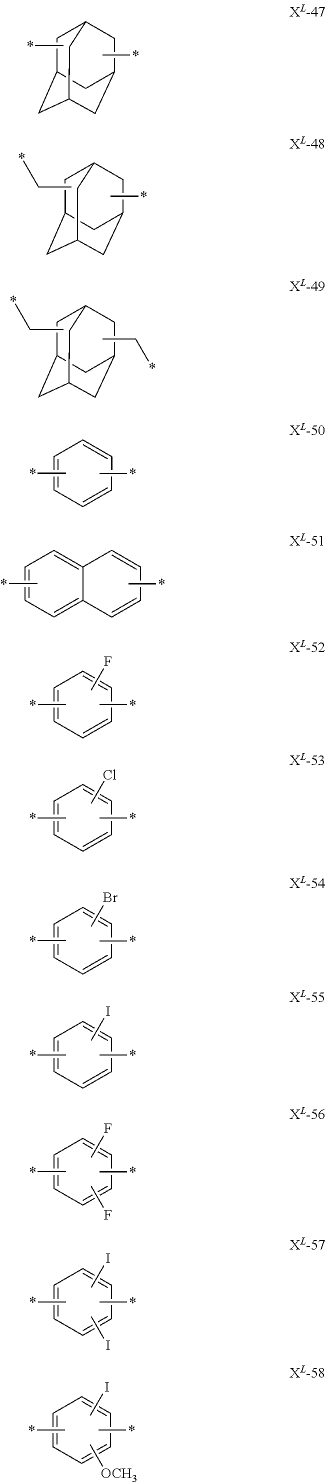

Of these, X^{L}-0 to X^{L}-22 and X^{L}-47 to X^{L}-58 are preferred.

In formula (A), R^{L1}, R^{L2} and R^{L3} are each independently a C₁-C₃₀, preferably C₁-C₁₀ hydrocarbyl group which may contain a heteroatom. Any two of R^{L1}, R^{L2} and R^{L3} may bond together to form a ring with the carbon atom to which they are attached. When they do not form a ring, at least one of R^{L1}, R^{L2} and R^{L3} has at least one structure selected from a multiple bond, alicyclic and aromatic ring. Specifically, at least one of R^{L1}, R^{L2} and R^{L3} is preferably a C₂-C₃₀ hydrocarbyl group containing a multiple bond, a C₃-C₃₀ hydrocarbyl group containing an alicyclic structure, or a C₆-C₃₀ hydrocarbyl group containing an aromatic ring structure. Some —CH₂— in the hydrocarbyl group may be replaced by —O— or —S—.

In formula (A), examples of the acid labile group of the formula: —C(R^{L1})(R^{L2})(R^{L3}) are shown below, but not limited thereto. Herein,*designates a point of attachment to the adjacent oxygen.

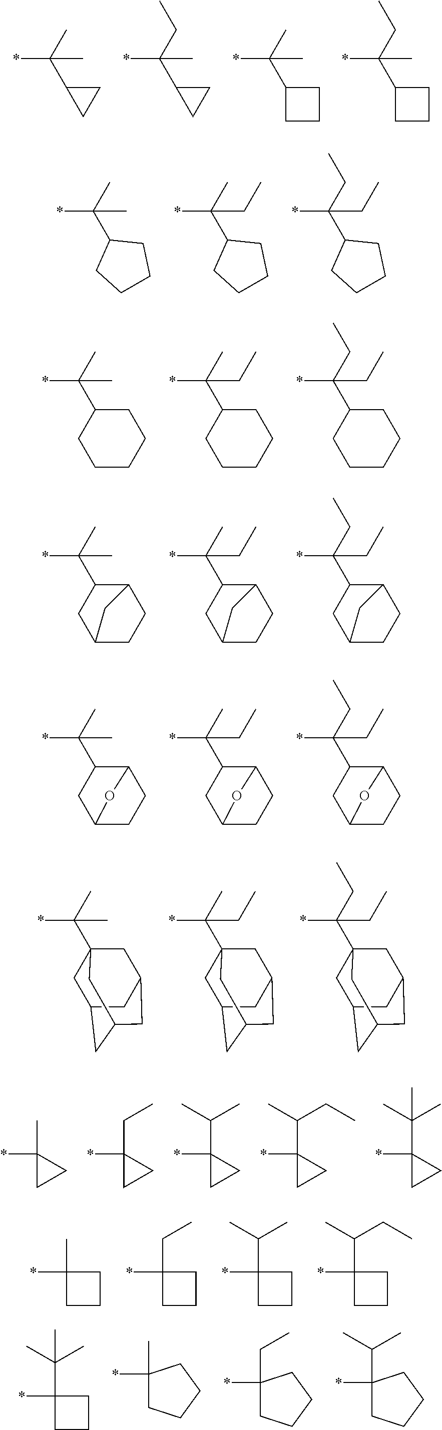

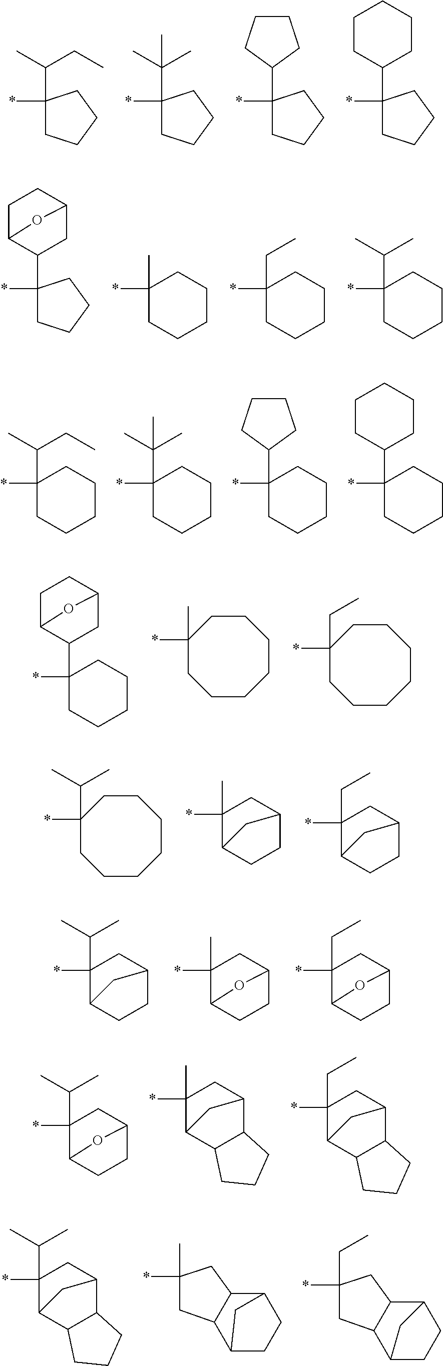

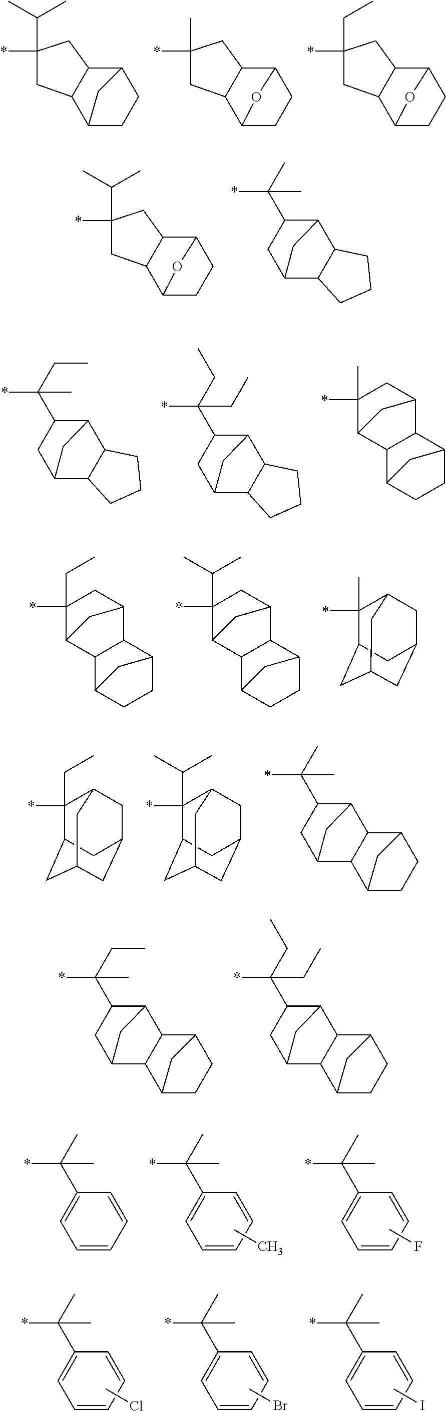

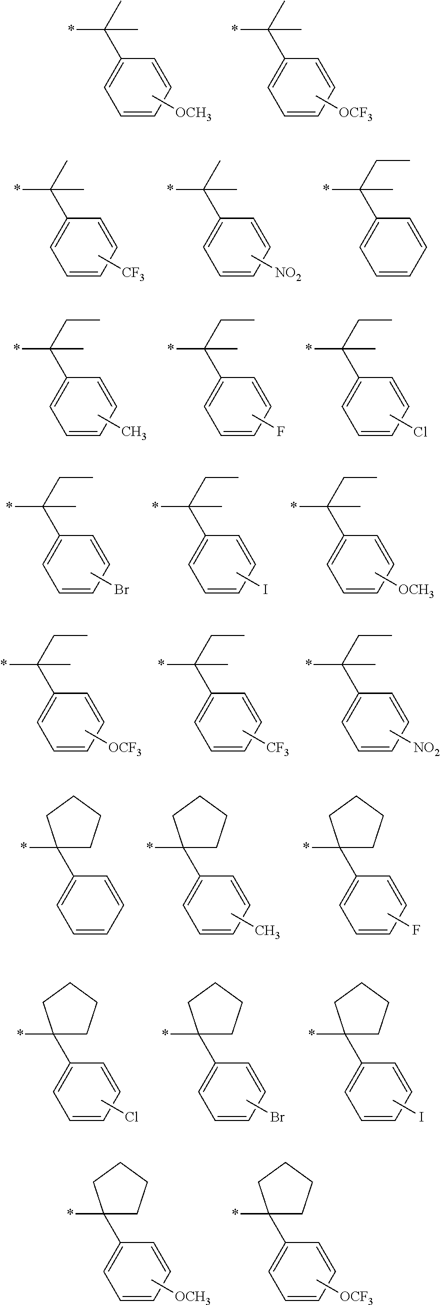

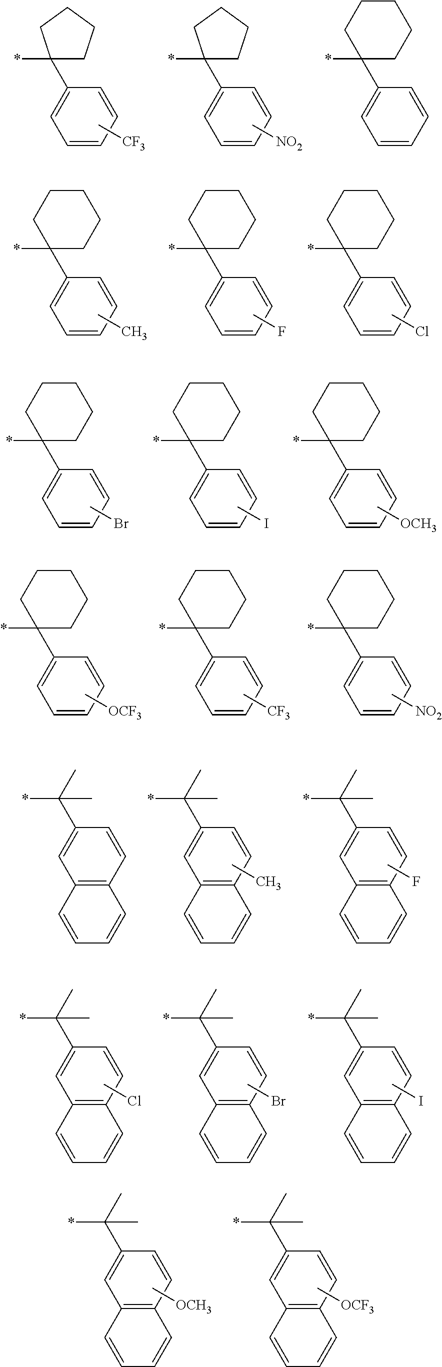

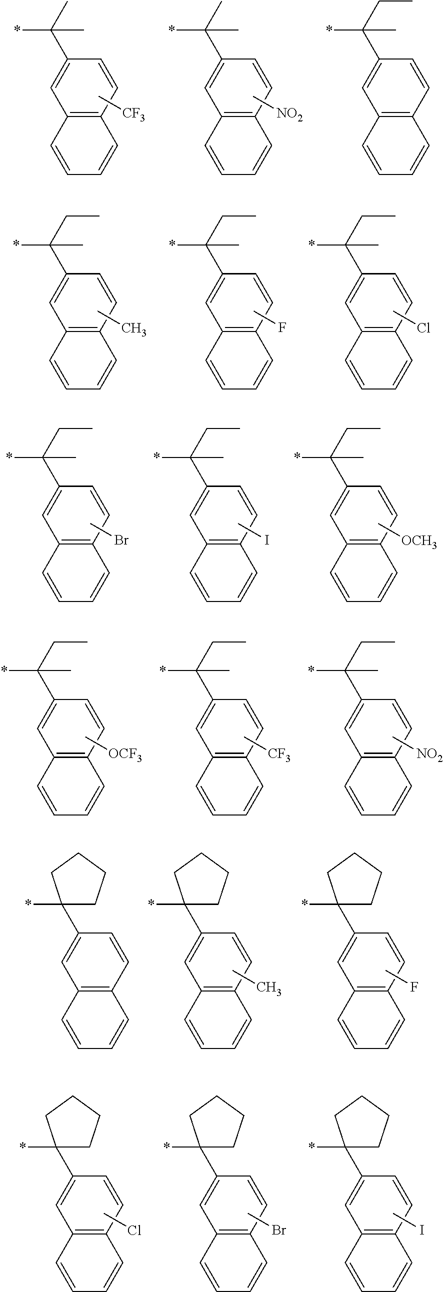

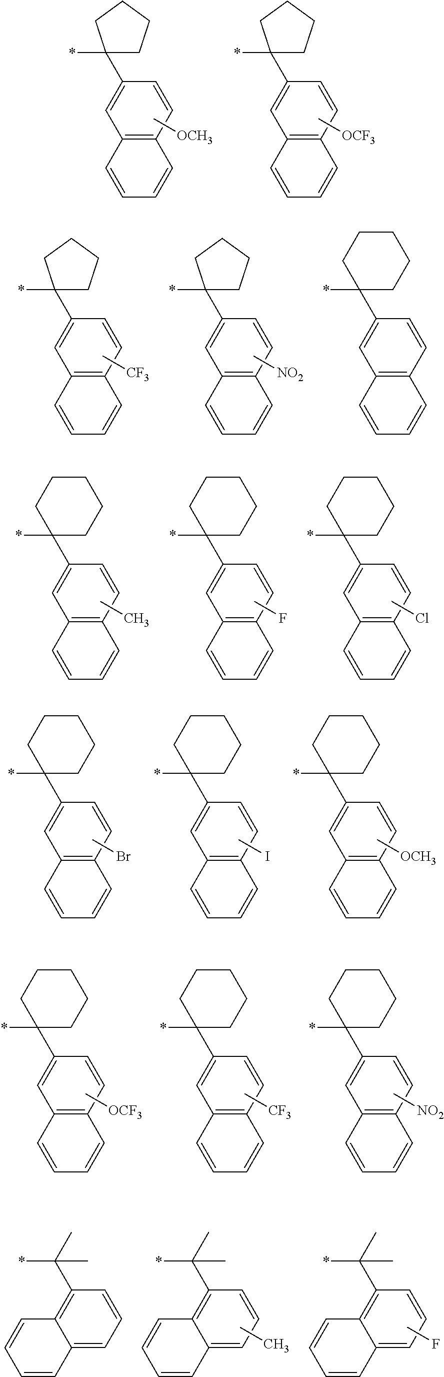

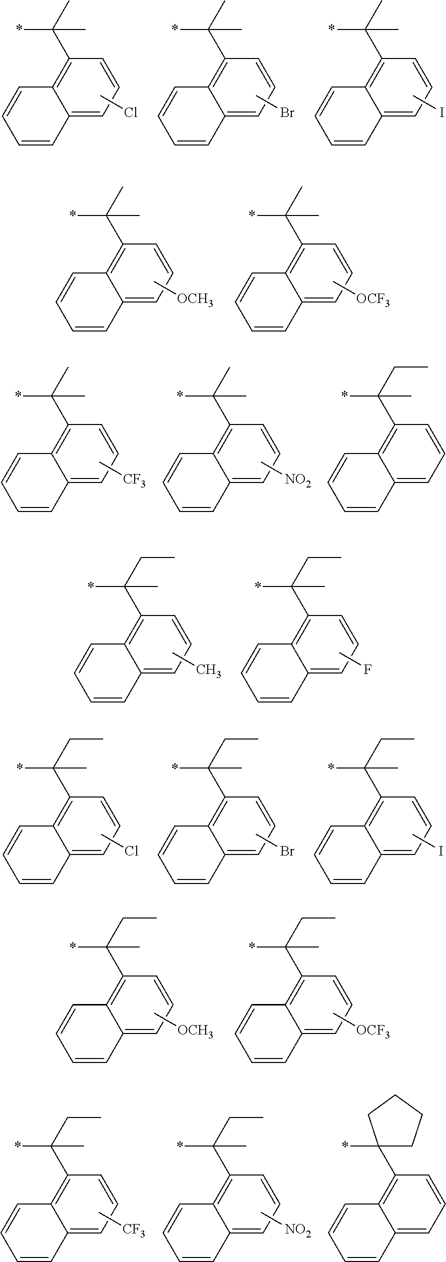

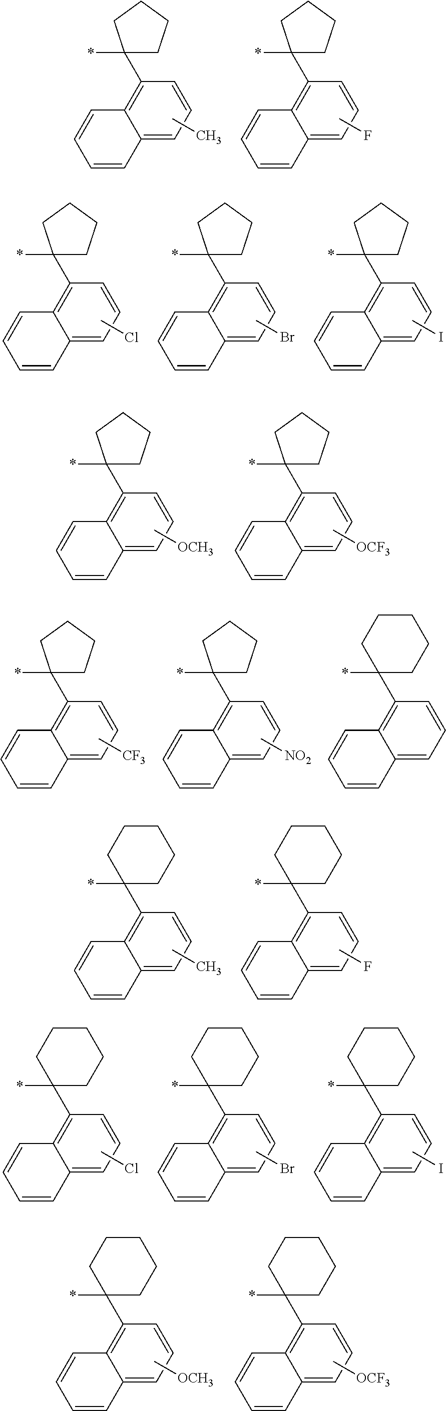

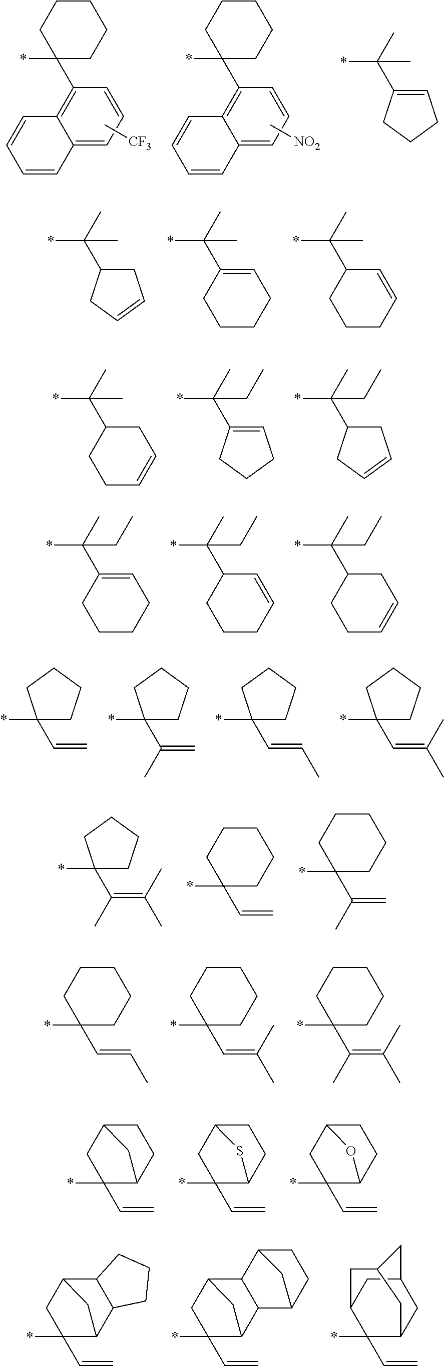

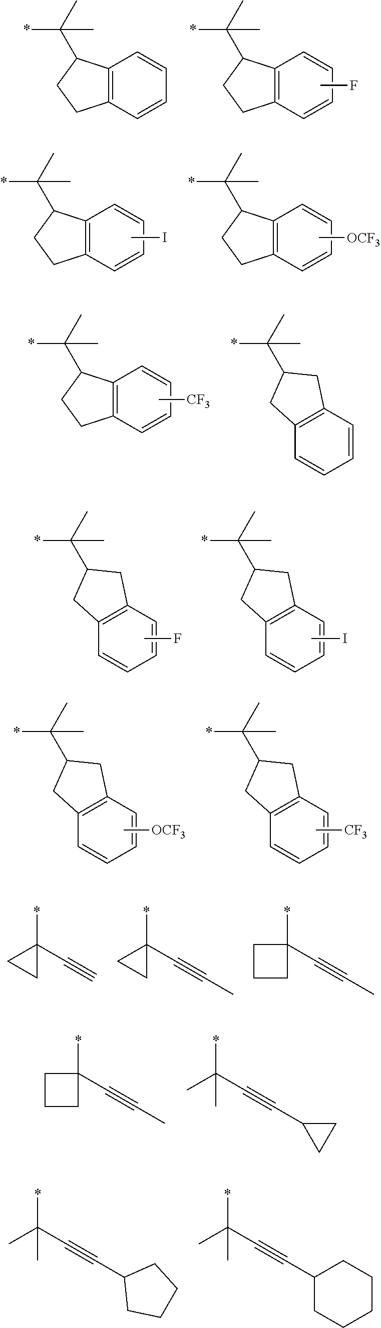

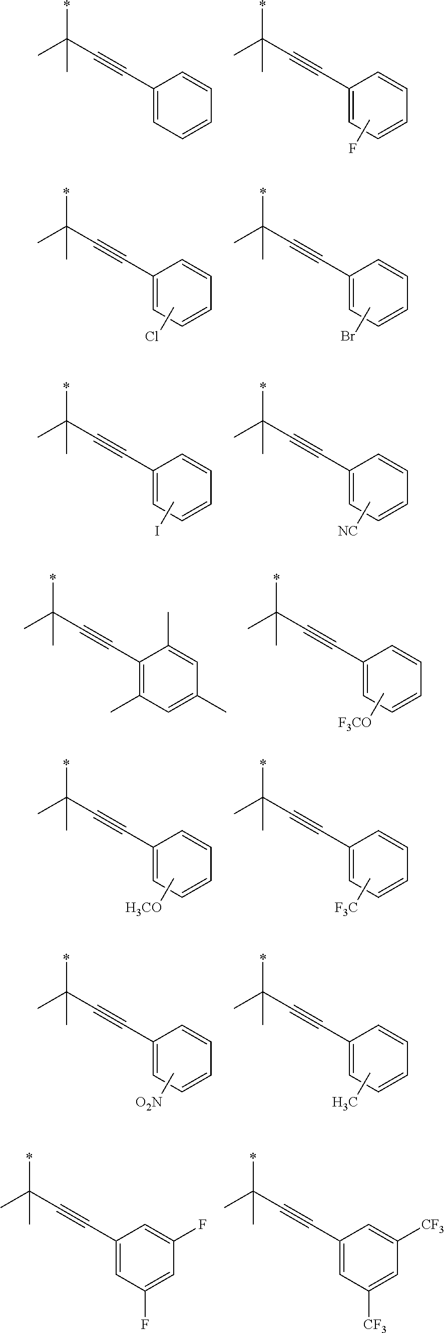

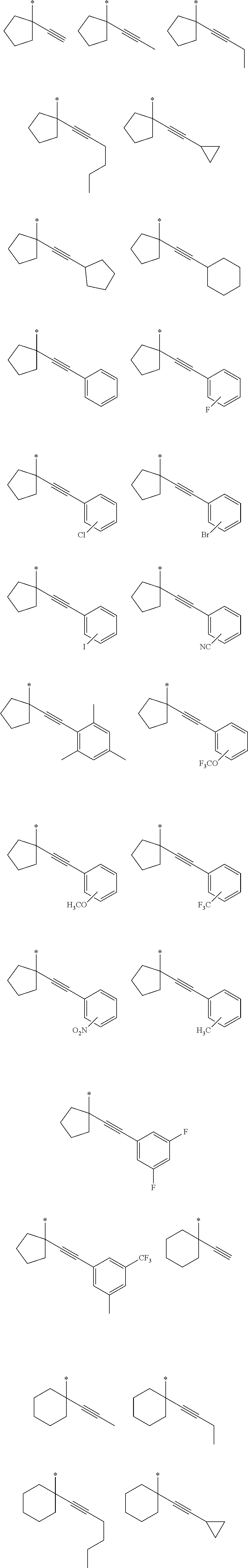

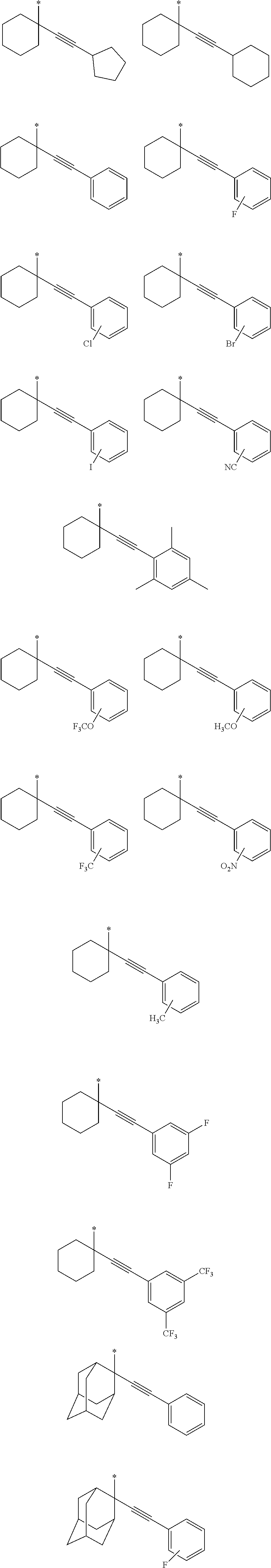

The preferred monomer of formula (A) has the formula (A1).

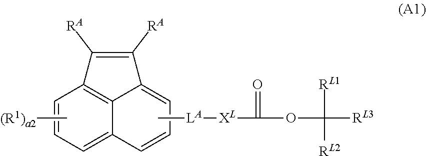

Herein a2, R^{A}, R¹, L^{A}, X^{L}, and R^{L1} to R^{L3} are as defined above.

The monomer of formula (A1) preferably has the formula (A2).

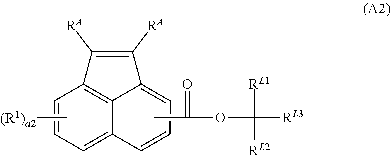

Herein a2, R^{A}, R¹, and R^{L1} to R^{L3} are as defined above.

Examples of the monomer having formula (A) are shown below, but not limited thereto.

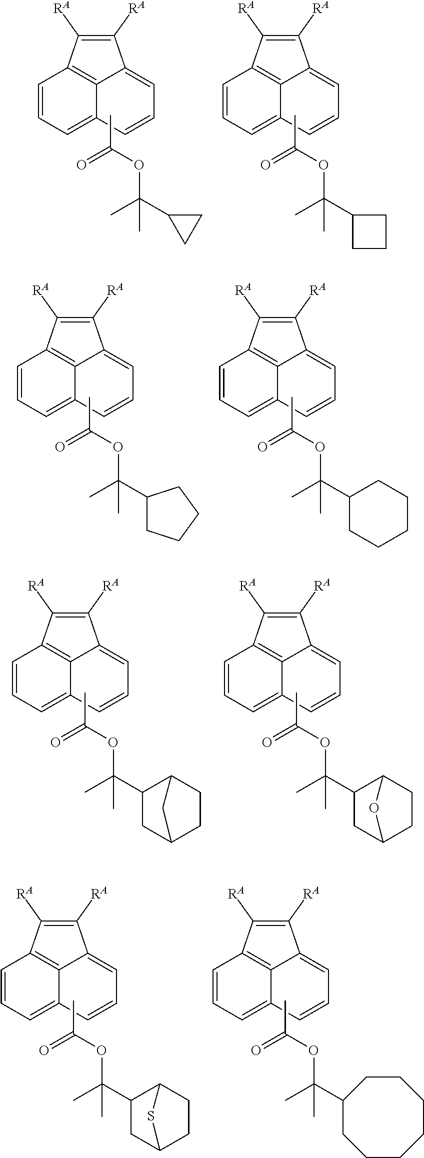

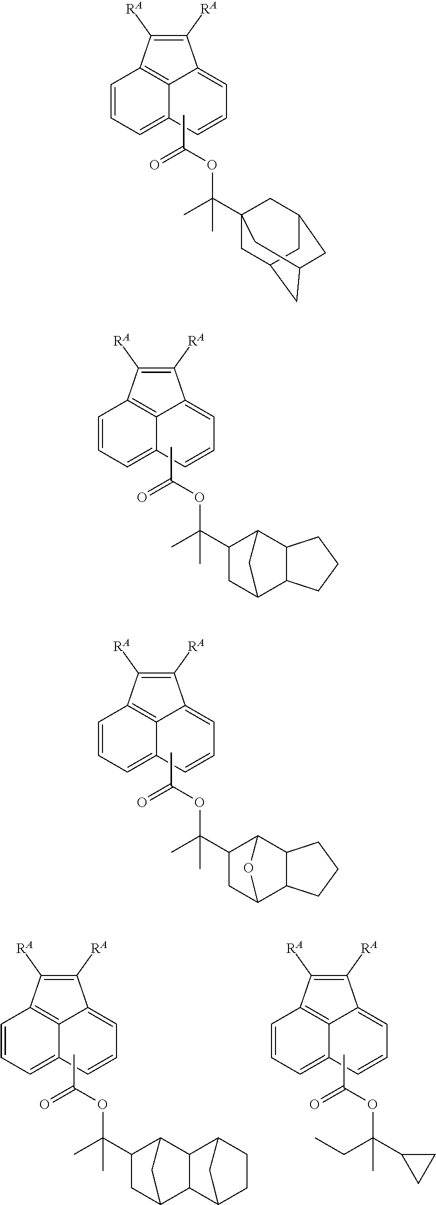

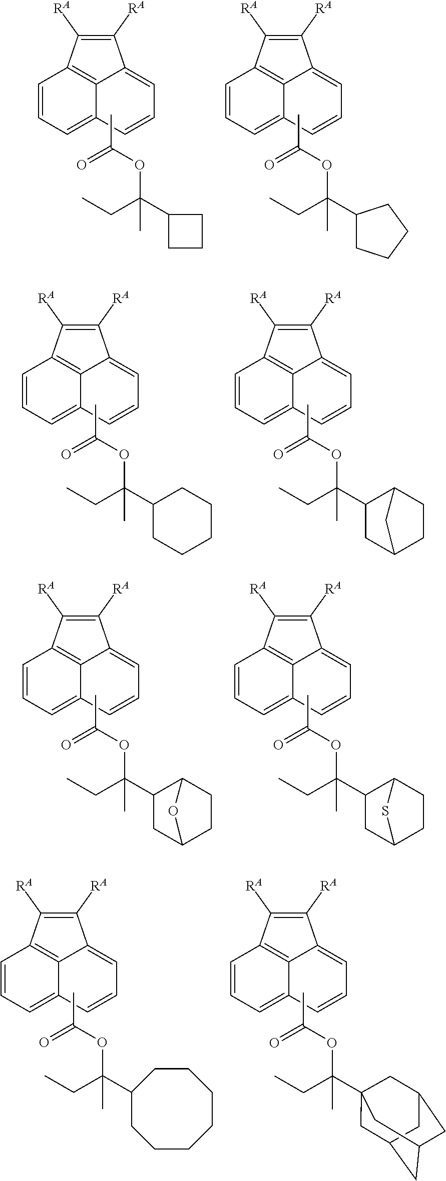

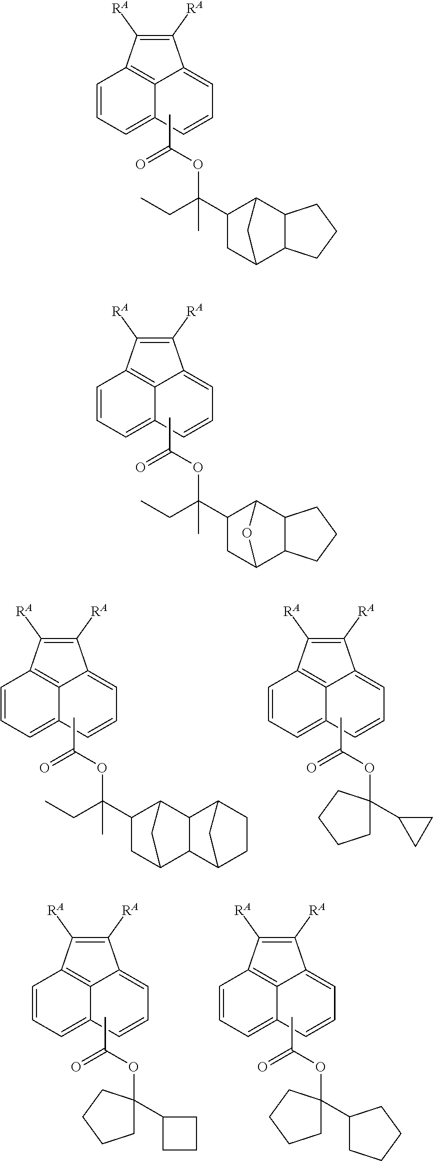

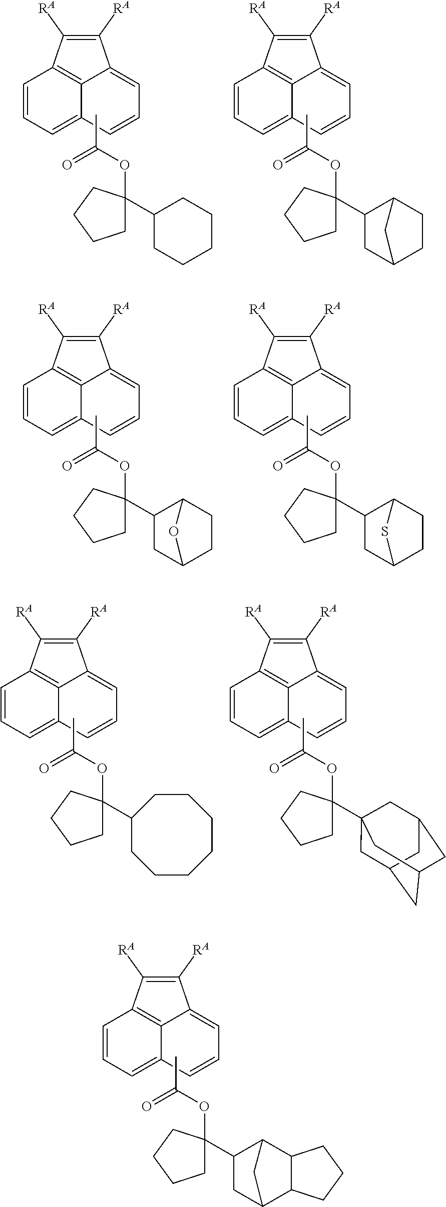

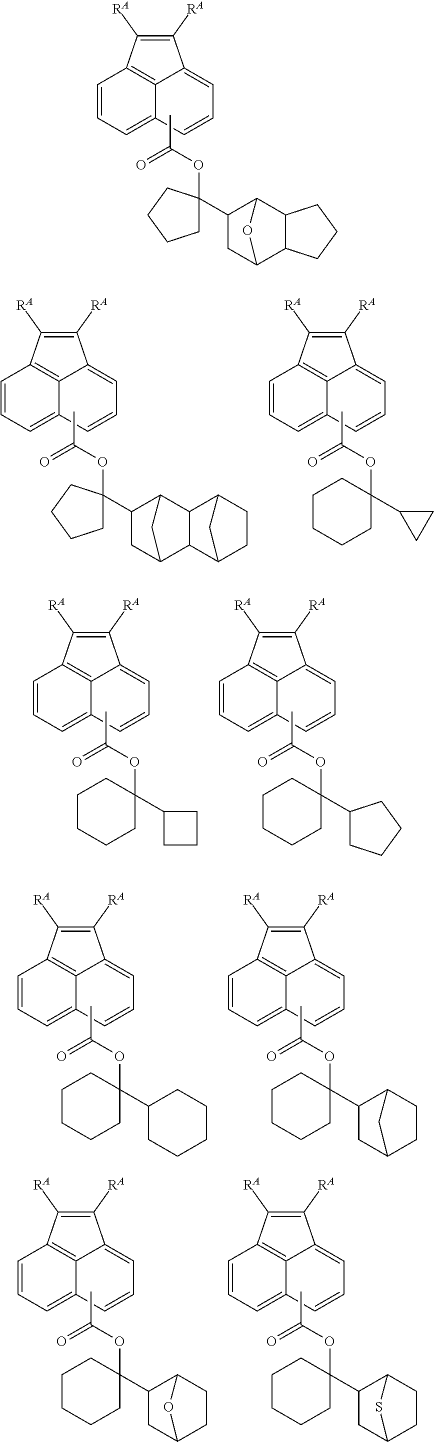

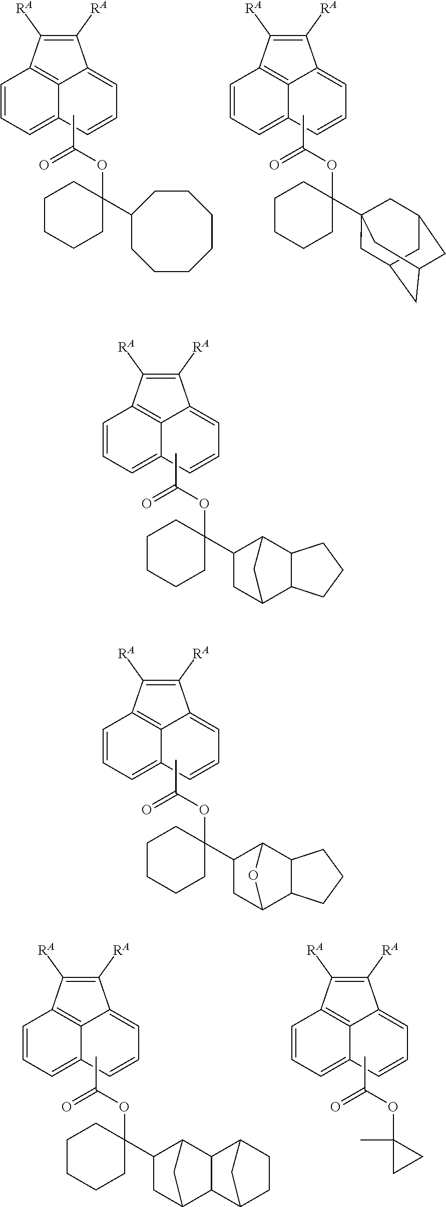

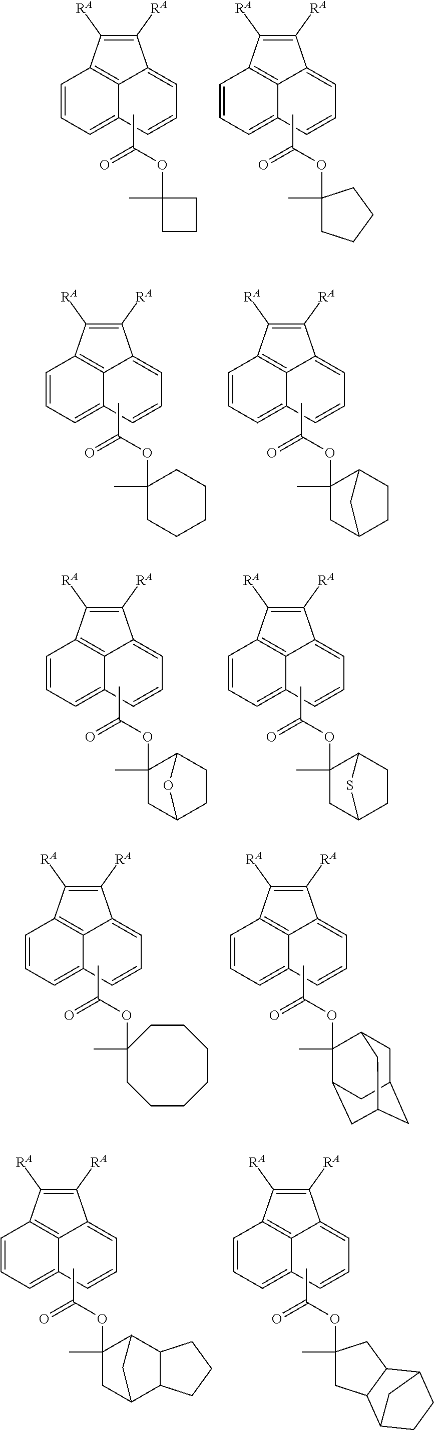

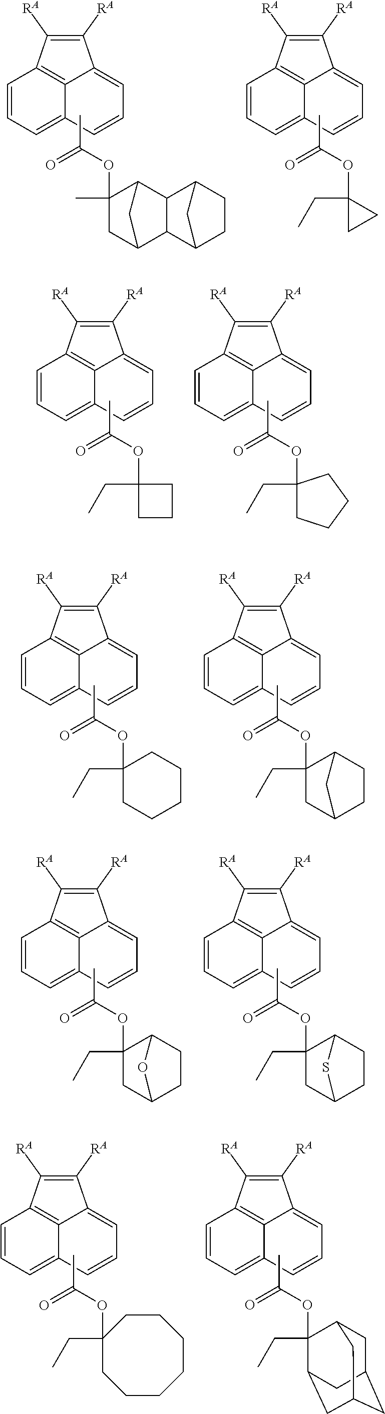

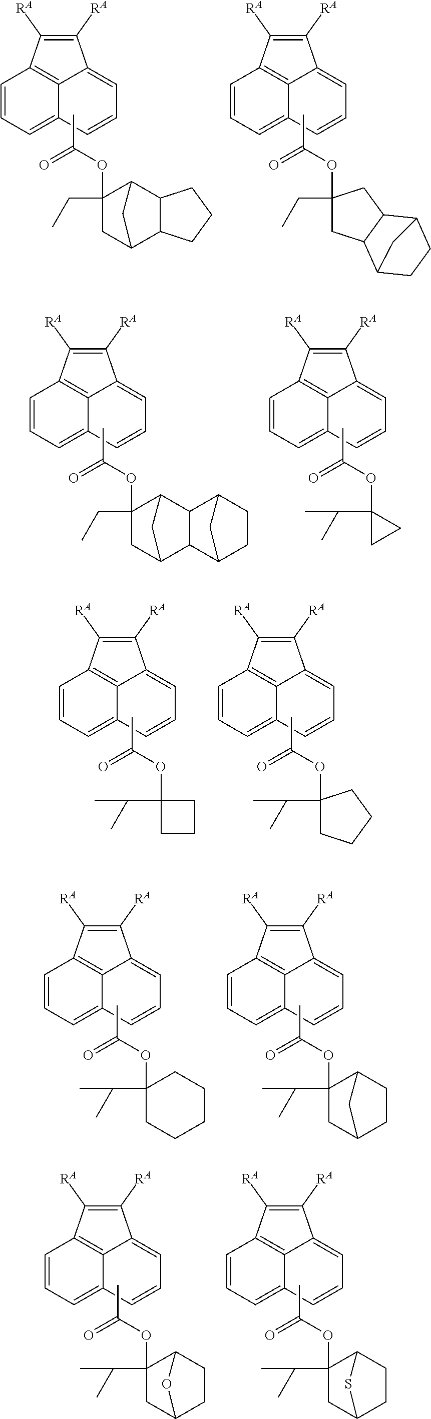

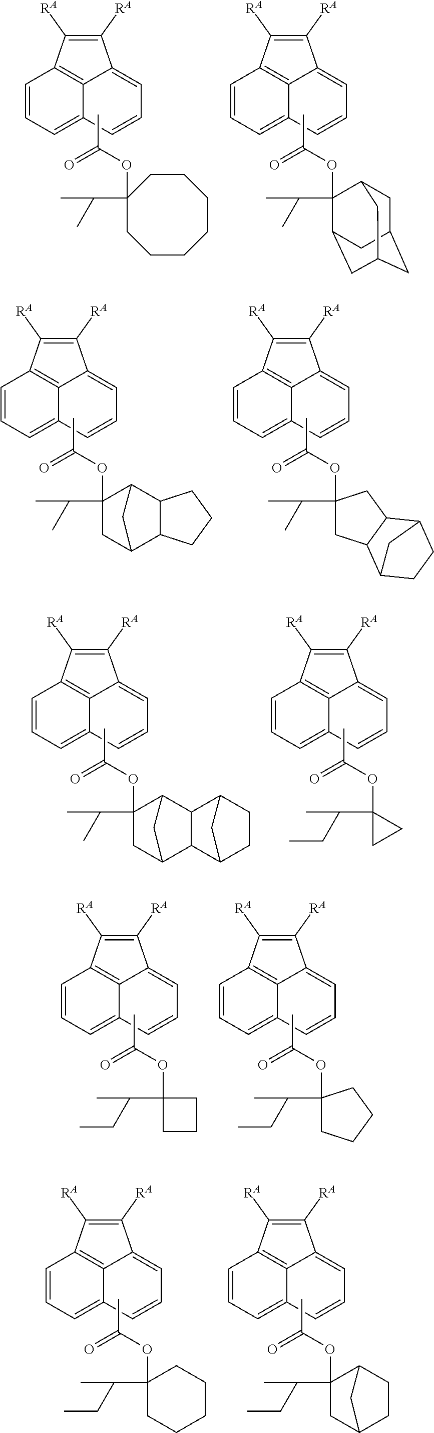

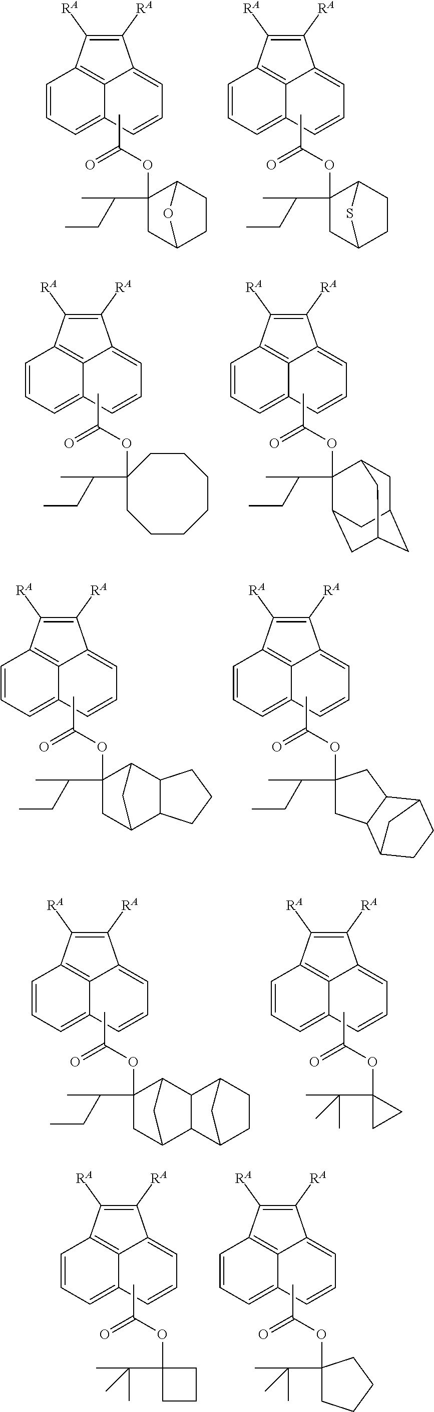

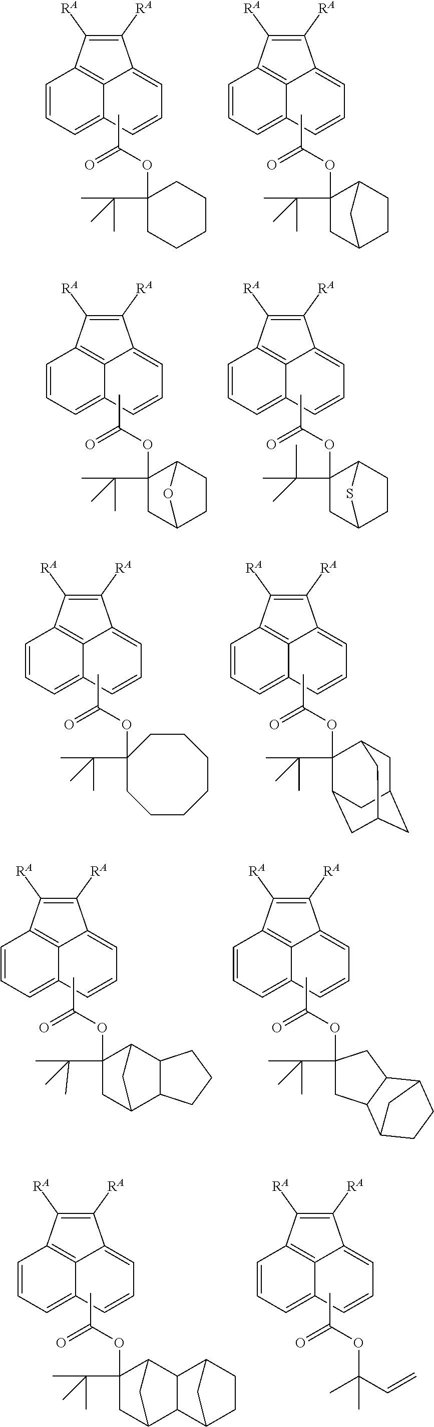

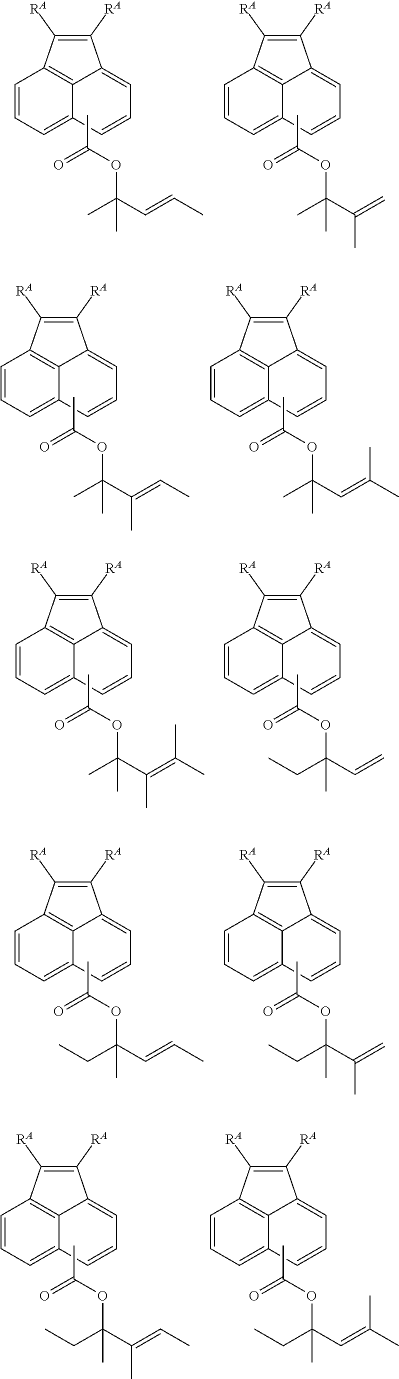

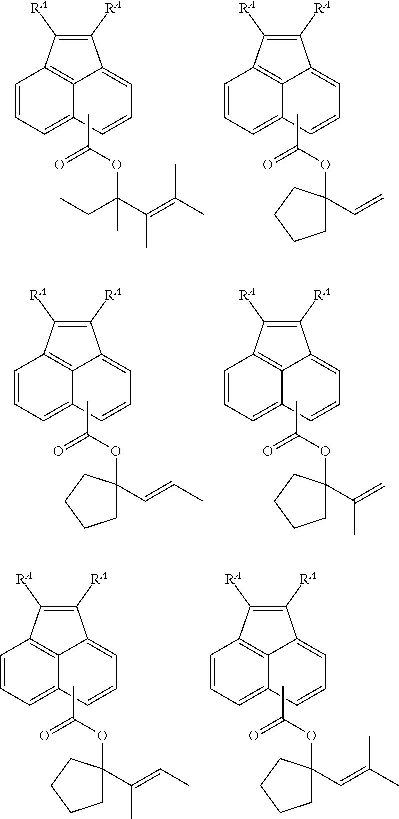

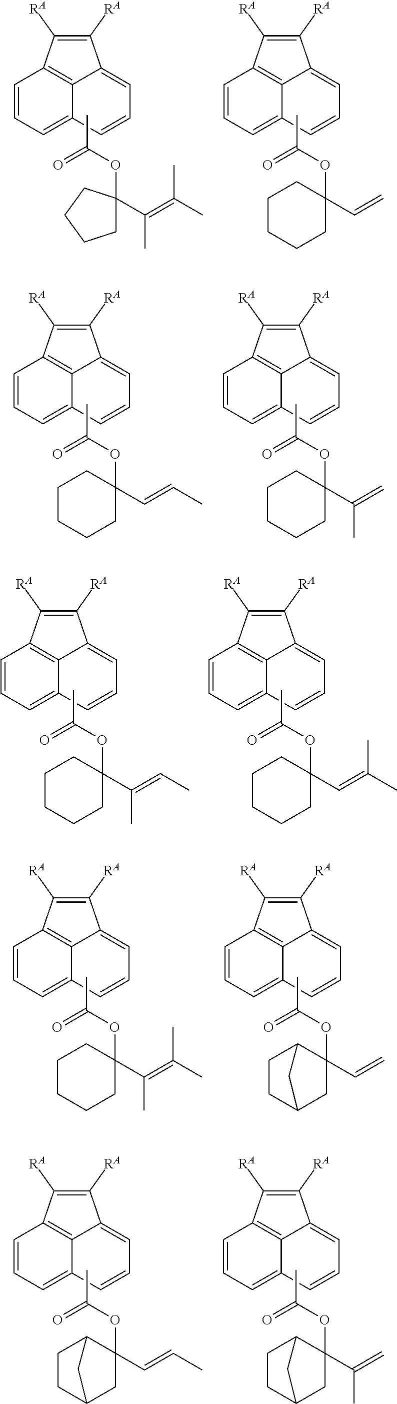

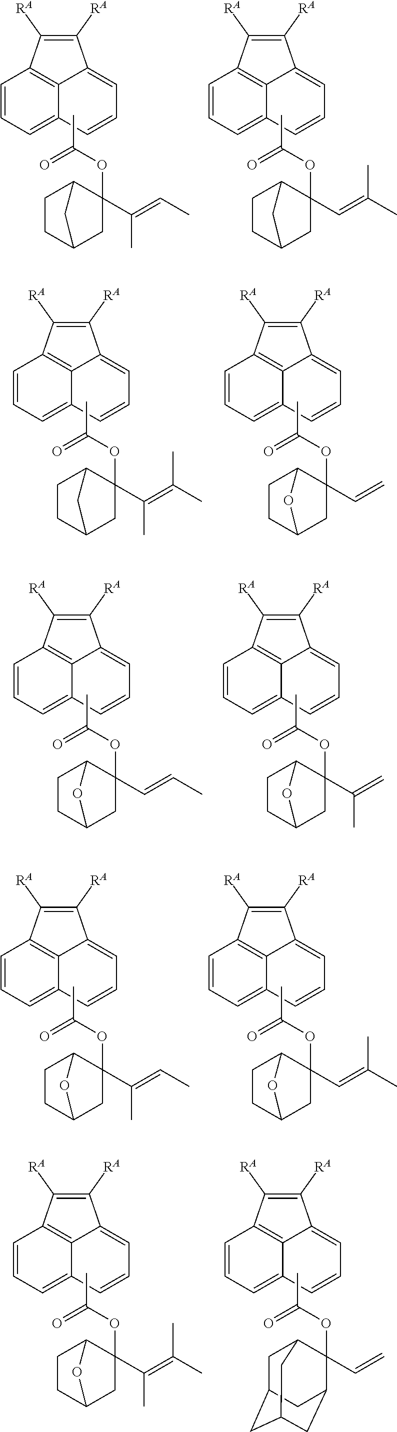

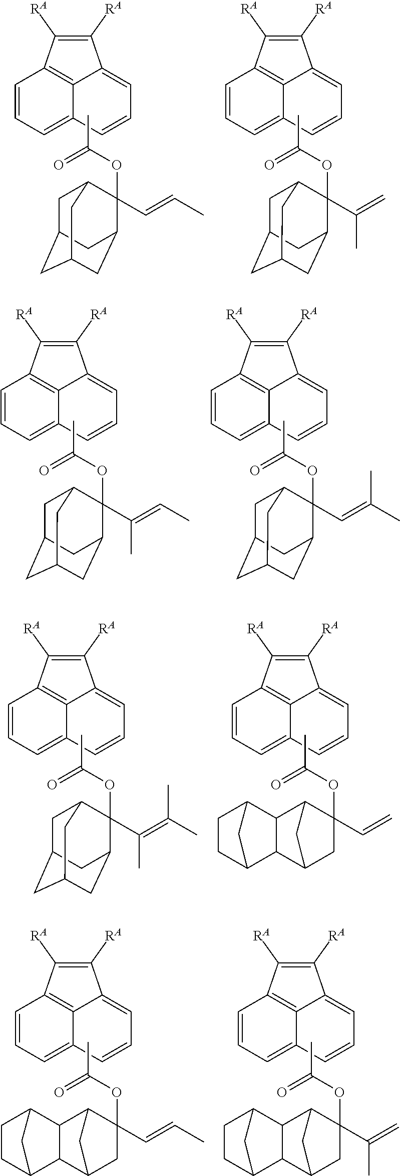

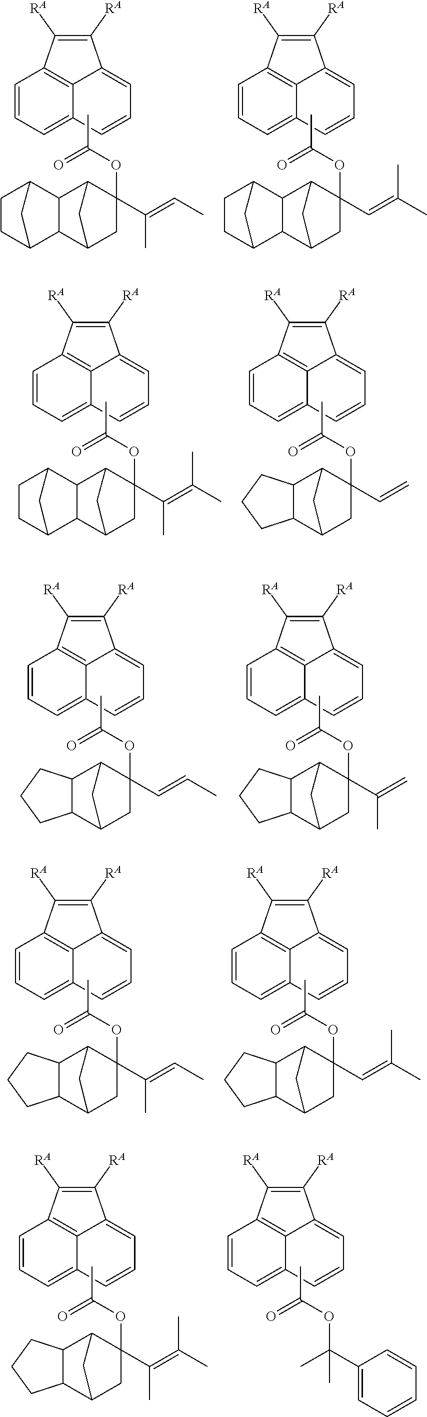

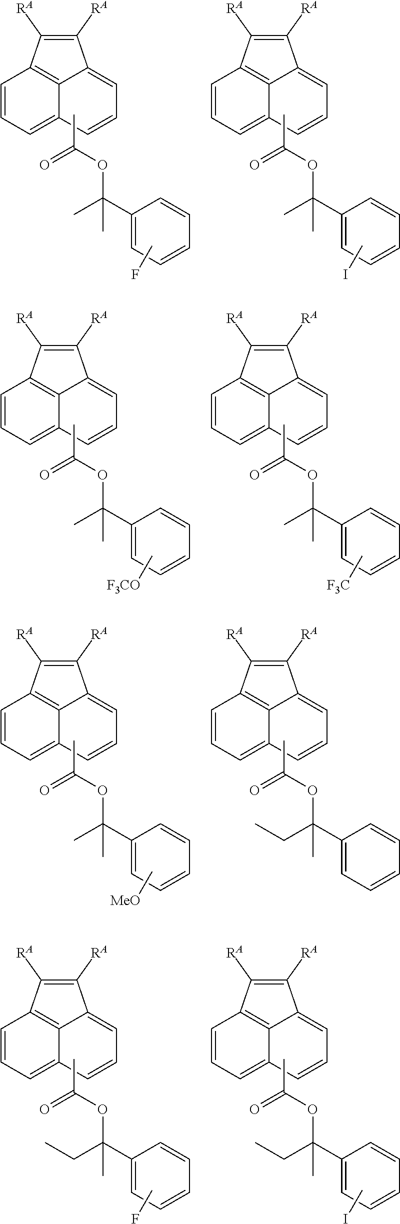

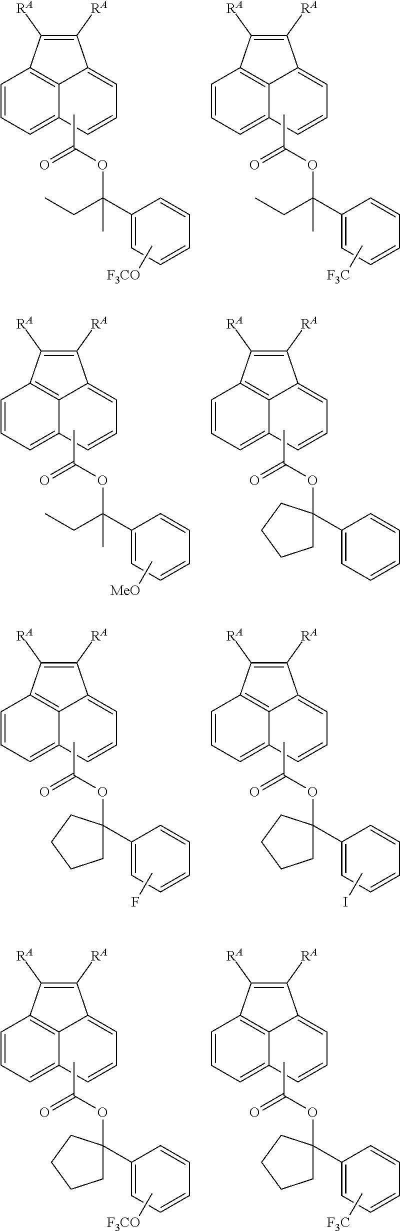

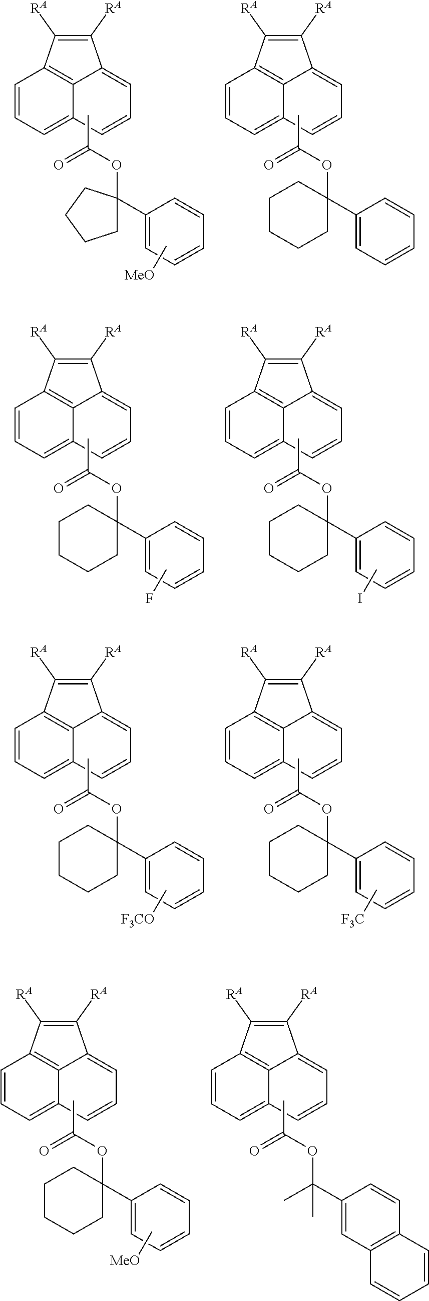

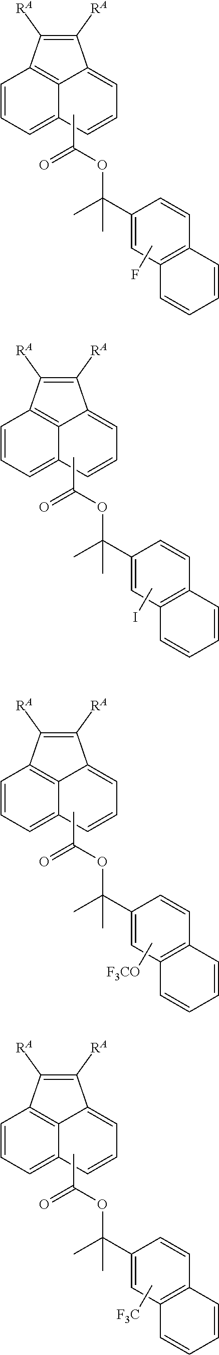

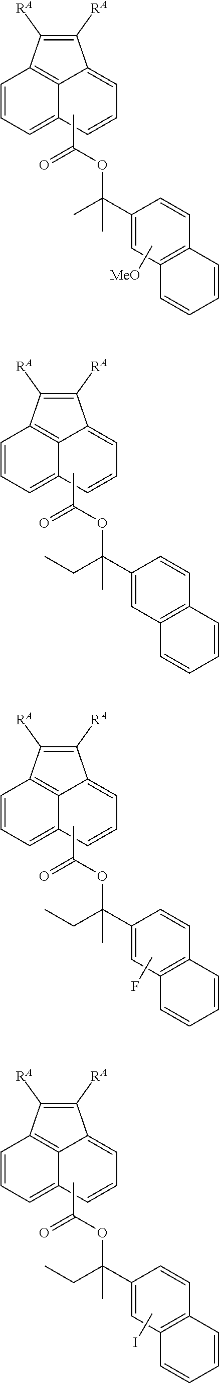

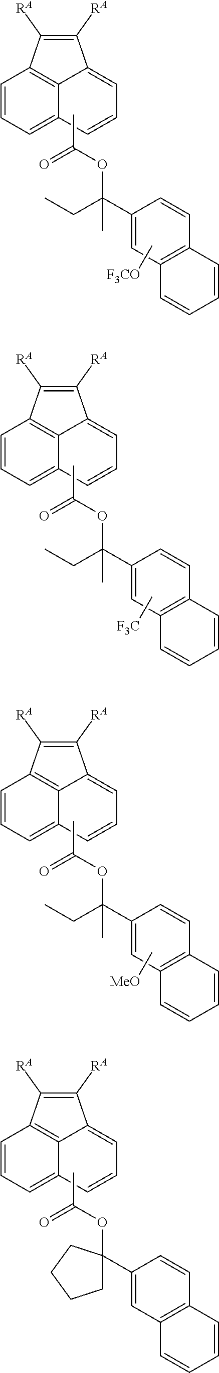

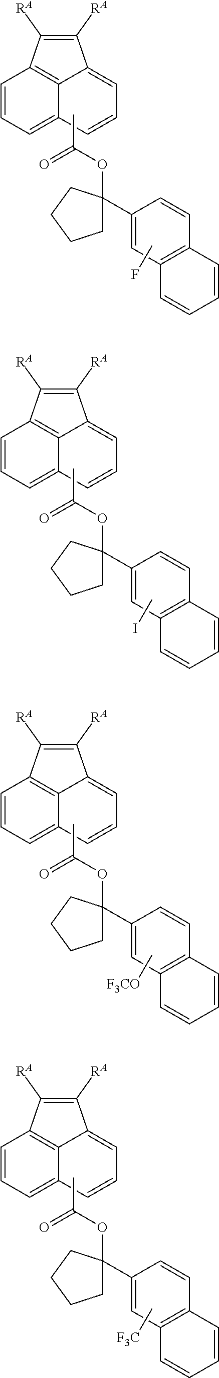

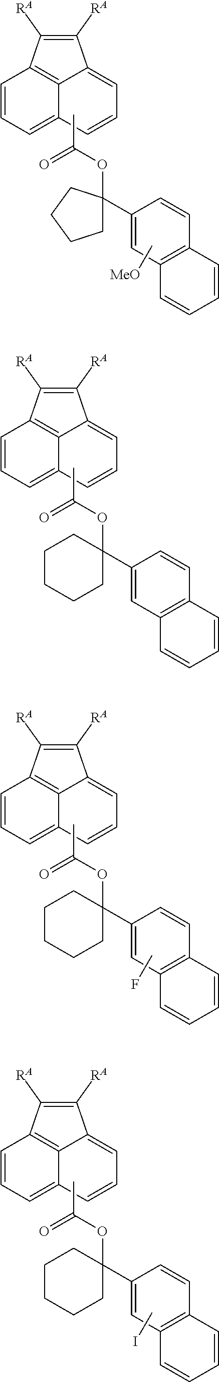

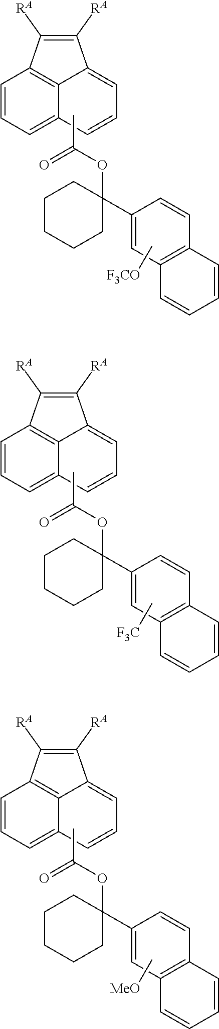

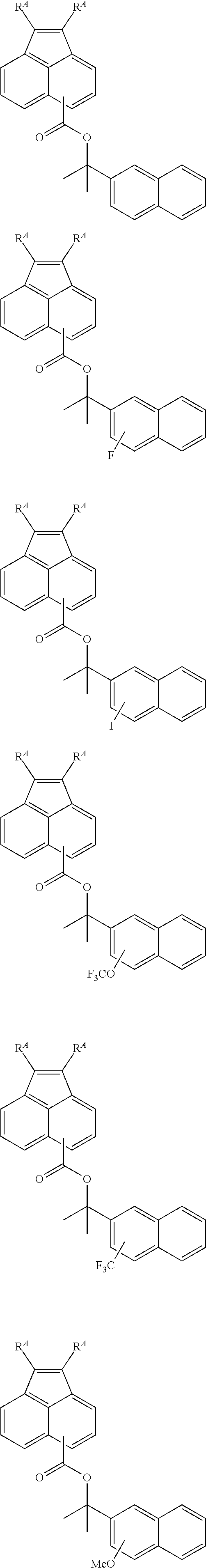

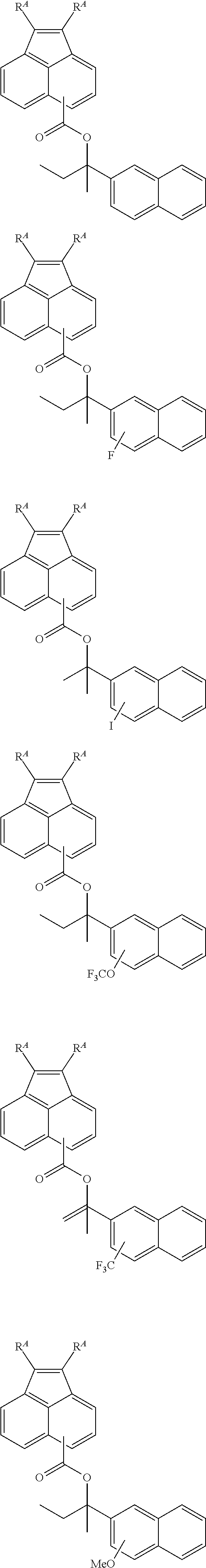

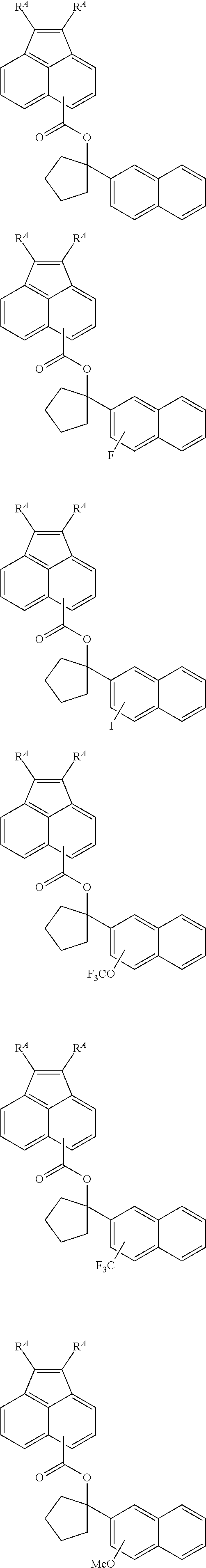

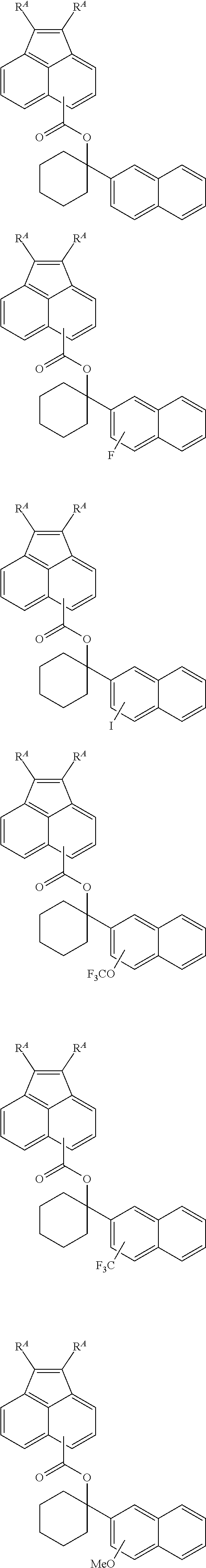

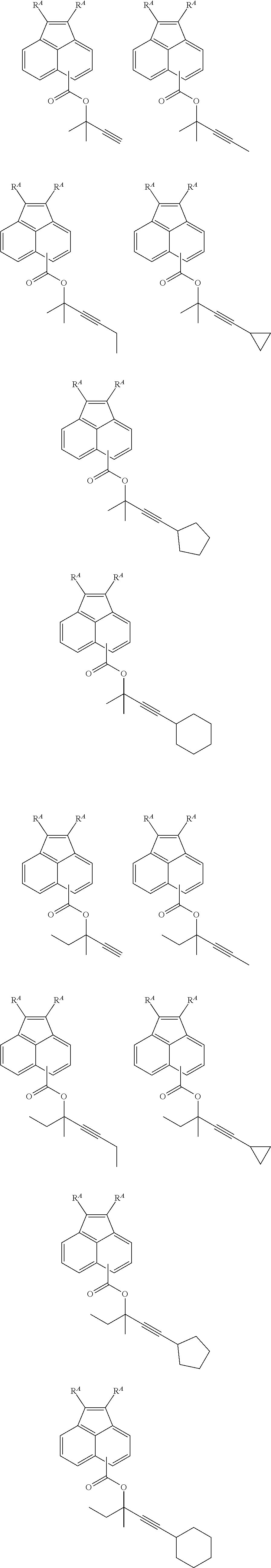

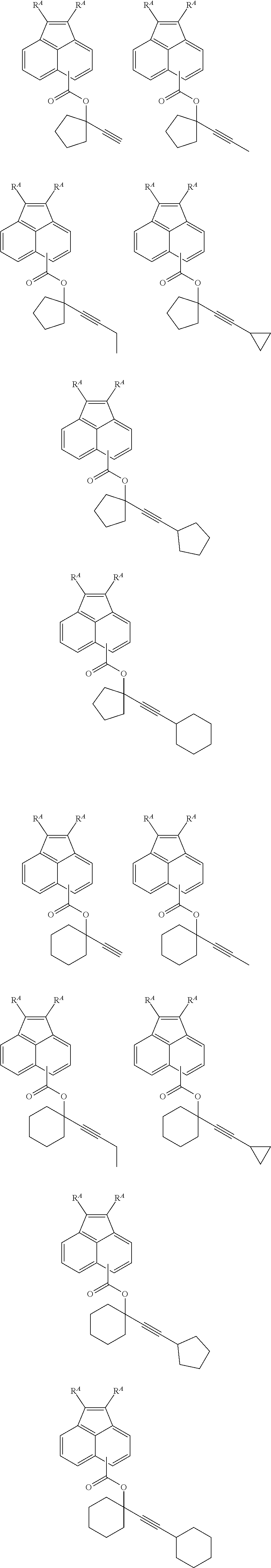

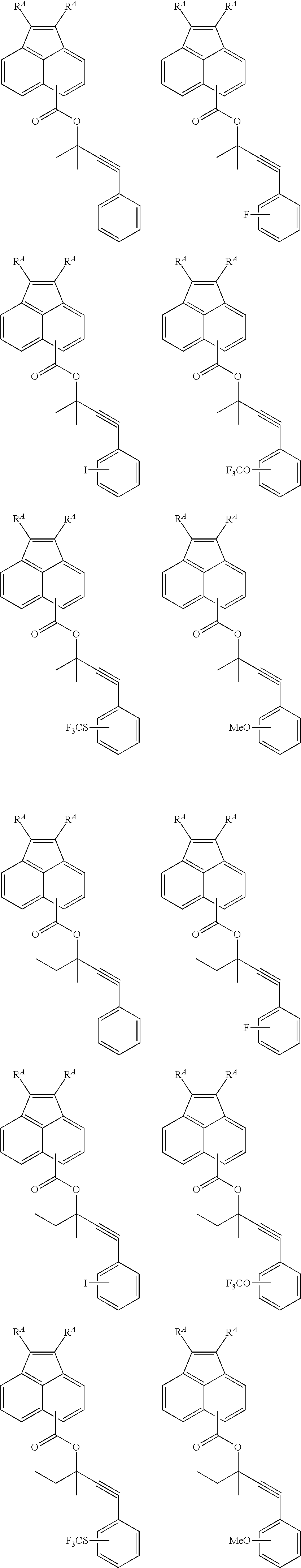

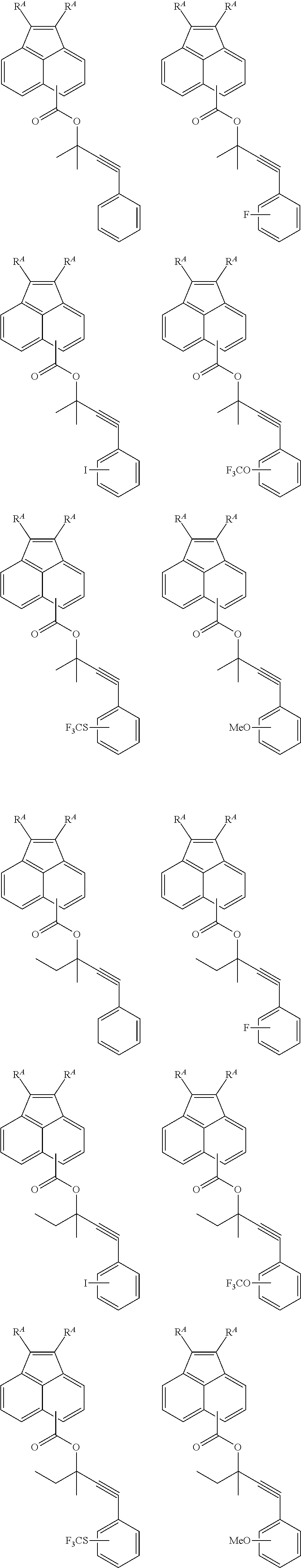

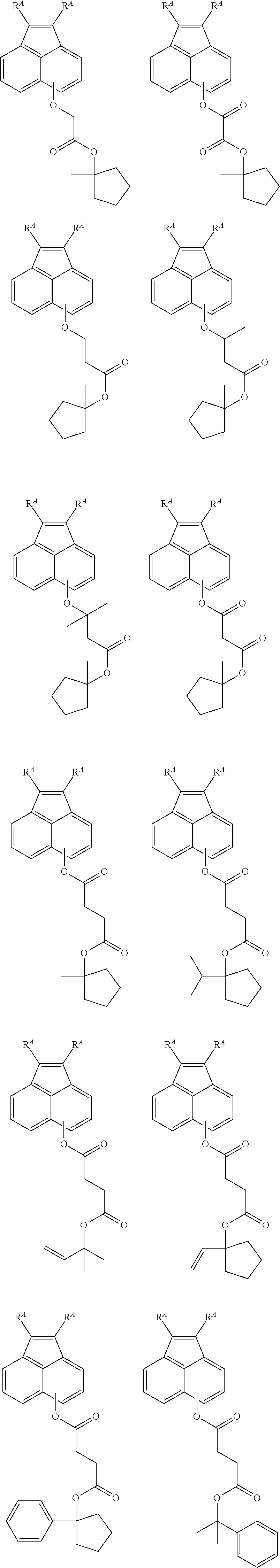

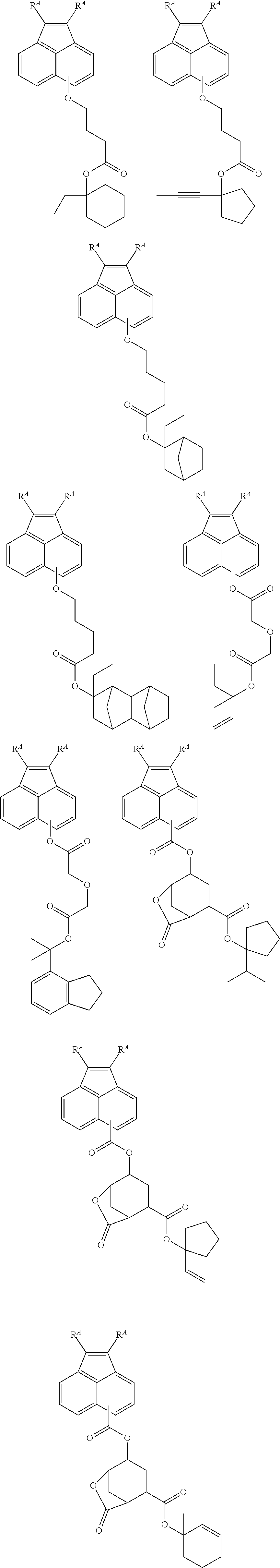

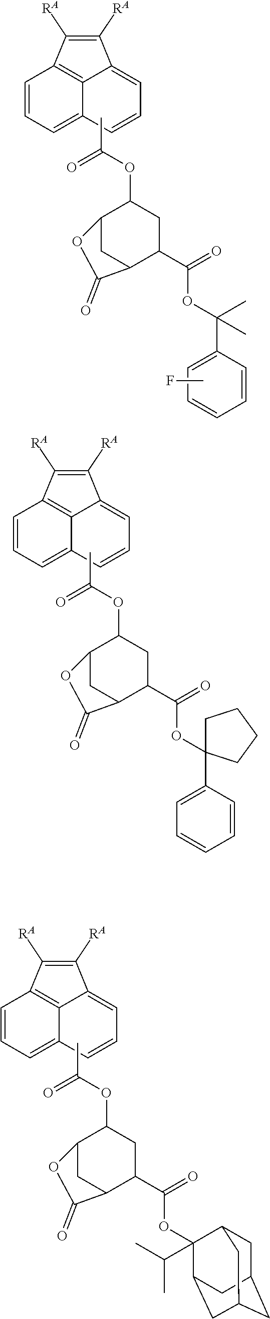

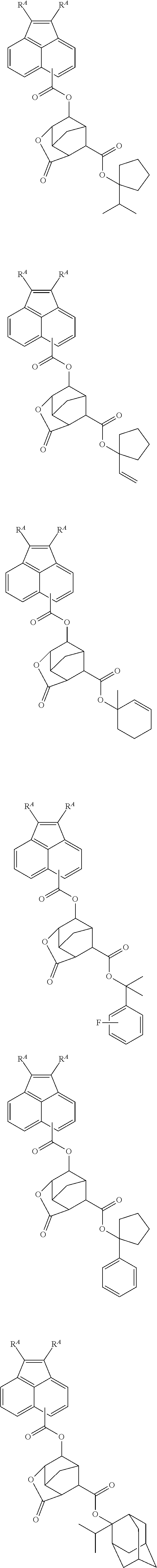

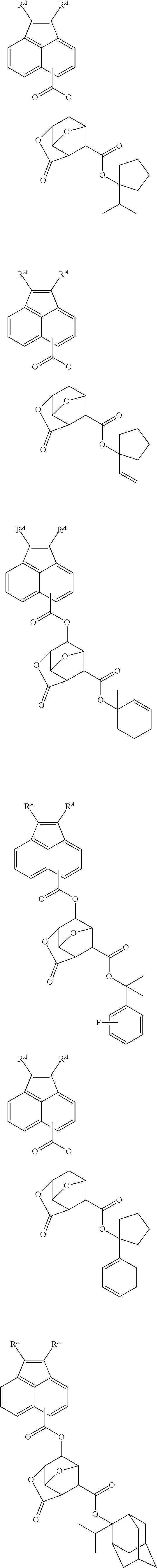

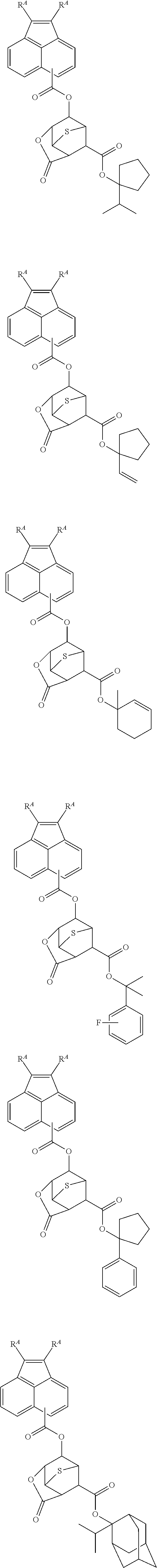

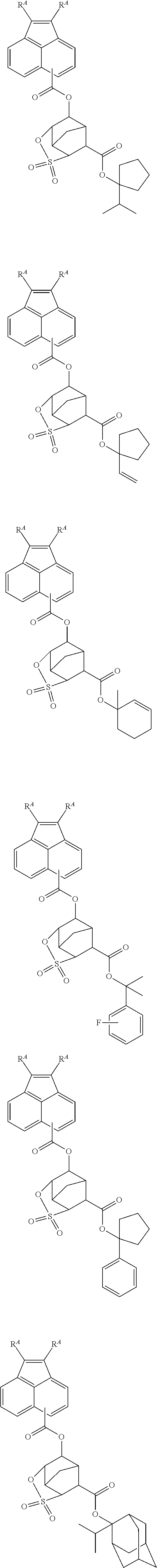

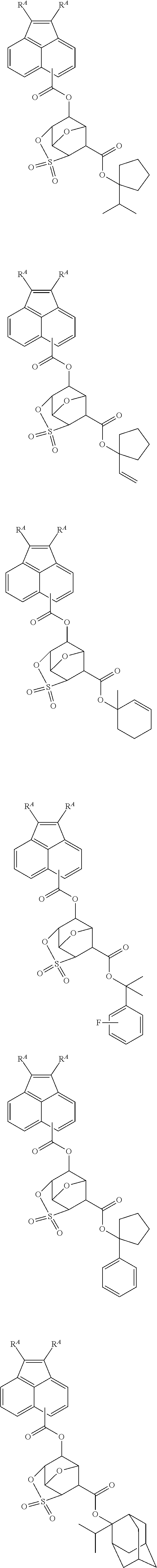

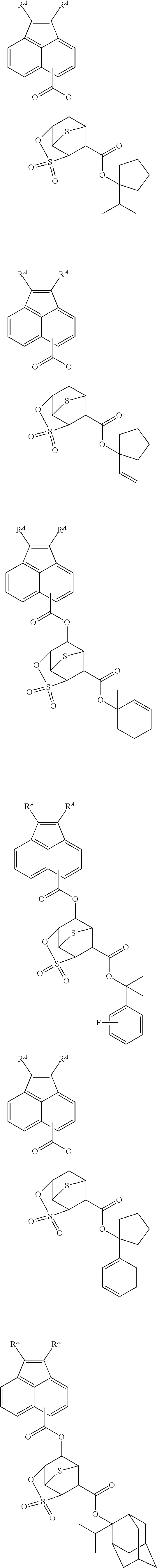

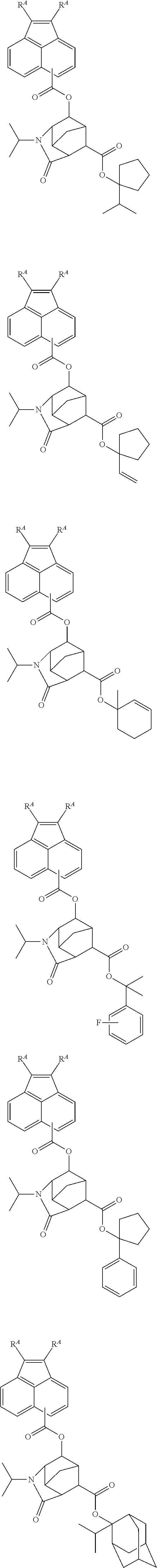

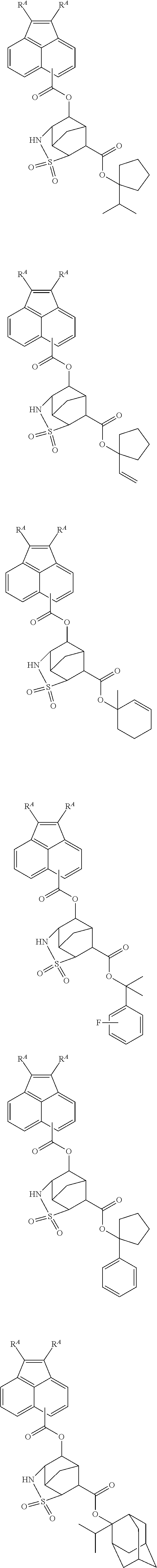

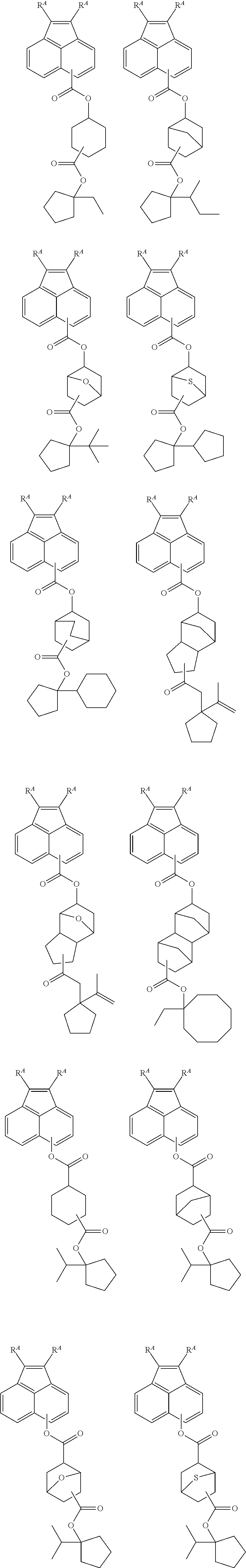

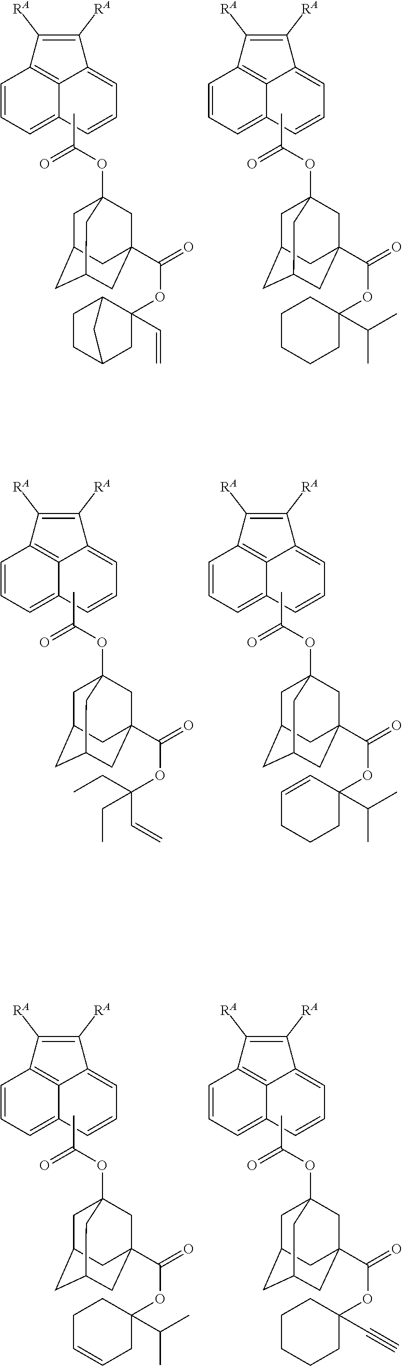

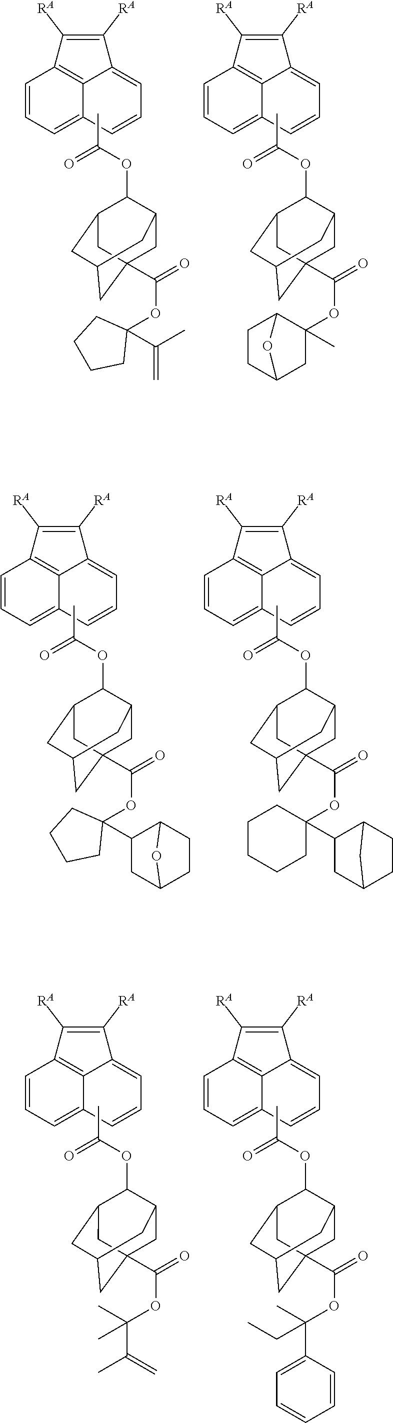

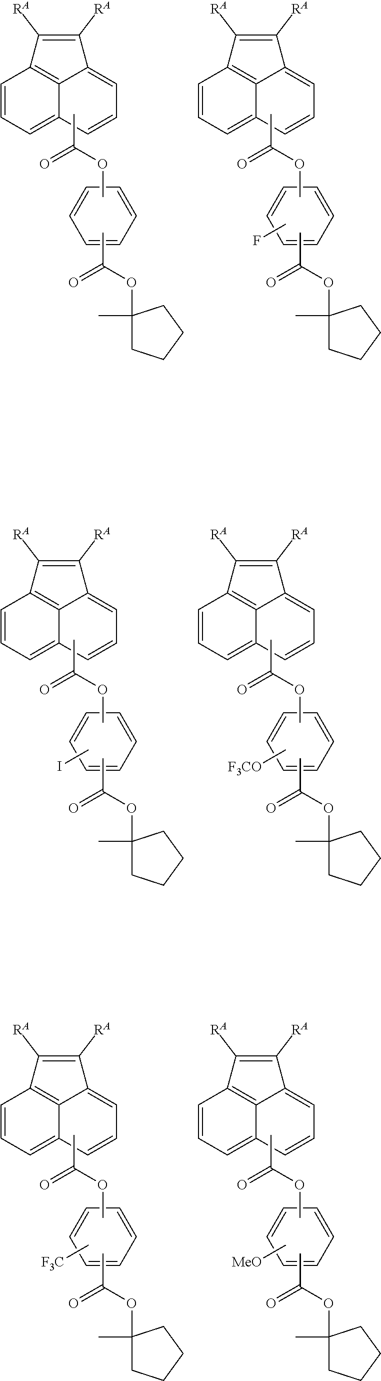

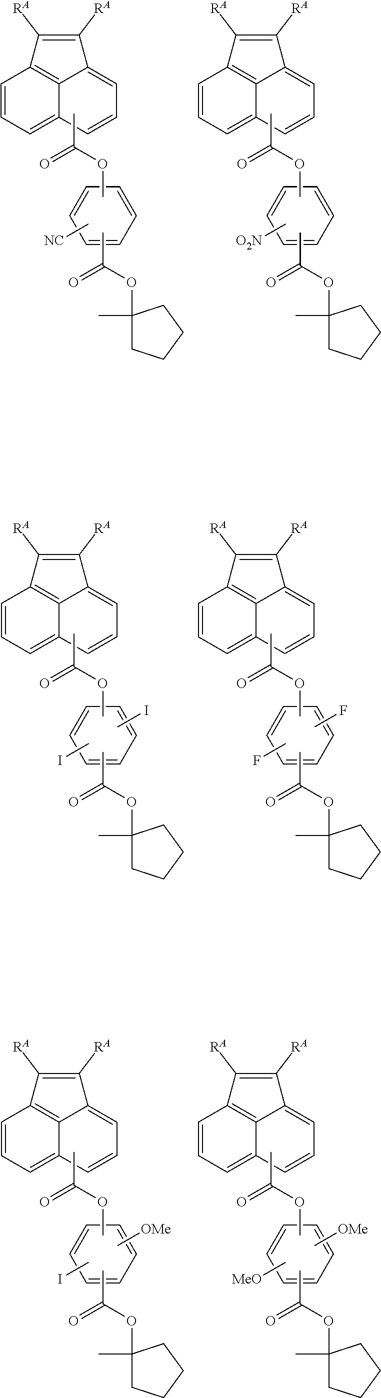

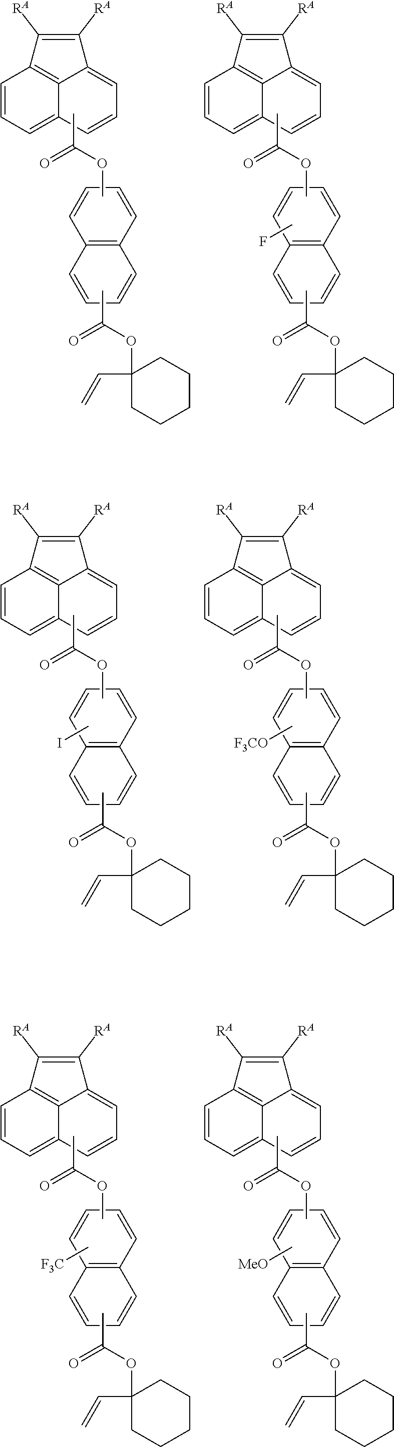

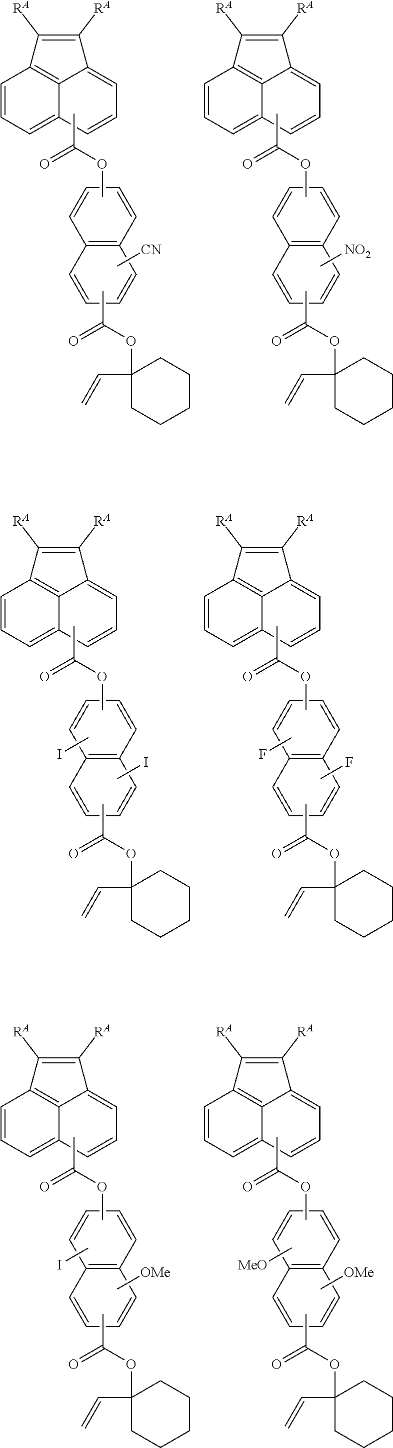

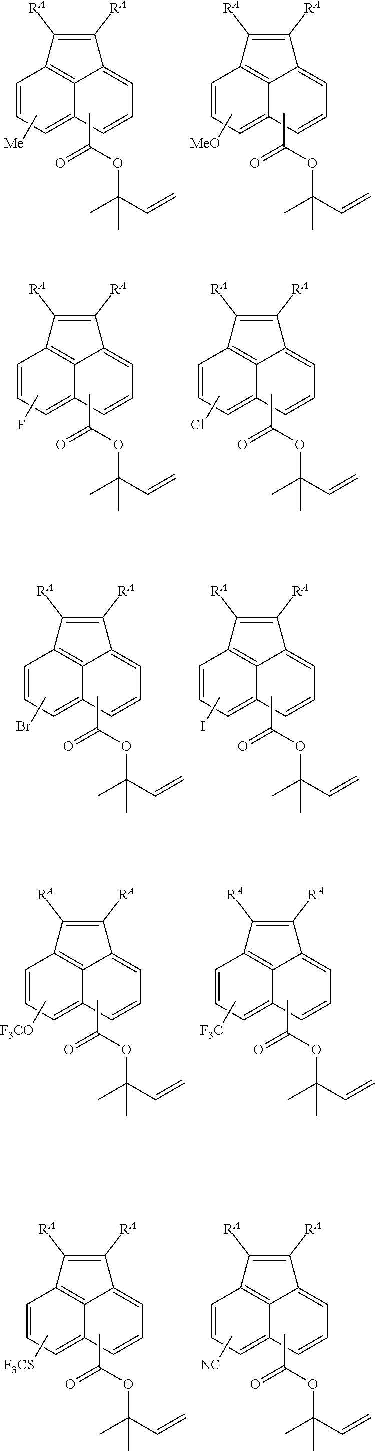

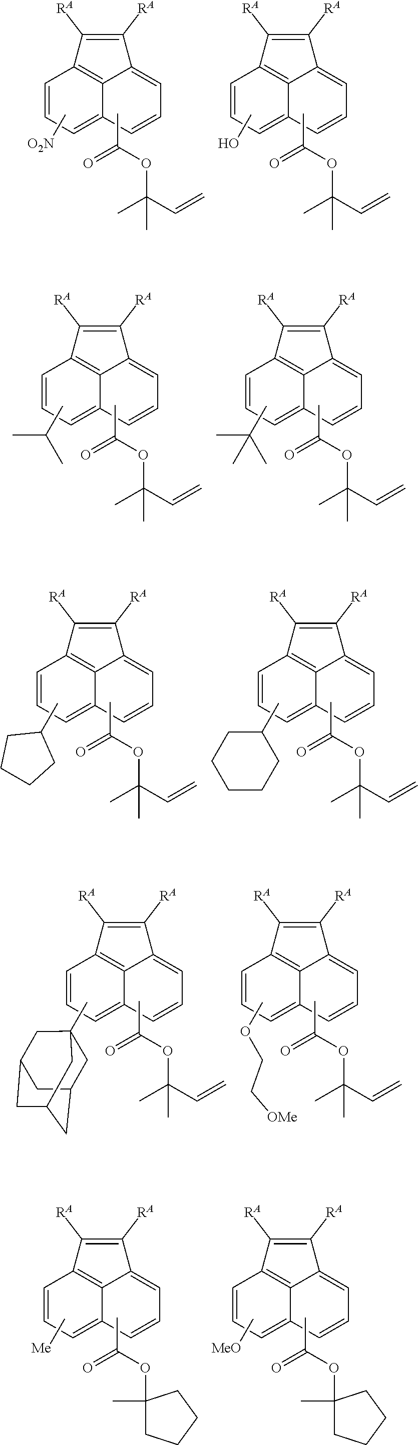

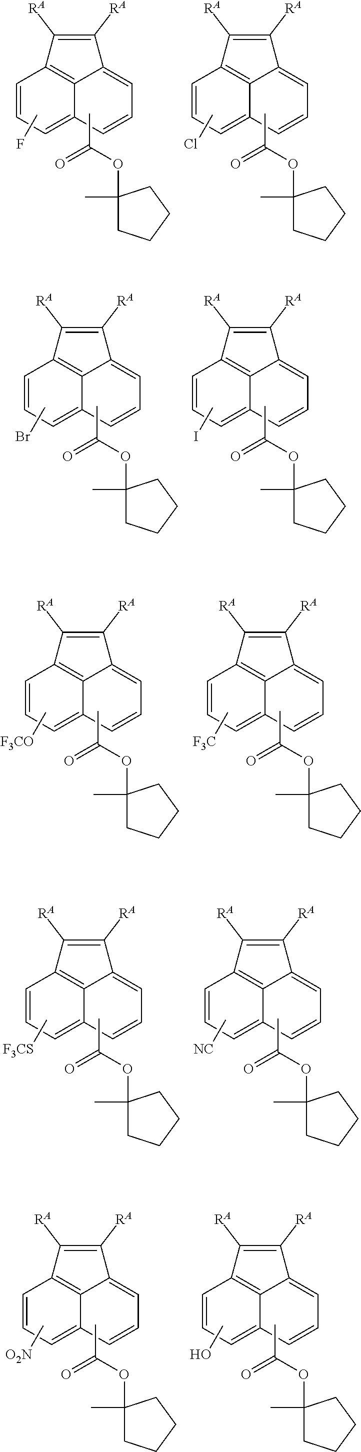

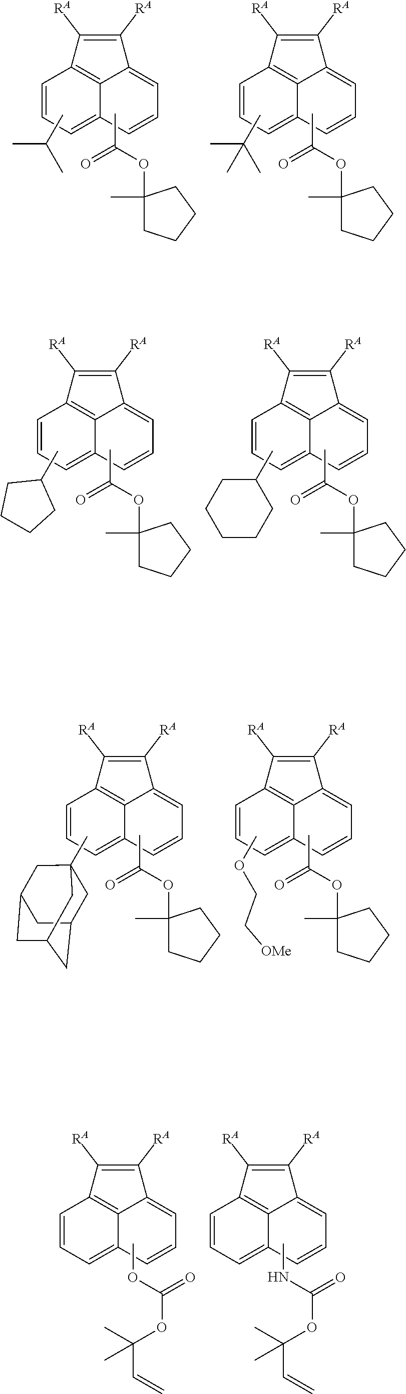

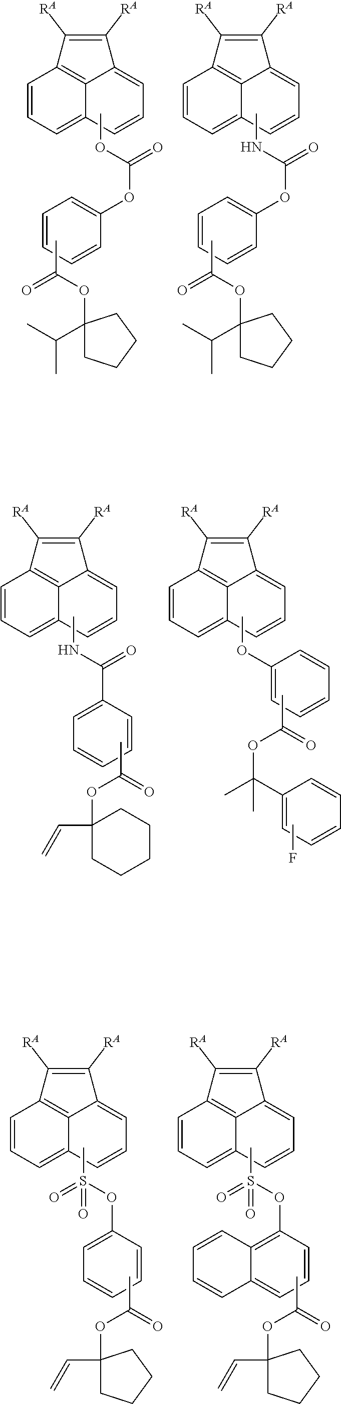

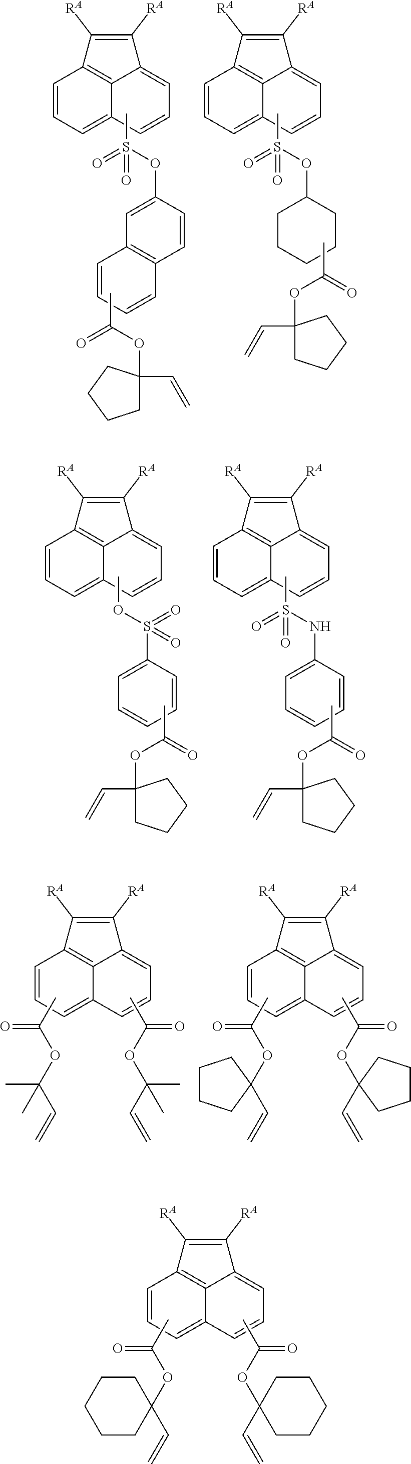

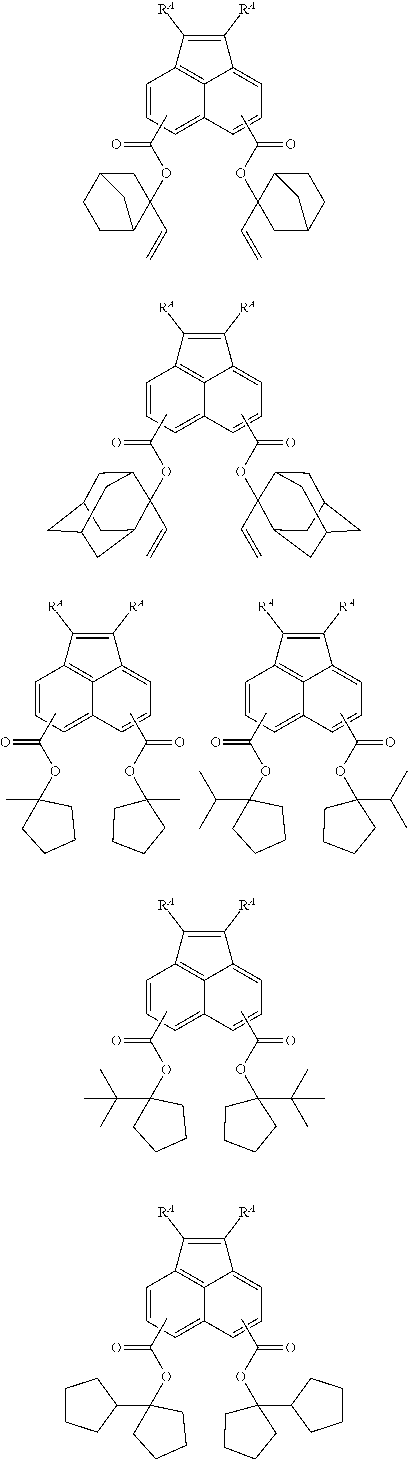

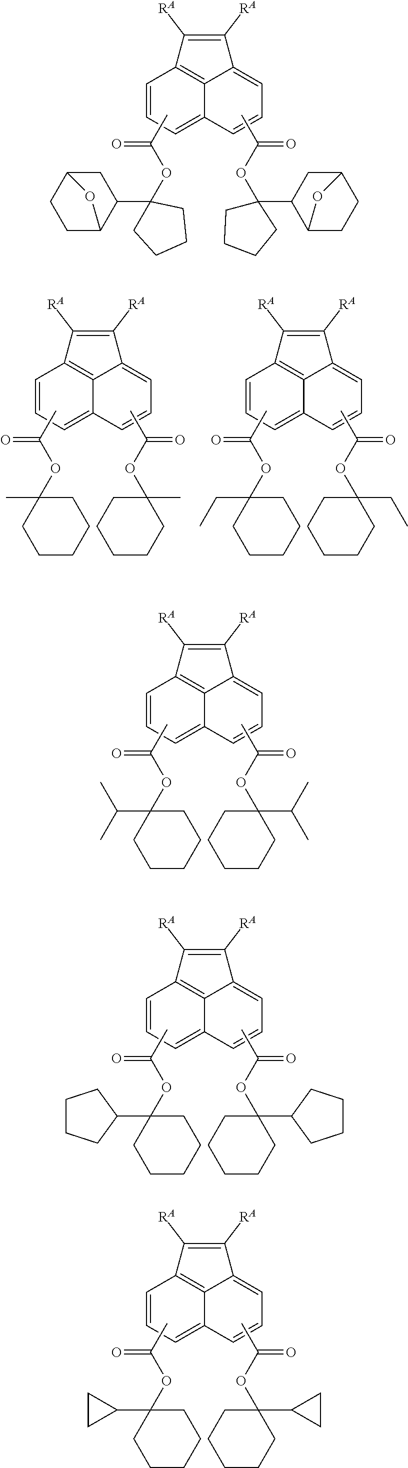

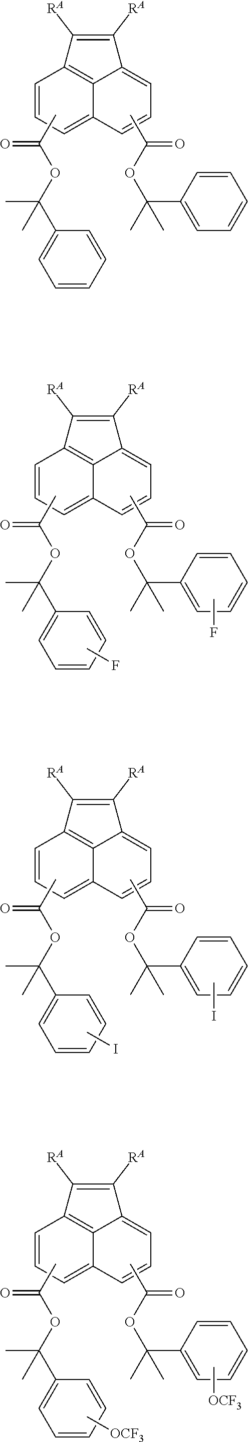

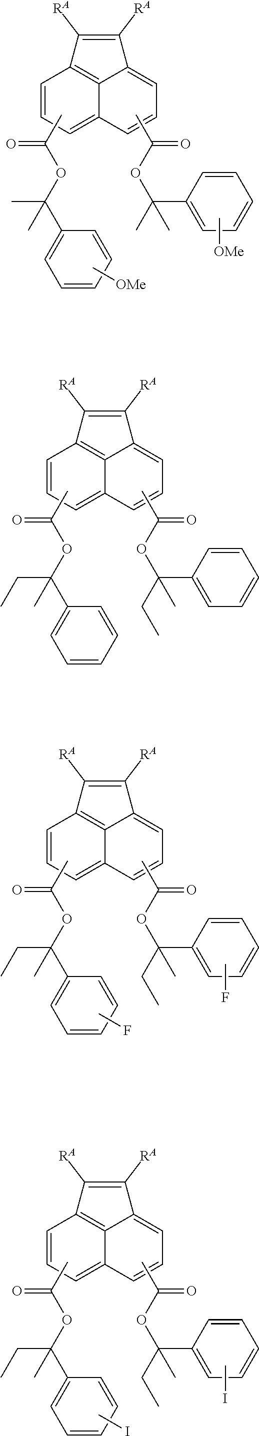

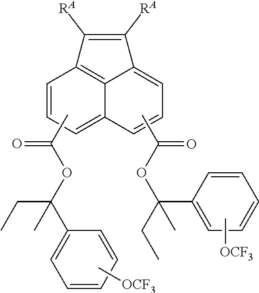

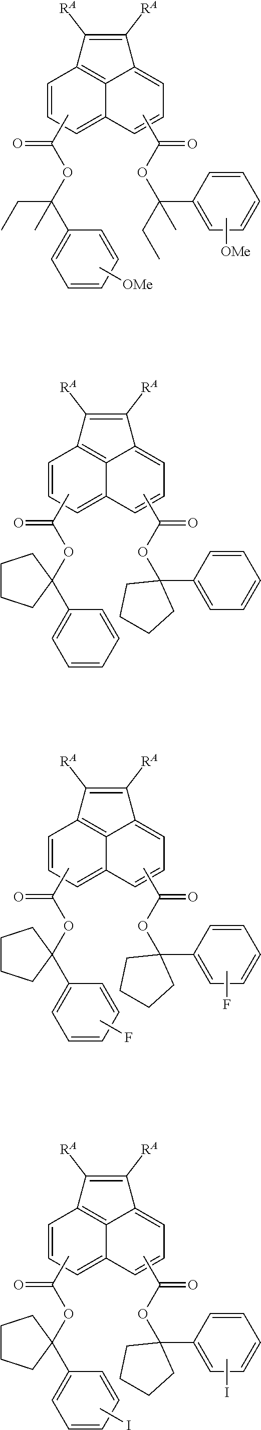

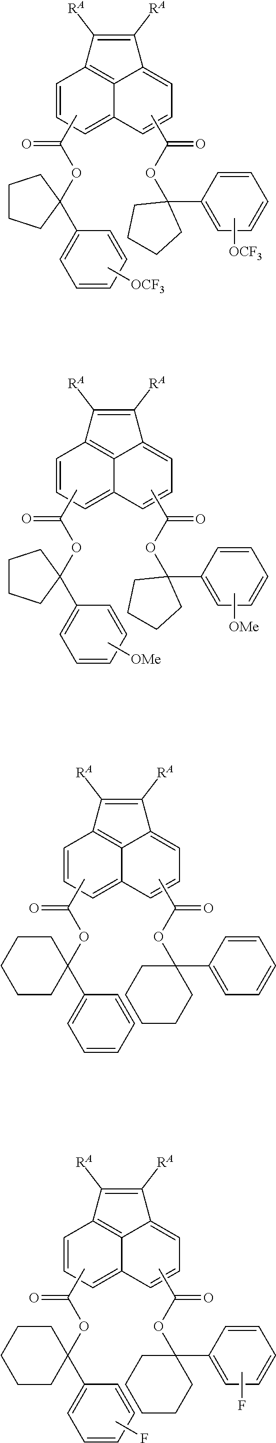

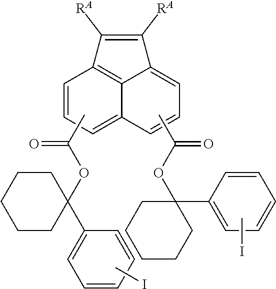

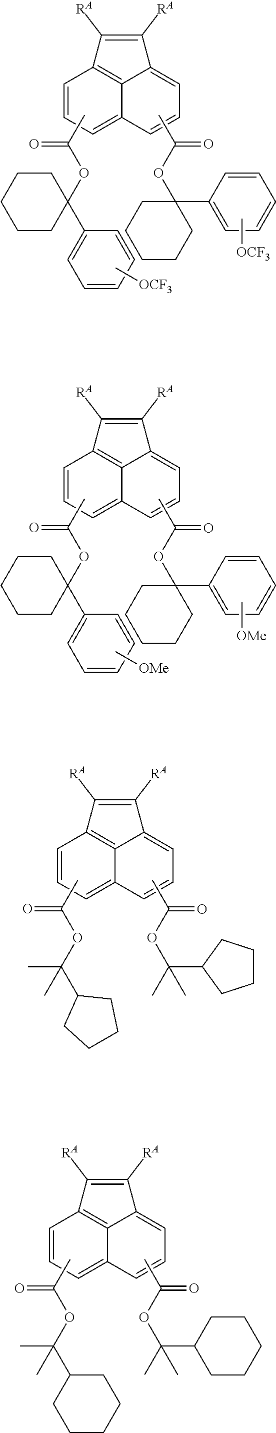

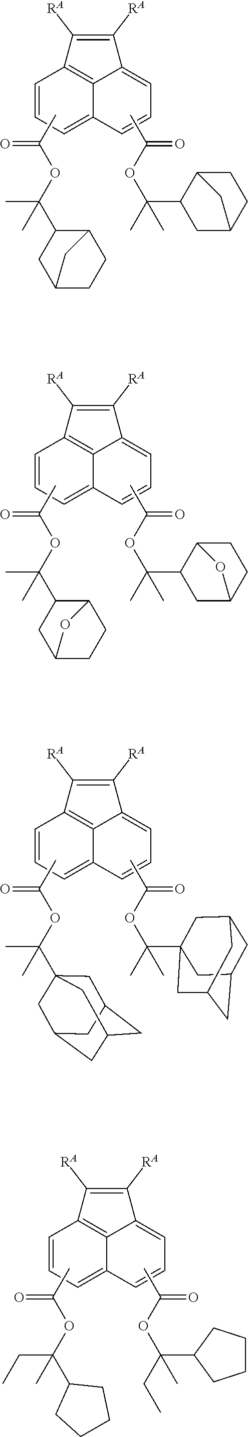

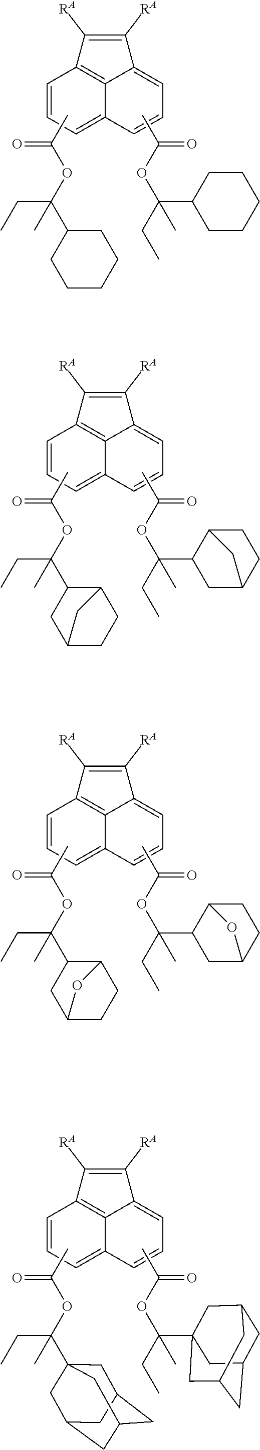

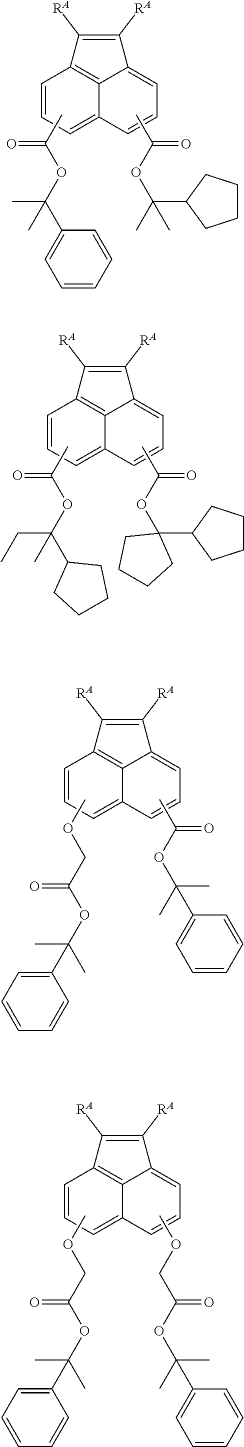

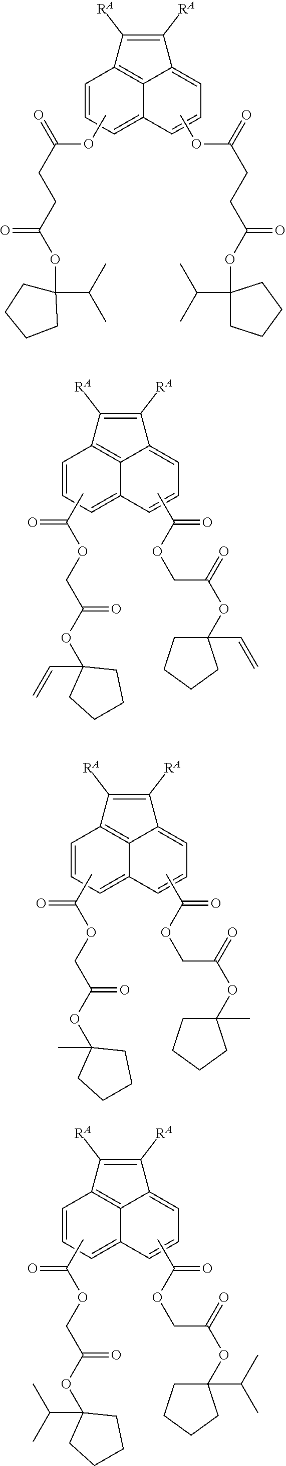

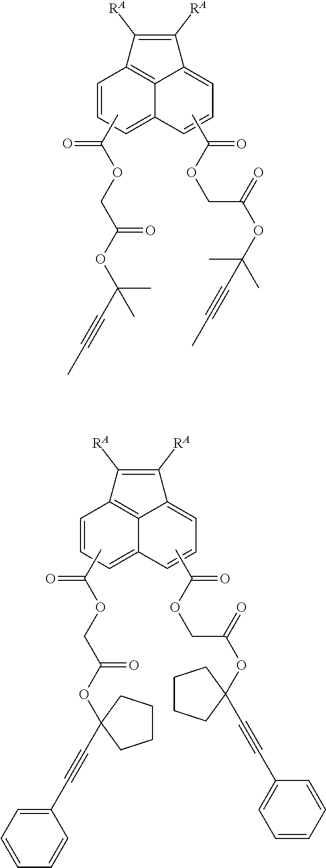

The inventive monomer can be synthesized by any well-known methods, for example, according to the following scheme.

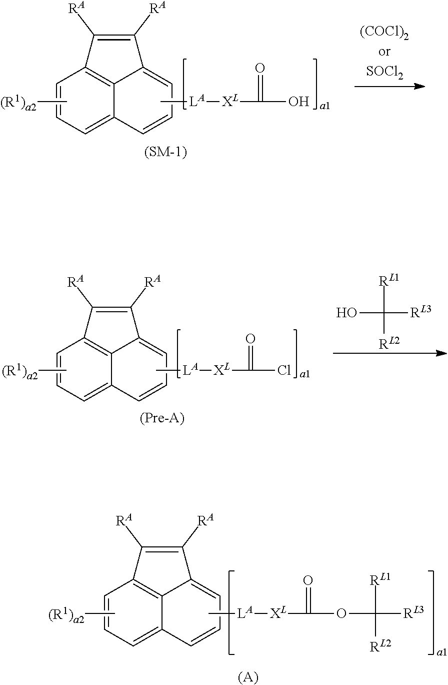

Herein a1, a2, R^{A}, R¹, L^{A}, X^{L}, and R^{L1} to R^{L3} are as defined above.

The first step is a reaction for converting carboxylic acid reactant (SM-1) to carboxylic chloride (Pre-A).

The reactant (SM-1) may be synthesized by a well-known synthesis technique or is commercially available. The reactant (SM-1) is suspended in an aromatic hydrocarbon solvent such as toluene or halogenated solvent such as methylene chloride, to which oxalyl chloride or thionyl chloride is added dropwise to produce carboxylic chloride (Pre-A). In the preparation step, the reaction can be accelerated by adding a small amount of N,N-dimethylformamide. For efficient preparation of carboxylic chloride, the system is preferably heated at room temperature to near the boiling point of the solvent. It is desirable from the aspect of yield to monitor the reaction by gas chromatography (GC) or silica gel thin layer chromatography (TLC) until the reaction is complete. Typically, the reaction time is 2 to 4 hours. The reaction solution is concentrated, obtaining the desired precursor, carboxylic chloride (Pre-A). If necessary, the compound may be purified by a standard technique such as distillation, chromatography or recrystallization.

The second step is esterification reaction of carboxylic chloride (Pre-A) with a tertiary alcohol to produce the target, monomer (A).

The esterification reaction may be performed according to the well-known formulation. The reaction is performed by dissolving the precursor, carboxylic chloride (Pre-A) and the corresponding tertiary alcohol in a solvent and adding dropwise an organic base thereto. Suitable solvents include hydrocarbon solvents such as toluene and hexane, polar aprotic solvents such as tetrahydrofuran and acetonitrile, and halogenated solvents such as methylene chloride and 1,2-dichloroethane. Suitable organic bases include triethylamine and pyridine. The reaction can be accelerated by adding 4-dimethylaminopyridine. The system may be heated if necessary for increasing the conversion rate. It is desirable from the aspect of yield to monitor the reaction by GC or silica gel TLC until the reaction is complete. Typically, the reaction time is 0.5 to 3 hours. The target, monomer (A) is recovered from the reaction mixture by standard aqueous workup. If necessary, the monomer may be purified by a standard technique such as distillation, chromatography or recrystallization.

The above-mentioned preparation method is merely exemplary and the method of preparing the inventive monomer is not limited thereto.

The inventive monomer is structurally characterized by having a robust acenaphthylene structure as a polymerizable group and an acid labile group of tertiary ester type bonded thereto. In the exposed region, the acid labile group of tertiary ester type undergoes deprotection reaction under the action of the acid generated by an acid generator, to create a carboxy group. The tertiary ester group having a cyclic structure or multiple bond is preferred. In the case of a tertiary ester group having a cyclic structure, reactivity with acid is enhanced by the driving force due to the elimination of steric hindrance. In the case of a tertiary ester group having a multiple bond, reactivity with acid is improved by the driving force due to the creation of a benzyl or allyl cation. Then the contrast between exposed and unexposed regions is increased. In the case of a positive resist composition using an alkaline developer, the exposed region where deprotection reaction has taken place is so improved in affinity to the alkaline developer that the exposed region is effectively removed by the developer. Since a tertiary ester structure in the form of a saturated chainlike alkyl group free of a cyclic structure or multiple bond is unlikely to achieve the above-mentioned effect, its reactivity with acid is not so high. The polymerizable group in the form of acenaphthylene structure serves to elevate the glass transition temperature (Tg) of the polymer because the fused aromatic ring has robustness. While the unexposed region must have resistance to alkaline developer, the polymer which is robust owing to the acenaphthylene structure exhibits resistance to alkaline developer. This prevents the resist pattern from peeling from the substrate and from collapsing and allows the resist pattern to have excellent etch resistance in the subsequent etching step. By virtue of the synergy of these effects, the chemically amplified resist composition comprising the inventive polymer forms a resist pattern having improved properties including a high dissolution contrast, reduced LWR of line patterns or improved CDU of hole patterns, collapse resistance, and etch resistance. The monomer or polymer is useful as a material for a resist composition, especially of positive tone, for forming small-size patterns.

### [Polymer]

Another embodiment of the invention is a polymer obtained from the monomer having formula (A). That is, the polymer comprises repeat units derived from the monomer having formula (A), which are also referred to as repeat units A. The repeat units A have the formula (A′).

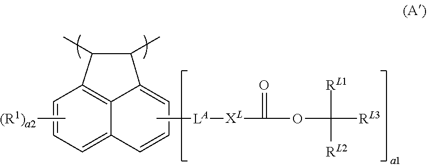

Herein, a1, a2, R^{A}, R¹, R^{L1} to R^{L3}, L^{A} and X^{L} are as defined above. The repeat units A may be of one type or two or more types.

The polymer may further contain repeat units having the formula (a1), which are also referred to as repeat units (a1), or repeat units having the formula (a2), which are also referred to as repeat units (a2).

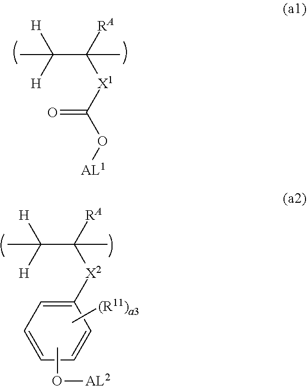

In formulae (a1) and (a2), R^{A} is each independently hydrogen, fluorine, methyl or trifluoromethyl. X¹ is a single bond, phenylene group, naphthylene group, *—C(═O)—O—X¹¹— or *—C(═O)—NH—X¹¹—. The phenylene or naphthylene group may be substituted with an optionally fluorinated C₁-C₁₀ alkoxy moiety or halogen. X¹¹ is a C₁-C₁₀ saturated hydrocarbylene group, phenylene or naphthylene group. The saturated hydrocarbylene group may contain a hydroxy moiety, ether bond, ester bond or lactone ring. X² is a single bond, *—C(═O)—O— or *—C(═O)—NH—. The asterisk (*) designates a point of attachment to the carbon atom in the backbone. R¹¹ is halogen, cyano group, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom. The subscript a3 is an integer of 0 to 4.

In formulae (a1) and (a2), AL¹ and AL² are each independently an acid labile group. Examples of the acid labile group include those described in U.S. Pat. No. 8,574,817 (JP-A 2013-080033) and U.S. Pat. No. 8,846,303 (JP-A 2013-083821).

Typical of the acid labile group are groups of the following formulae (AL-1) to (AL-3).

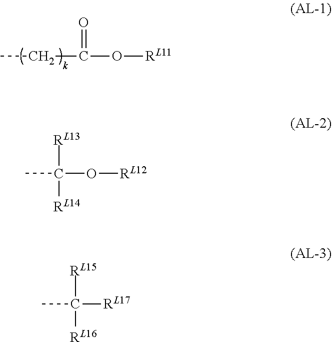

In formulae (AL-1) and (AL-2), R^{L11} and R^{L12} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ hydrocarbyl groups are preferred.

In formula (AL-1), k is an integer of 0 to 10, preferably 1 to 5.

In formula (AL-2), R^{L13} and R^{L14} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L12}, R^{L13} and R^{L14} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L15}, R^{L16} and R^{L17} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Any two of R^{L15}, R^{L16} and R^{L17} may bond together to form a C₃-C₂₀ ring with the carbon atom to which they are attached. The ring preferably contains 4 to 16 carbon atoms and is typically alicyclic.

Examples of repeat unit (a1) are shown below, but not limited thereto. Herein R^{A} and AL¹ are as defined above.

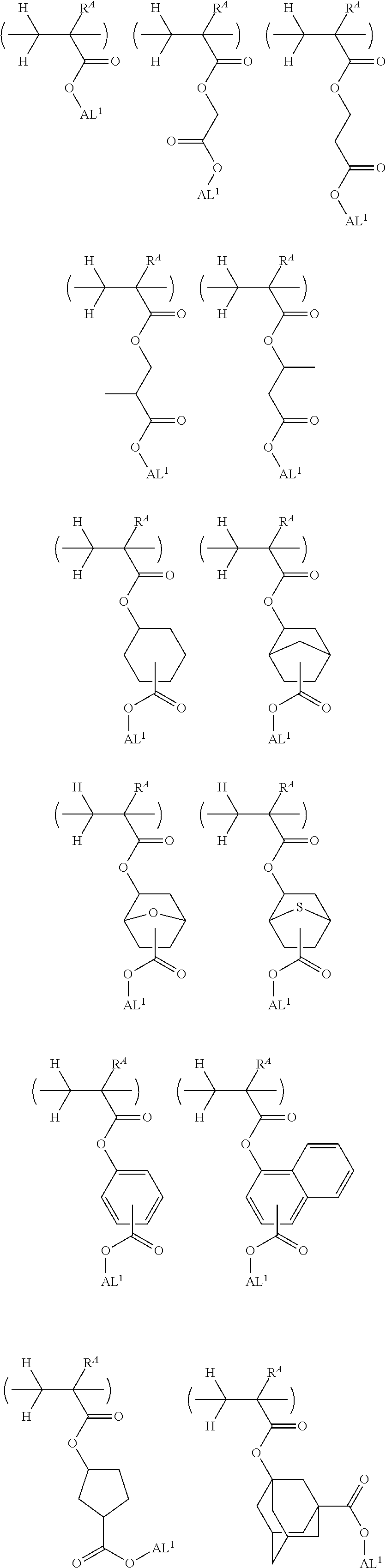

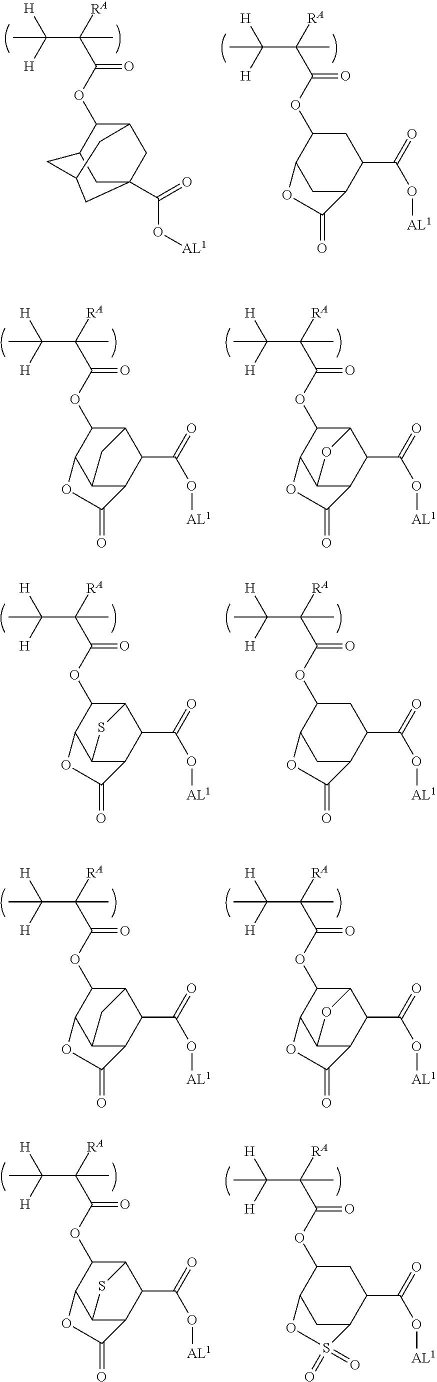

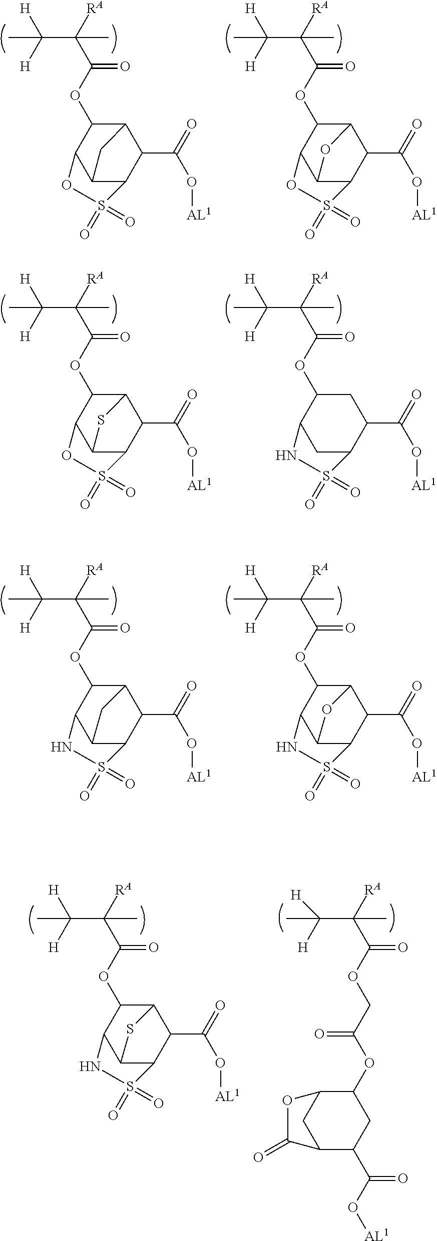

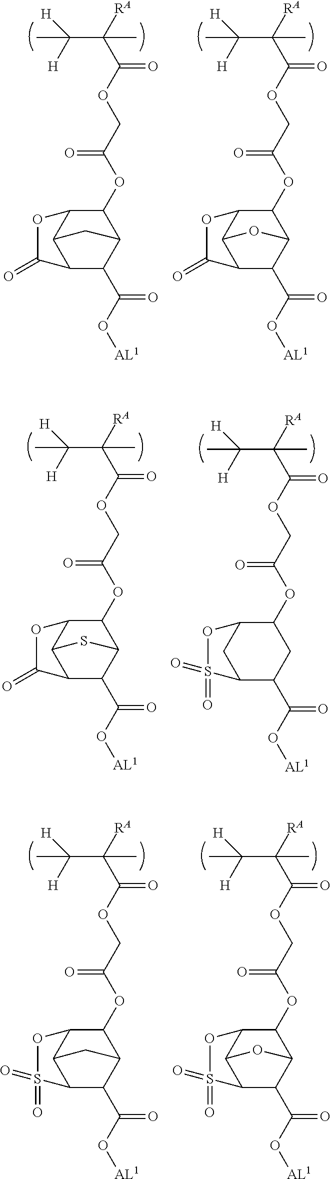

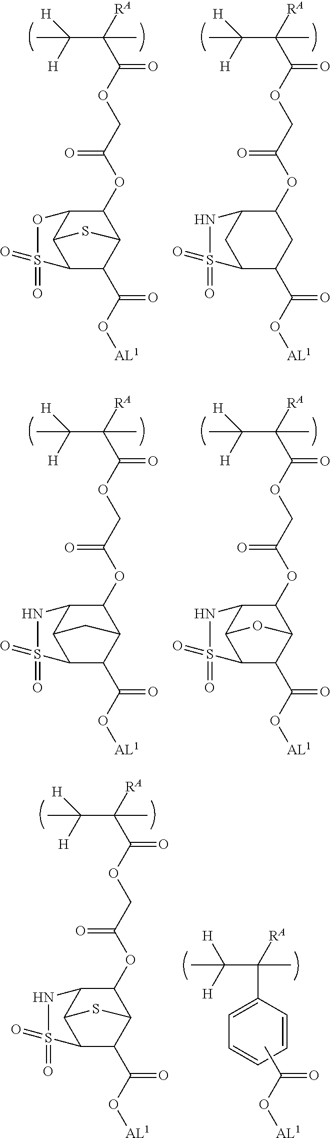

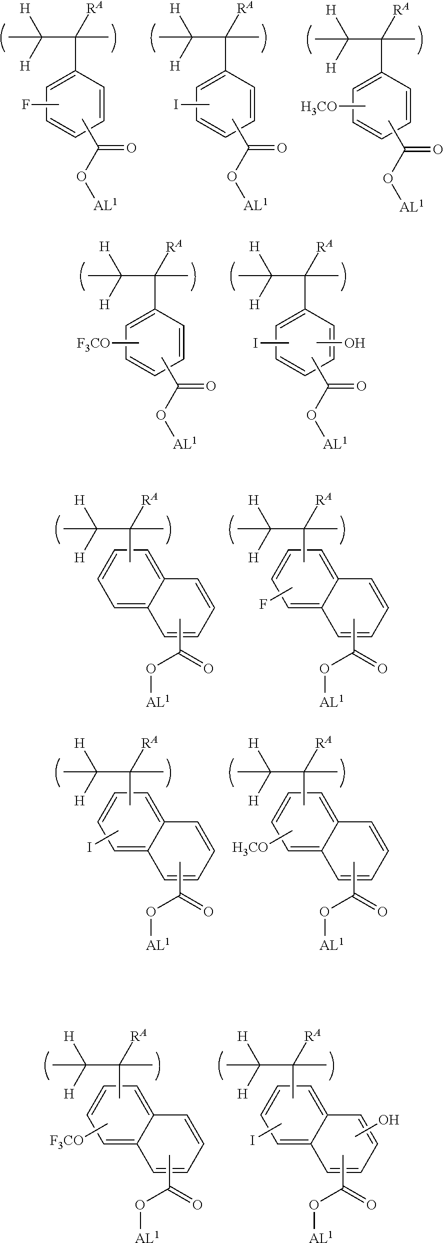

Examples of repeat unit (a2) are shown below, but not limited thereto. Herein R^{A} and AL² are as defined above.

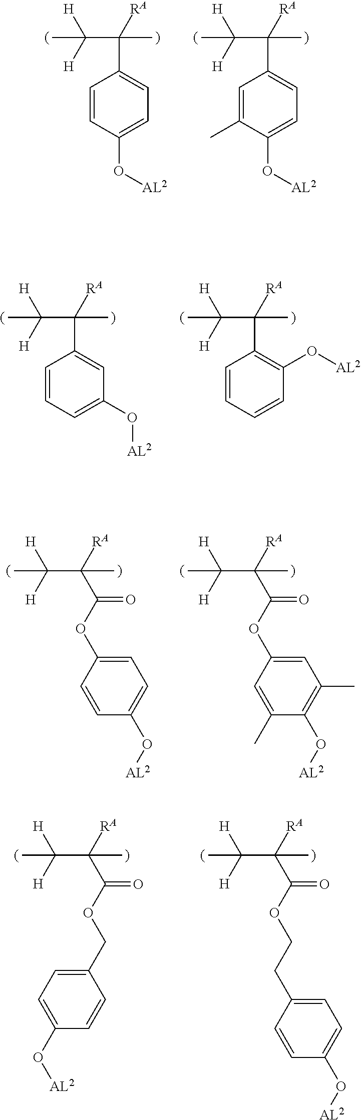

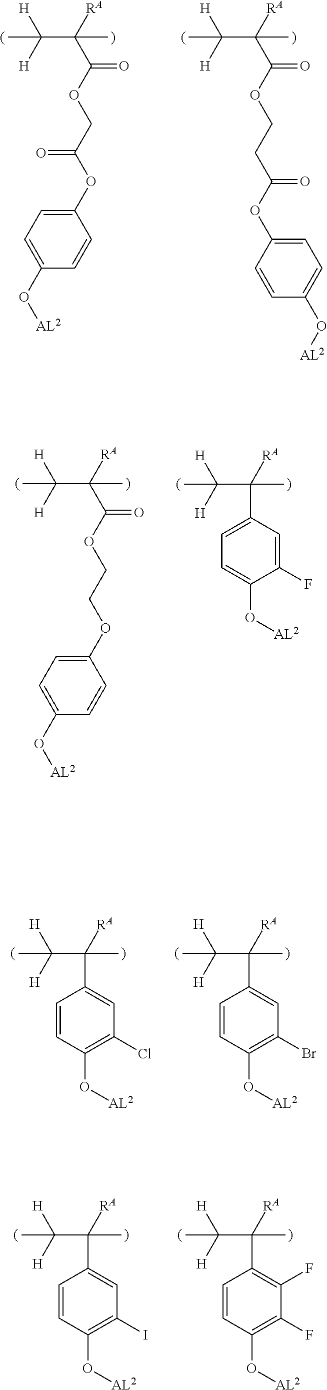

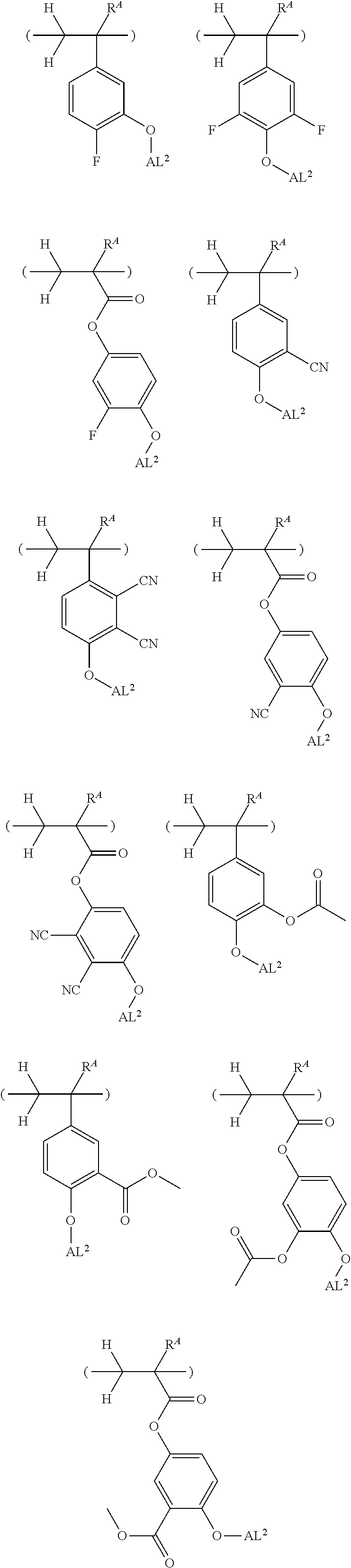

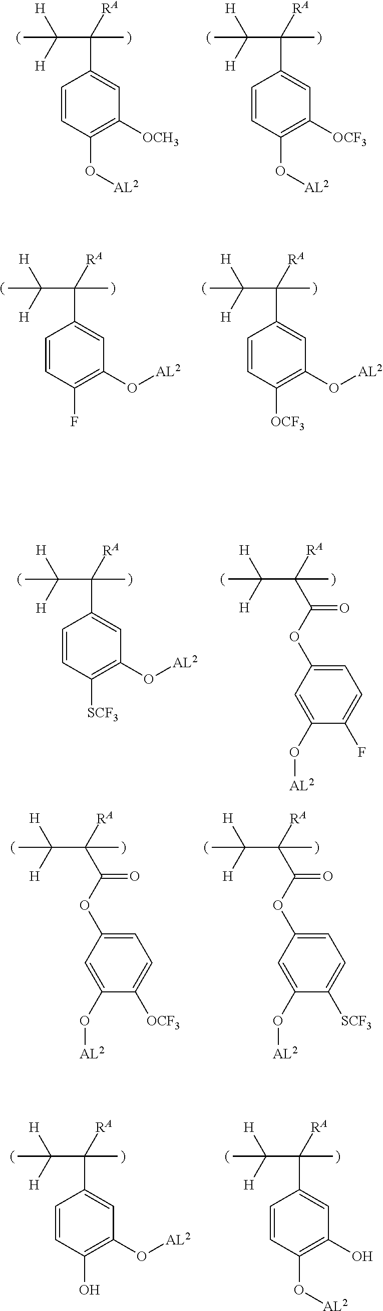

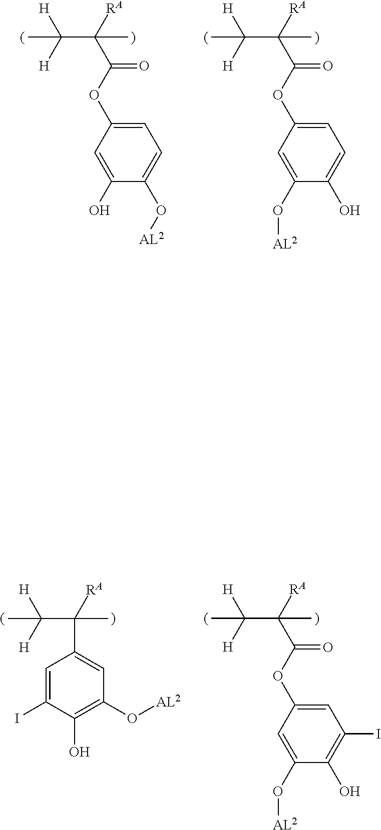

The polymer may further contain repeat units having the formula (b1), which are also referred to as repeat units (b1), or repeat units having the formula (b2), which are also referred to as repeat units (b2).

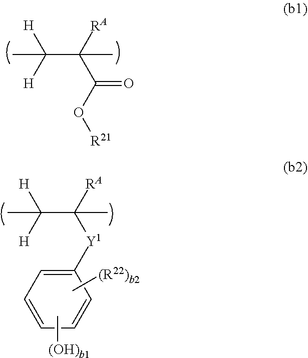

In formulae (b1) and (b2), R^{A} is each independently hydrogen, fluorine, methyl or trifluoromethyl. Y¹ is a single bond or *—C(═O)—O— wherein * designates a point of attachment to the carbon atom in the backbone. R²¹ is hydrogen or a C₁-C₂₀ group which contains at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—). R²² is halogen, hydroxy, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. The subscript b1 is an integer of 1 to 4, b2 is an integer of 0 to 4, and b1+b2 is from 1 to 5.

Examples of the repeat unit (b1) are shown below, but not limited thereto. Herein R^{A} is as defined above.

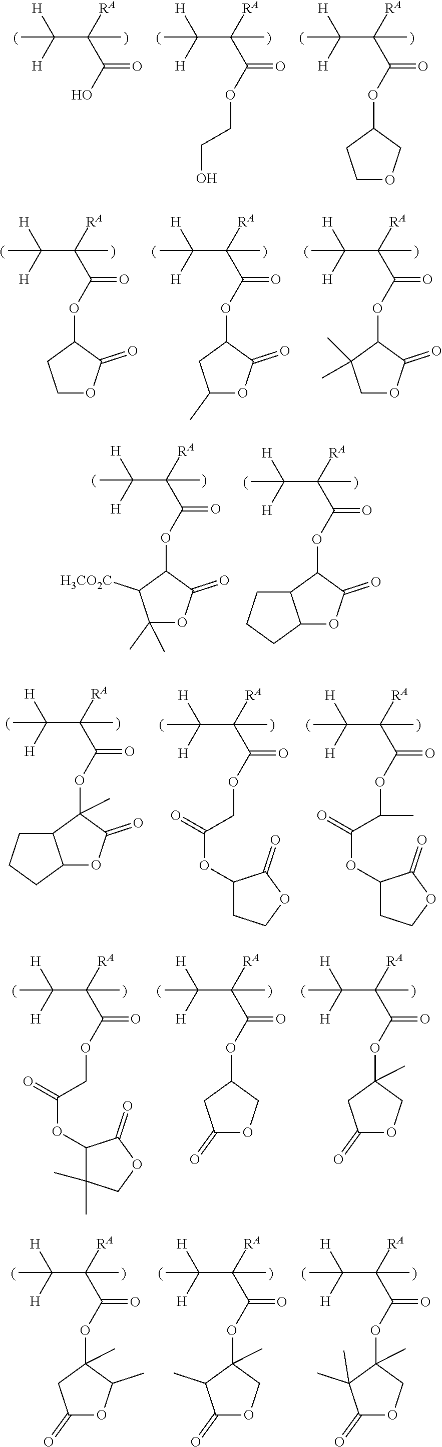

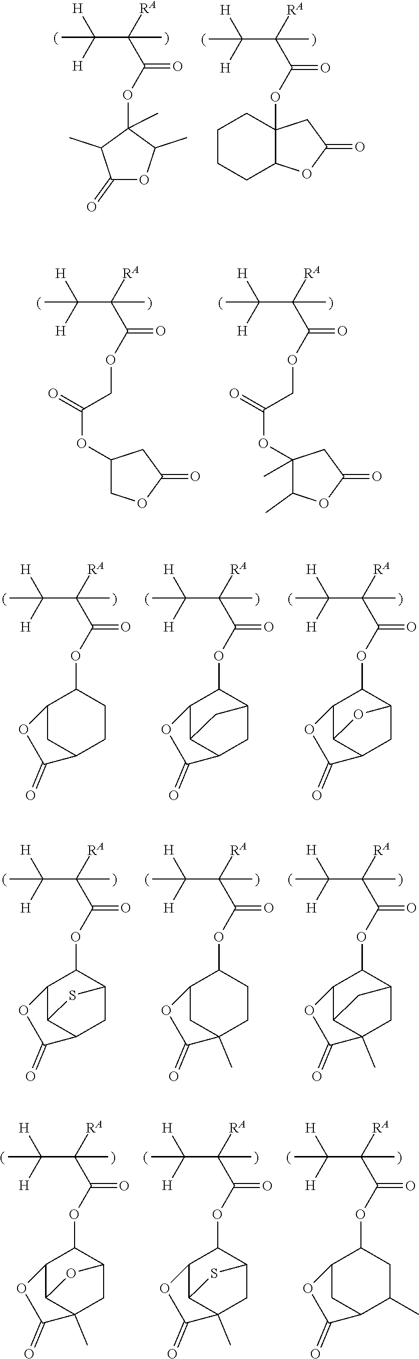

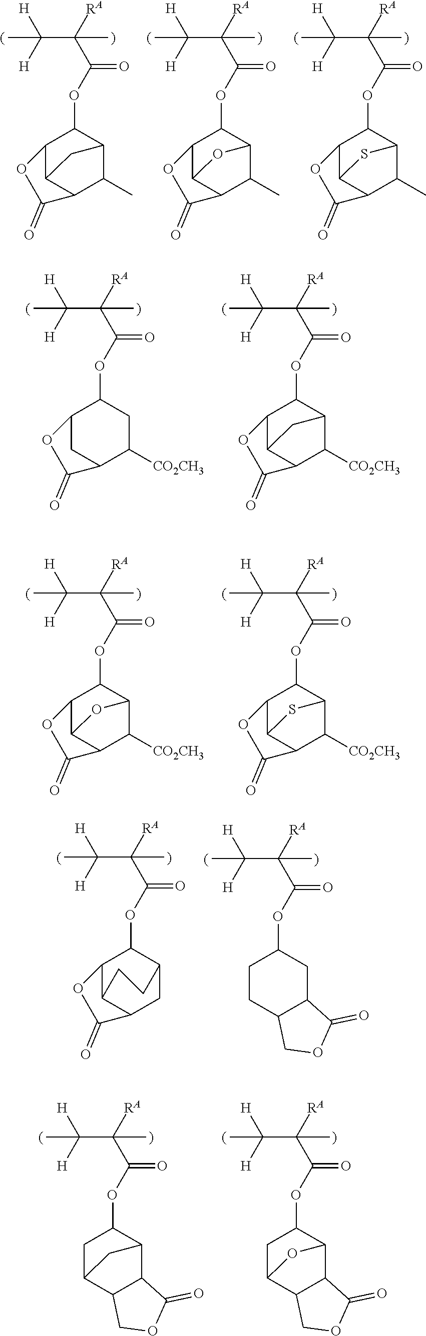

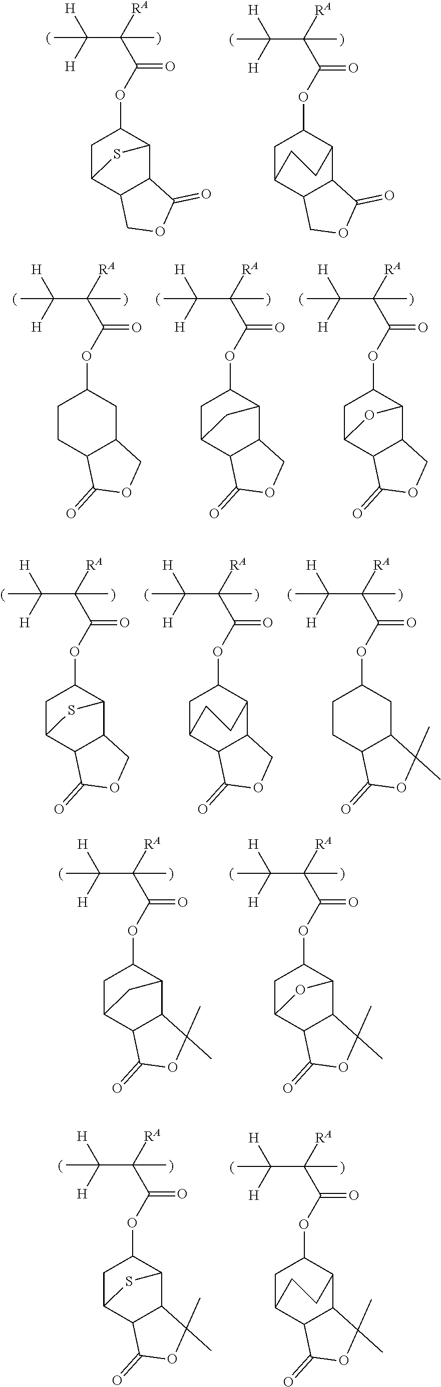

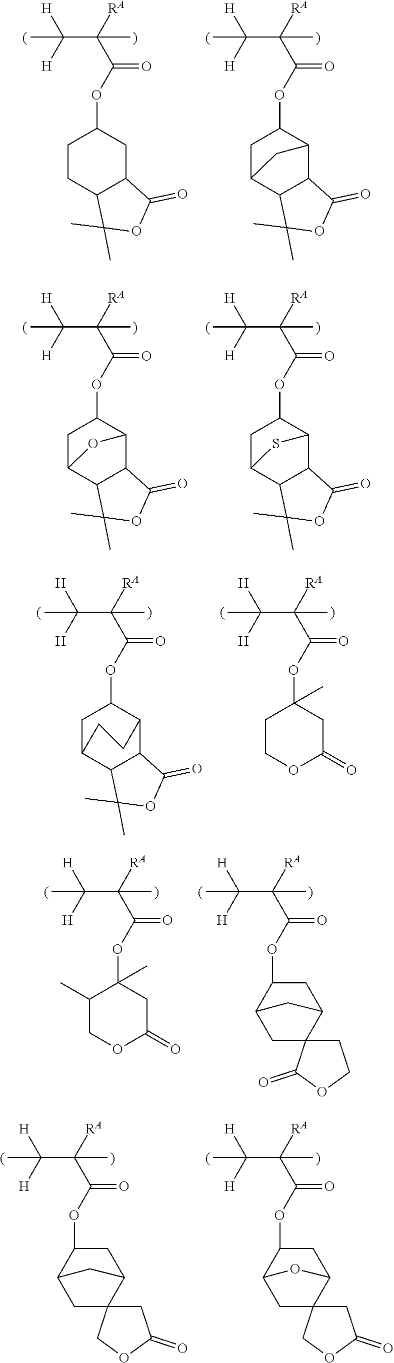

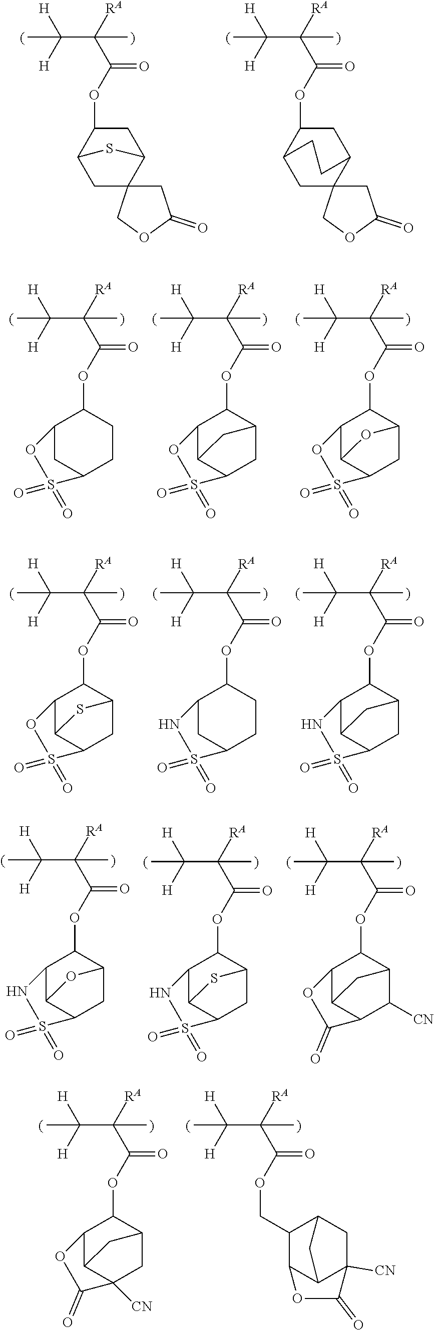

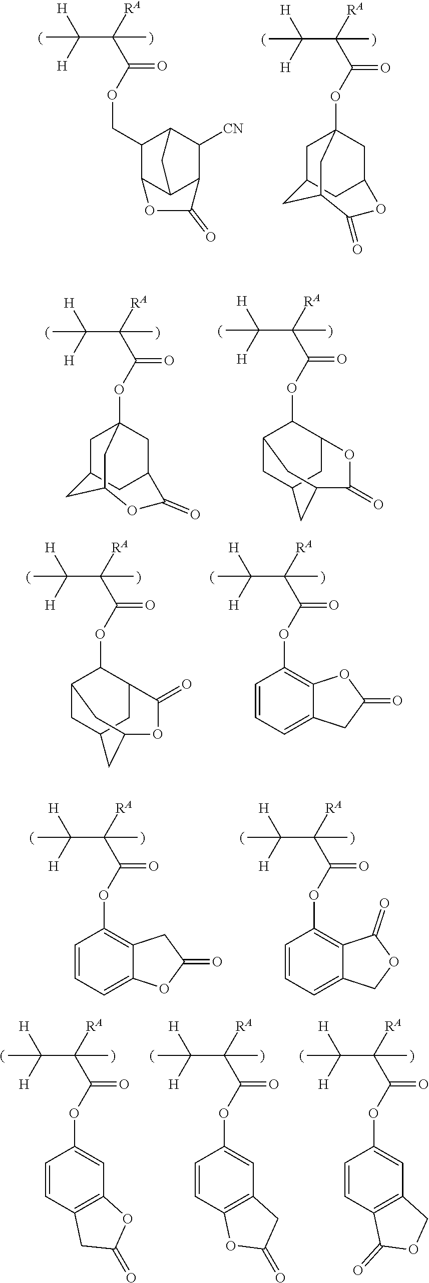

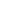

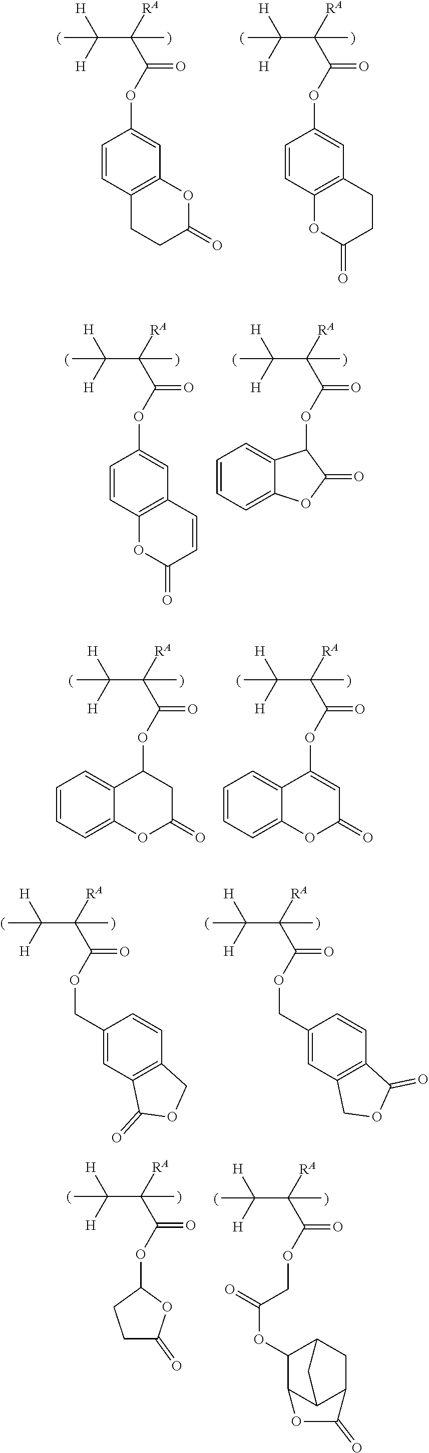

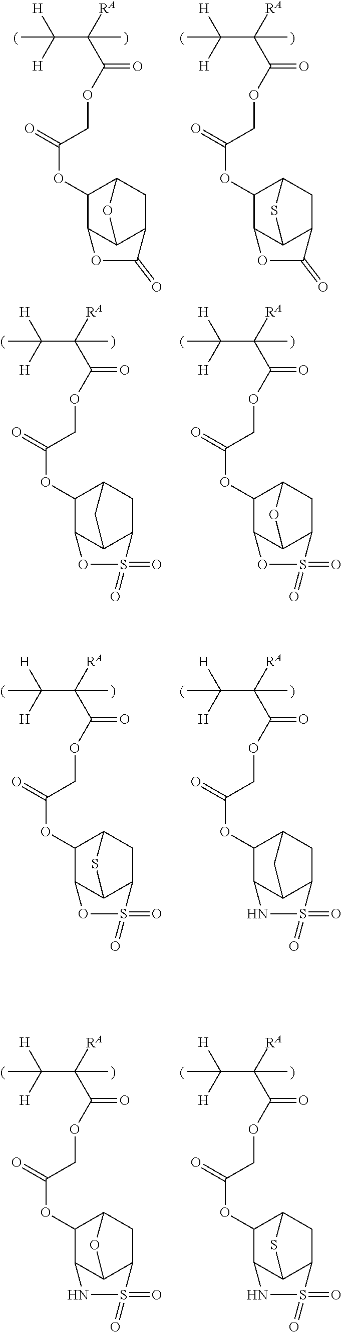

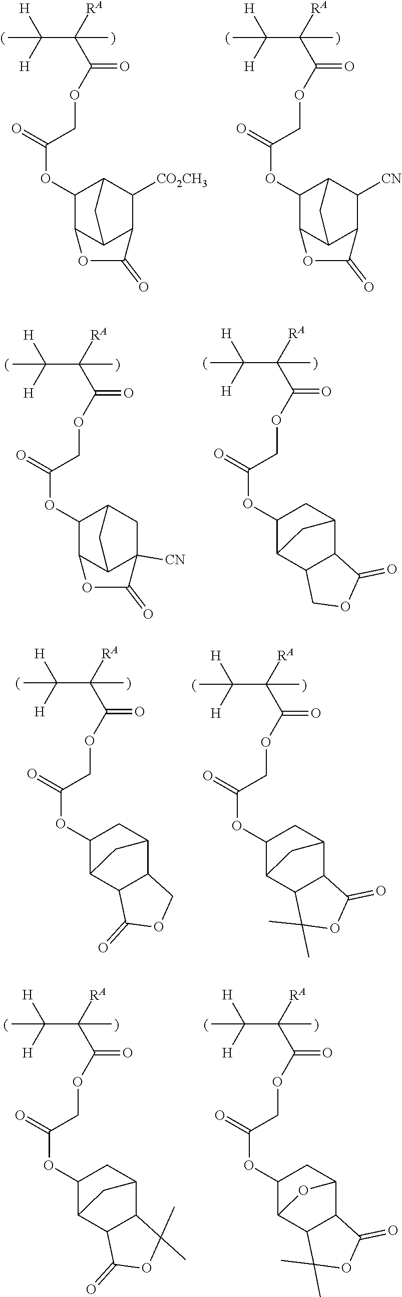

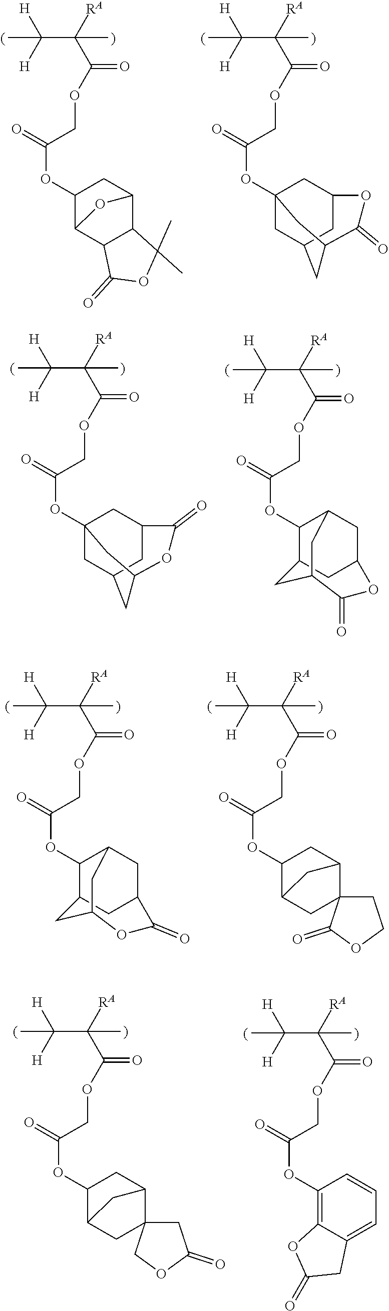

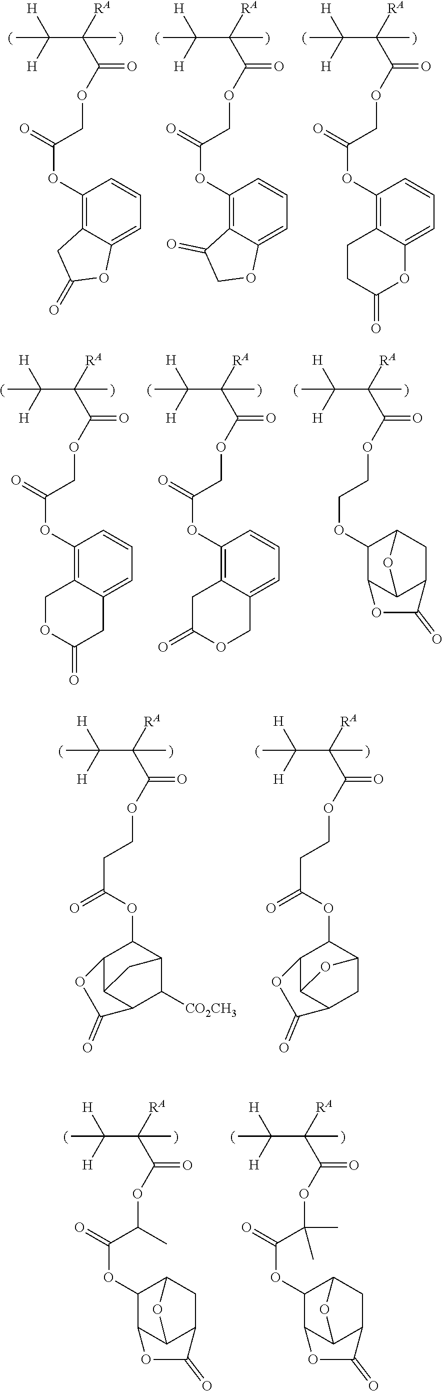

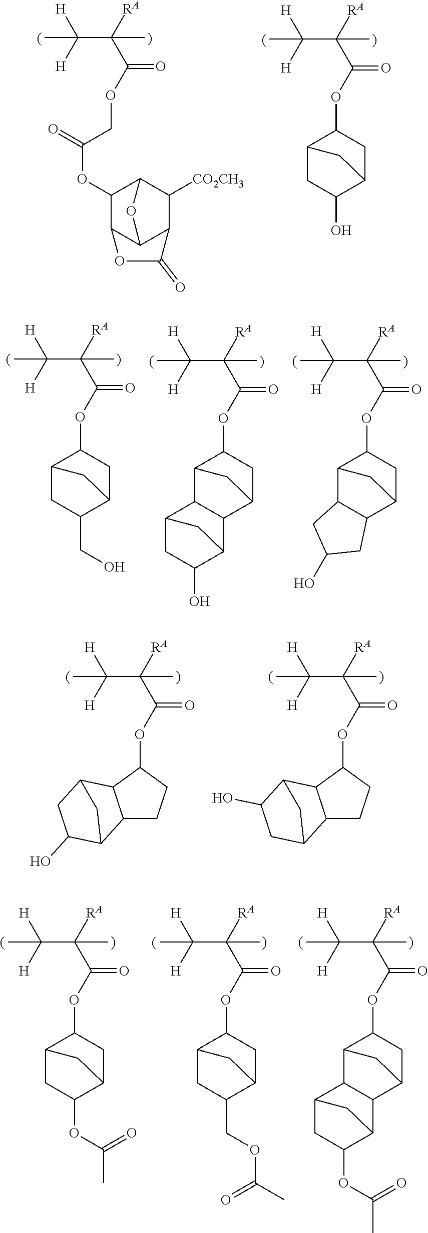

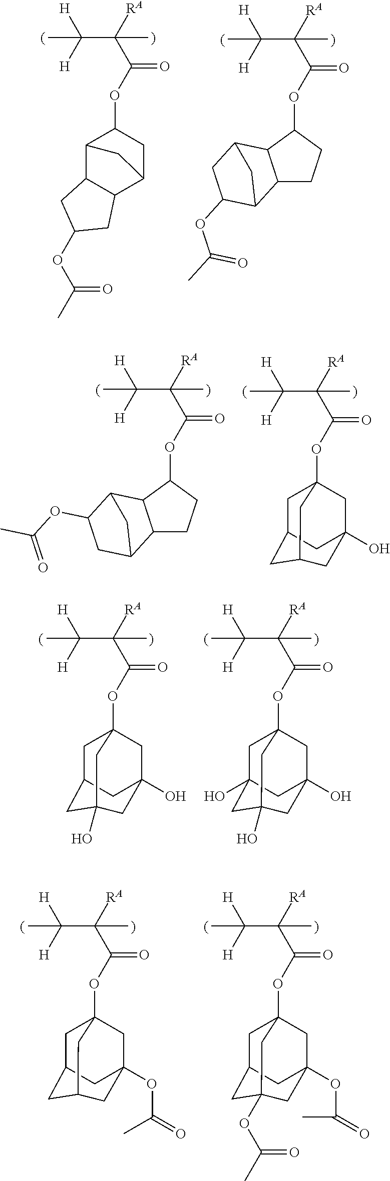

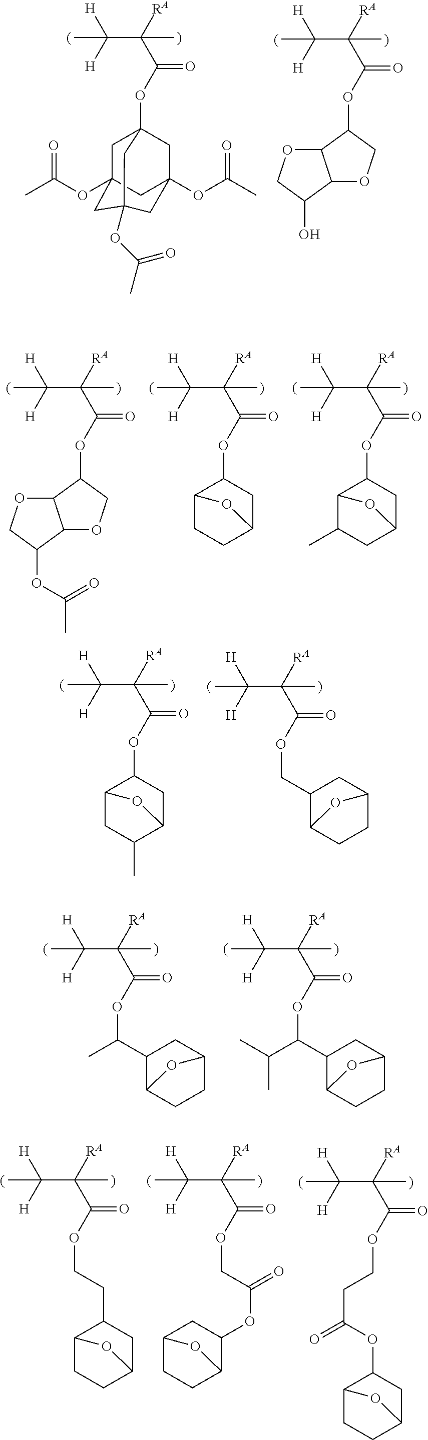

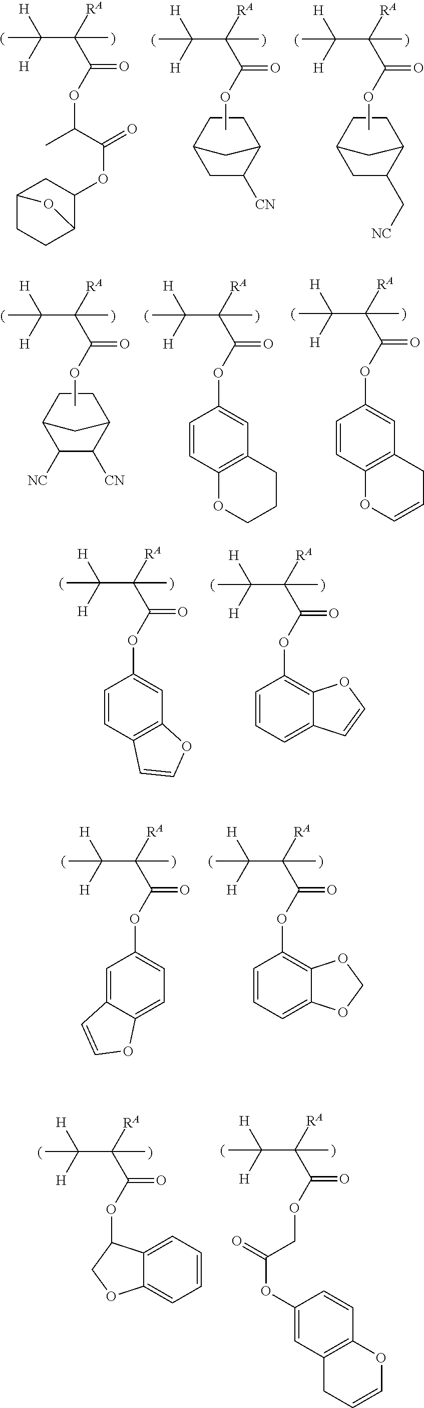

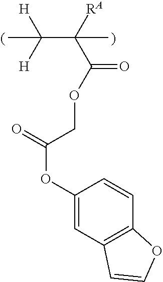

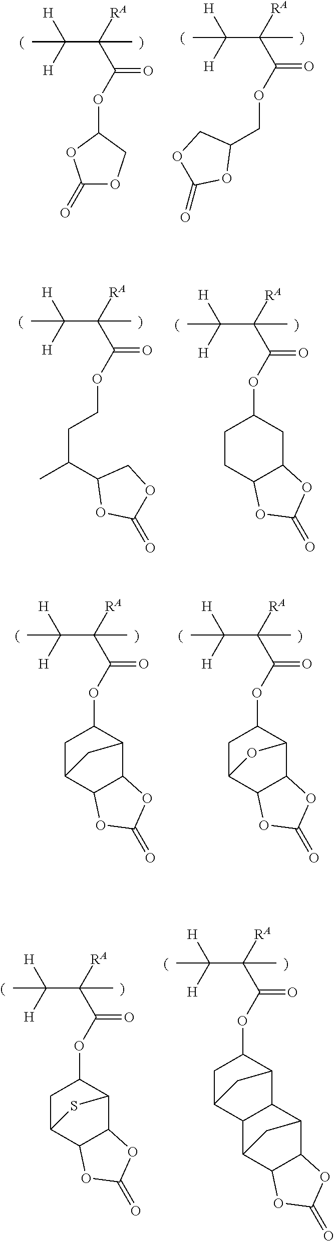

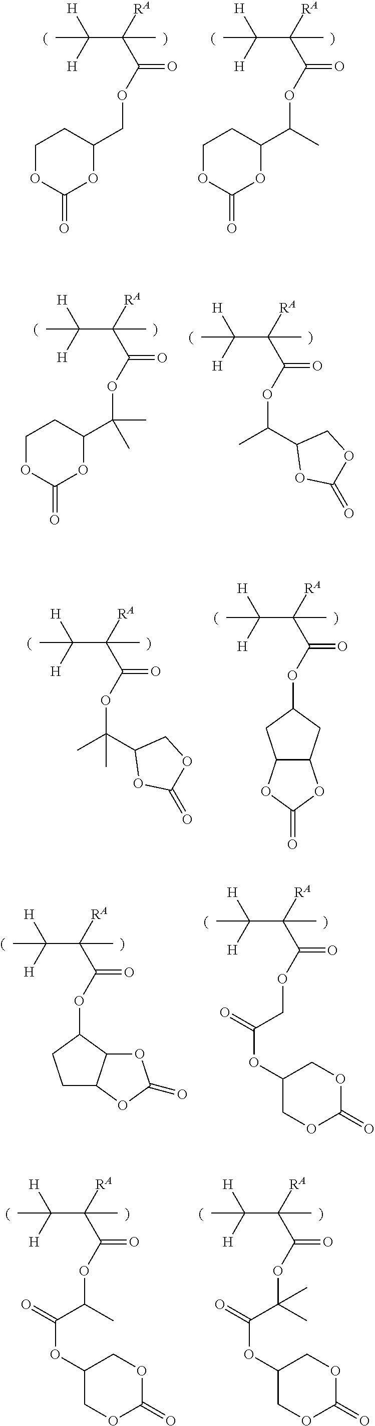

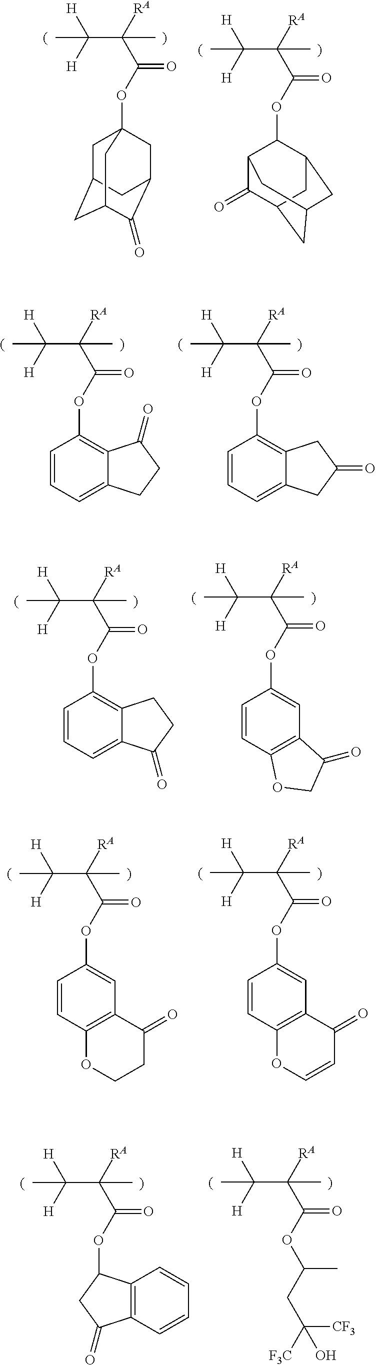

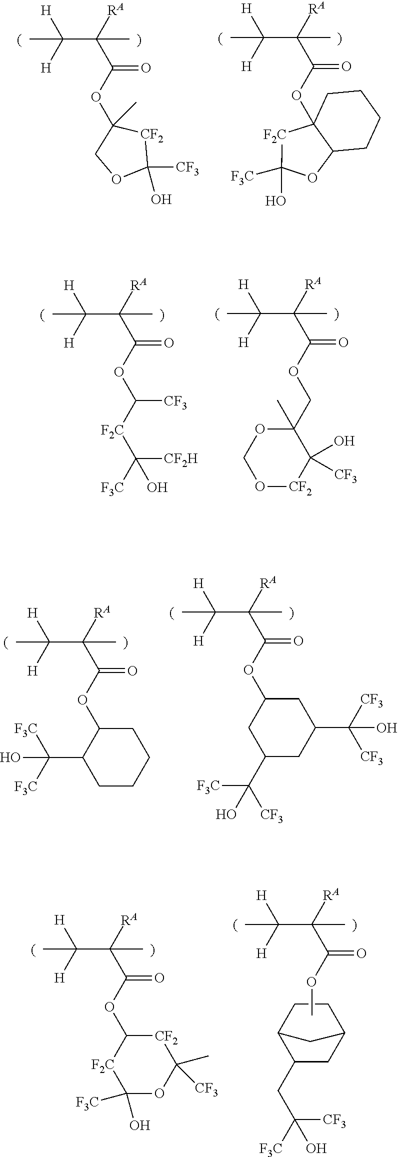

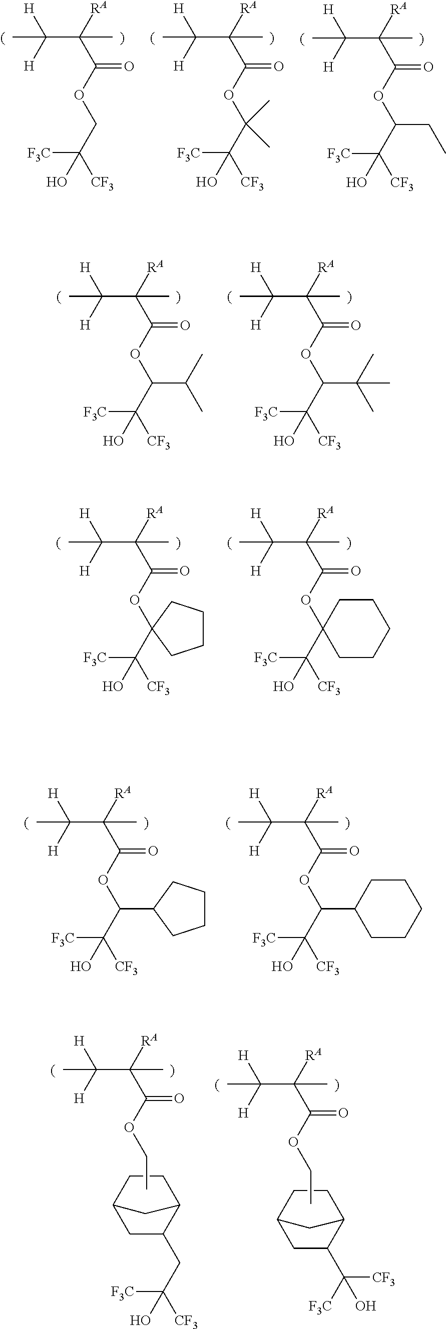

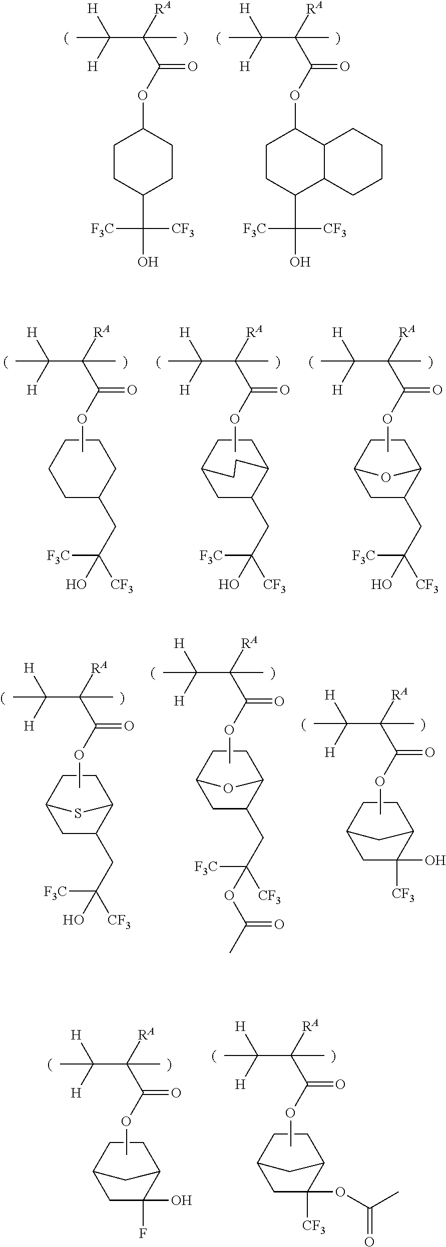

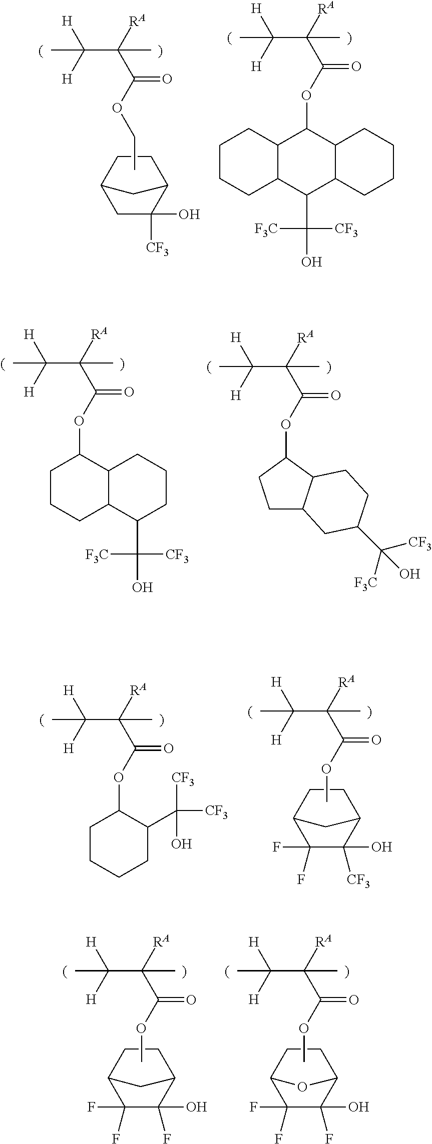

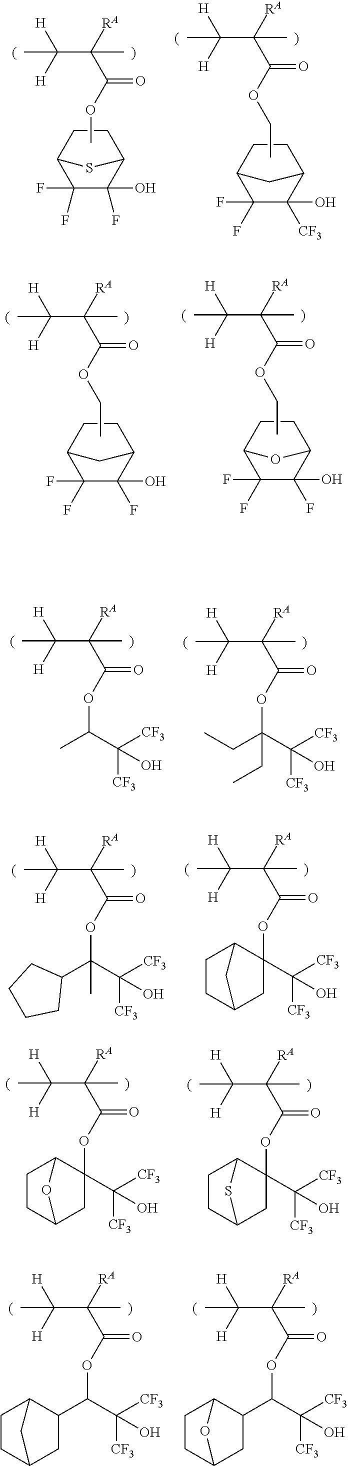

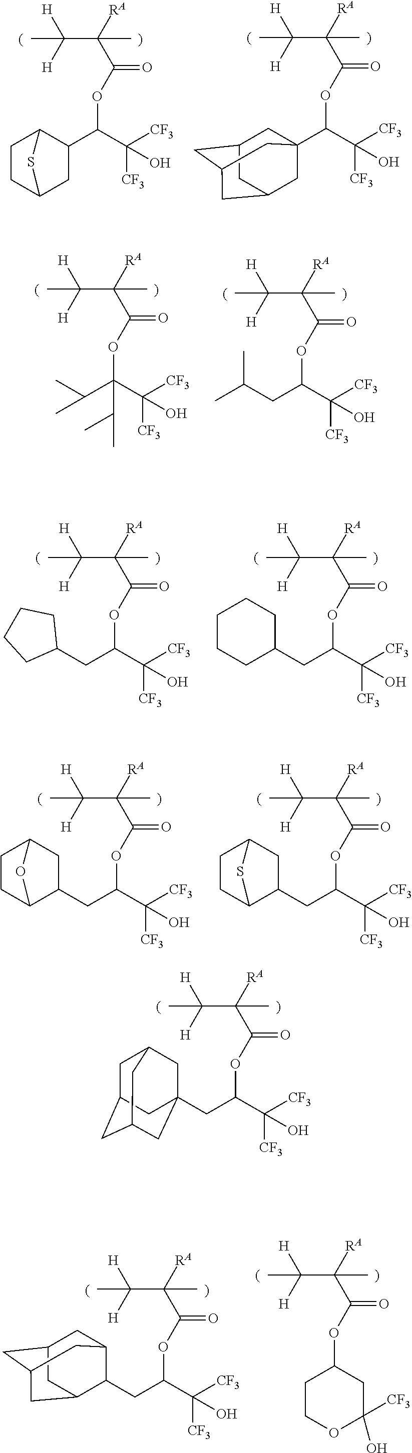

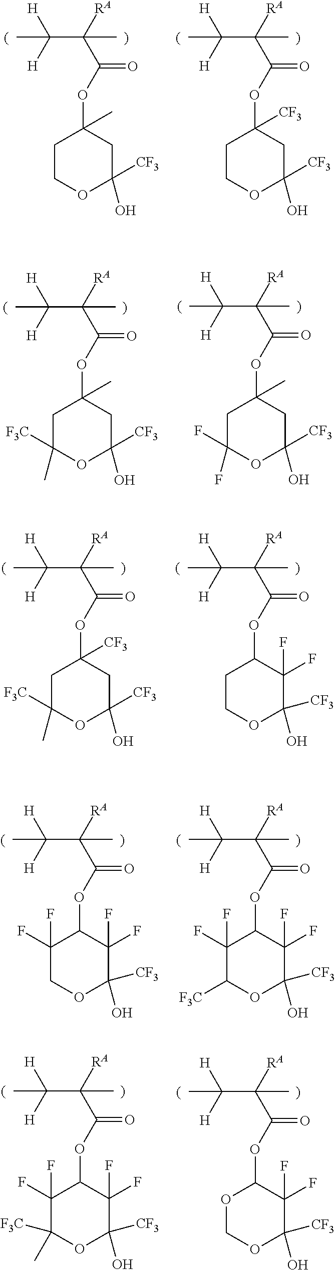

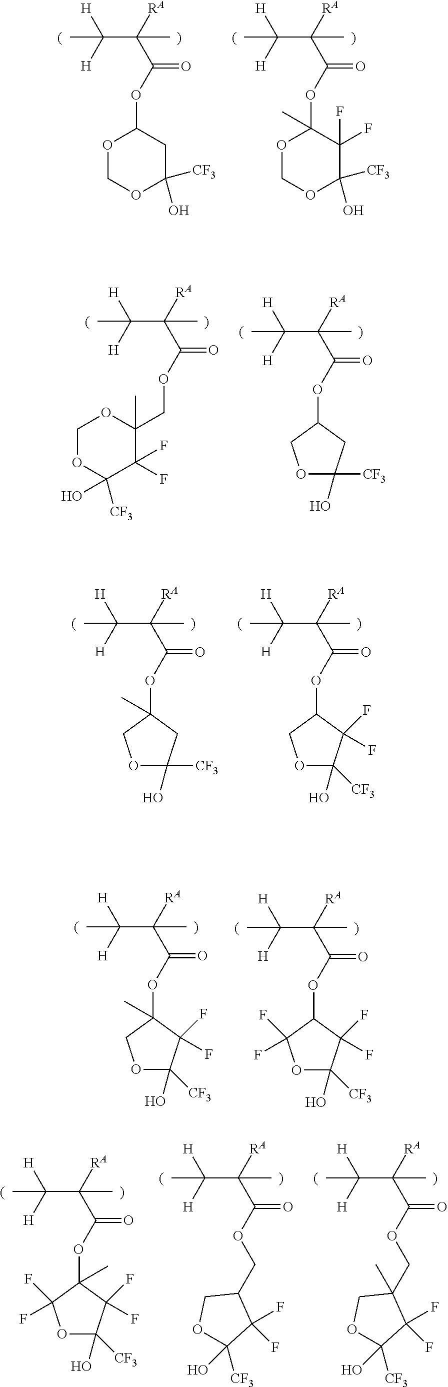

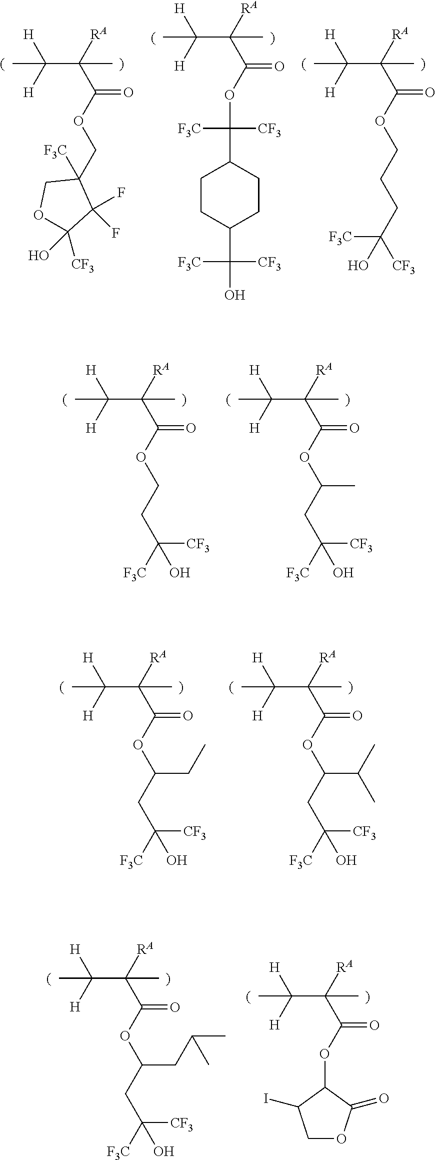

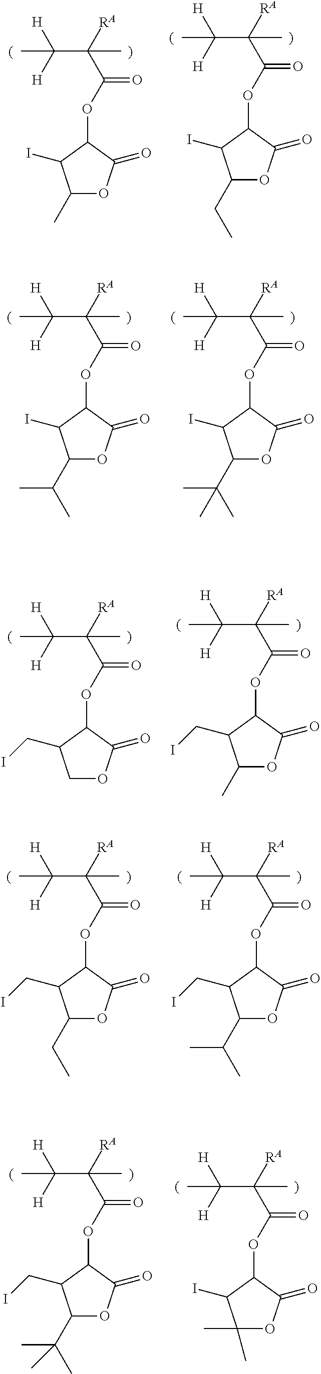

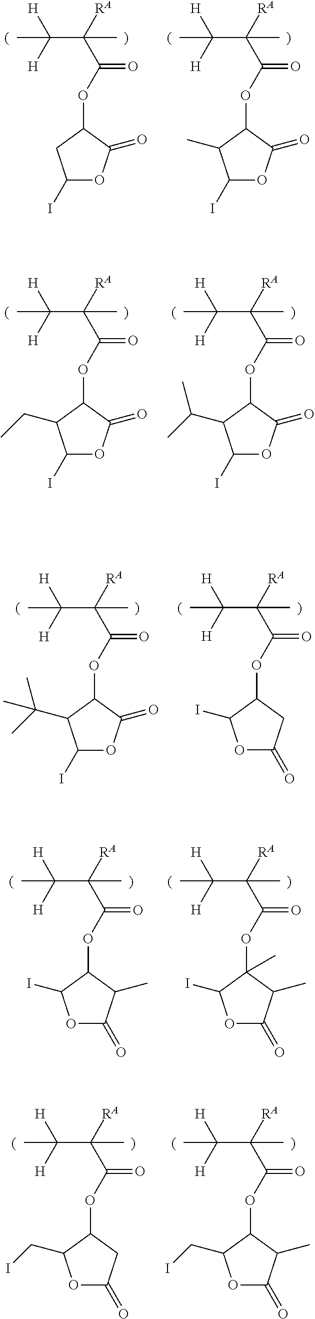

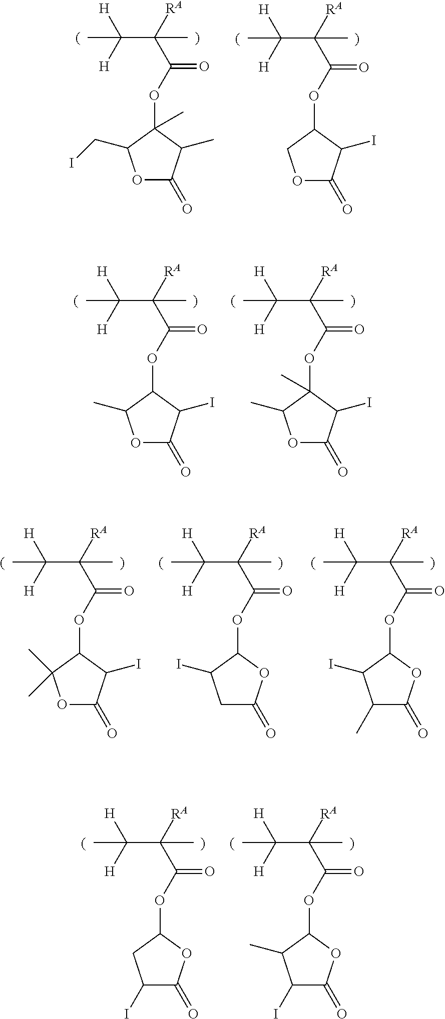

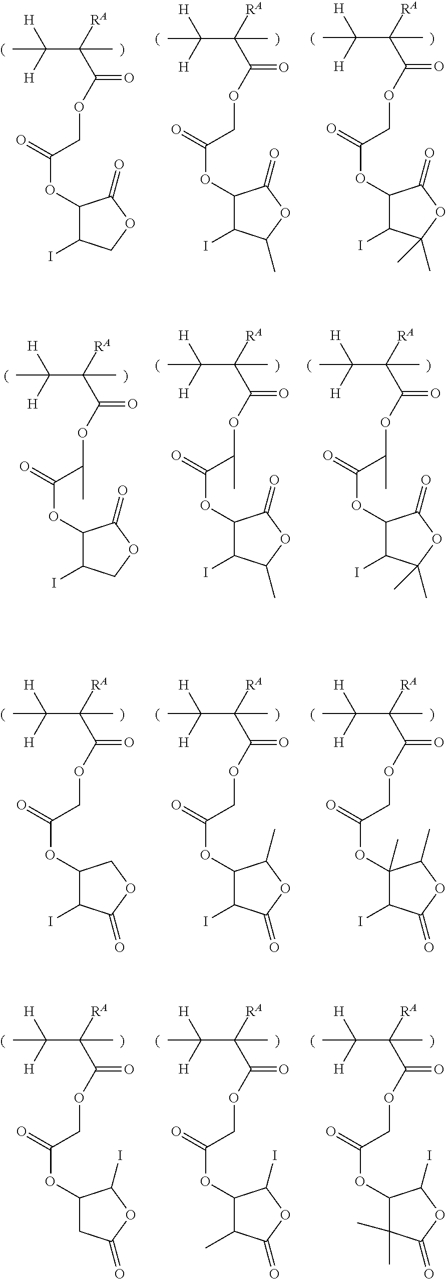

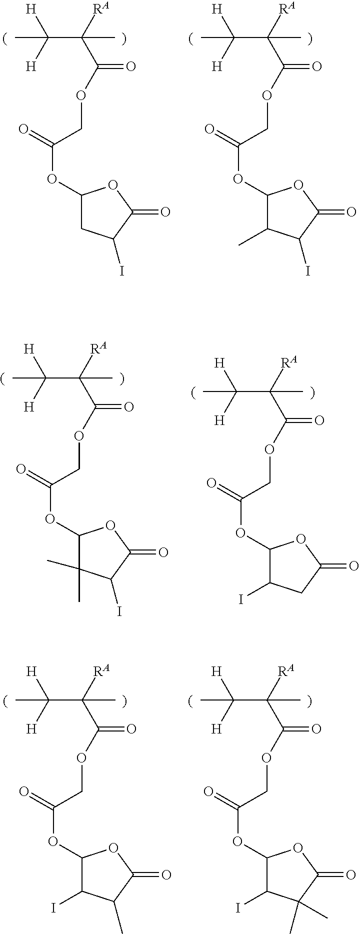

Examples of the repeat unit (b2) are shown below, but not limited thereto. Herein R^{A} is as defined above.

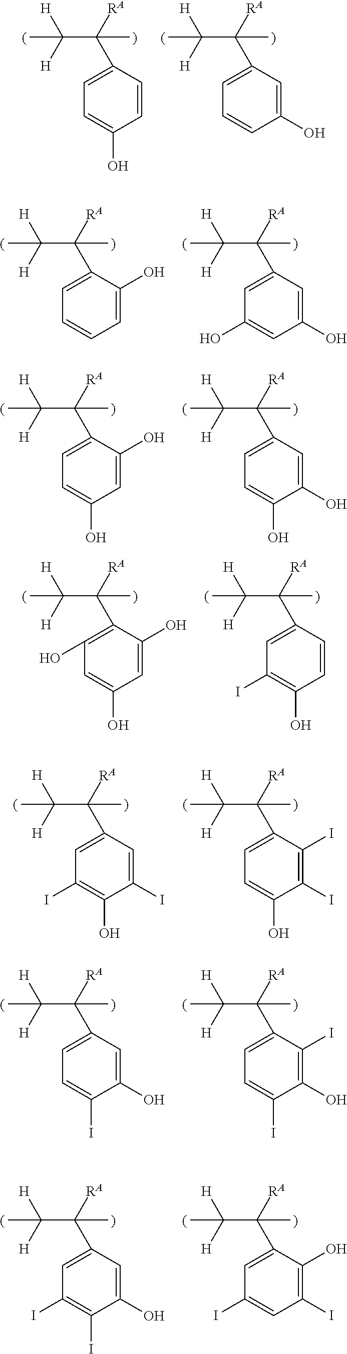

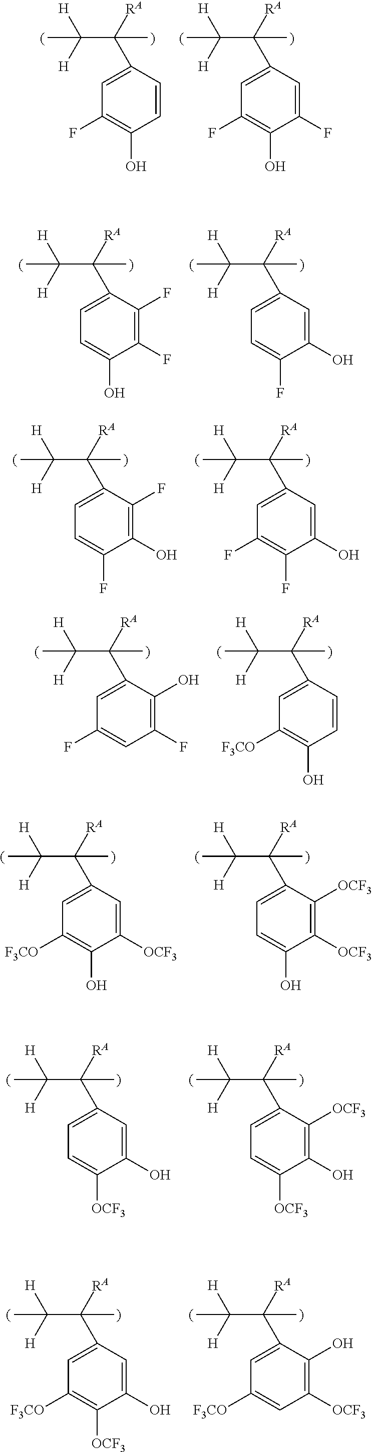

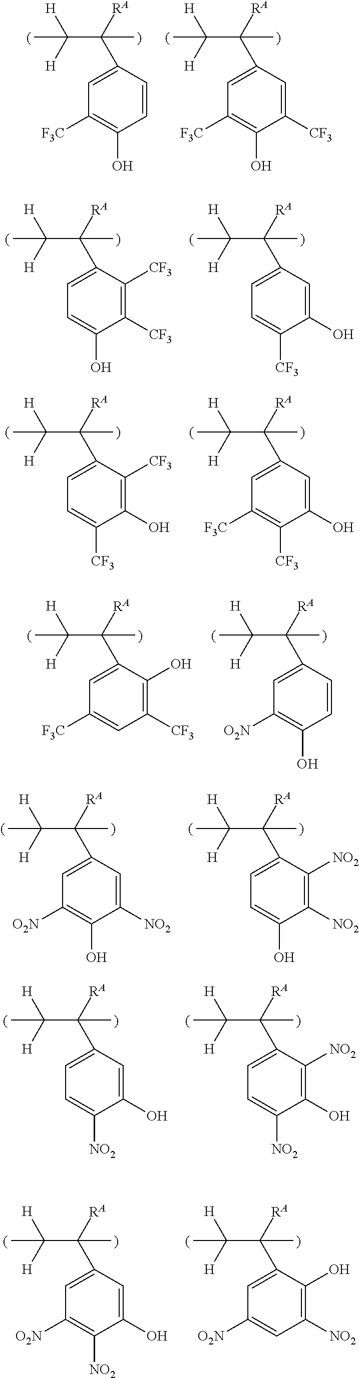

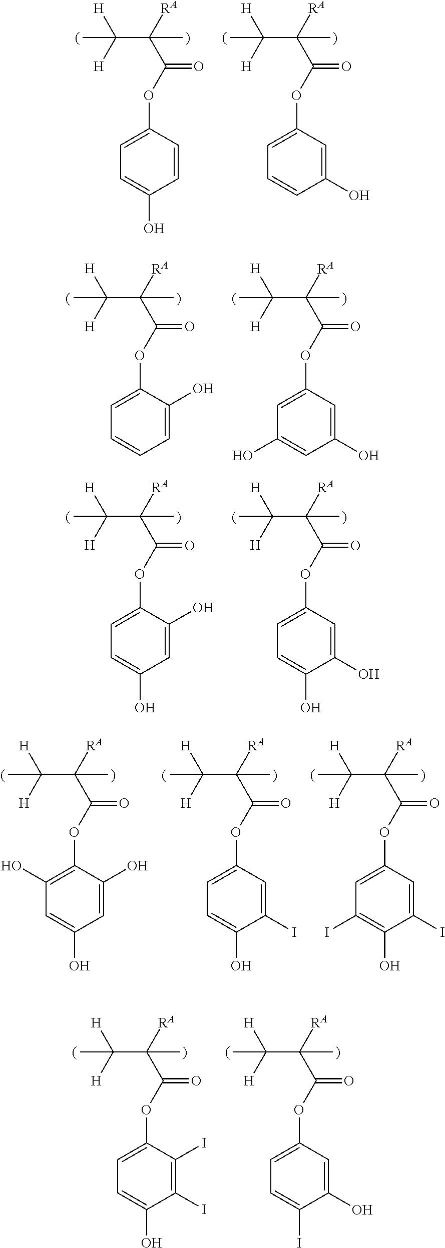

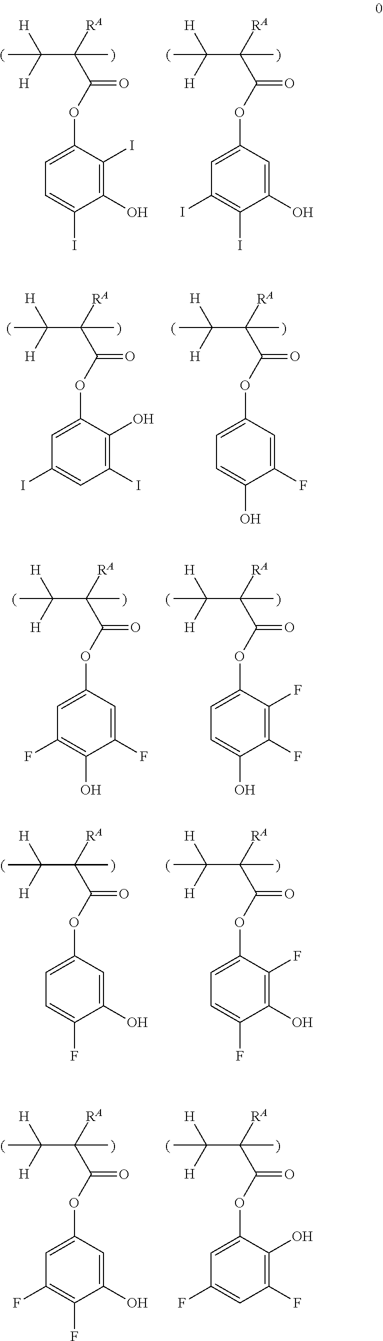

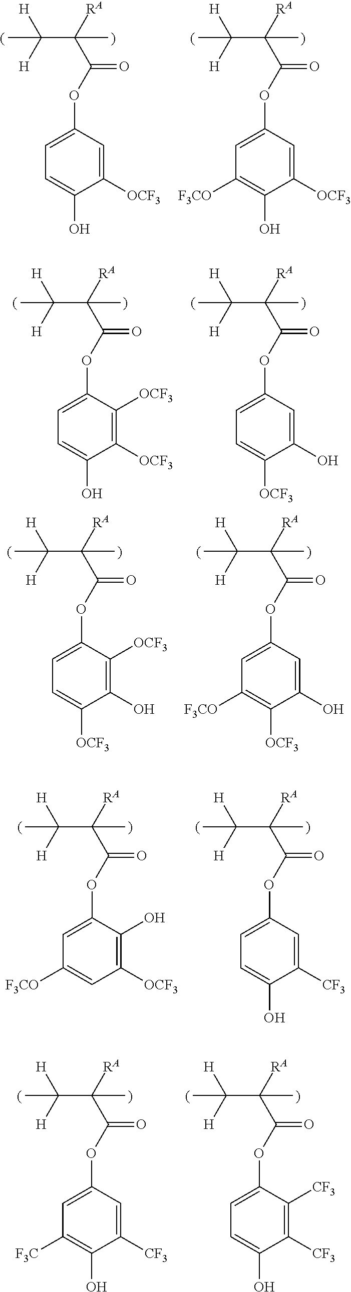

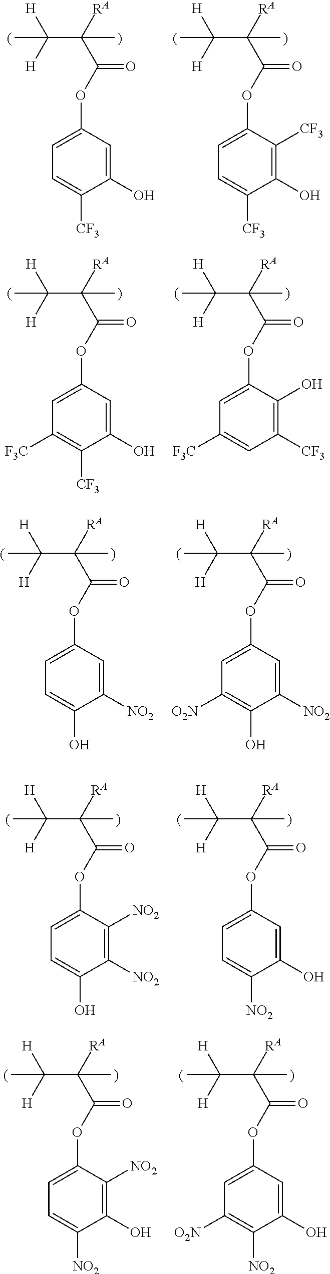

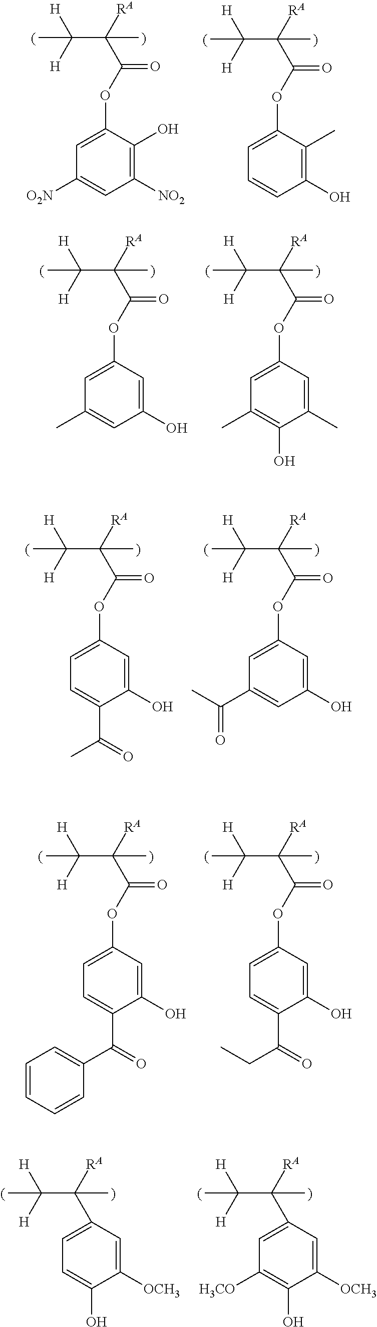

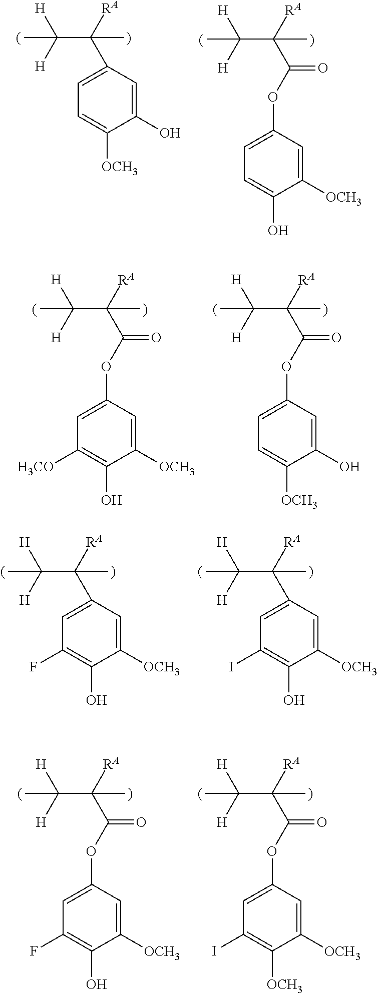

Of the repeat units (b1) and (b2), those units having a lactone ring as the polar group are preferred in the ArF lithography and those units having a phenolic site are preferred in the KrF, EB and EUV lithography.

The polymer may further comprise repeat units of at least one type selected from repeat units having the formulae (c1) to (c4), which are simply referred to as repeat units (c1) to (c4). These repeat units function as a photoacid generator bound to the polymer backbone.

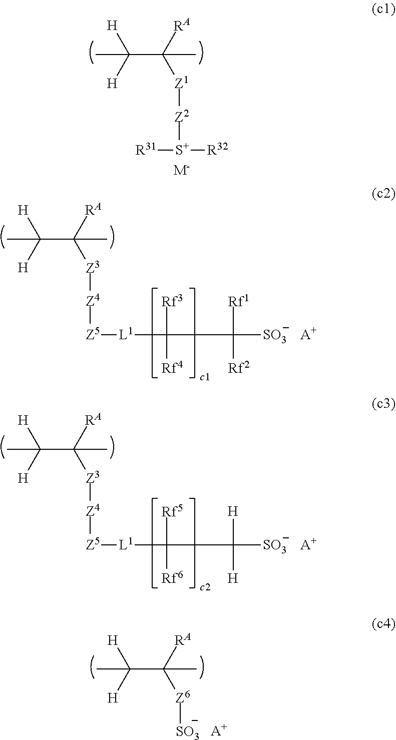

In formulae (c1) to (c4), R^{A} is each independently hydrogen, fluorine, methyl or trifluoromethyl. Z¹ is a single bond or phenylene group. Z² is **—C(═O)—O—Z²¹—, **—C(═O)—N(H)—Z²¹— or **—O—Z²¹—. Z²¹ is a C₁-C₆ aliphatic hydrocarbylene group, a phenylene group or a divalent group obtained by combining the foregoing, which may contain a carbonyl moiety, ester bond, ether bond or hydroxy moiety. Z³ is each independently a single bond, phenylene, naphthylene, or *—C(═O)—Z³¹—. Z³¹ is a C₁-C₁₀ aliphatic hydrocarbylene group which may contain a hydroxy moiety, ether bond, ester bond or lactone ring, or phenylene or naphthylene group. Z⁴ is each independently a single bond, ***—Z⁴¹—C(═O)—O—, ***—C(═O)—NH—Z⁴¹—, or ***—O—Z⁴¹. Z⁴¹ is a C₁-C₂₀ hydrocarbylene group which may contain a heteroatom. Z⁵ is each independently a single bond, ****—Z⁵¹—C(═O)—O—, ****—C(═O)—NH—Z⁵¹—, or ****—O—Z⁵¹. Z⁵¹ is a C₁-C₂₀ hydrocarbylene group which may contain a heteroatom. Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹—, or *—O—Z⁶¹. Z⁶¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety. Herein,*designates a point of attachment to the carbon atom in the backbone, ** designates a point of attachment to Z¹, *** designates a point of attachment to Z³, and **** designates a point of attachment to Z⁴.

The aliphatic hydrocarbylene group represented by Z²¹, Z³¹ and Z⁶¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene group represented by Z⁴¹ and Z⁵¹ may be saturated or unsaturated and straight, branched or cyclic. Examples thereof are shown below, but not limited thereto.

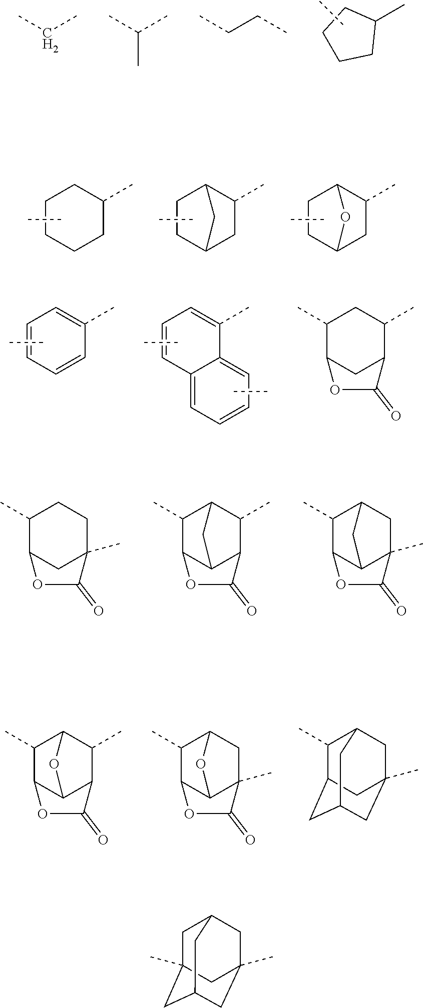

In formula (c1), R³¹ and R³² are each independently a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl, naphthyl and thienyl; C₇-C₂₀ aralkyl groups such as benzyl, 1-phenylethyl, and 2-phenylethyl, and combinations thereof. Of these, aryl groups are preferred. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

R³¹ and R¹² may bond together to form a ring with the sulfur atom to which they are attached. Examples of the ring are as will be exemplified later for the ring that R^{ct1} and R^{ct2} in formula (cation-1) form with the sulfur atom to which they are attached.

Examples of the cation in repeat unit (c1) are given below, but not limited thereto. Herein R^{A} is as defined above.

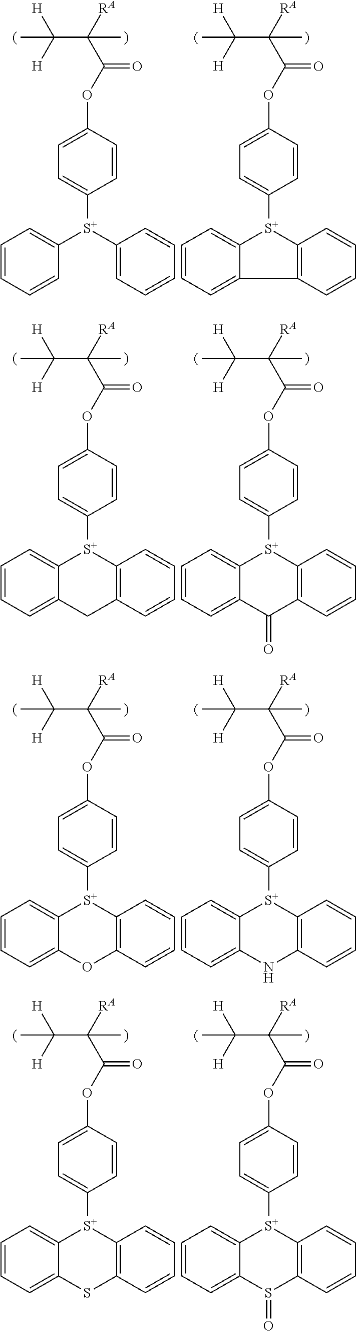

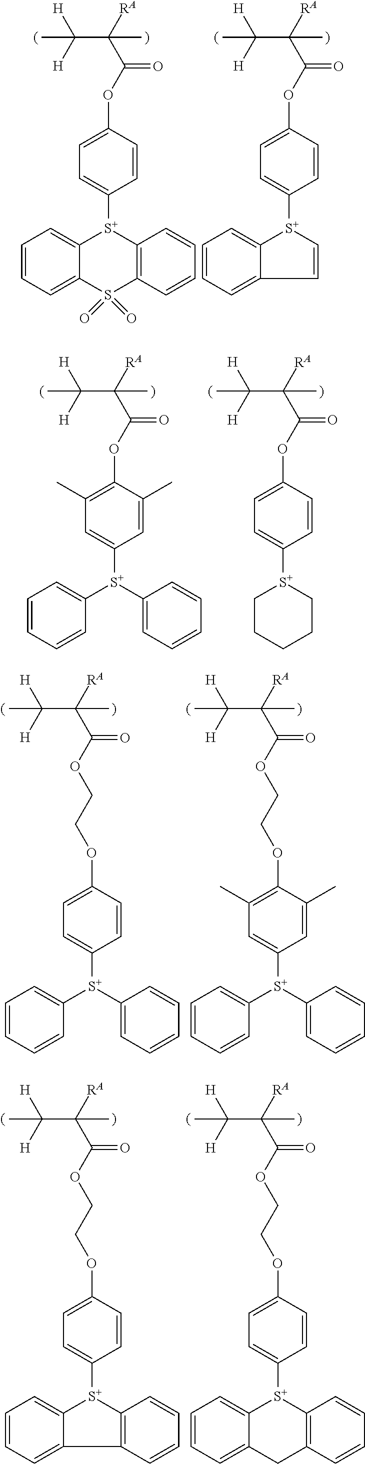

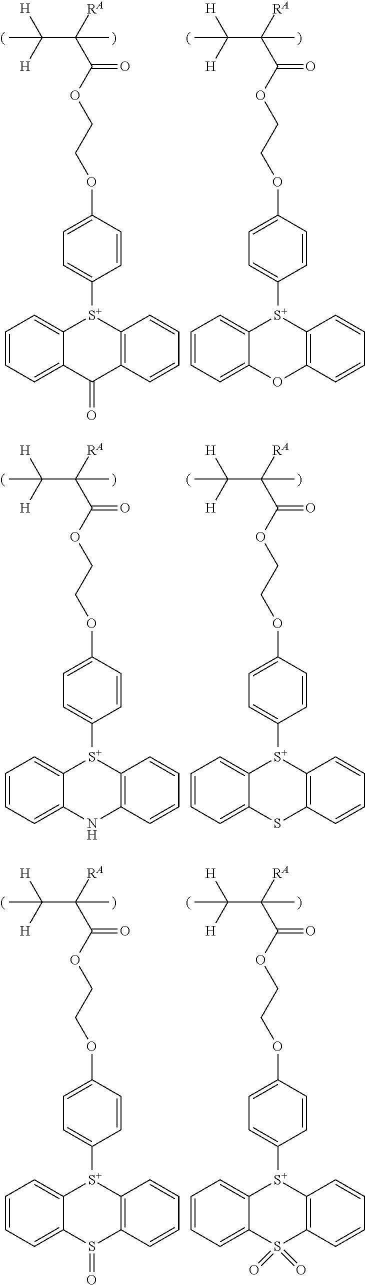

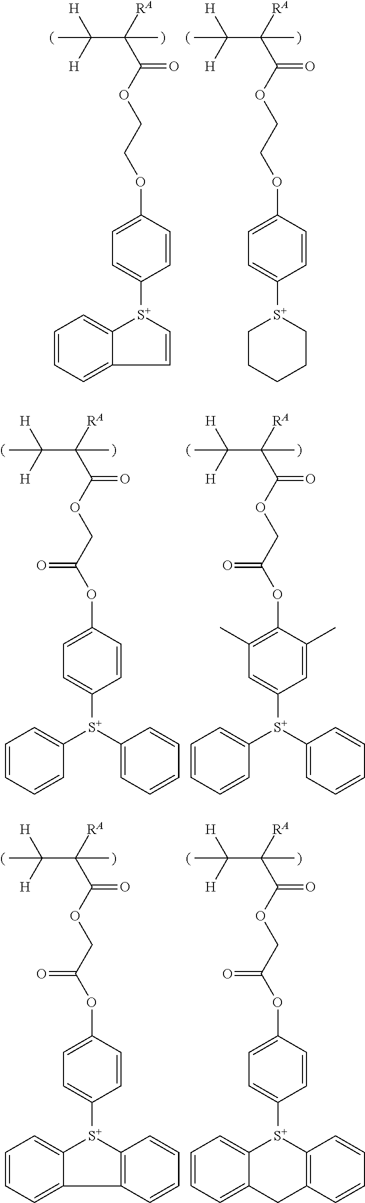

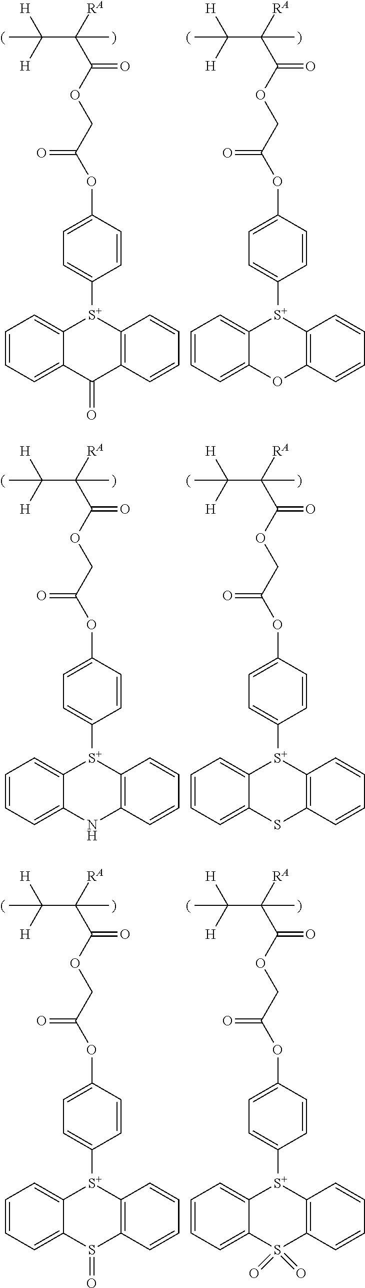

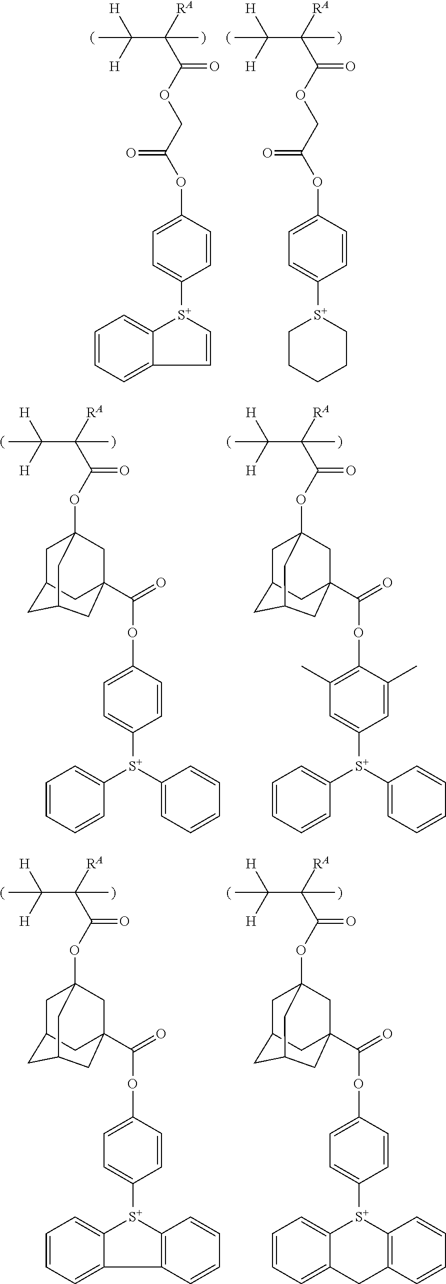

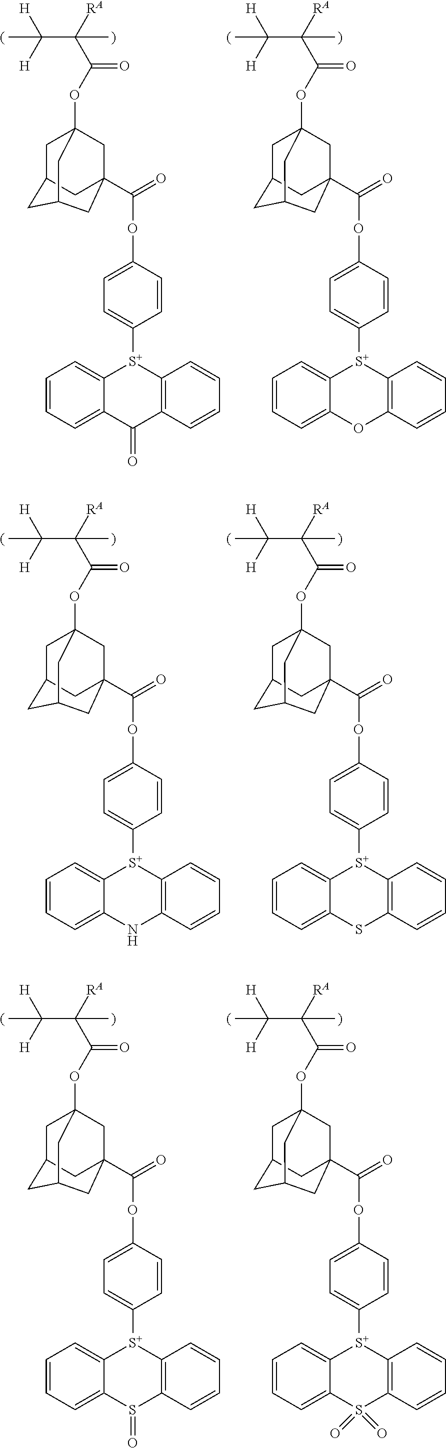

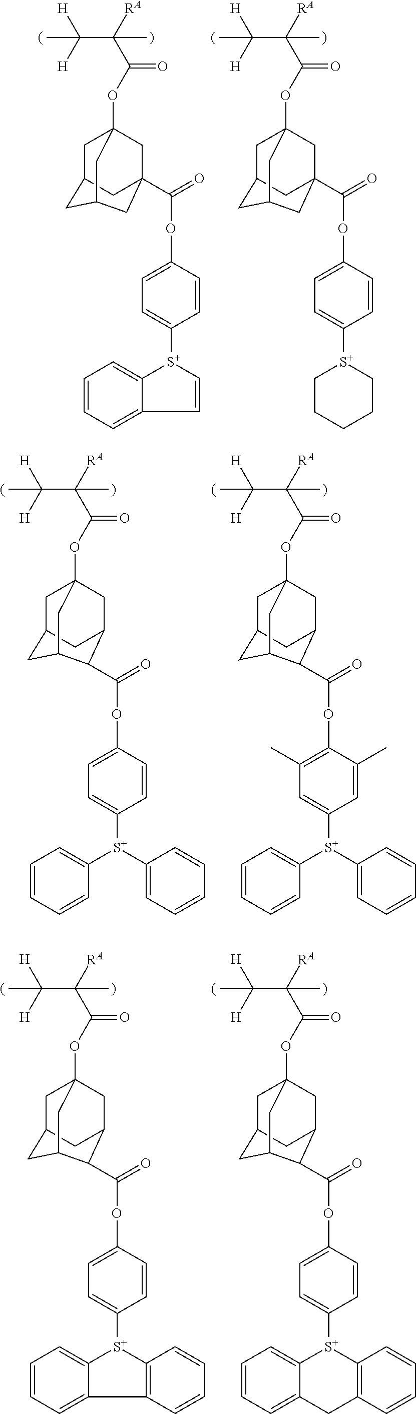

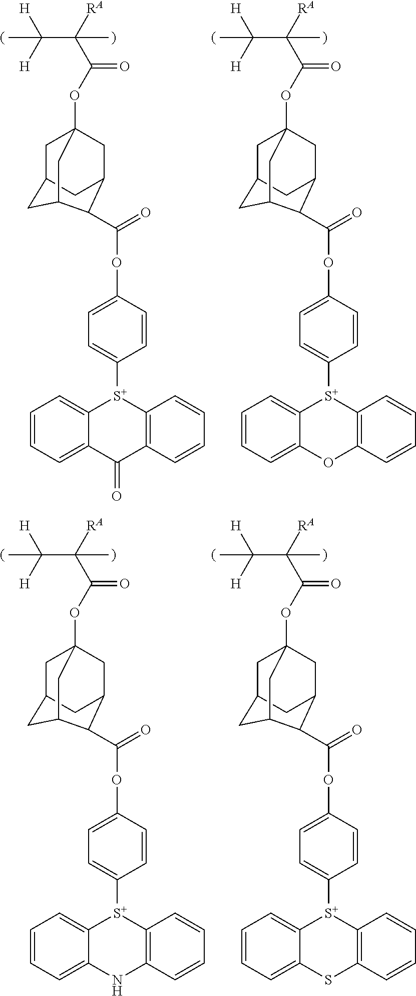

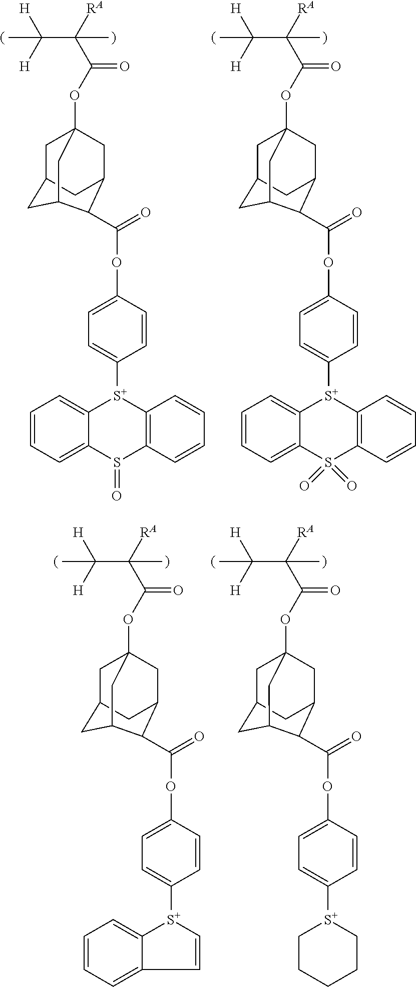

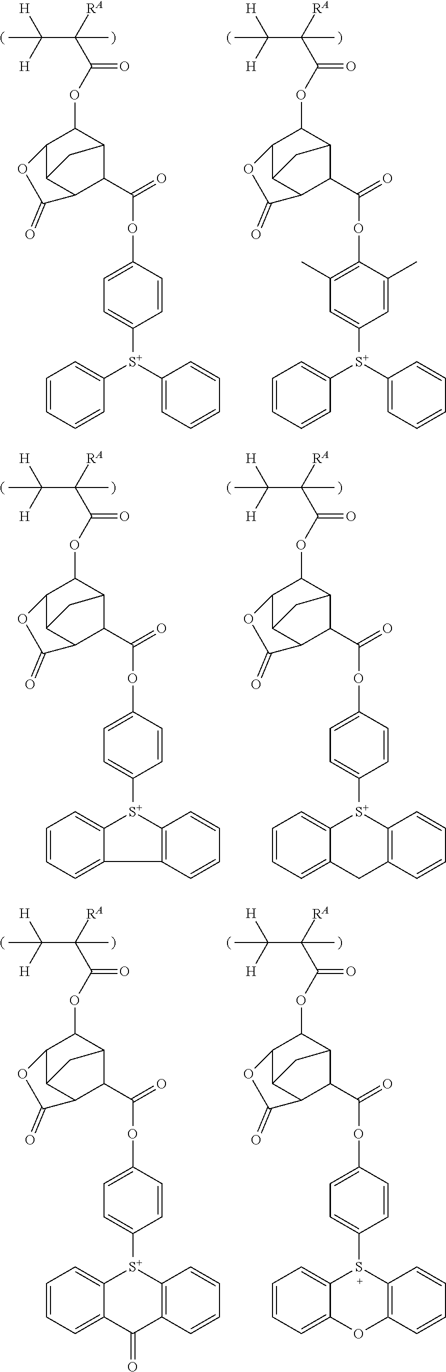

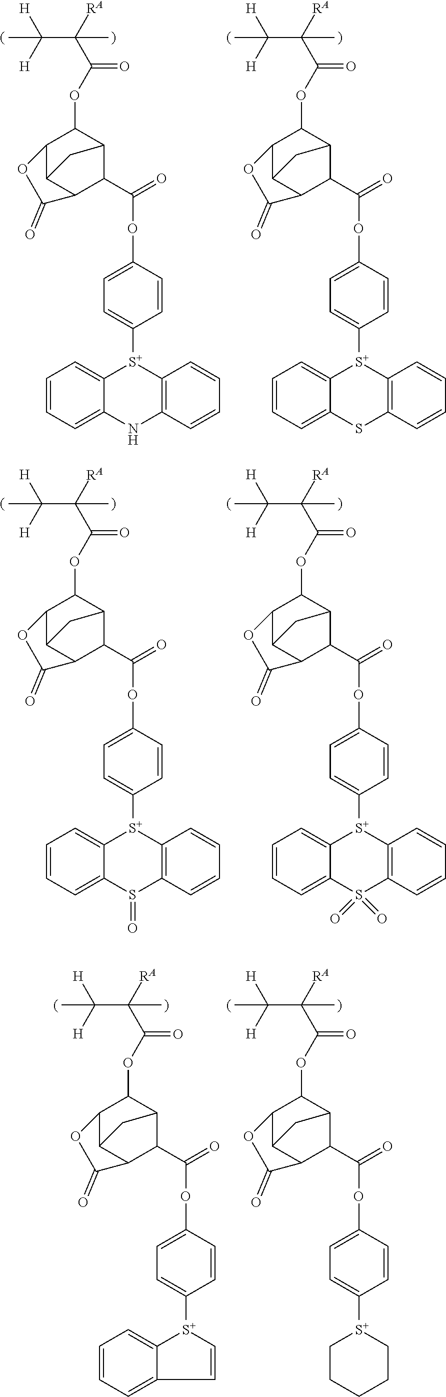

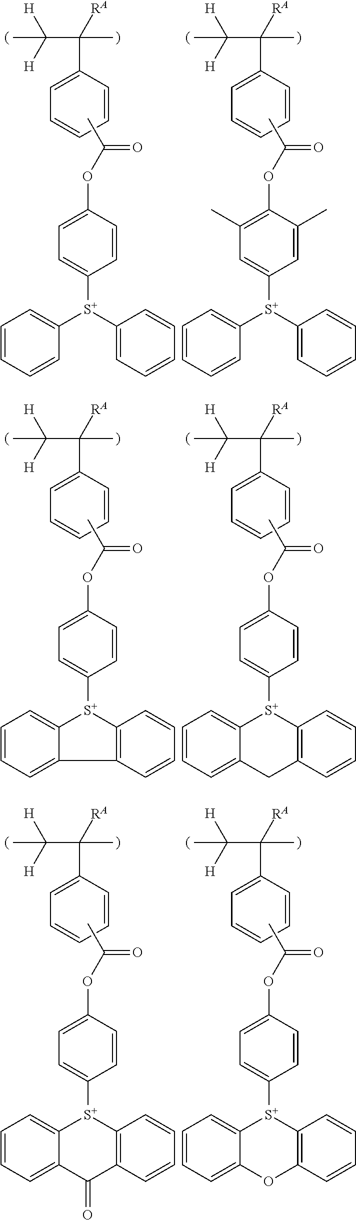

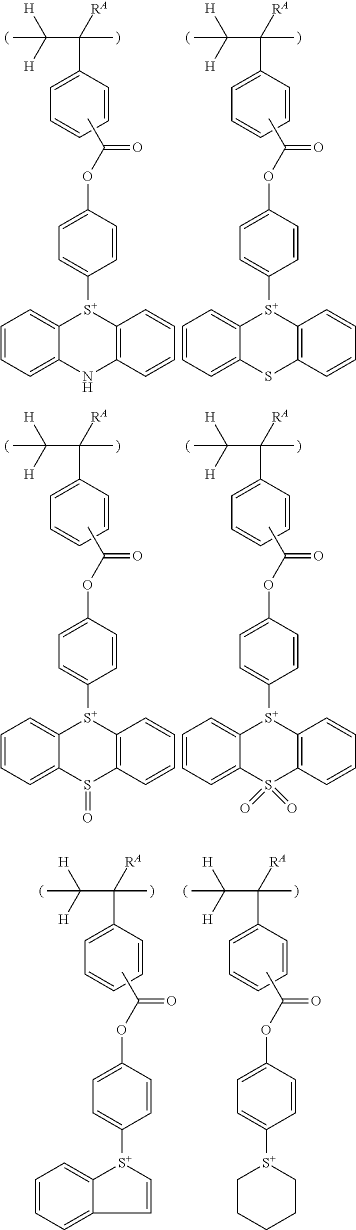

In formula (c1), M⁻ is a non-nucleophilic counter ion. Halide, sulfonate, imide and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Anions having the following formulae (c1-1) to (c1-4) are also useful as the non-nucleophilic counter ion.

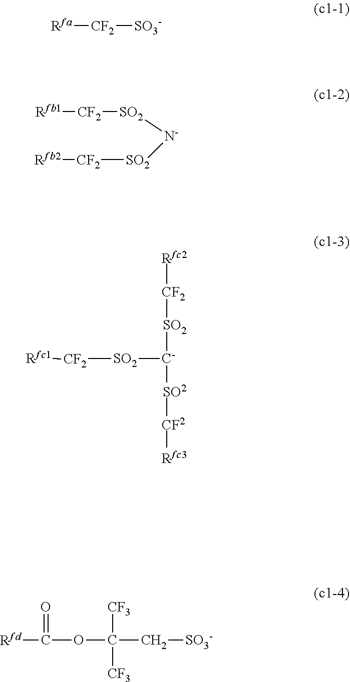

In formula (c1-1), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified below for the hydrocarbyl group R^{fa1} in formula (c1-1-1).

Of the anions of formula (c1-1), an anion having the formula (c1-1-1) is preferred.

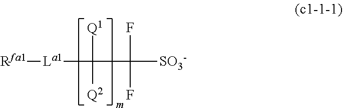

In formula (c1-1-1), Q¹ and Q² are each independently hydrogen, fluorine or a C₁-C₆ fluorinated saturated hydrocarbyl group. It is preferred for solvent solubility that at least one of Q¹ and Q² be trifluoromethyl. The subscript m is an integer of 0 to 4, most preferably 1.

R^{fa1} is a C₁-C₃₅ hydrocarbyl group which may contain a heteroatom. As the heteroatom, oxygen, nitrogen, sulfur and halogen atoms are preferred, with oxygen being most preferred. Of the hydrocarbyl groups, those groups of 6 to 30 carbon atoms are preferred from the aspect of achieving a high resolution in forming patterns of small feature size. The hydrocarbyl group may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₅ unsaturated aliphatic hydrocarbyl groups such as 2-propenyl and 3-cyclohexenyl; C₆-C₃₅ aryl groups such as phenyl, 1-naphthyl, 2-naphthyl and 9-fluorenyl; and C₇-C₃₅ aralkyl groups such as benzyl and diphenylmethyl, and combinations thereof.

In the foregoing hydrocarbyl groups, some or all hydrogen may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (c1-1-1), L^{a1} is a single bond, ether bond, ester bond, sulfonate ester bond, carbonate bond or carbamate bond. From the aspect of synthesis, an ether bond or ester bond is preferred, with the ester bond being more preferred.

Examples of the anion having formula (c1-1) are shown below, but not limited thereto. Herein Q¹ is as defined above.

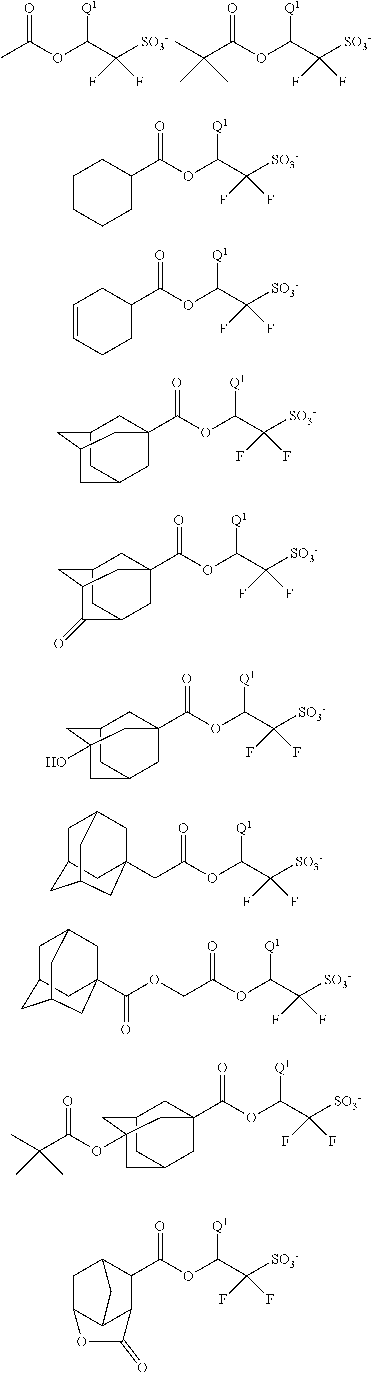

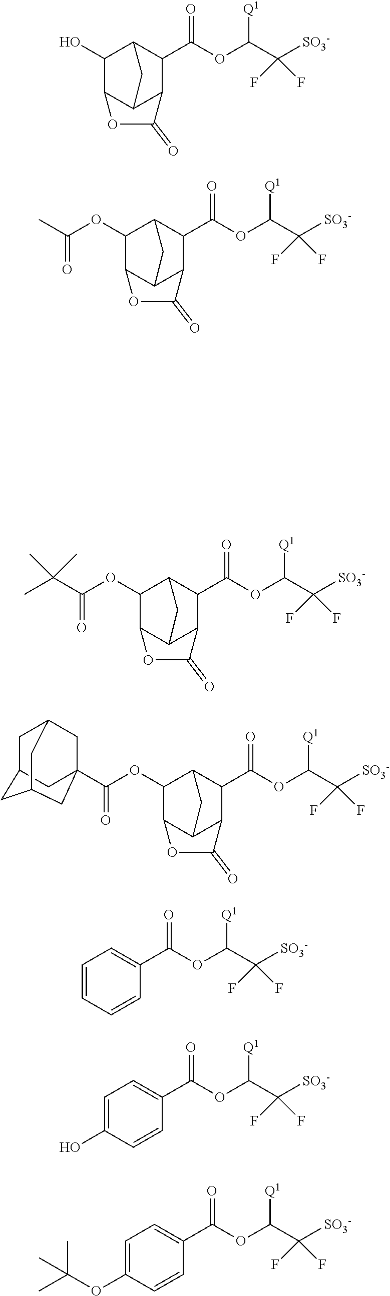

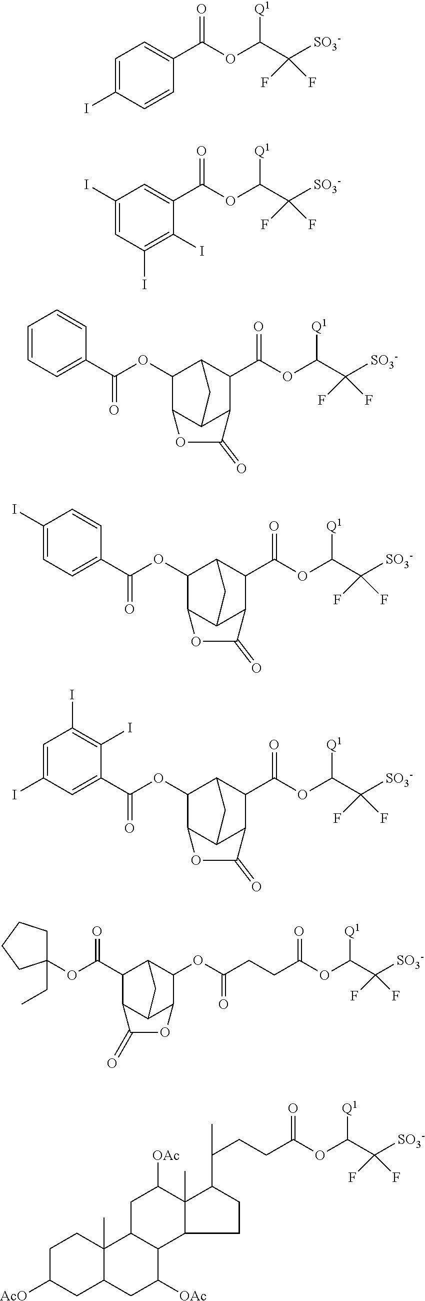

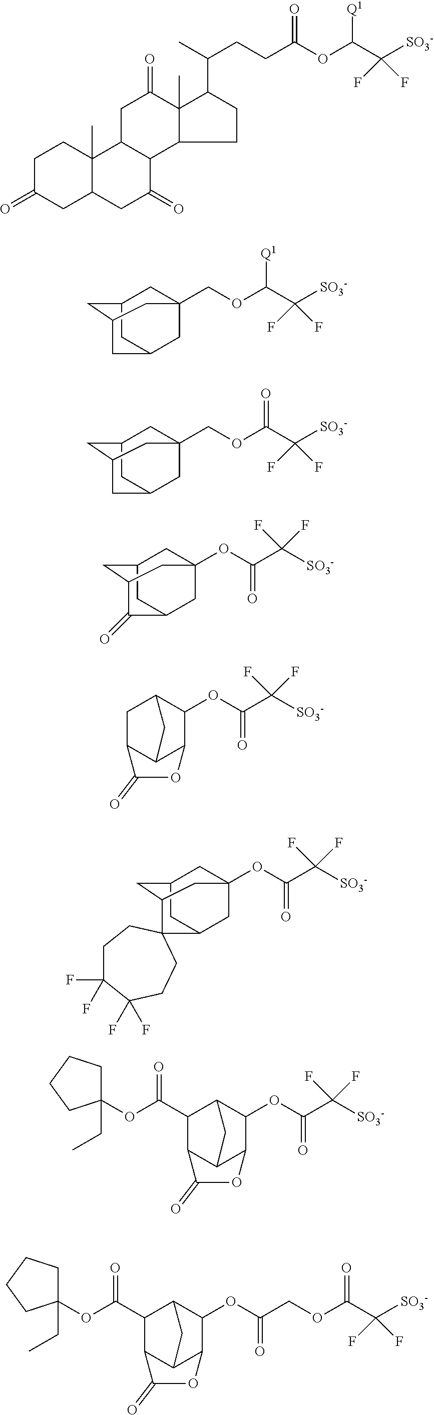

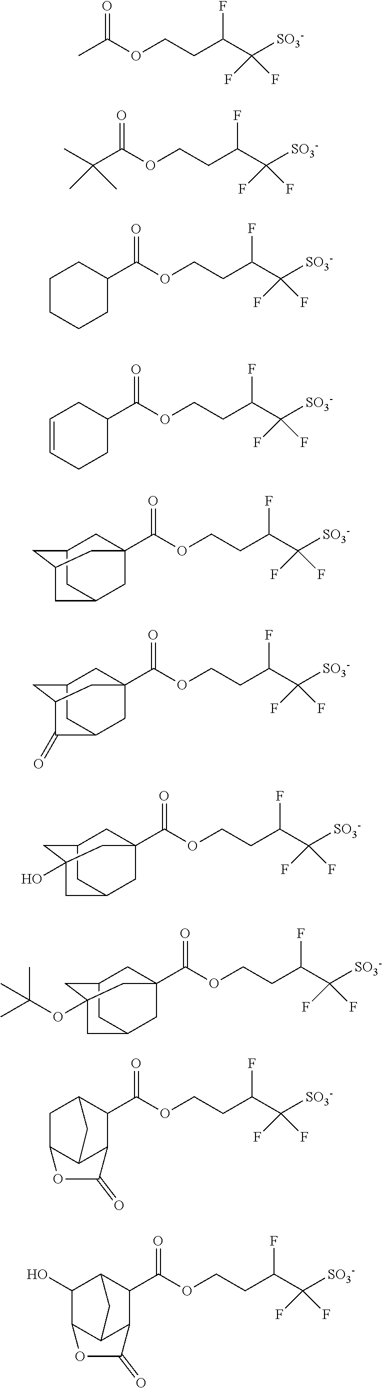

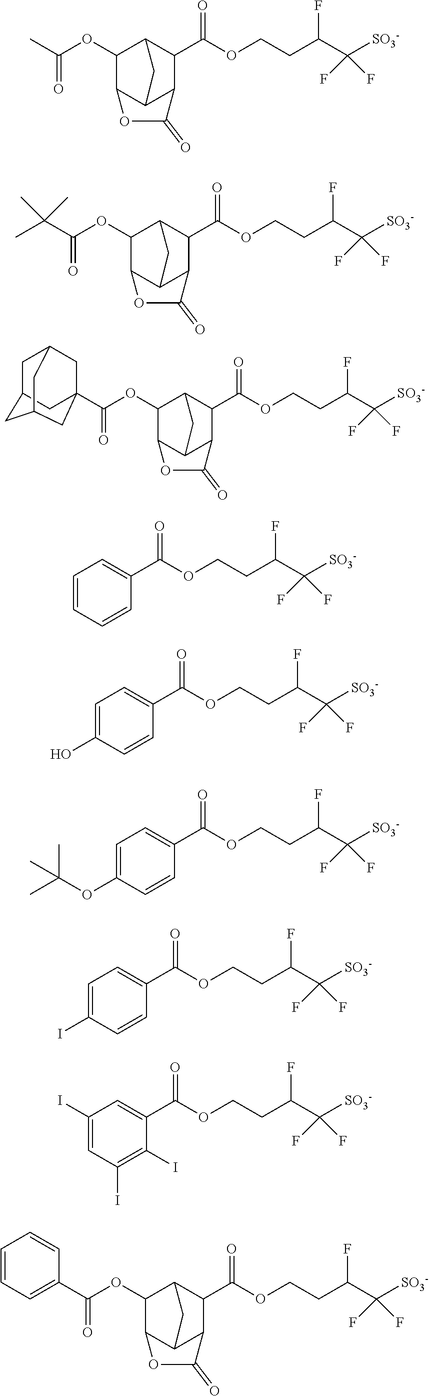

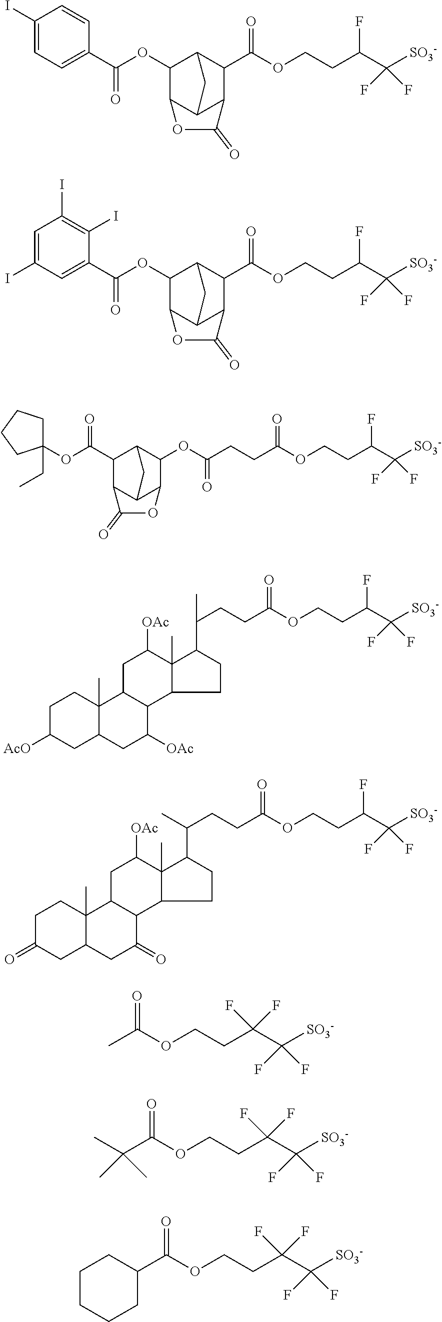

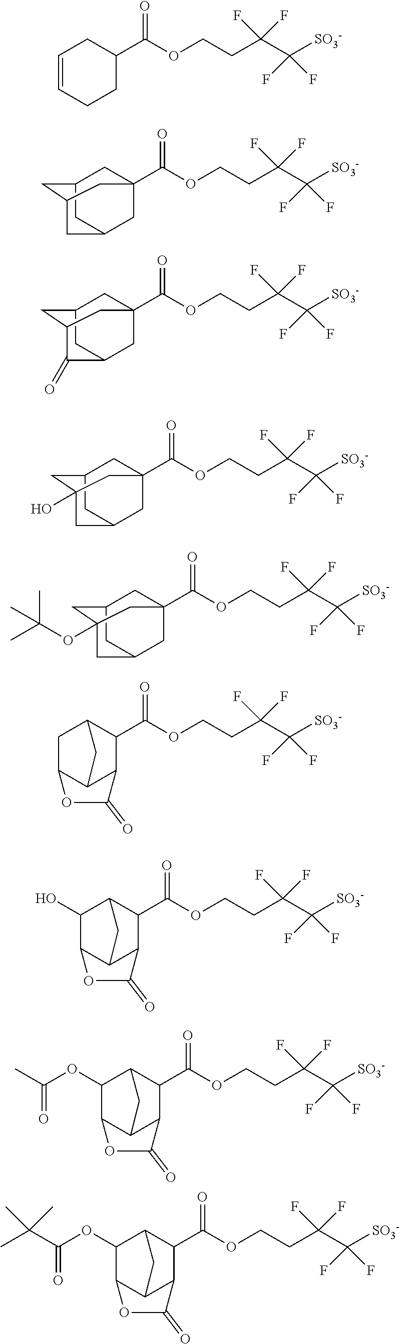

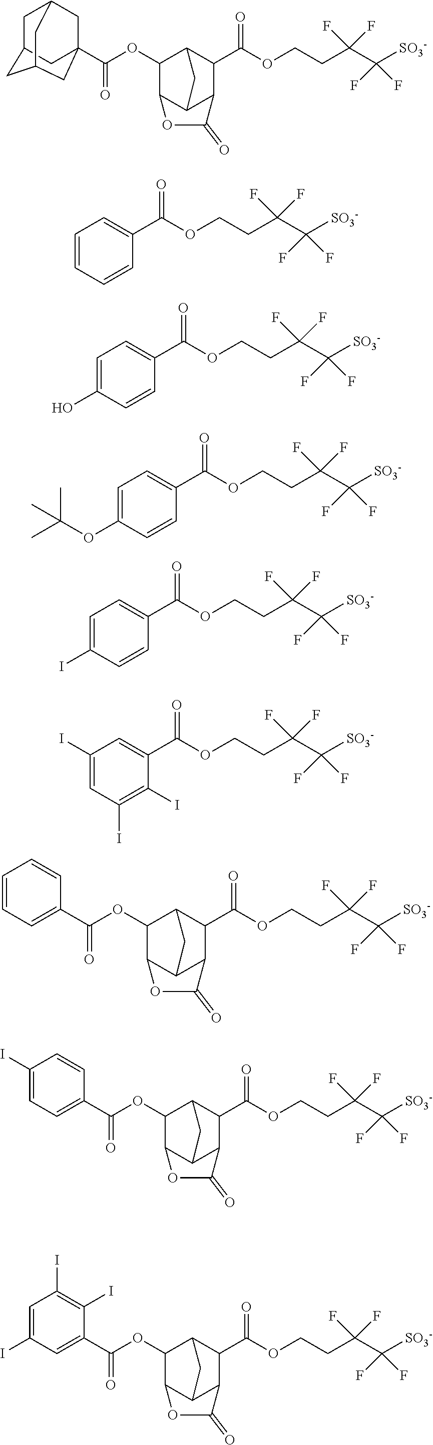

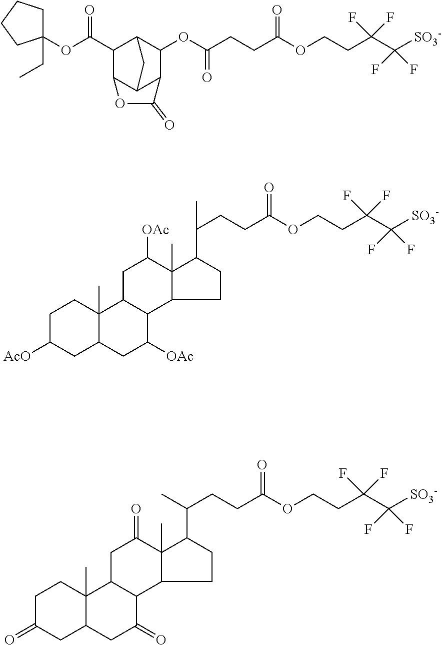

In formula (c1-2), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N—SO₂—CF₂— to which they are attached. It is preferred that a combination of R^{fb1} and R^{fb2} be a fluorinated ethylene or fluorinated propylene group.

In formula (c1-3), R^{fc1}, R^{fc2} and Rf^{fc3} are each independently fluorine or a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1} in formula (c1-1-1). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C—SO₂—CF₂— to which they are attached. It is preferred that a combination of R^{fc1} and R^{fc2} be a fluorinated ethylene or fluorinated propylene group.

In formula (c1-4), R^{fd} is a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{fa1}

Examples of the anion having formula (c1-4) are shown below, but not limited thereto.

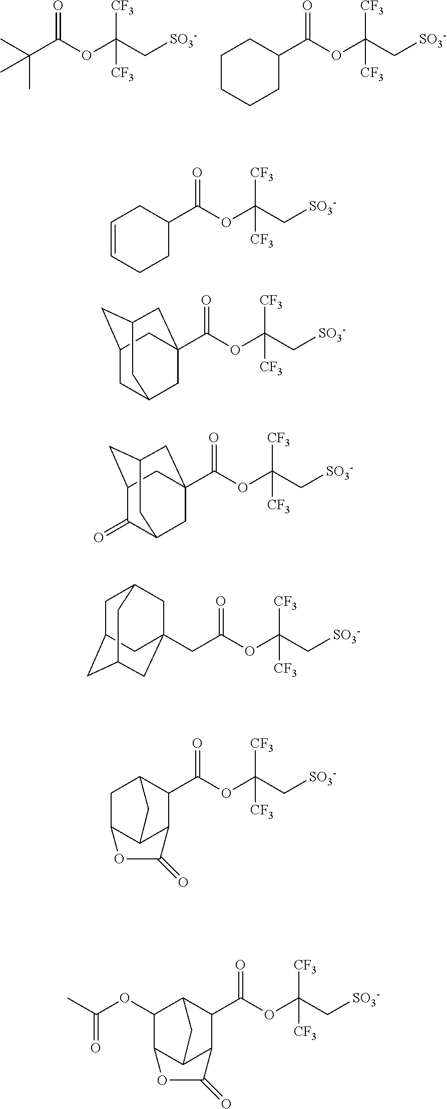

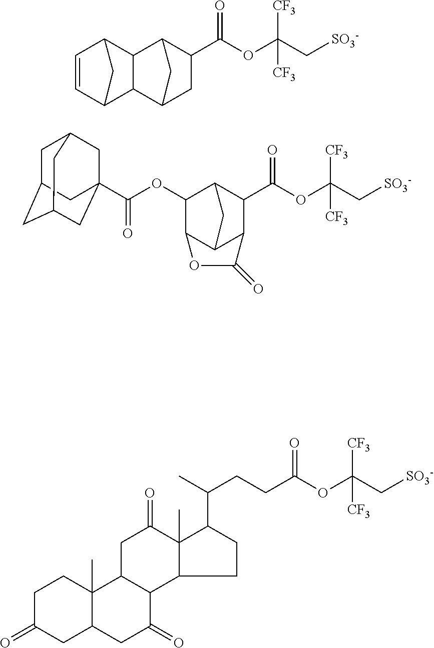

Anions having an iodized or brominated aromatic ring are also useful as the non-nucleophilic counter ion. These anions have the formula (c1-5).

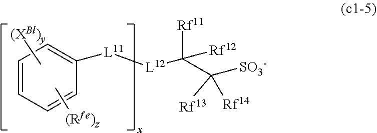

In formula (c1-5), x is an integer of 1 to 3, y is an integer of 1 to 5, z is an integer of 0 to 3, and y+z is from 1 to 5. Preferably, y is 1, 2 or 3, more preferably 2 or 3, and z is an integer of 0 to 2.

X^{BI} is iodine or bromine. A plurality of X^{BI} may be identical or different when x and/or y is 2 or more.

L¹¹ is a single bond, ether bond, ester bond, or a C₁-C₆ saturated hydrocarbylene group which may contain an ether bond or ester bond. The saturated hydrocarbylene group may be straight, branched or cyclic.

L¹² is a single bond or a C₁-C₂₀ divalent linking group when x=1, or a C₁-C₂₀ (x+1)-valent linking group when x=2 or 3. The linking group may contain an oxygen, sulfur or nitrogen atom.

R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, hydroxy, amino or ether bond, or —N(R^{fcA})(R^{fcB}), N(R^{fcC})—C(═O)—R^{fcD} or —N(R^{fcC})—C(═O)—O—R^{fcD}. R^{fcA} and R^{fcB} are each independently hydrogen or a C₁-C₆ saturated hydrocarbyl group. R^{fcC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{fcD} is a C₁-C₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{fcC})—C(═O)—R^{fcD}, —N(R^{fcC})—C(═O)—R^{fcD}, fluorine, chlorine, bromine, methyl or methoxy.

Rf¹¹ to Rf¹⁴ are each independently hydrogen, fluorine or trifluoromethyl, at least one of Rf¹¹ to Rf¹⁴ is fluorine or trifluoromethyl. Rf¹¹ and Rf¹², taken together, may form a carbonyl group. More preferably, both Rf¹³ and Rf¹⁴ are fluorine.

Examples of the anion having formula (c1-5) are shown below, but not limited thereto. X^{BI} is as defined above.

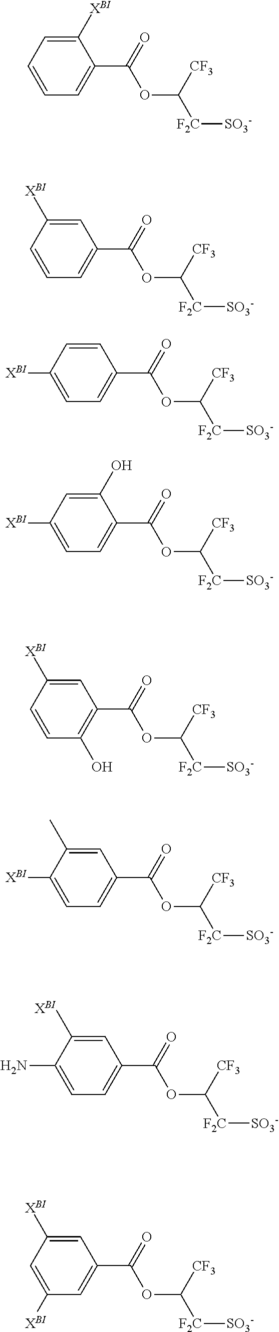

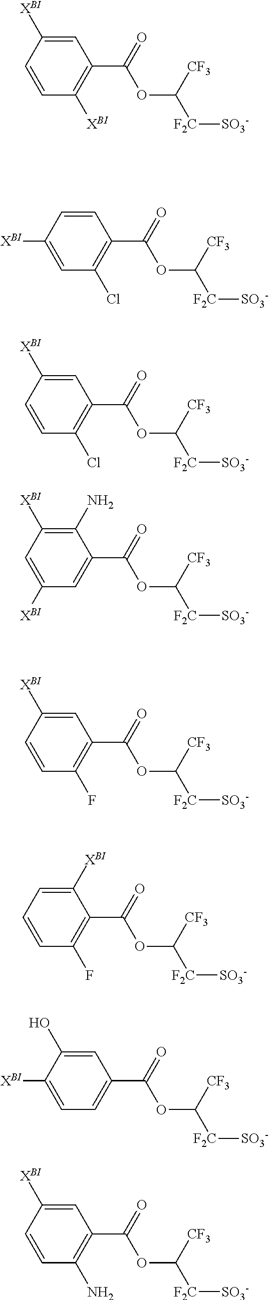

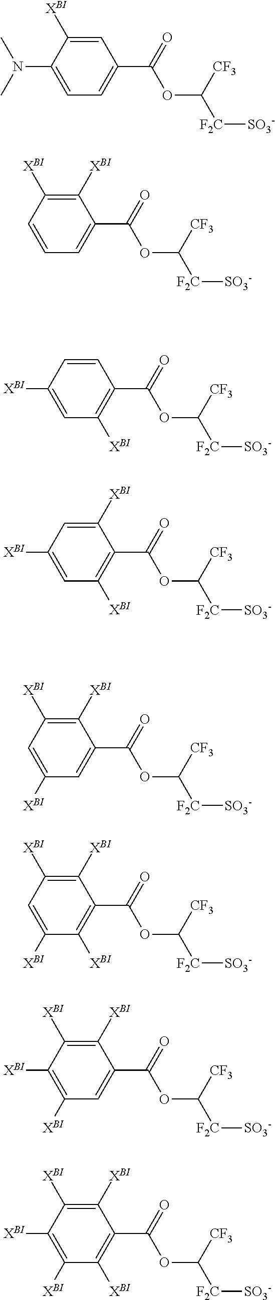

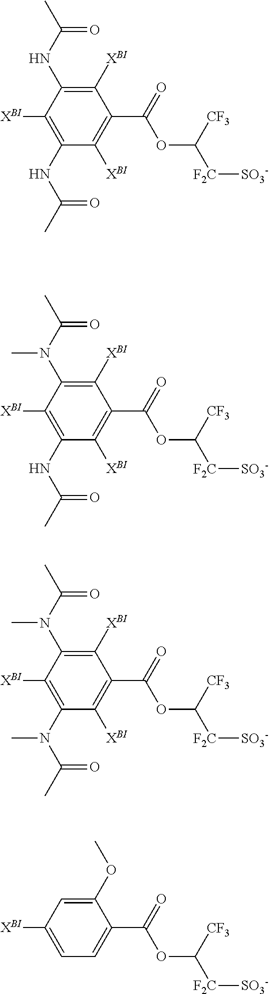

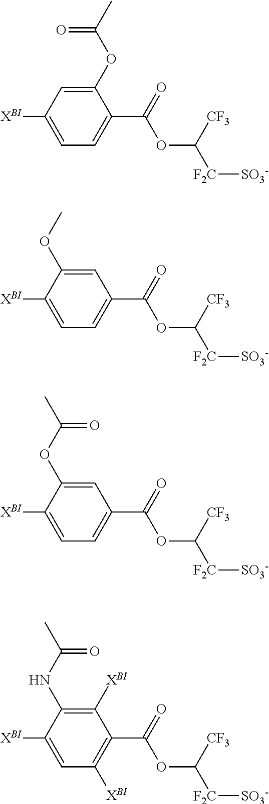

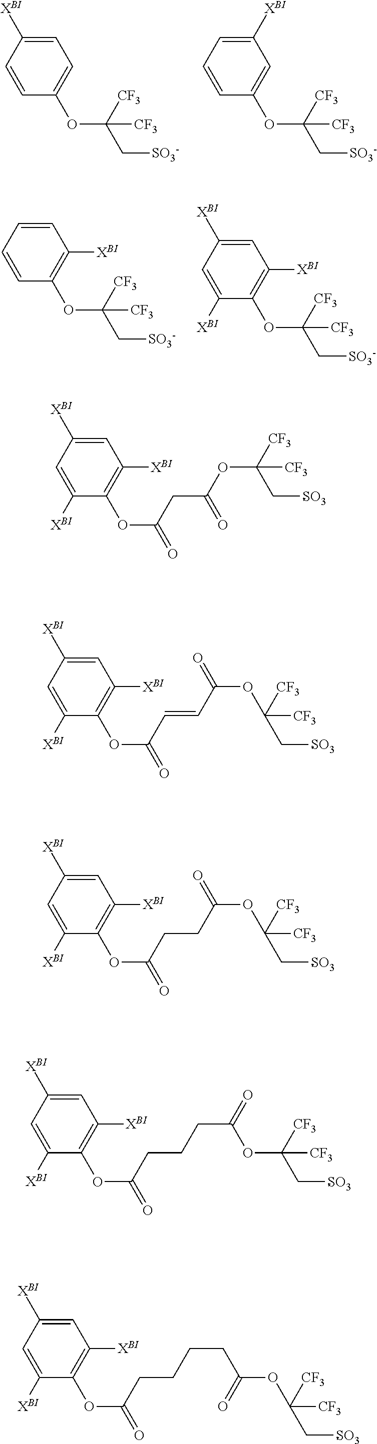

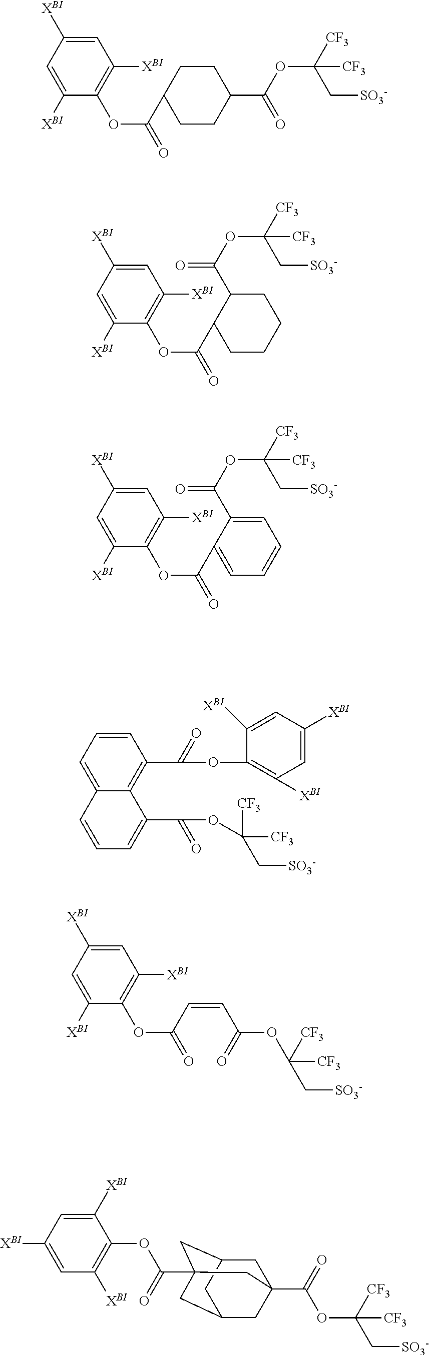

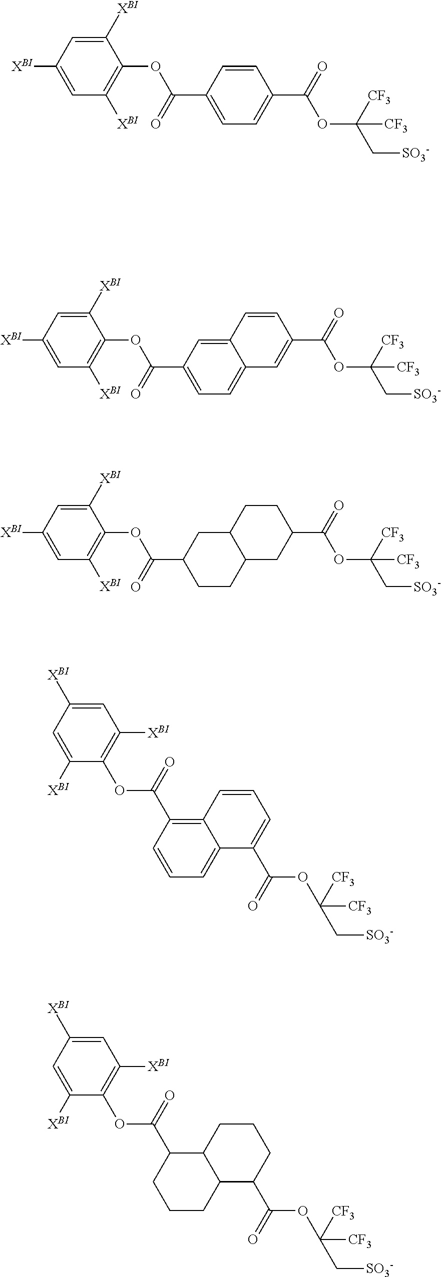

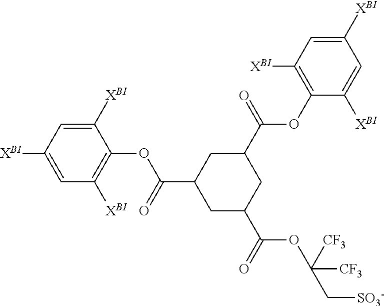

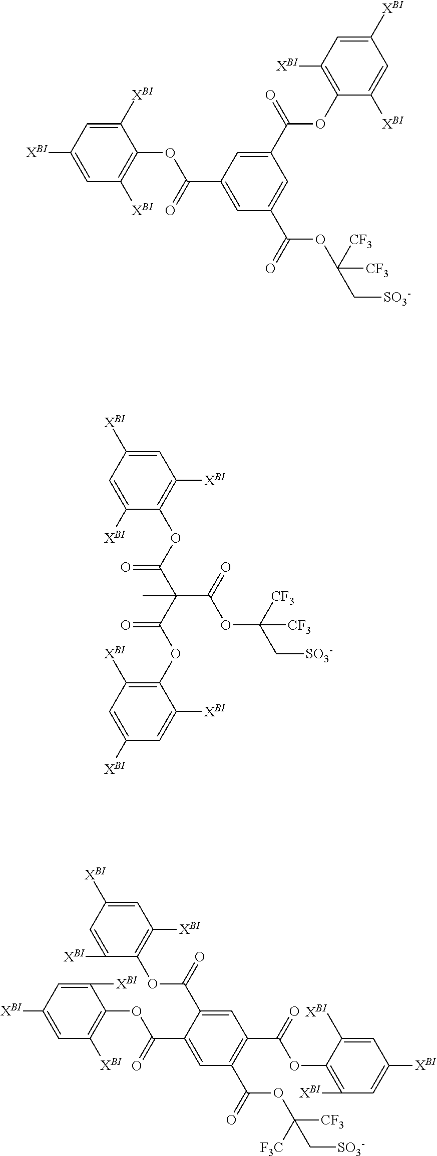

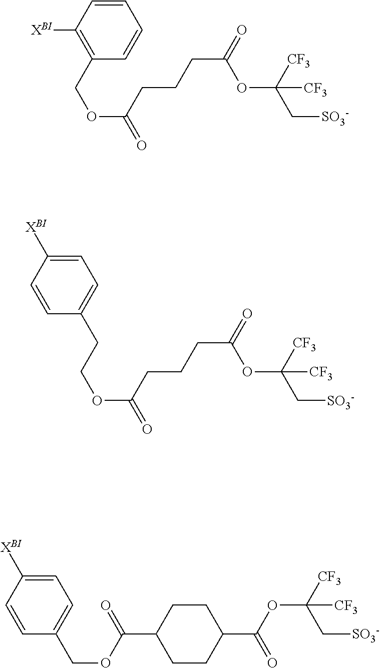

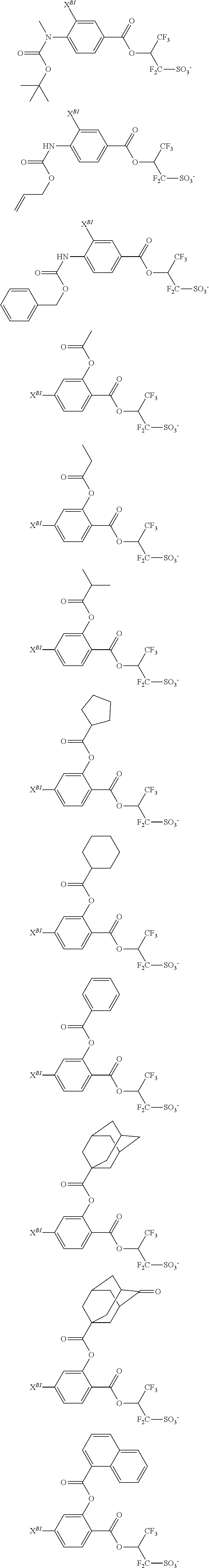

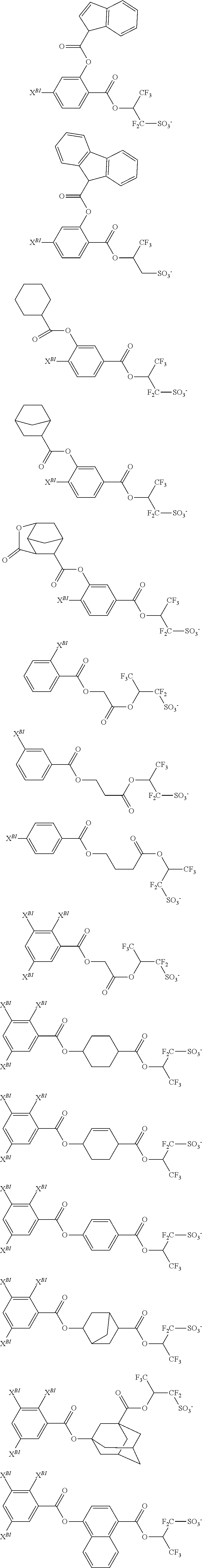

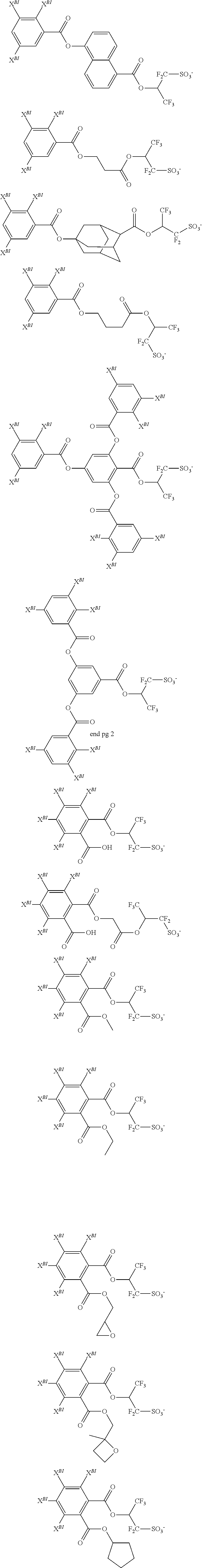

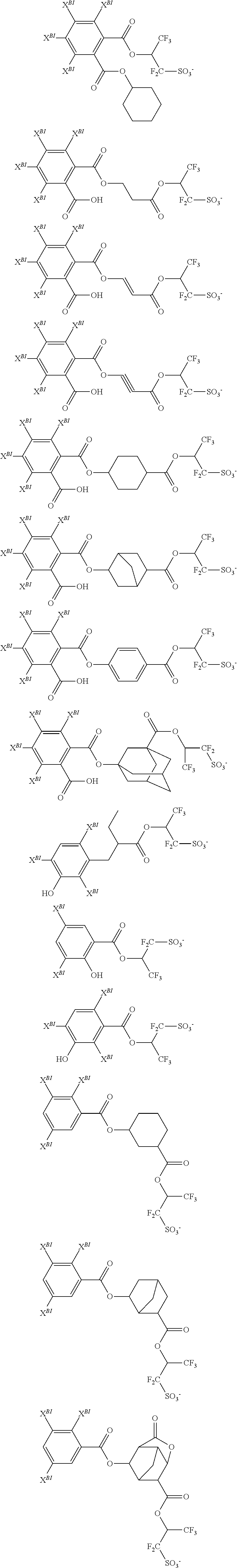

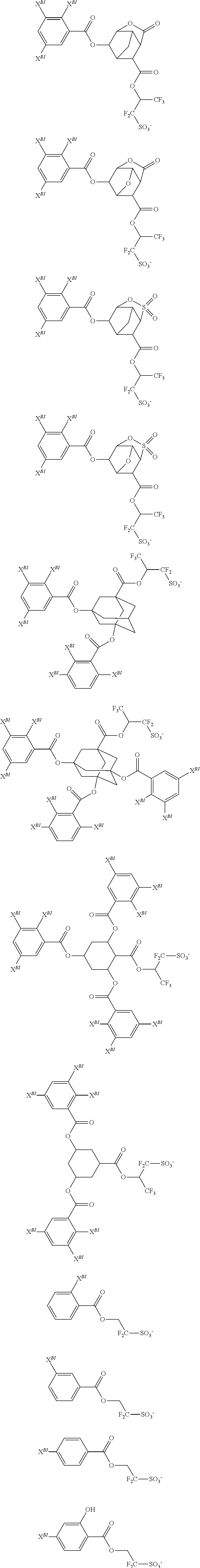

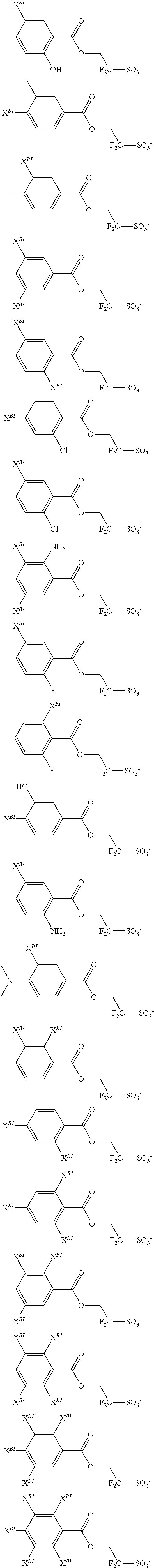

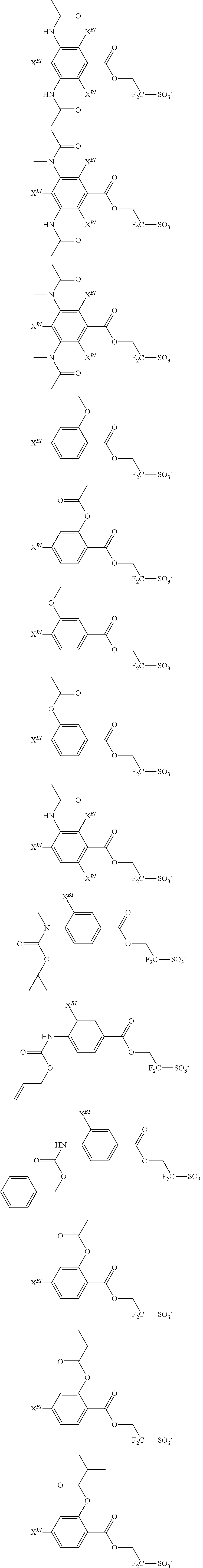

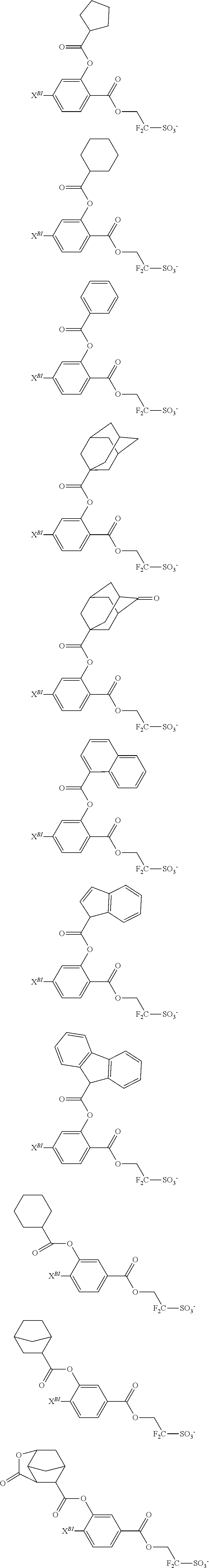

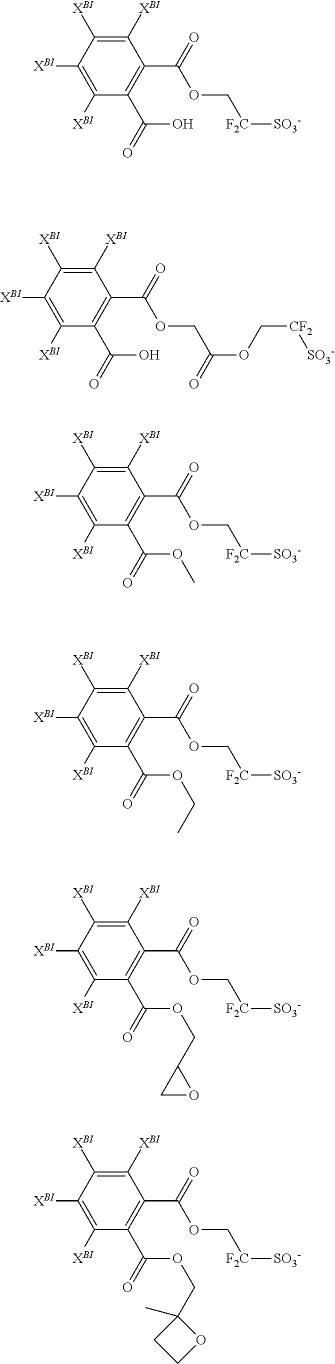

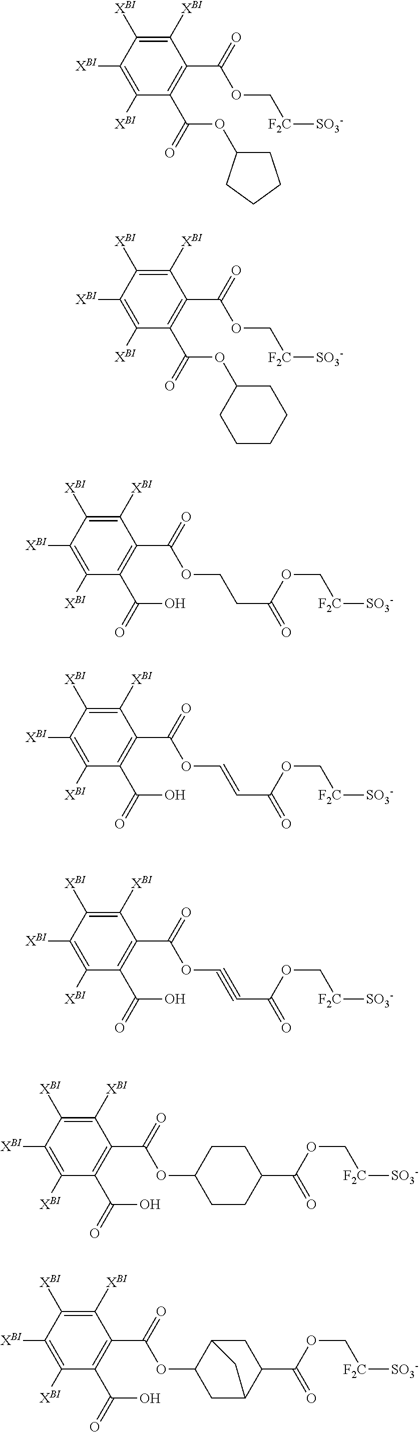

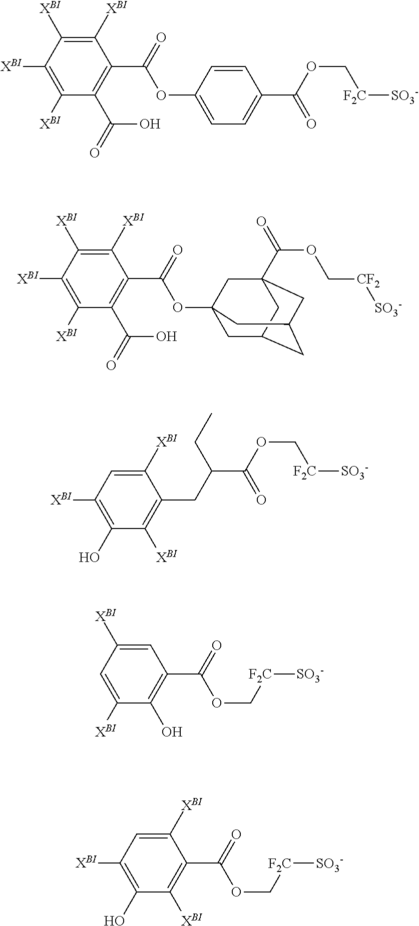

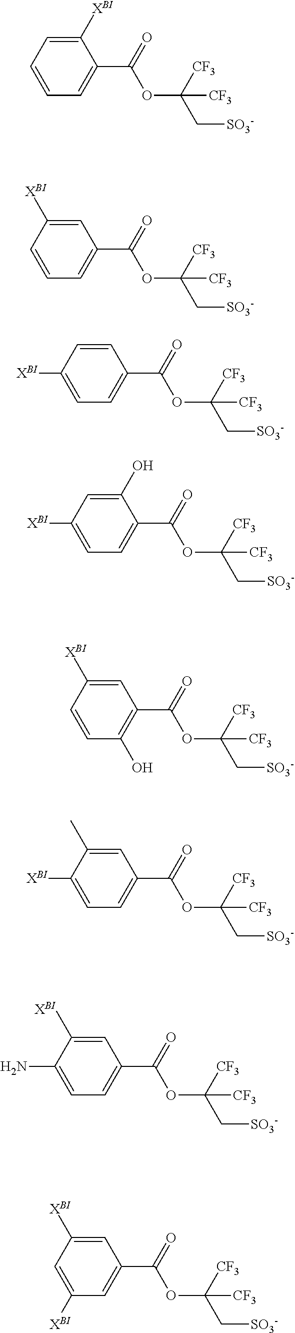

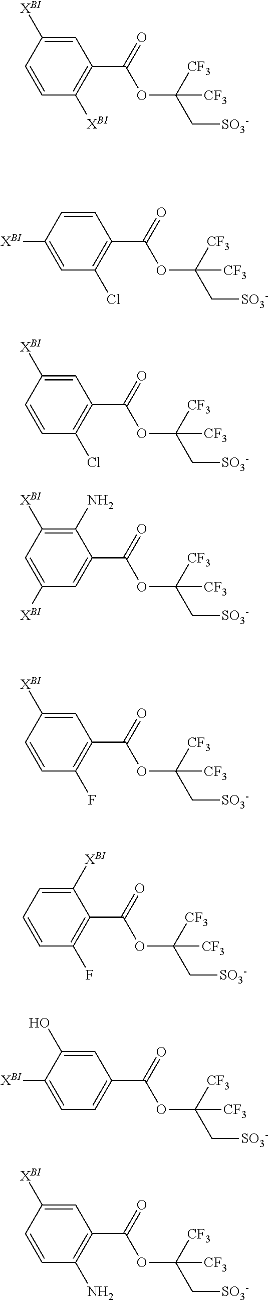

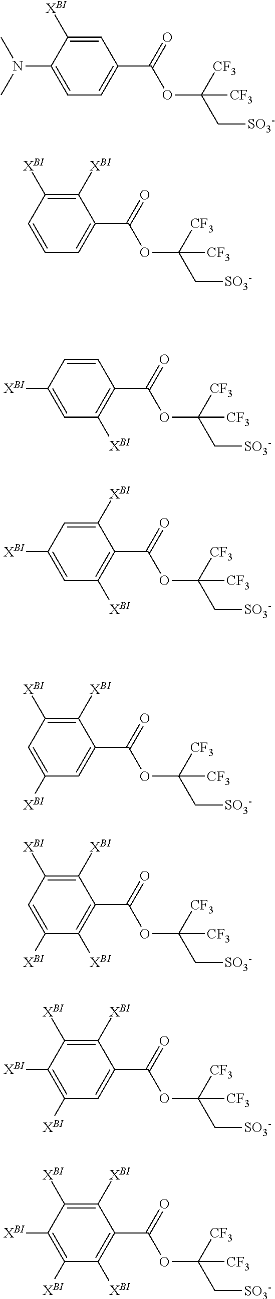

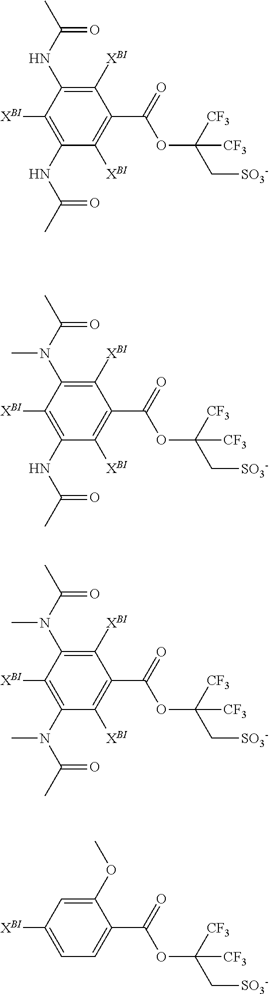

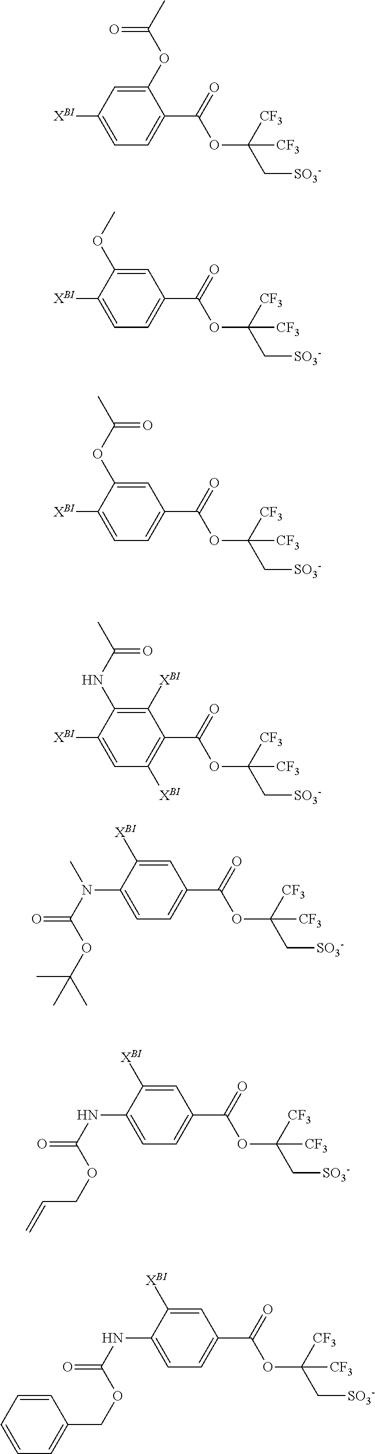

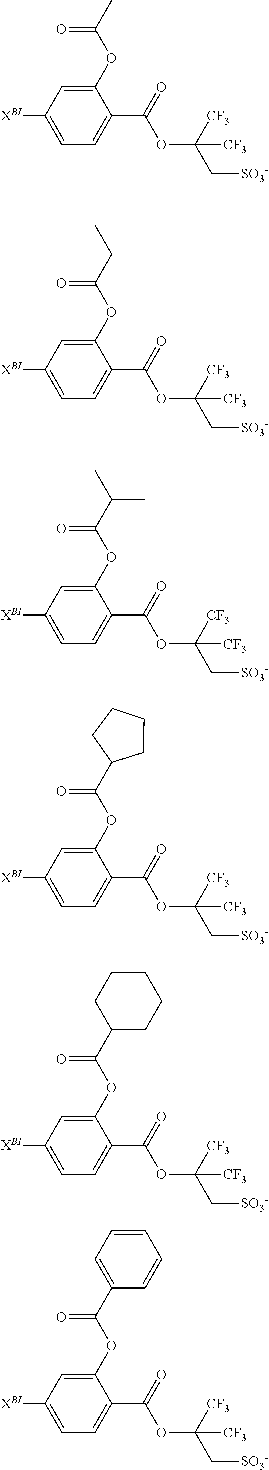

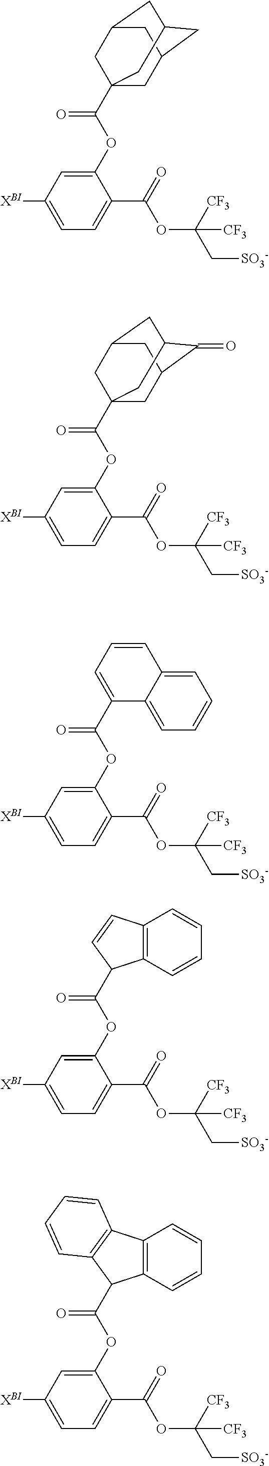

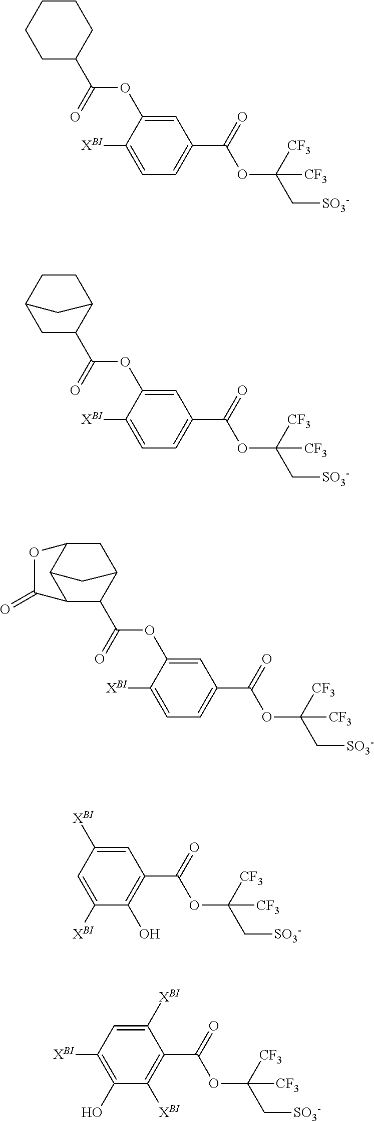

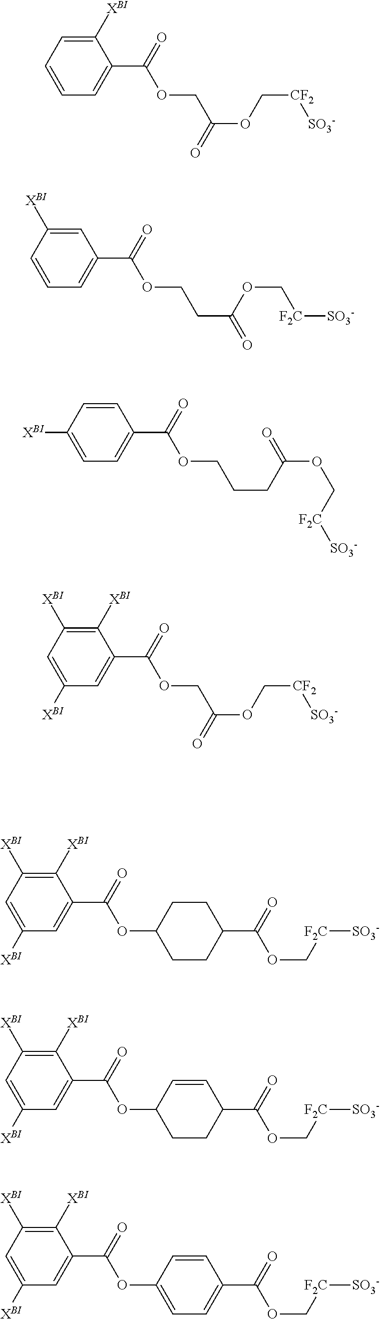

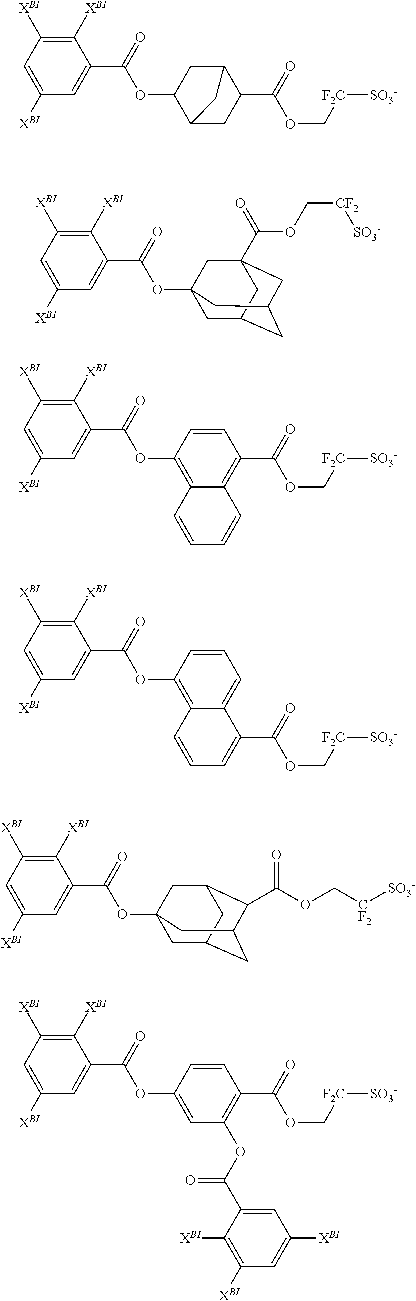

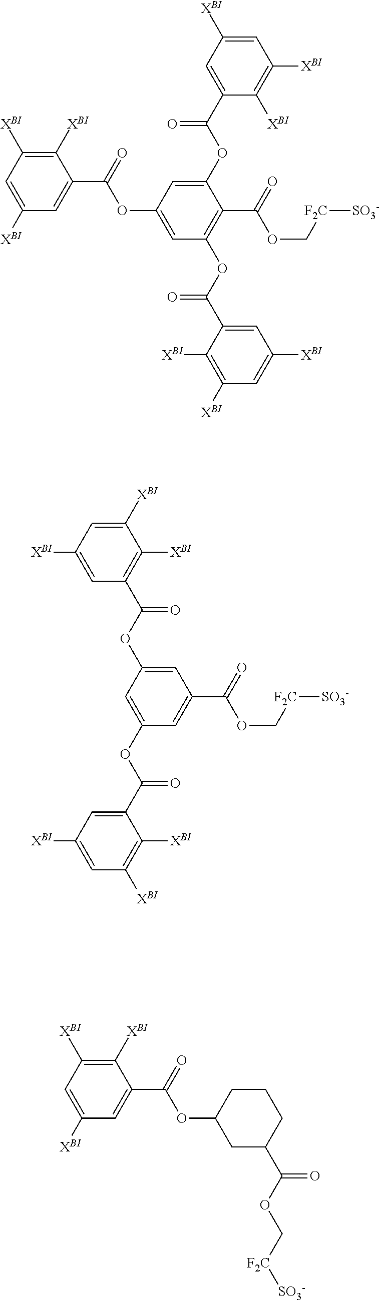

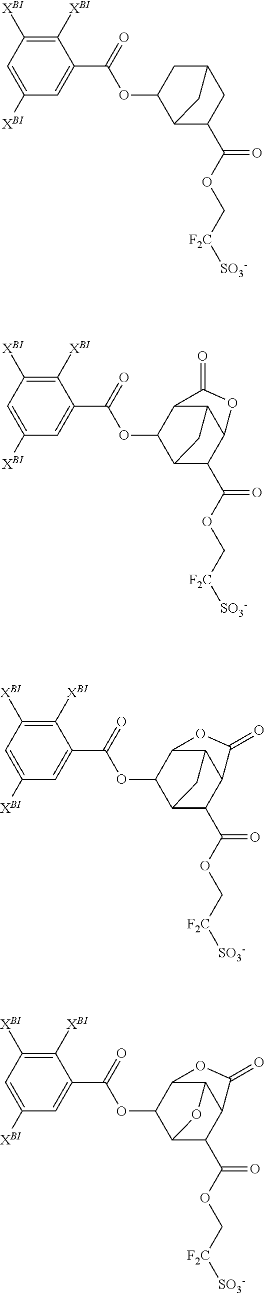

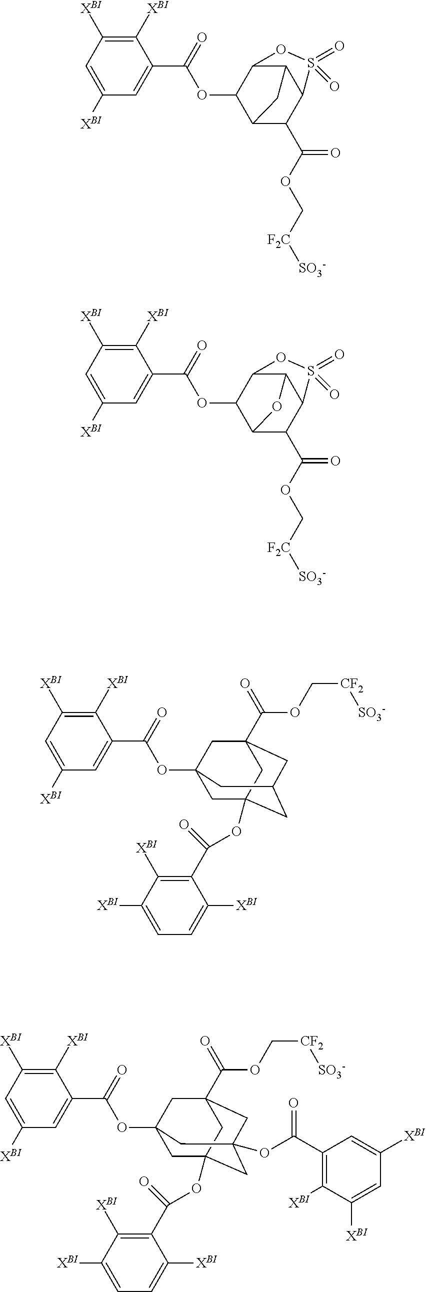

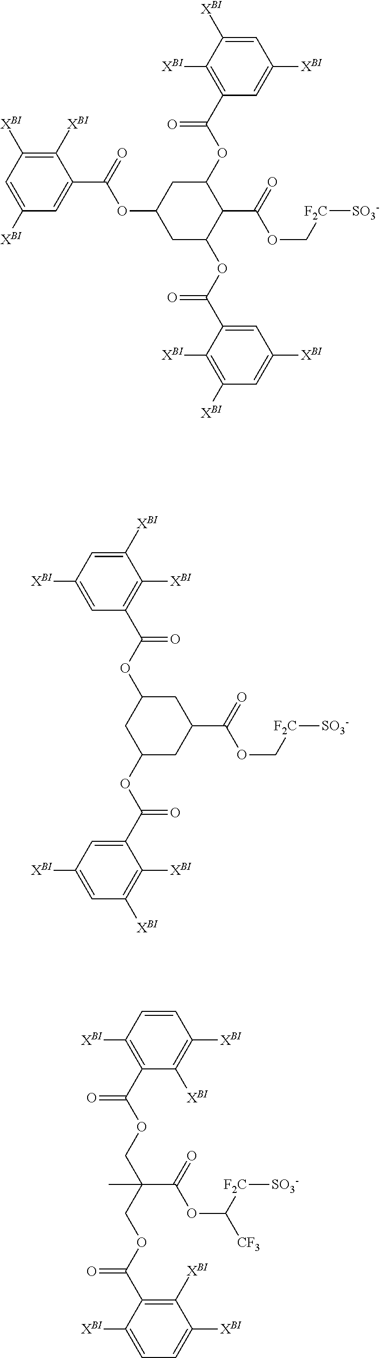

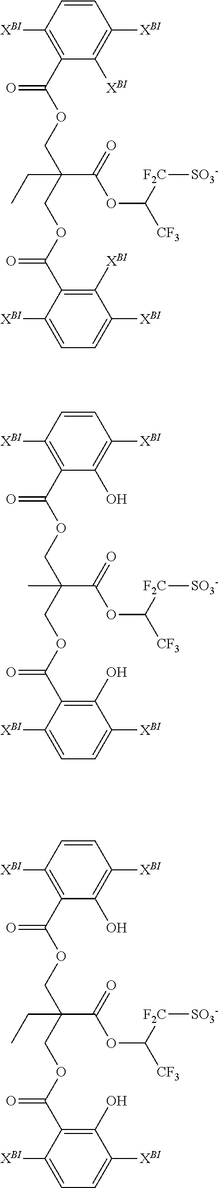

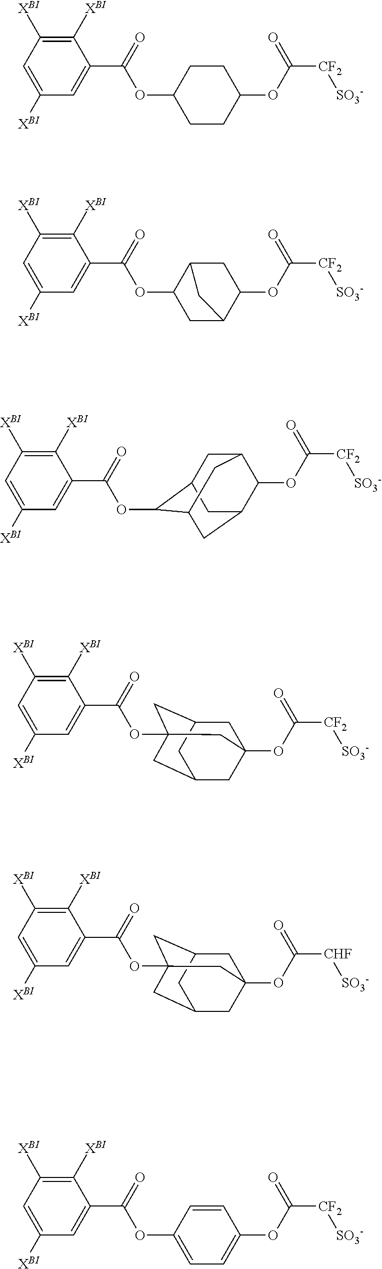

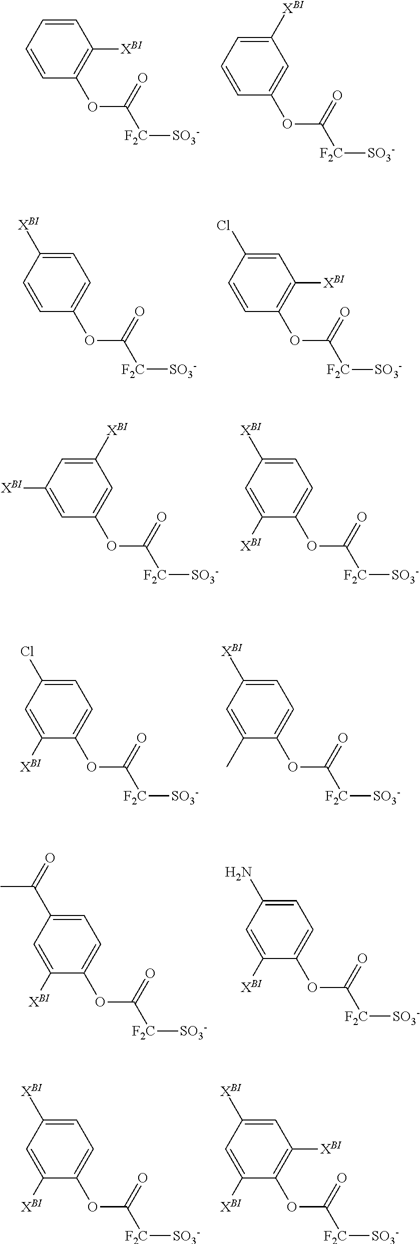

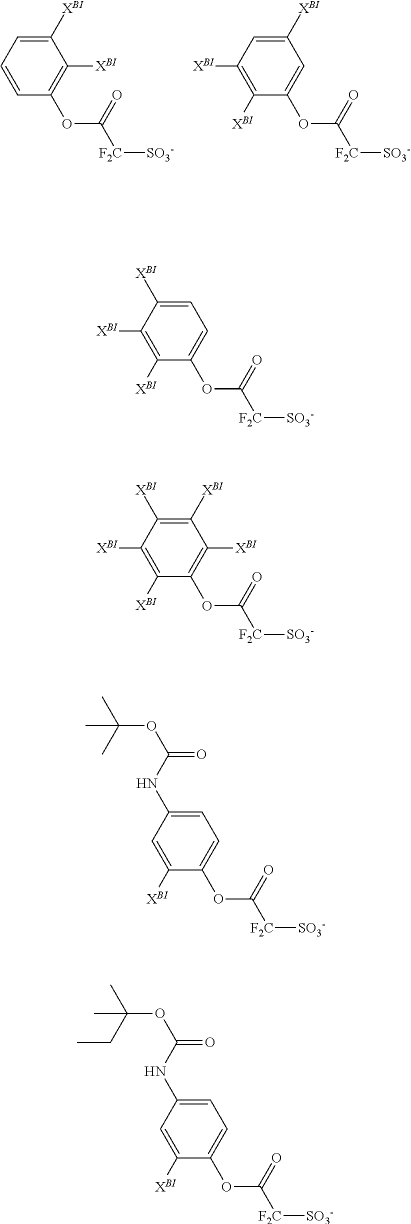

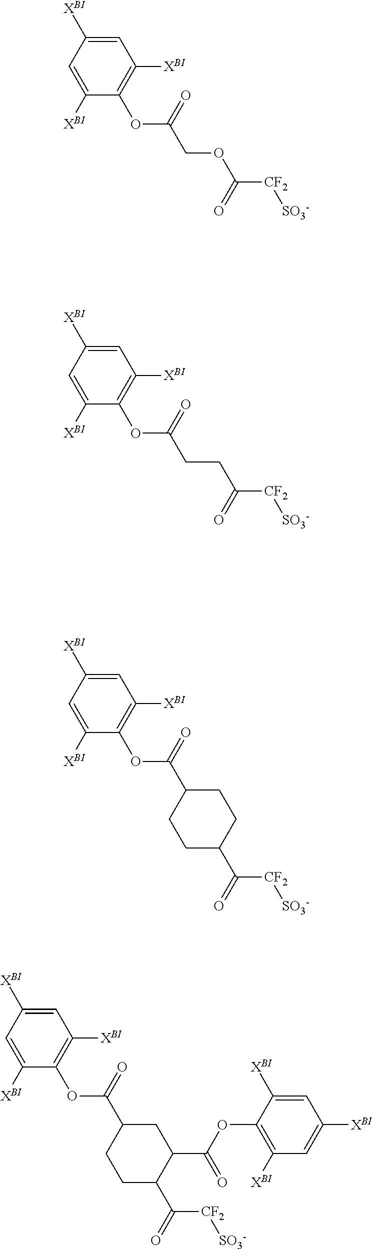

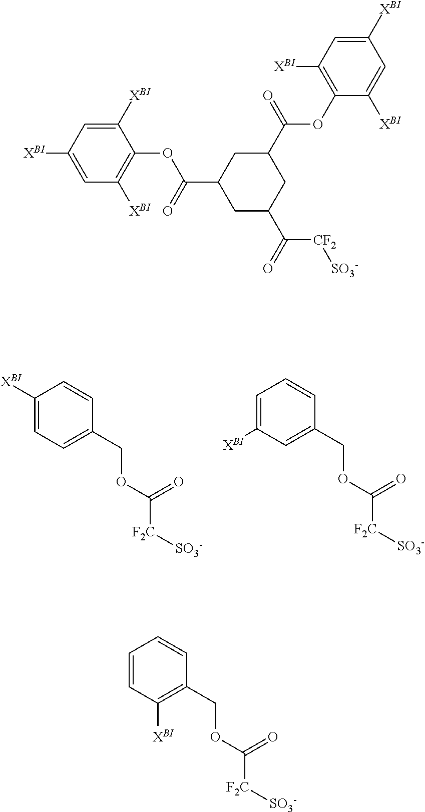

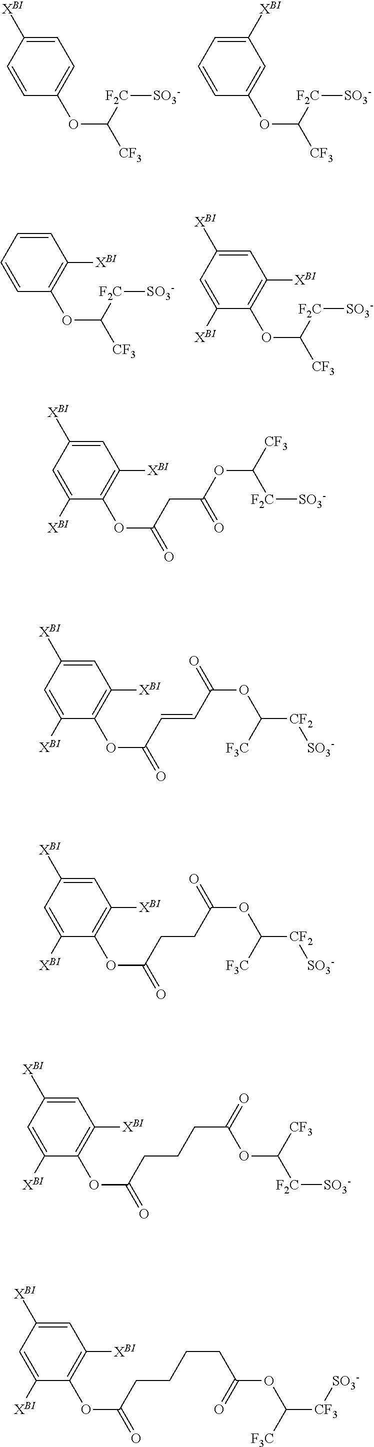

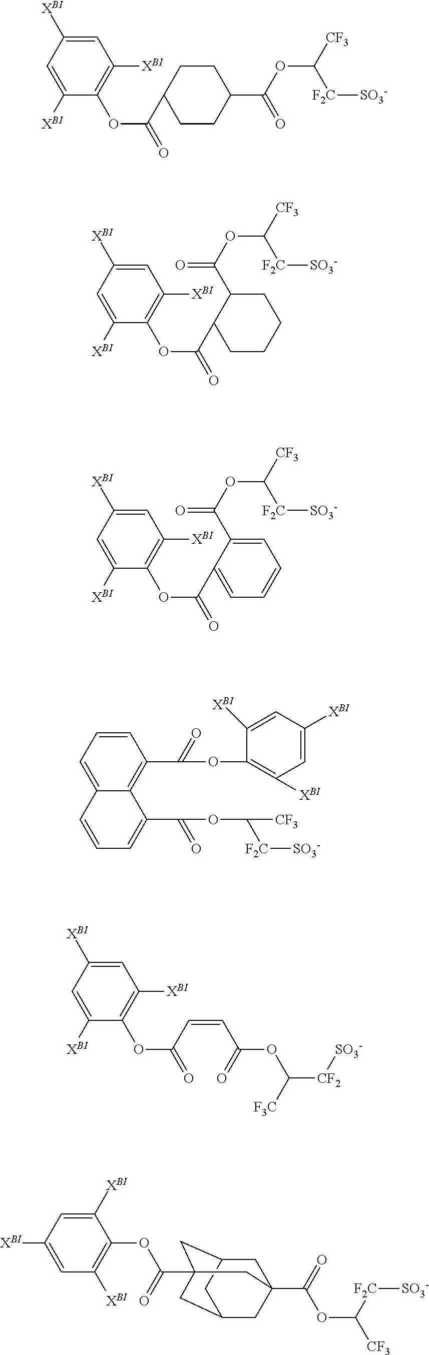

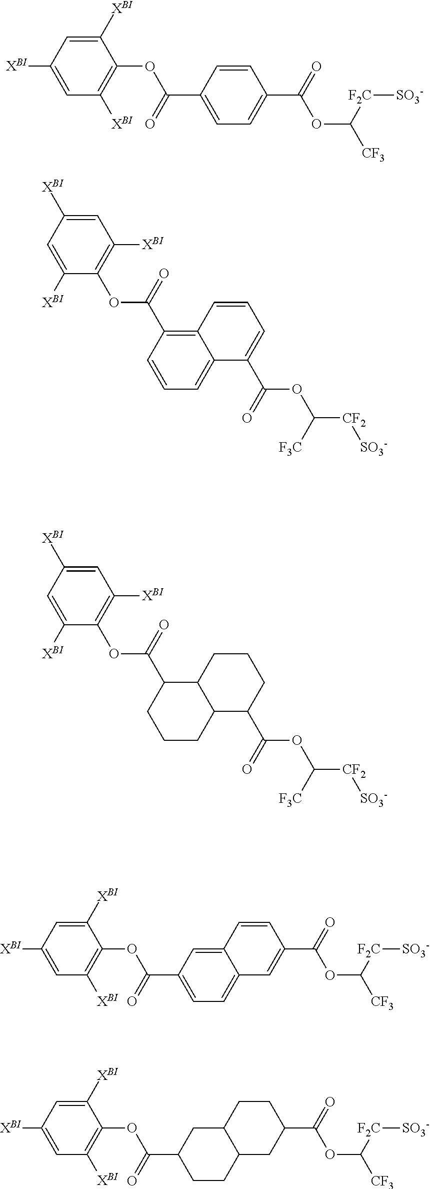

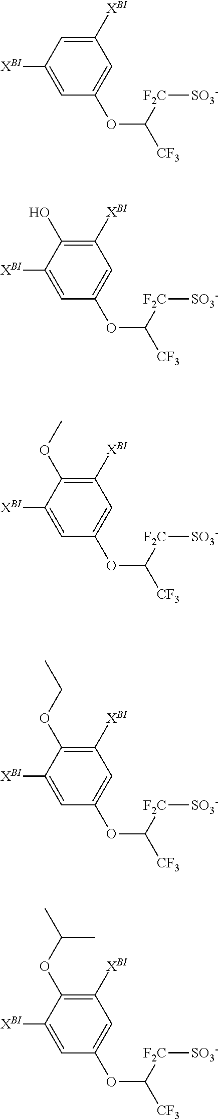

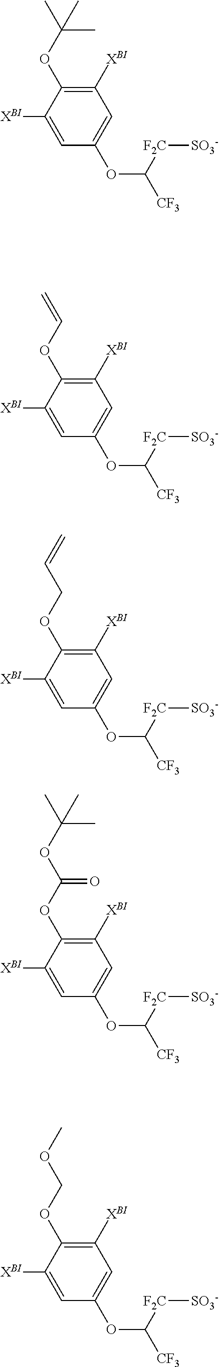

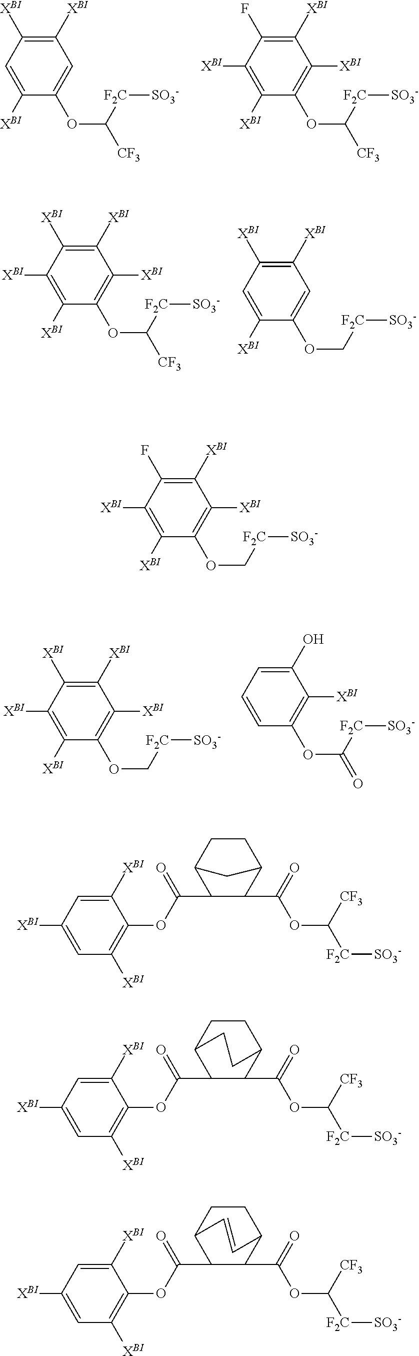

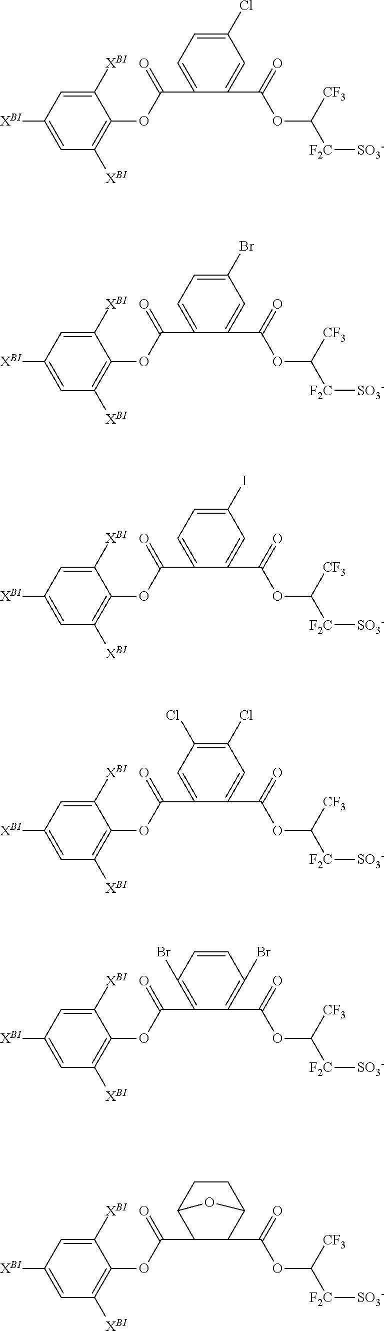

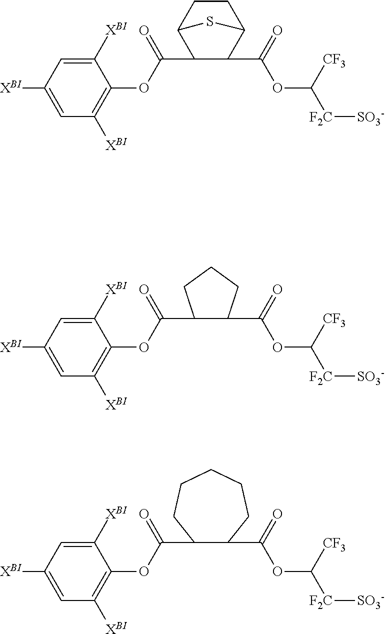

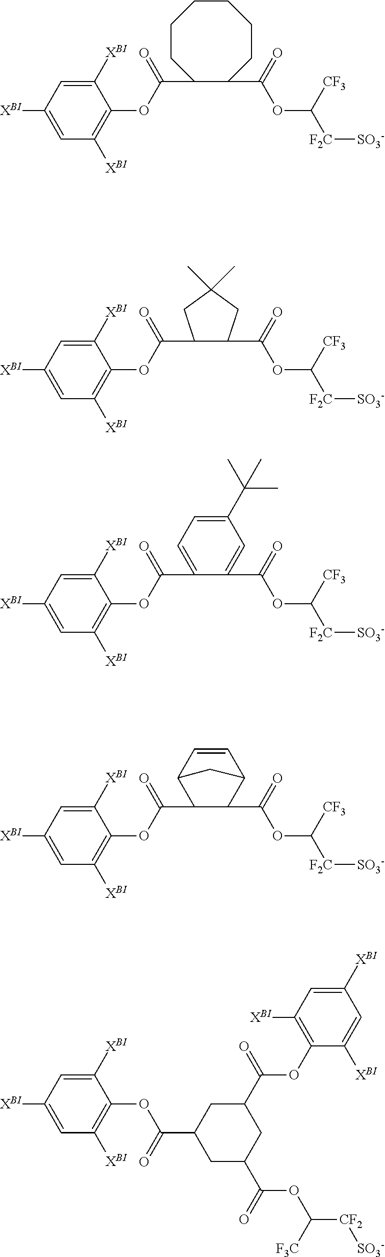

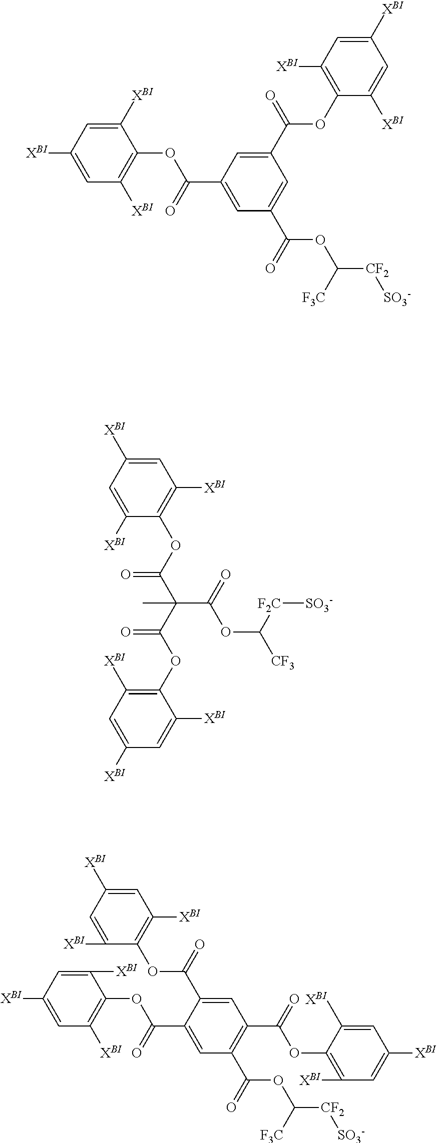

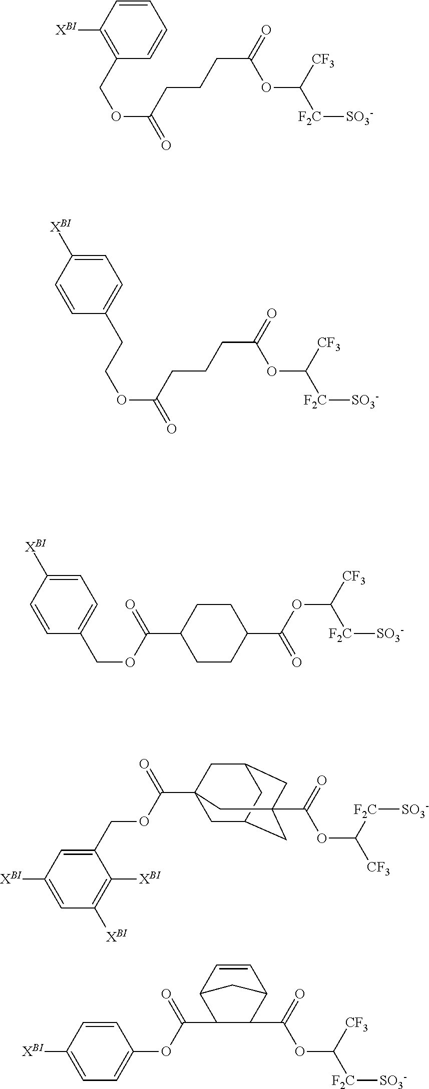

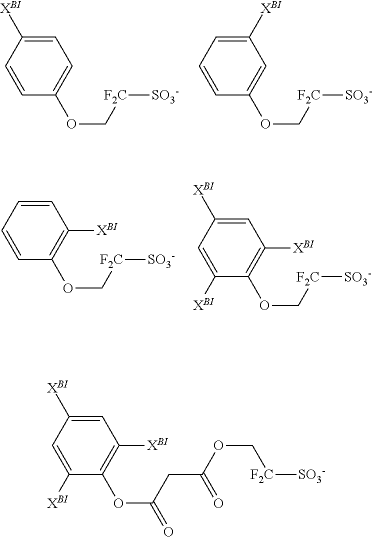

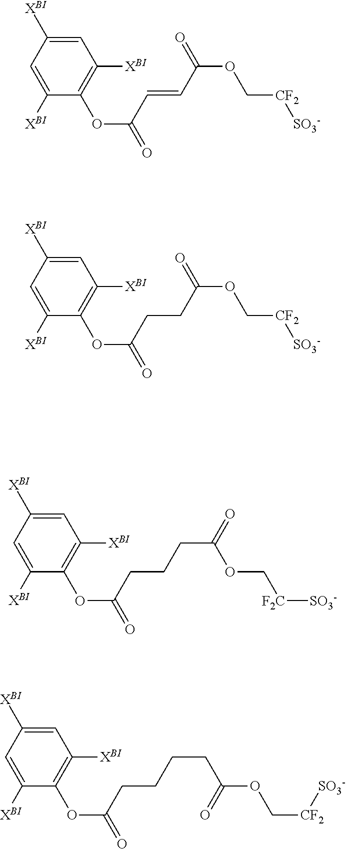

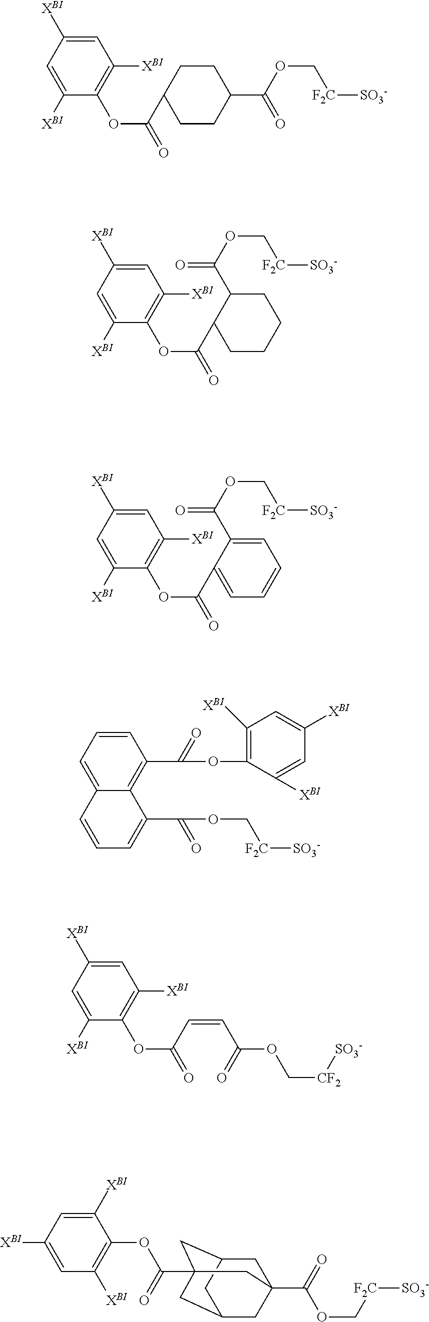

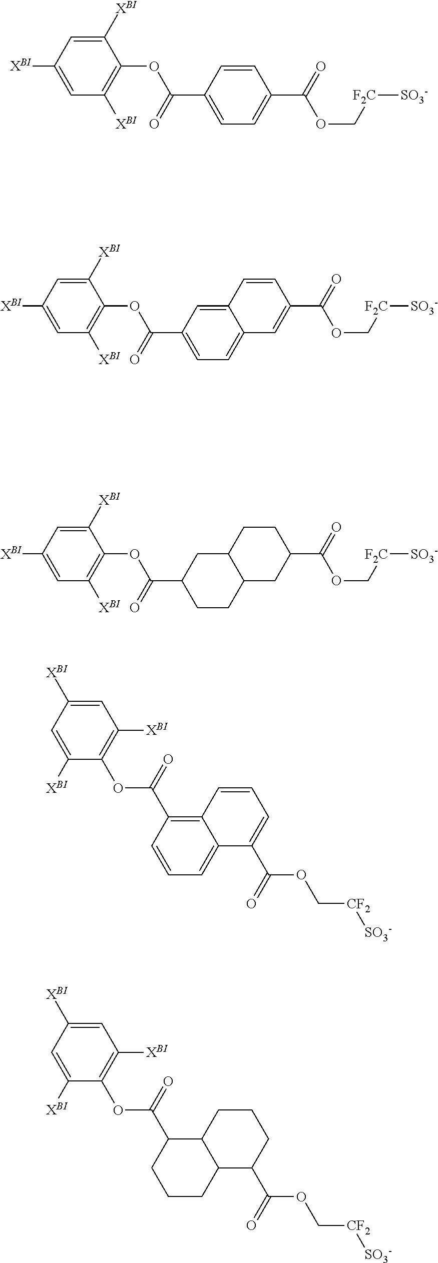

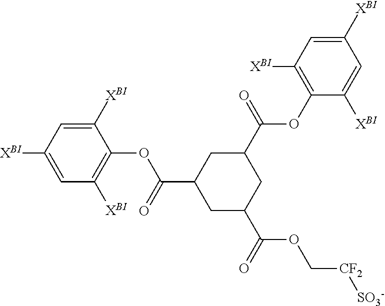

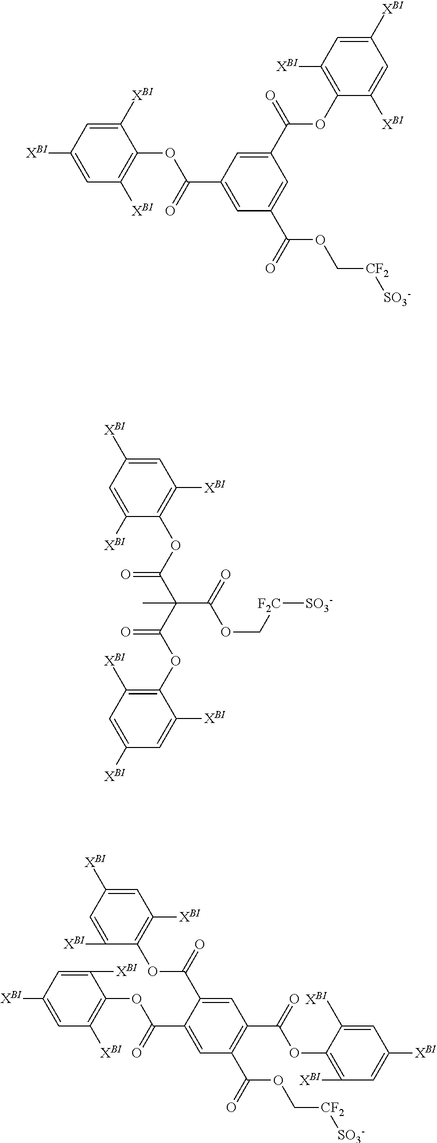

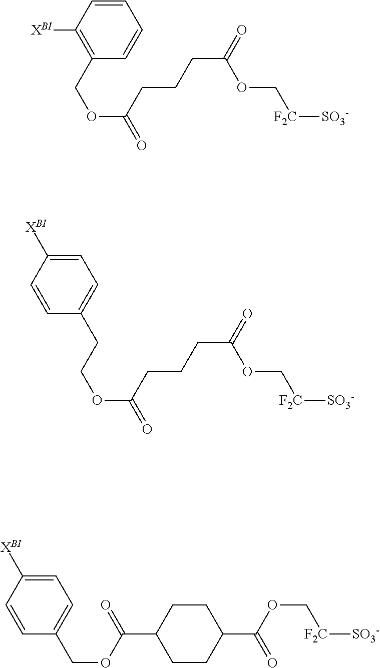

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-070692, anions having a cyclic ether group as described in JP-A 2018-180525 and JP-A 2021-035935, and anions as described in JP-A 2018-092159.

Further useful examples of the non-nucleophilic counter ion include bulky fluorine-free benzenesulfonic acid anions as described in JP-A 2006-276759, JP-A 2015-117200, JP-A 2016-065016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-024989.

In formulae (c2) and (c3), L¹ is a single bond, ether bond, ester bond, carbonyl, sulfonate ester bond, carbonate bond or carbamate bond. From the aspect of synthesis, an ether bond, ester bond or carbonyl is preferred, with the ester bond or carbonyl being more preferred.

In formula (c2), Rf¹ and Rf² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine or a C₁-C₆ fluorinated saturated hydrocarbyl group. It is preferred for solvent solubility that at least one of Rf³ and Rf⁴ be trifluoromethyl.

In formula (c3), Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In formulae (c2) and (c3), c1 and c2 are each independently an integer of 0 to 3, preferably 1.

Examples of the anion in repeat unit (c2) are shown below, but not limited thereto. R^{A} is as defined above.

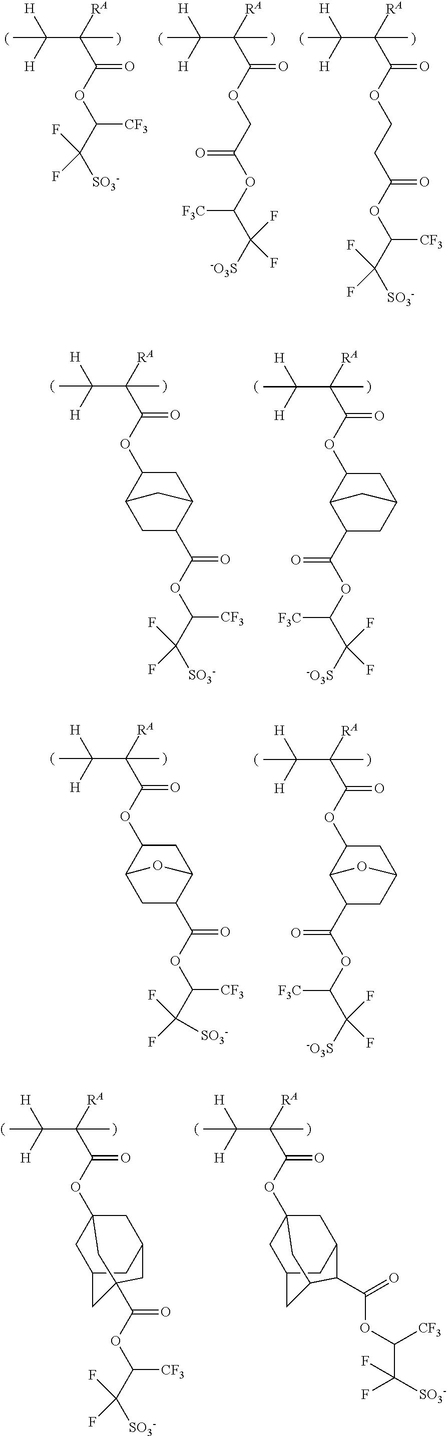

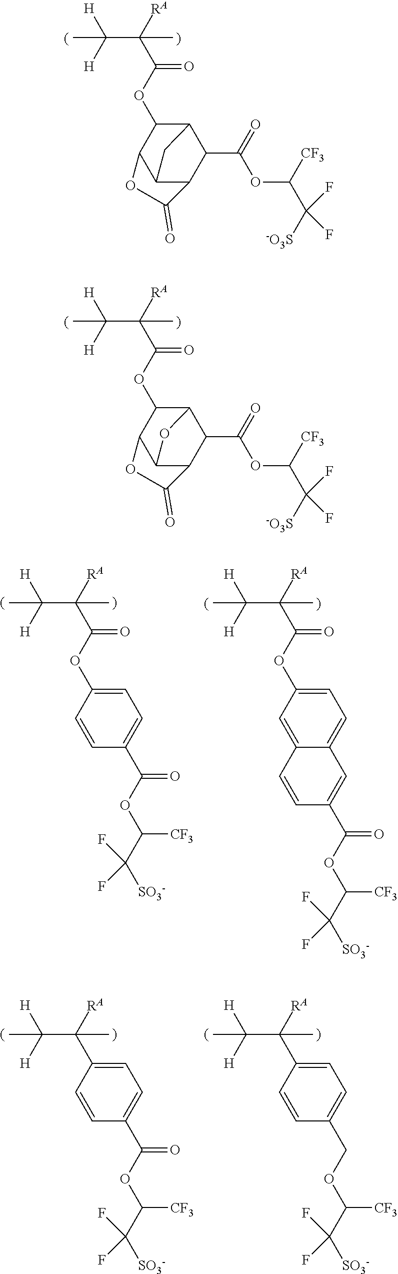

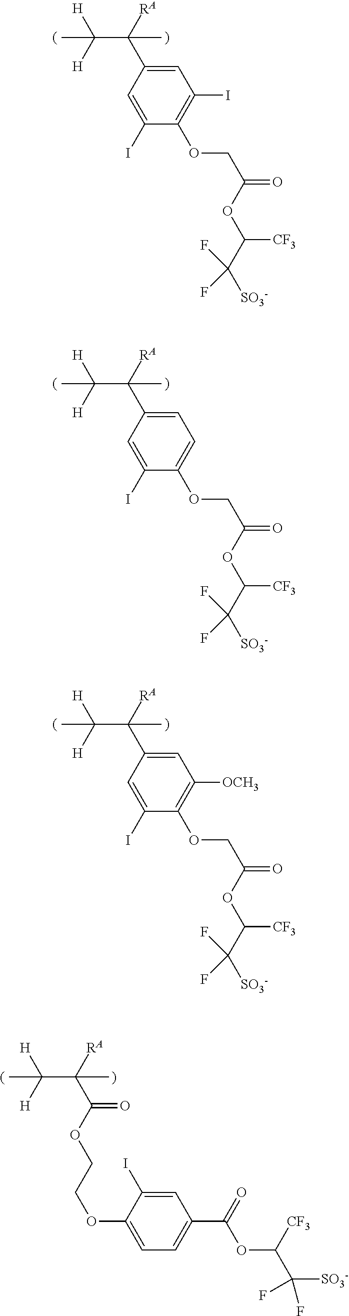

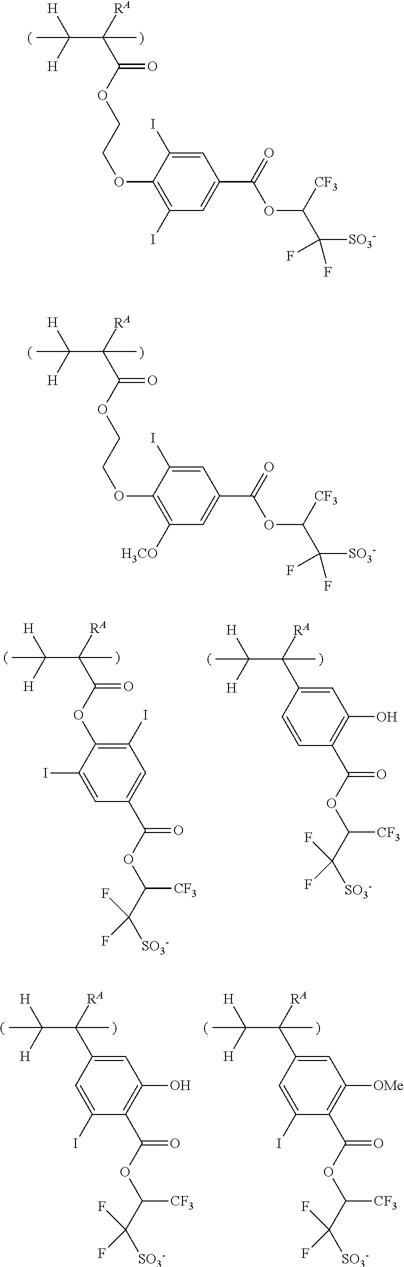

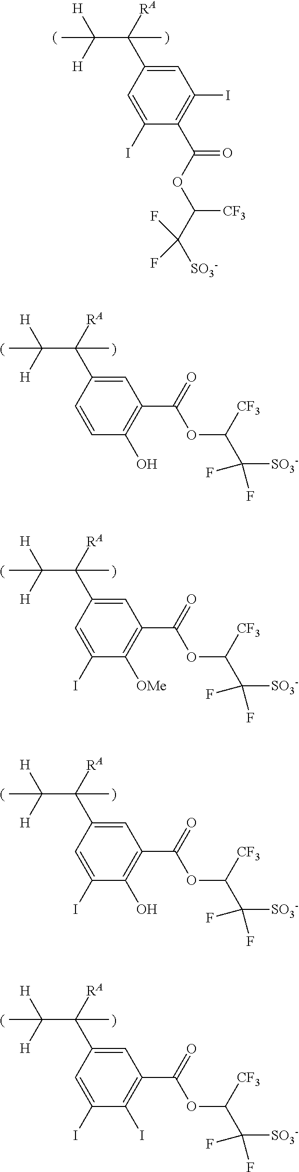

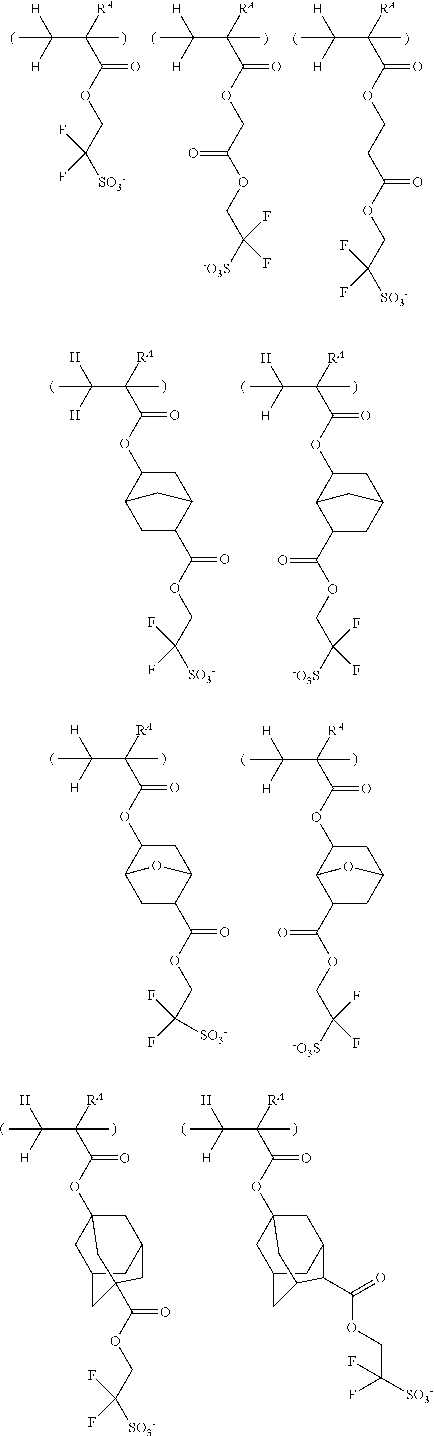

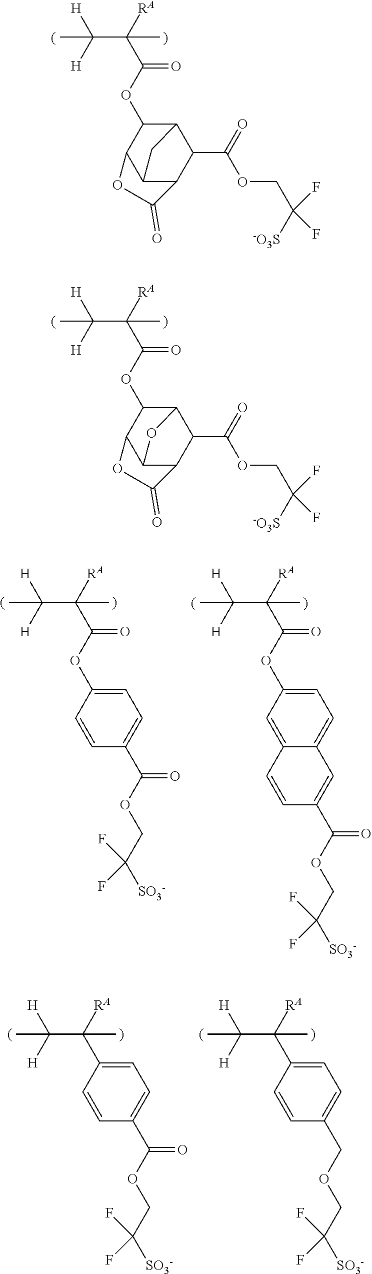

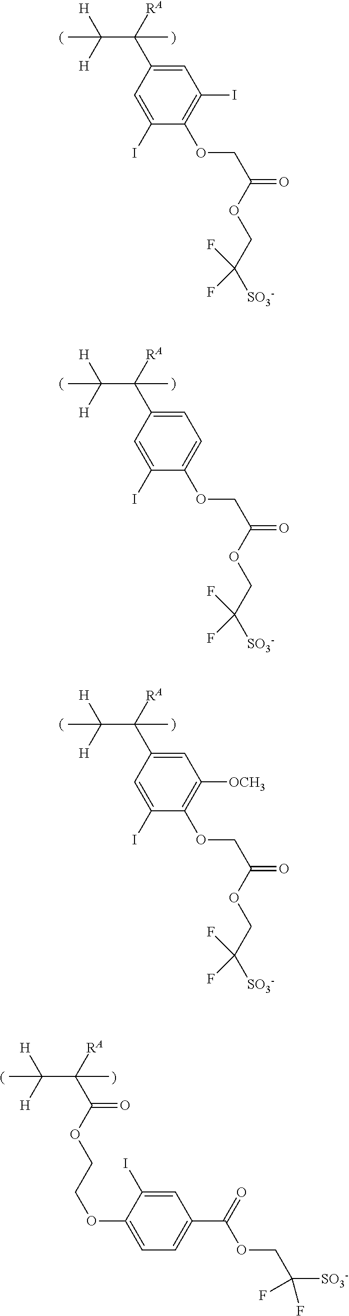

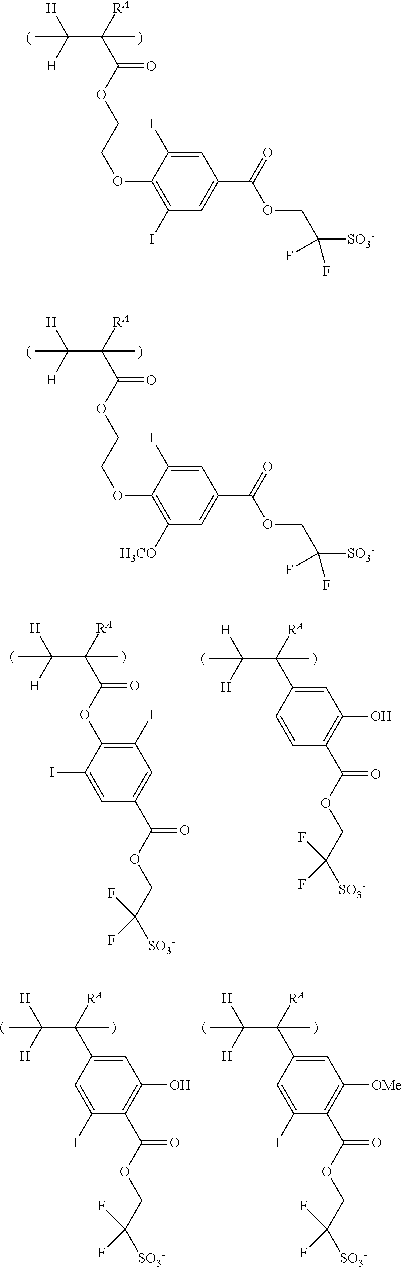

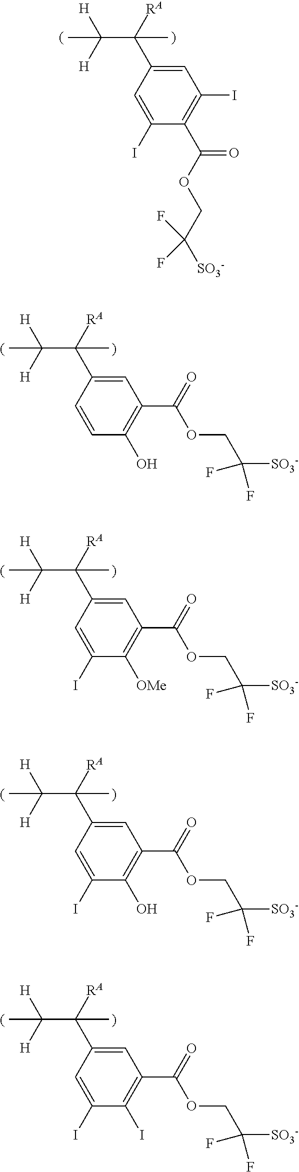

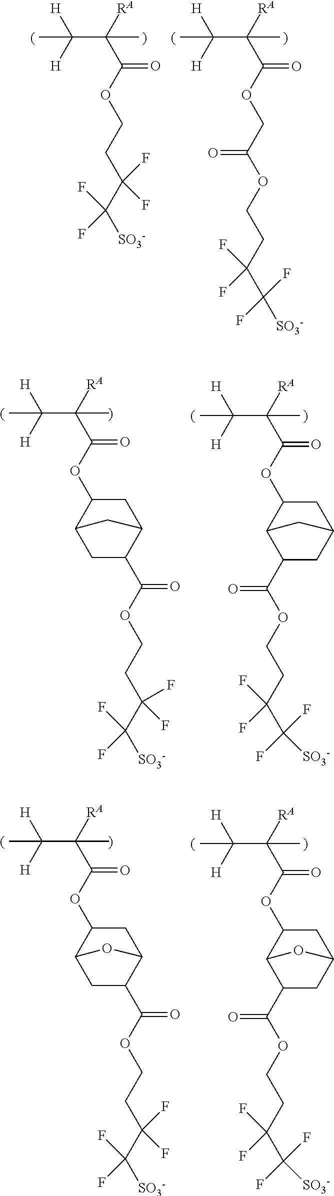

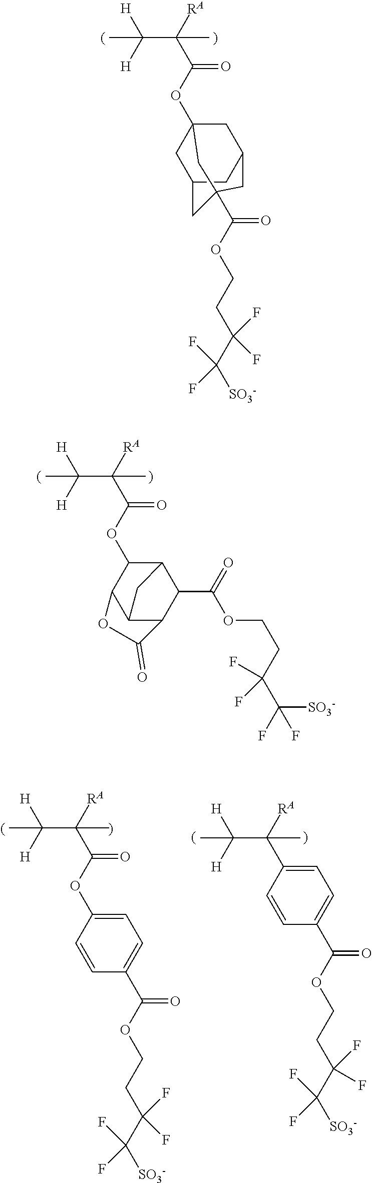

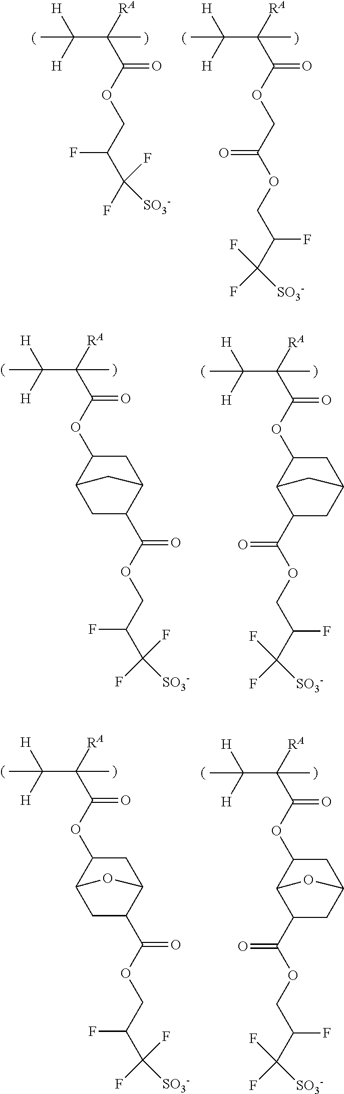

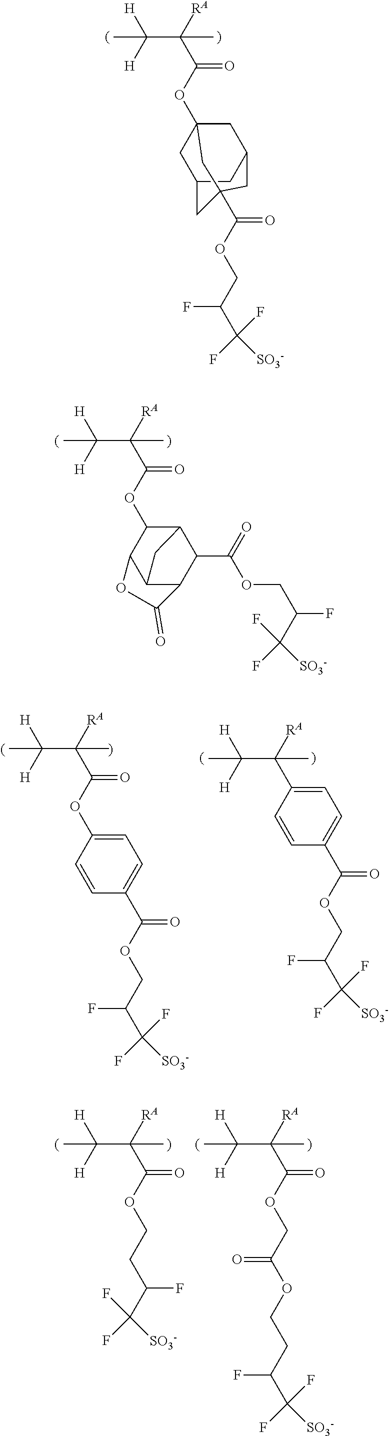

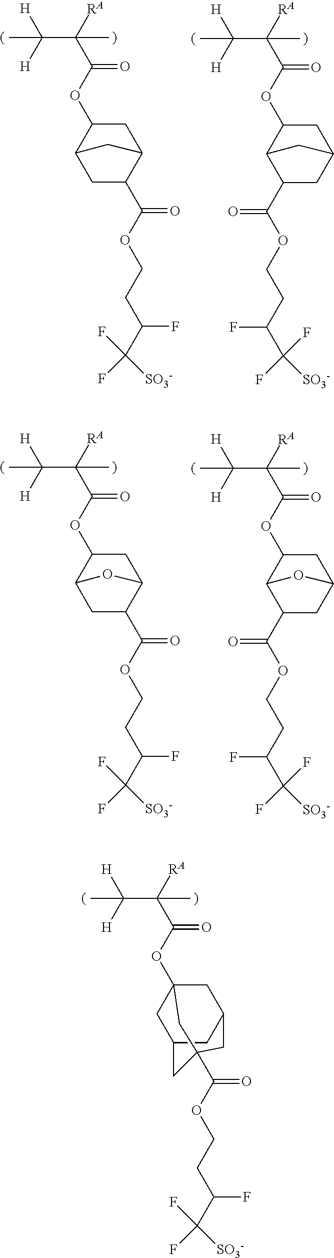

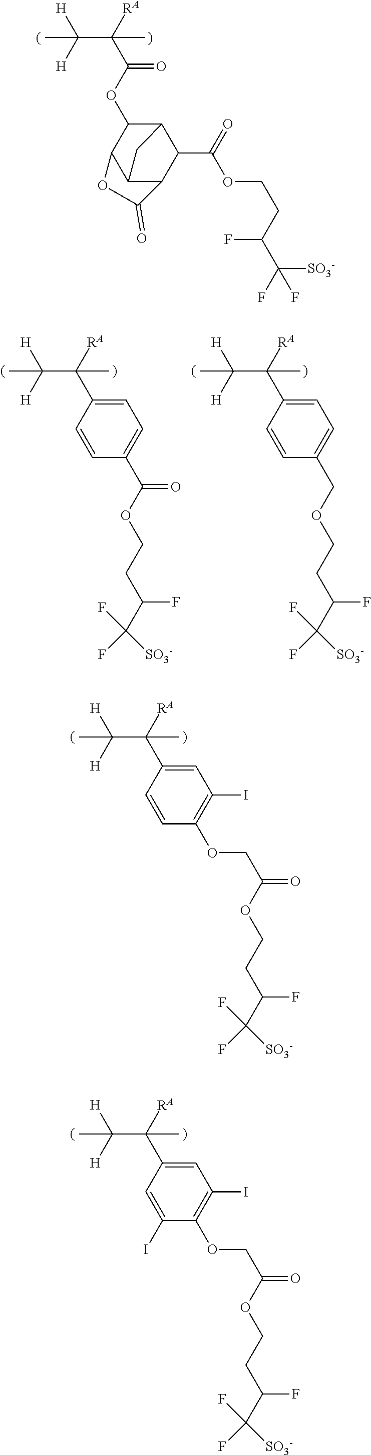

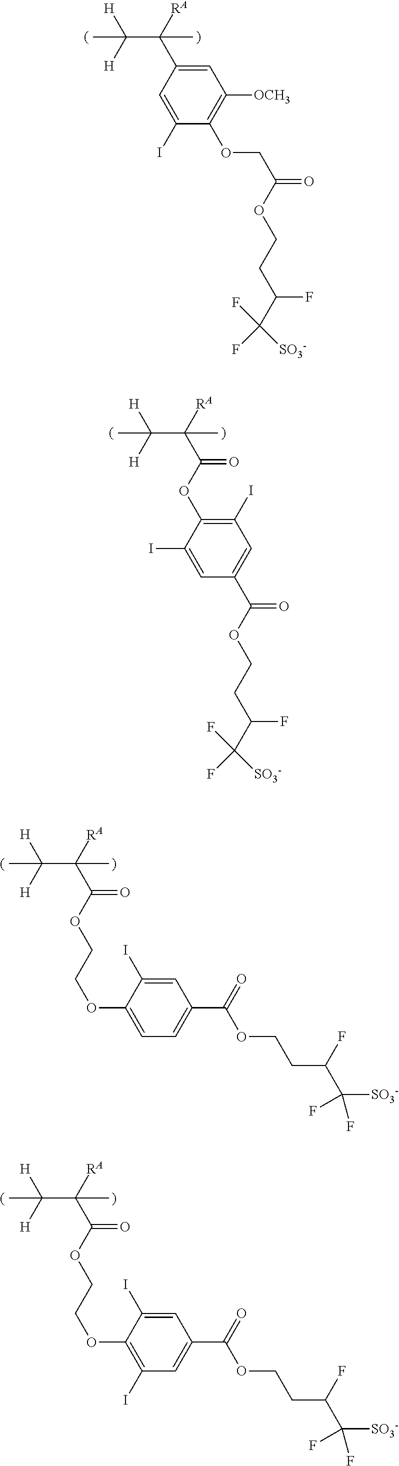

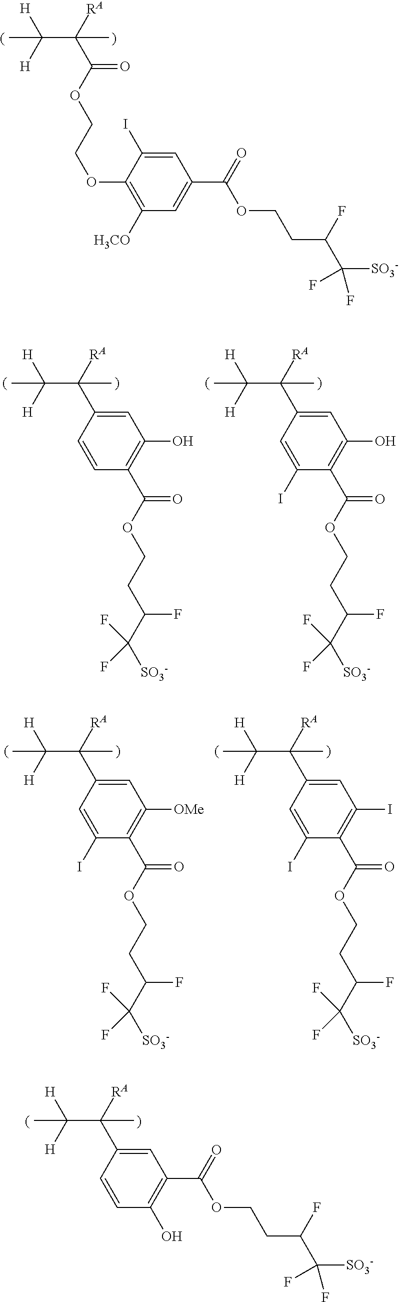

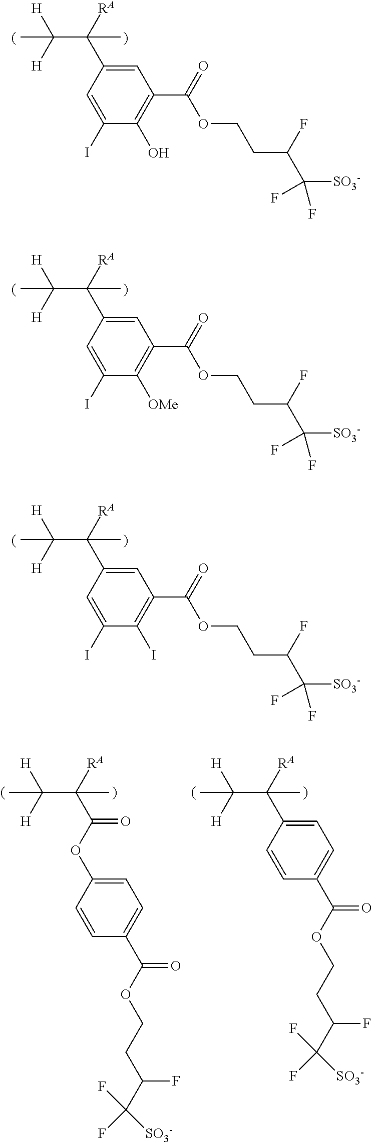

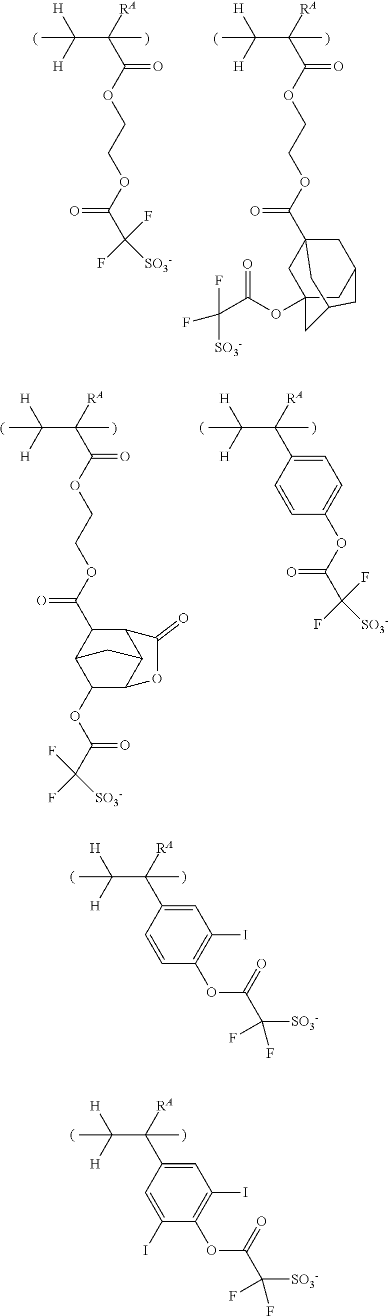

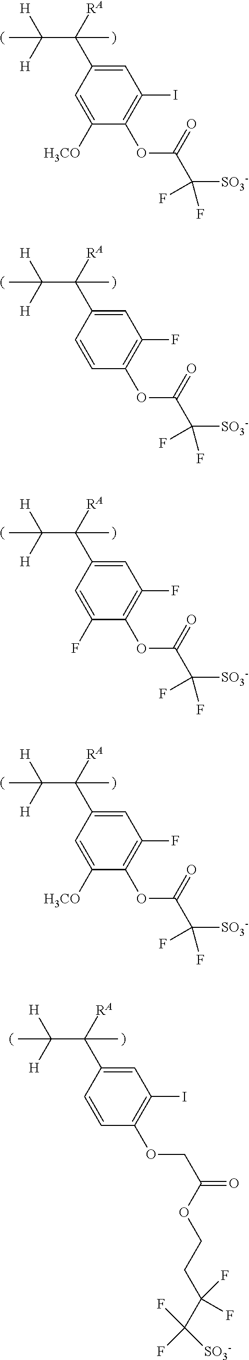

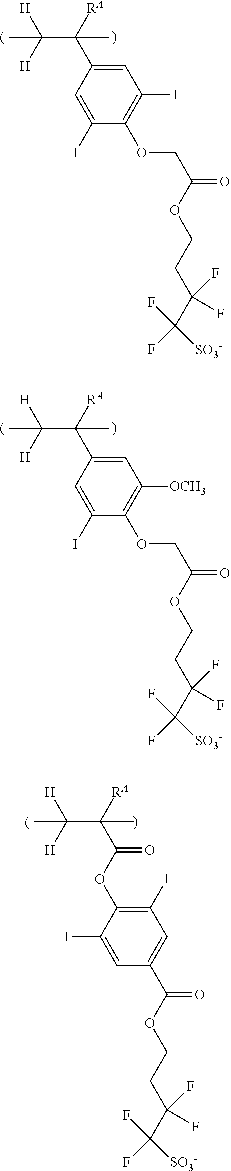

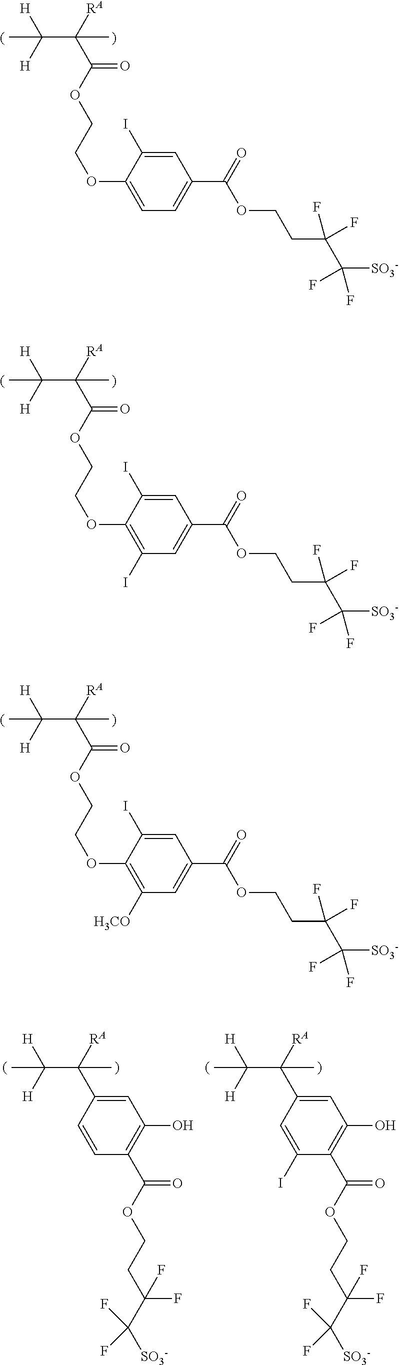

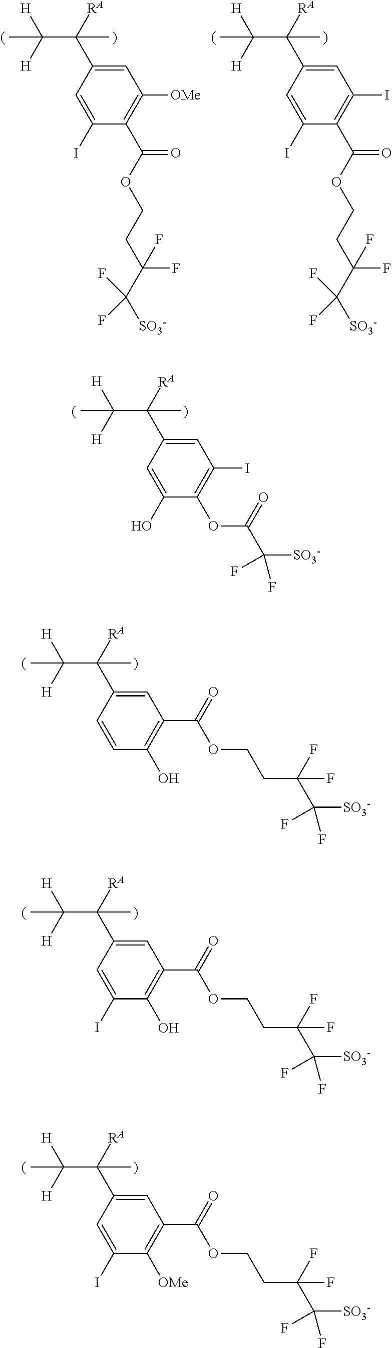

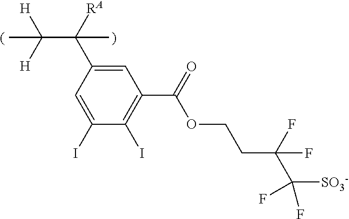

Examples of the anion in repeat unit (6) are shown below, but not limited thereto. R^{A} is as defined above.

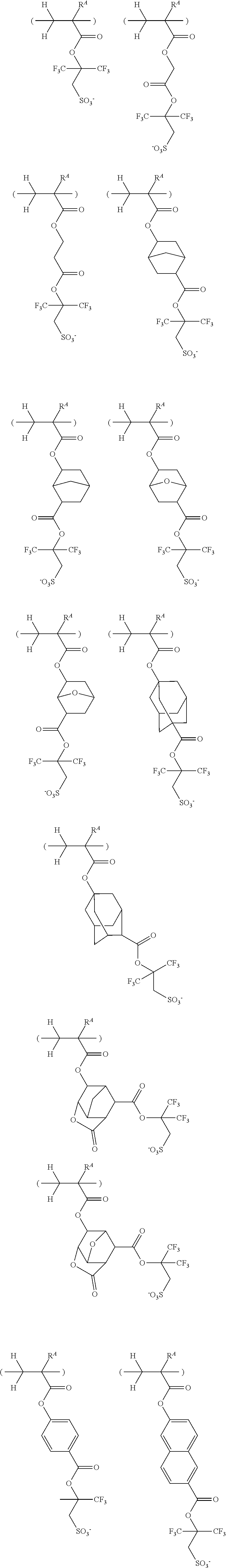

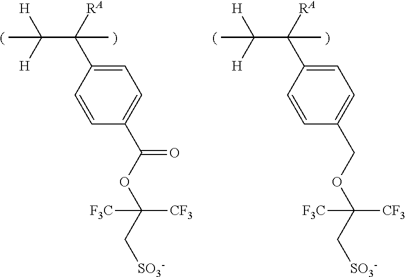

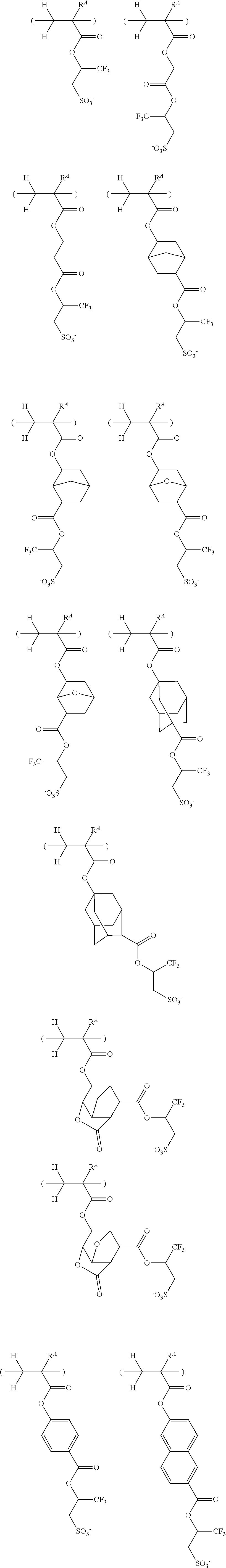

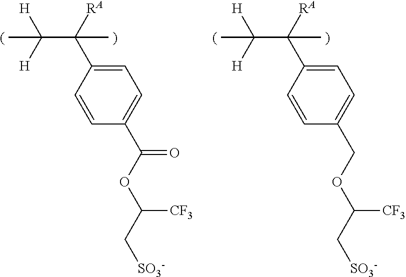

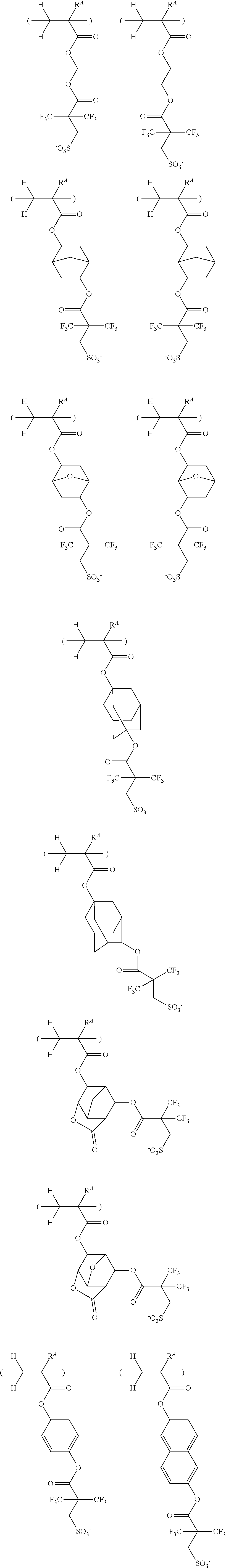

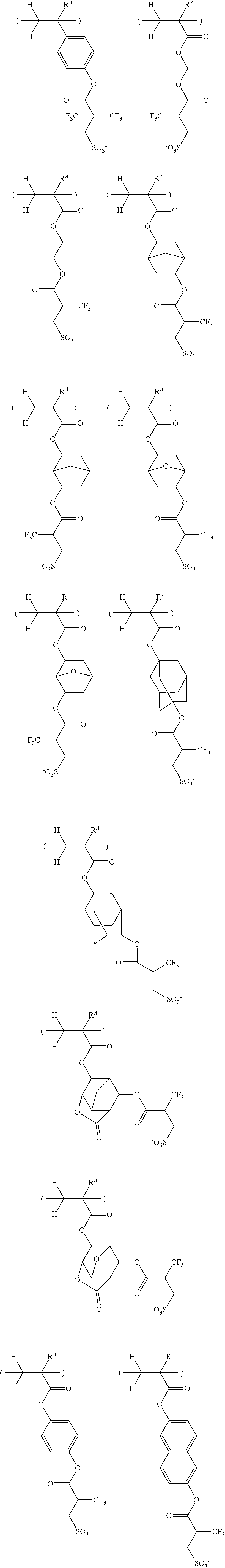

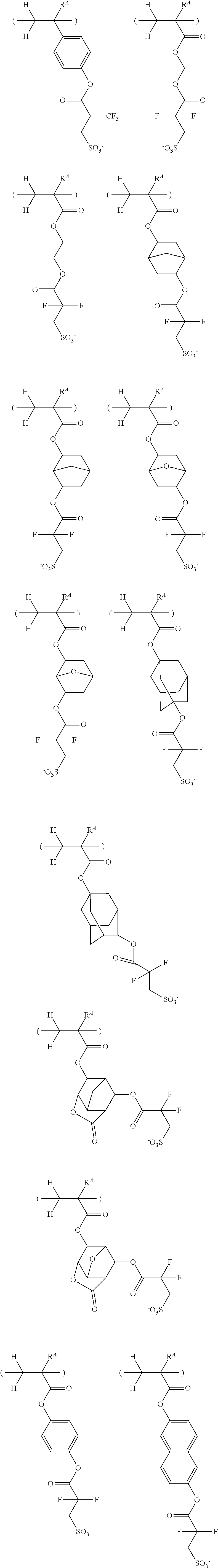

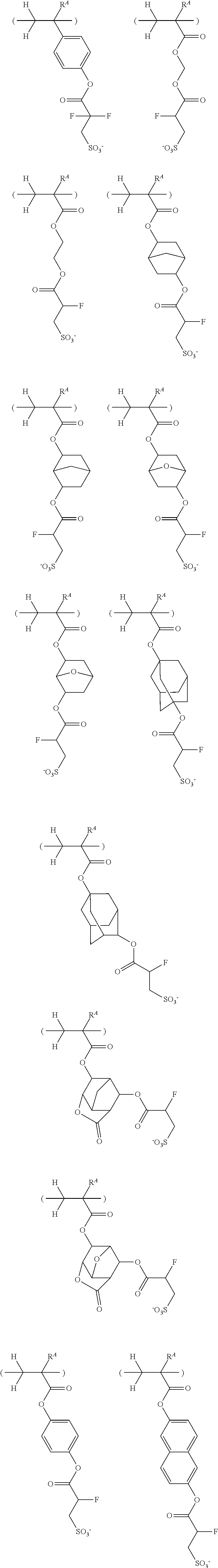

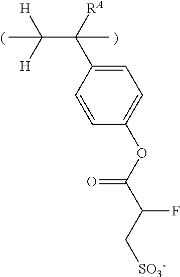

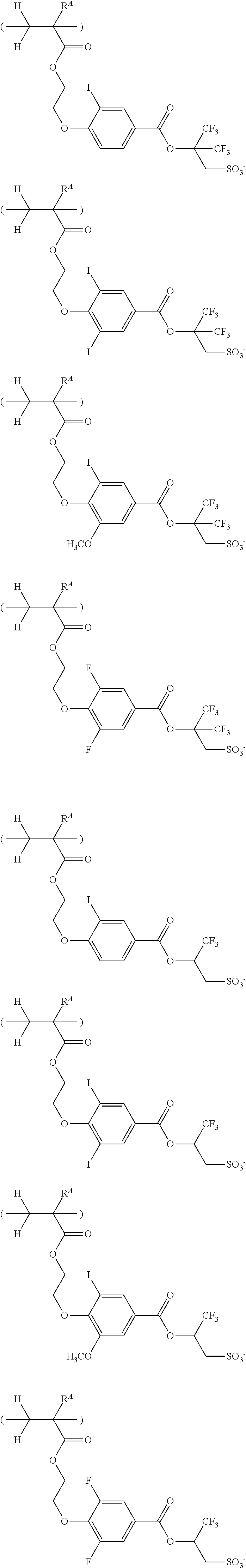

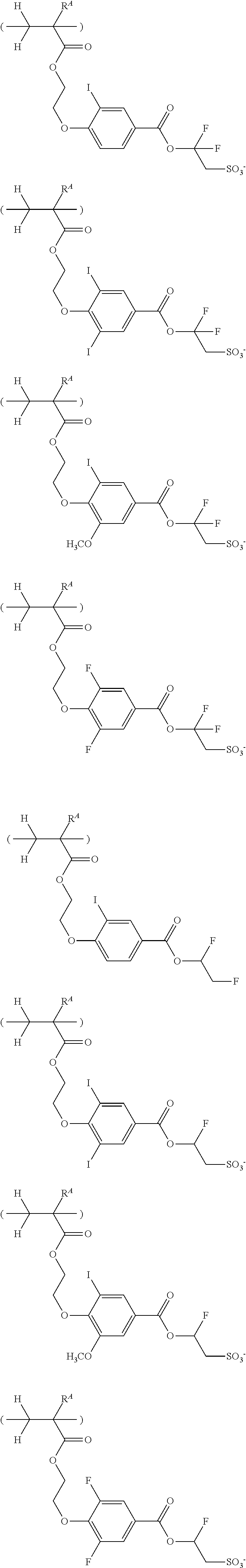

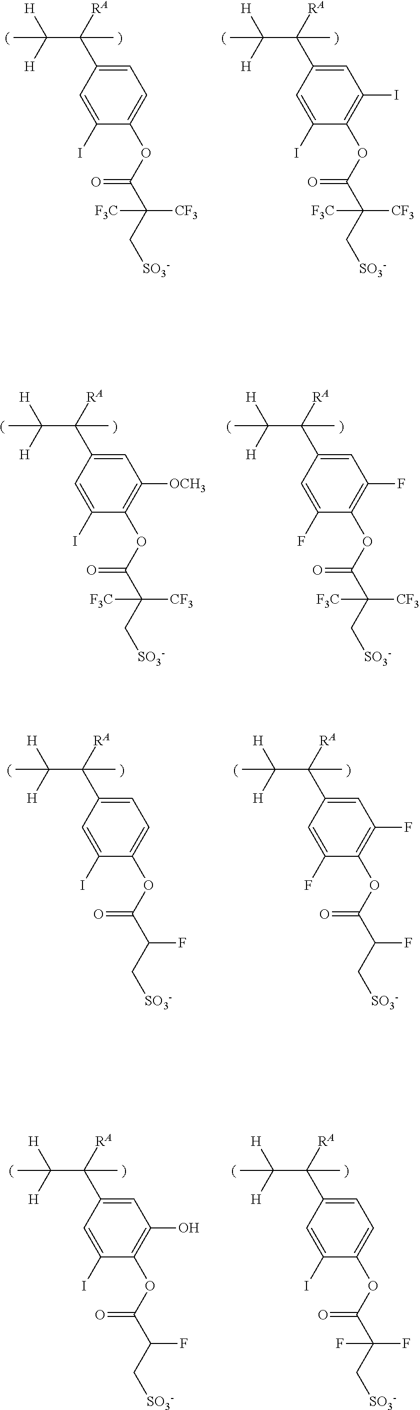

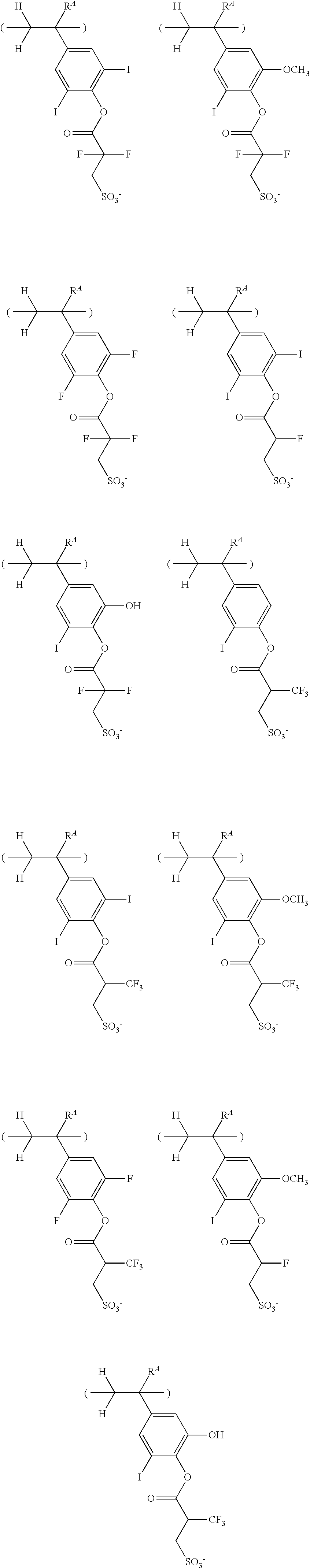

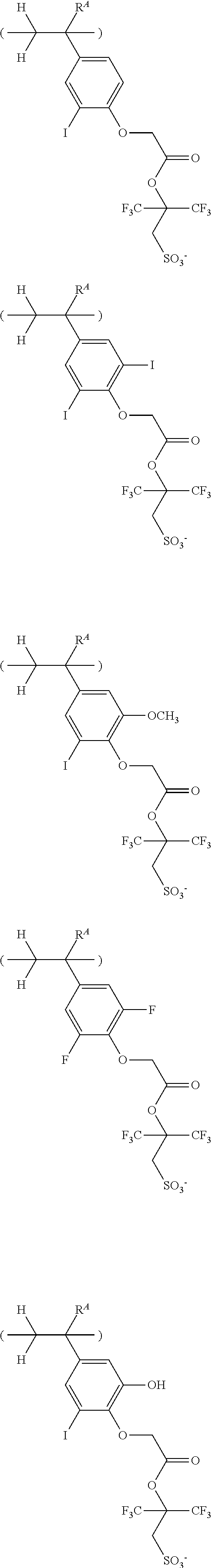

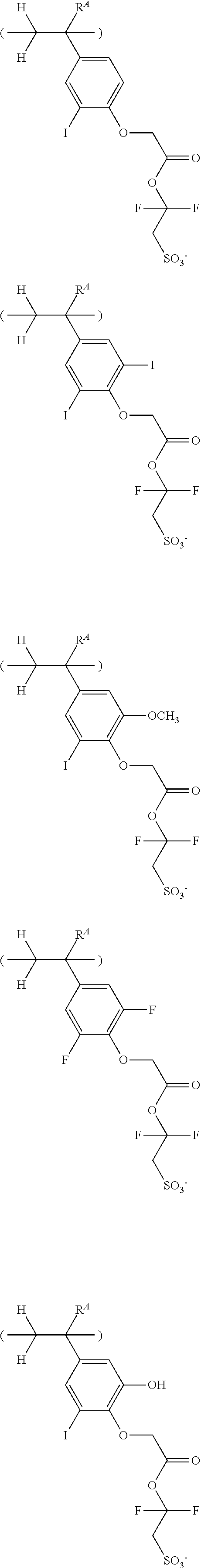

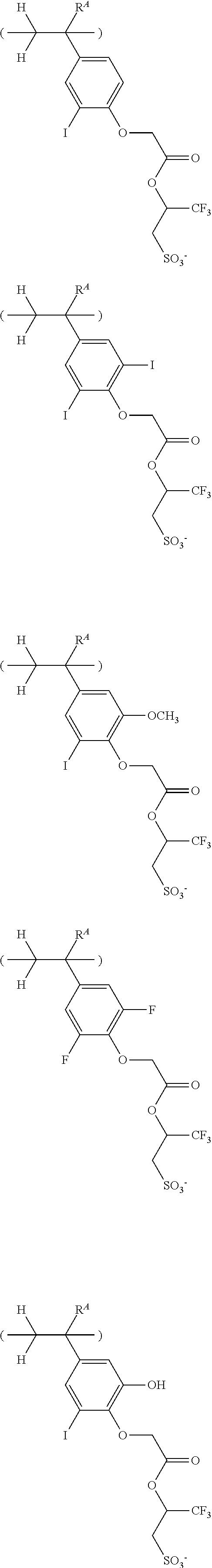

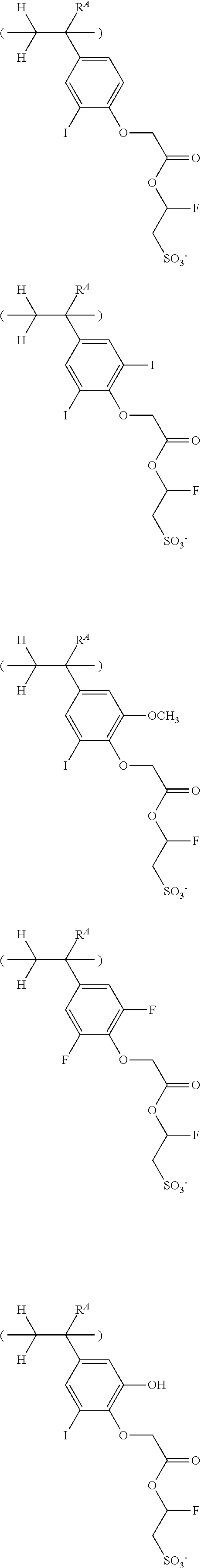

Examples of the anion in repeat unit (c4) are shown below, but not limited thereto. R^{A} is as defined above.

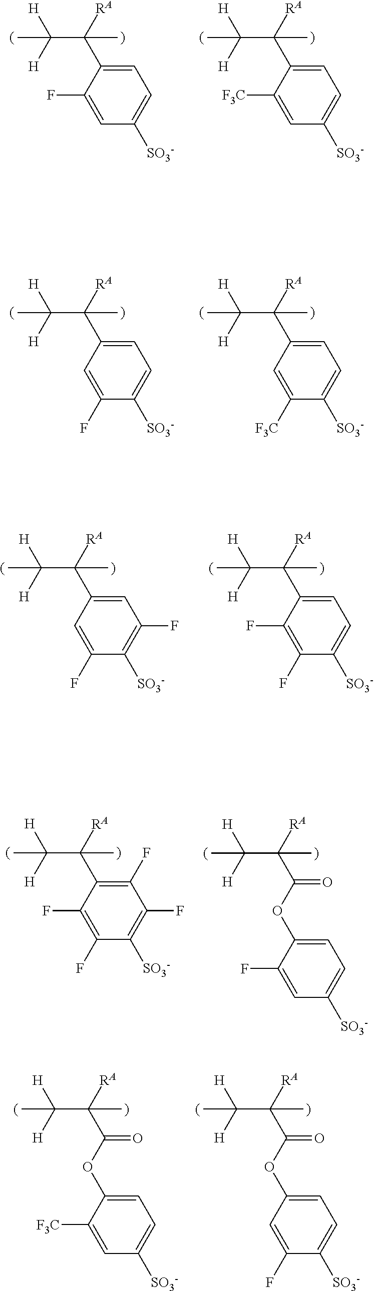

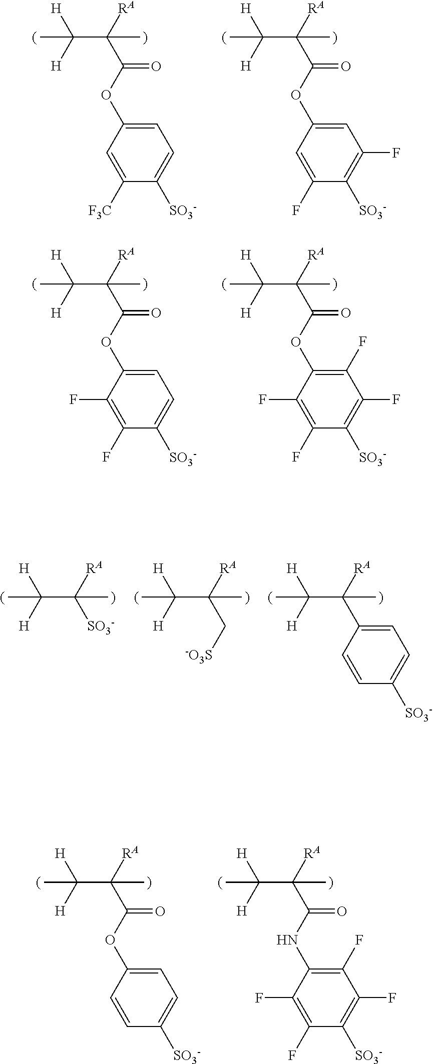

In formulae (c2) to (c4), A⁺ is an onium cation. Suitable onium cations include ammonium, sulfonium and iodonium cations, with the sulfonium and iodonium cations being preferred.

Of the onium cations, a sulfonium cation having the formula (cation-1) or an iodonium cation having the formula (cation-2) is preferred.

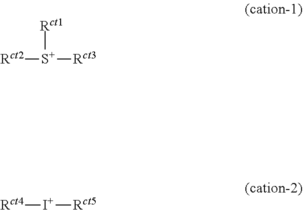

In formulae (cation-1) and (cation-2), R^{ct1} to R^{ct5} are each independently halogen or a C₁-C₃₀ hydrocarbyl group which may contain a heteroatom. Suitable halogen atoms include fluorine, chlorine, bromine and iodine. The hydrocarbyl group may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl; C₃-C₃₀ cyclic saturated hydrocarbyl groups such as cyclopropyl, cyclopentyl, cyclohexyl, cyclopropylmethyl, 4-methylcyclohexyl, cyclohexylmethyl, norbornyl, adamantyl; C₂-C₃₀ alkenyl groups such as vinyl, 1-propenyl, 2-propenyl, butenyl, hexenyl; C₃-C₃₀ cyclic unsaturated hydrocarbyl groups such as cyclohexenyl; C₆-C₃₀ aryl groups such as phenyl, naphthyl, thienyl; C₇-C₃₀ aralkyl groups such as benzyl, 1-phenylethyl, 2-phenylethyl, and combinations thereof. Inter alia, the aryl groups are preferred. In the hydrocarbyl groups, some hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety.

Also, R^{ct1} and R^{ct2} may bond together to form a ring with the sulfur atom to which they are attached. Exemplary structures of the ring are shown below.

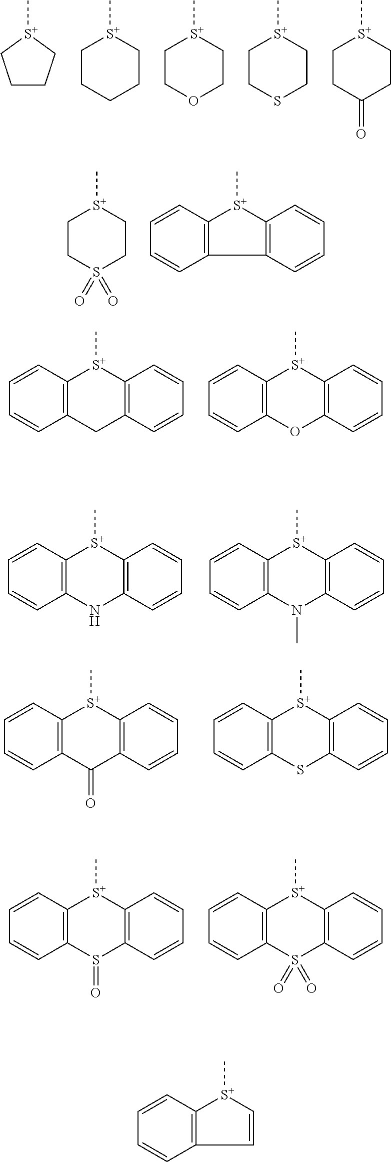

The broken line designates a point of attachment to R^{ct3}.

Examples of the sulfonium cation having formula (cation-1) are shown below, but not limited thereto.

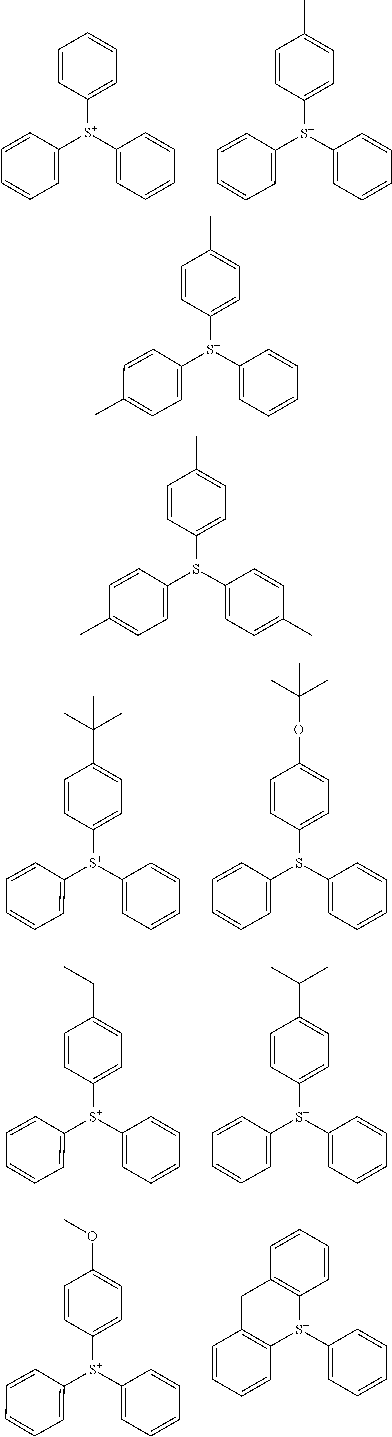

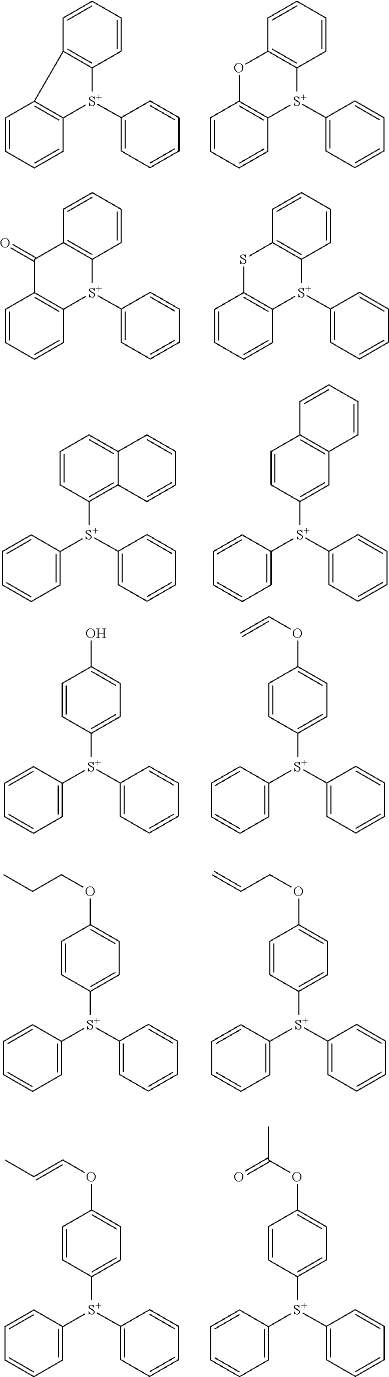

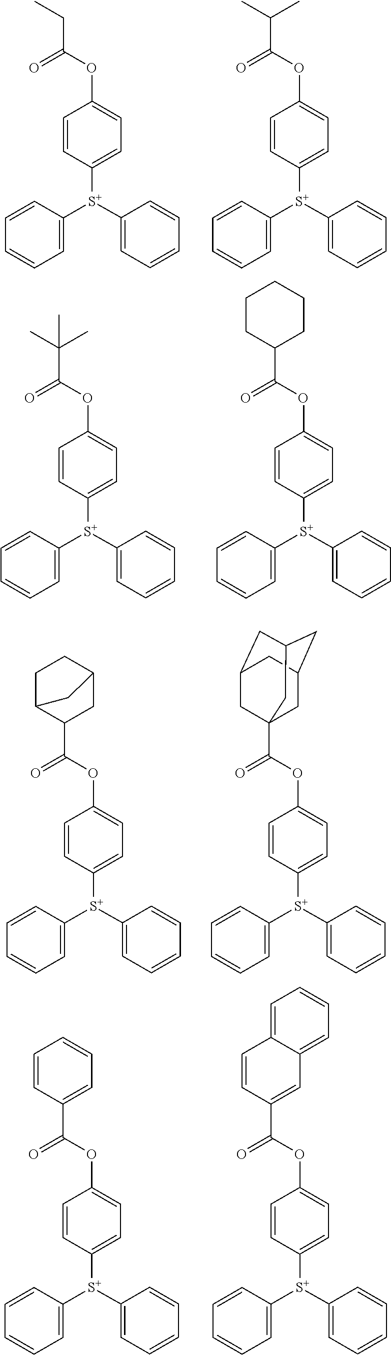

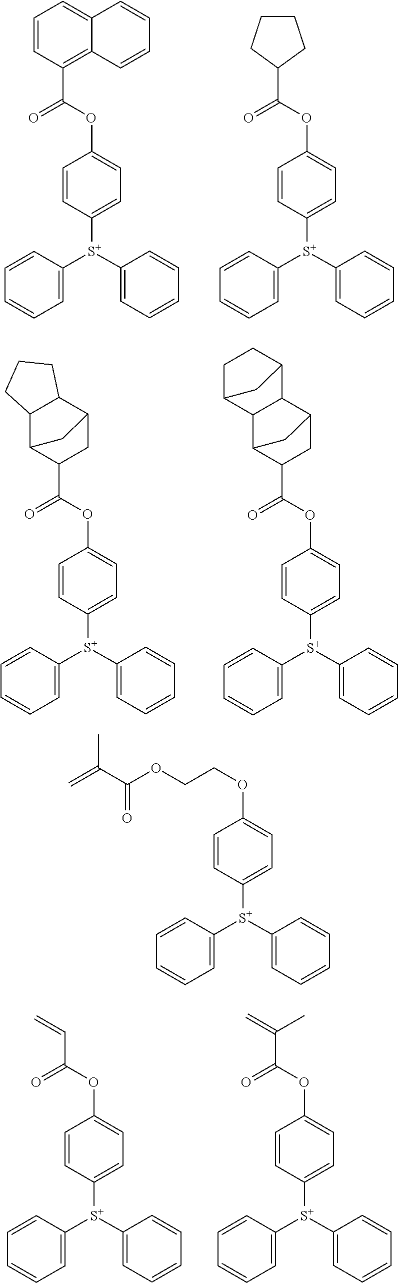

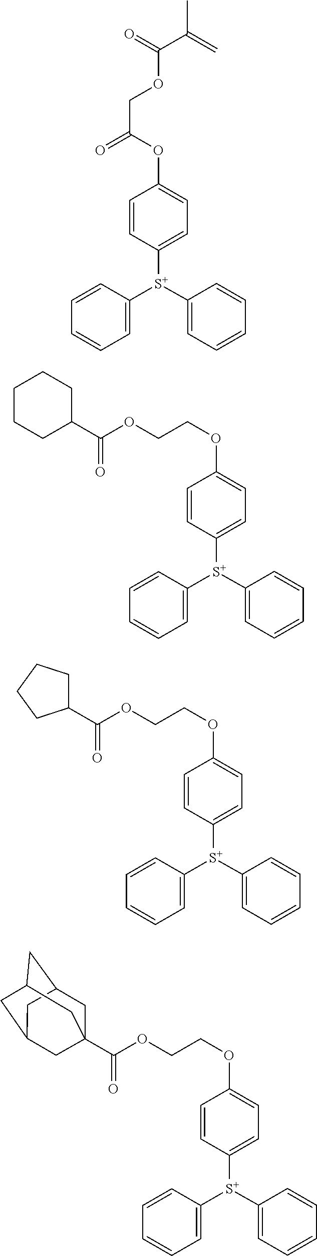 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

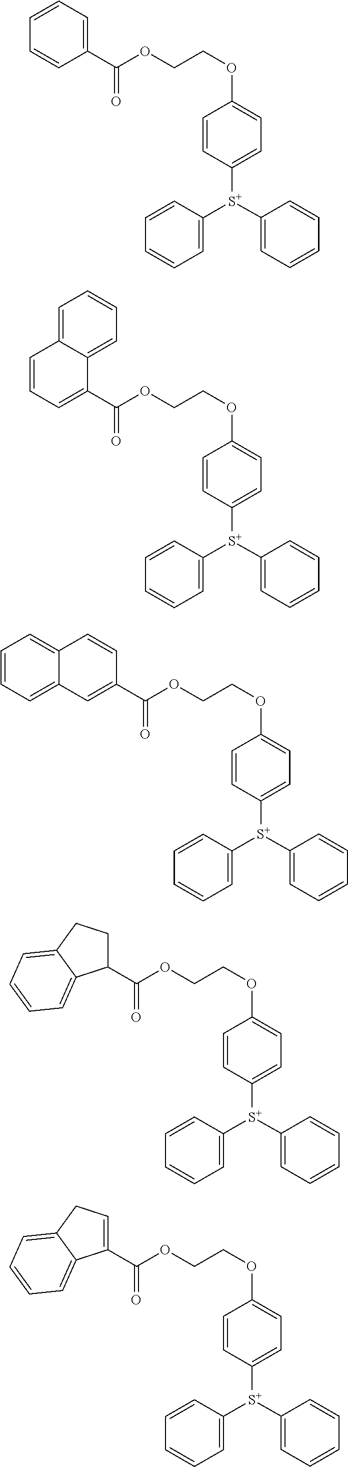 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

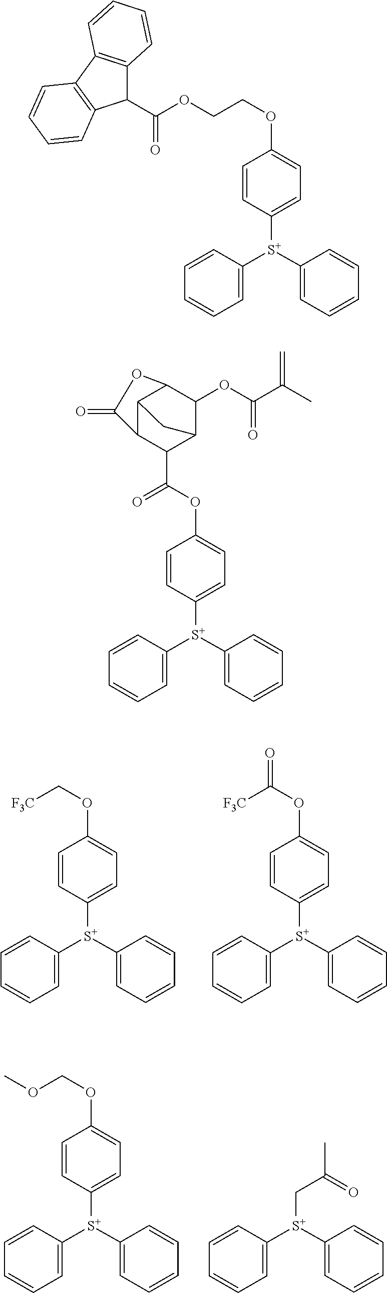 [SMILES: O=C(OCCOc1ccc([S+](c2ccccc2)c2ccccc2)cc1)C1c2ccccc2-c2ccccc21 ; C=C(C)C(=O)OC1C2CC3C1OC(=O)C3C2C(=O)Oc1ccc([S+](c2ccccc2)c2ccccc2)cc1 ; FC(F)(F)COc1ccc([S+](c2ccccc2)c2ccccc2)cc1 ; O=C(Oc1ccc([S+](c2ccccc2)c2ccccc2)cc1)C(F)(F)F ; COCOc1ccc([S+](c2ccccc2)c2ccccc2)cc1 ; CC(=O)C[S+](c1ccccc1)c1ccccc1]

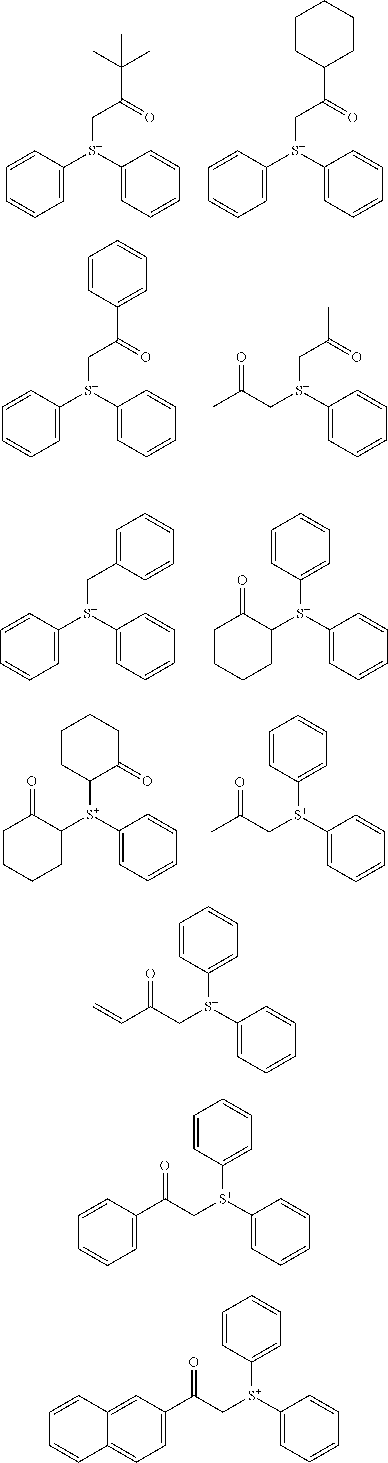

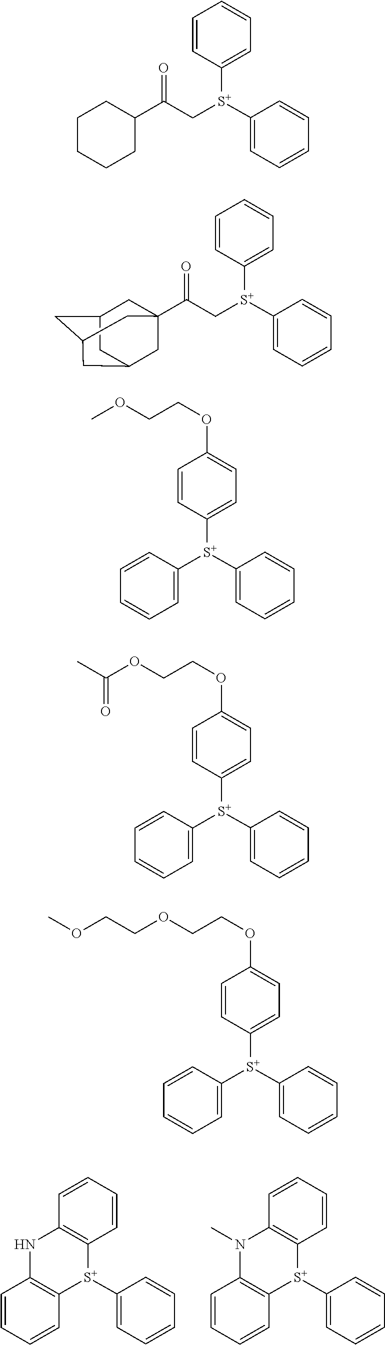

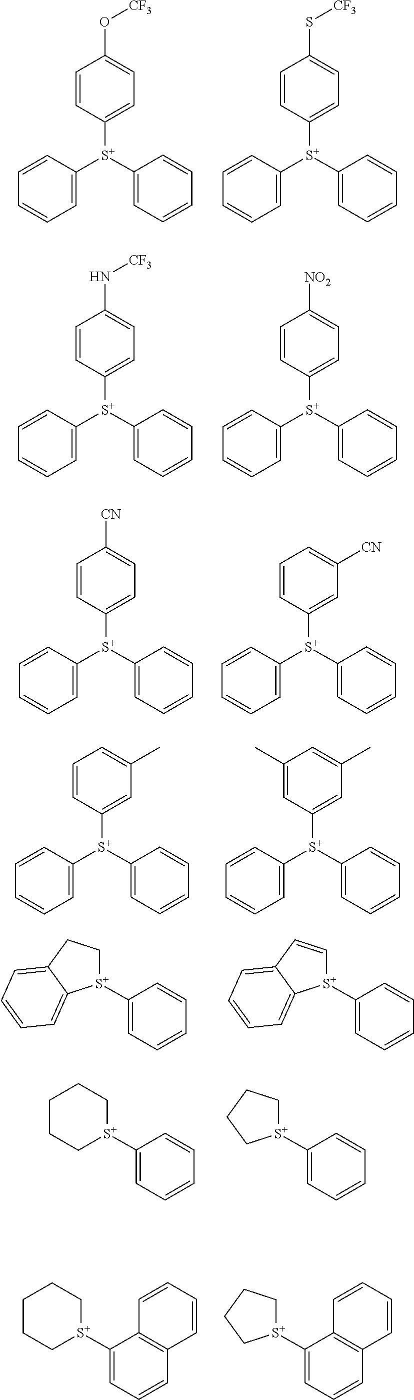

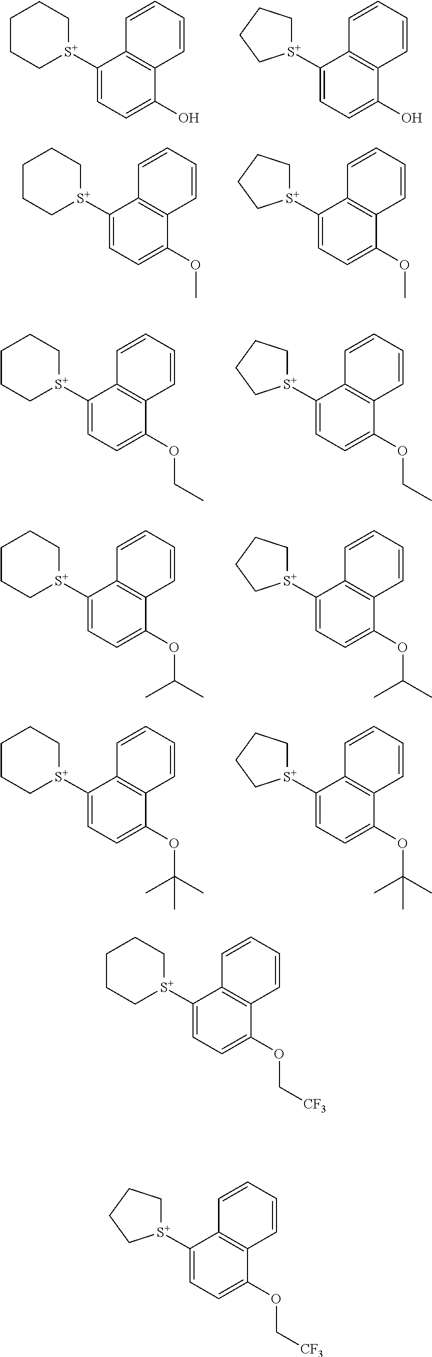

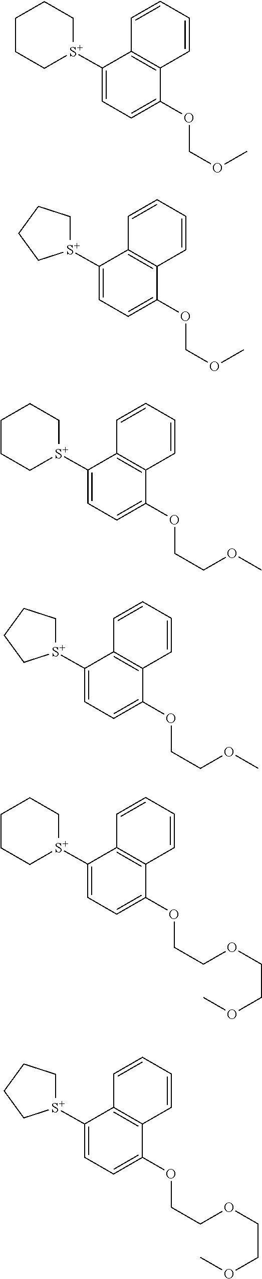 [SMILES: COCOc1ccc([S+]2CCCCC2)c2ccccc12 ; COCOc1ccc([S+]2CCCC2)c2ccccc12 ; COCCOc1ccc([S+]2CCCCC2)c2ccccc12 ; COCCOc1ccc([S+]2CCCC2)c2ccccc12 ; COCCOCCOc1ccc([S+]2CCCCC2)c2ccccc12 ; COCCOCCOc1ccc([S+]2CCCC2)c2ccccc12]

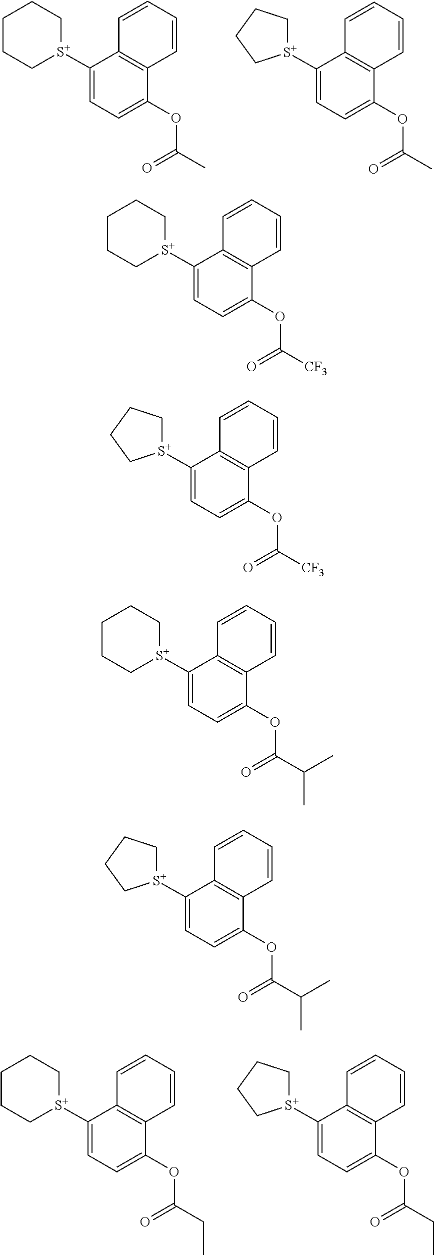

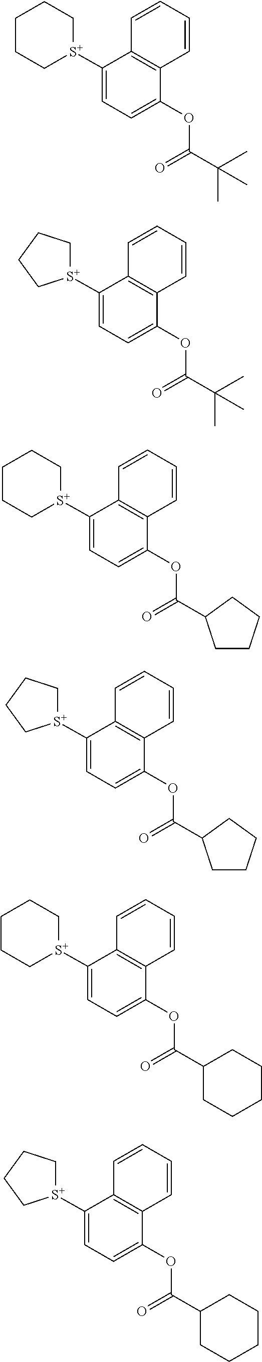 [SMILES: CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1 ; O=C(Oc1ccc([S+]2CCCCC2)c2ccccc12)C1CCCCC1 ; O=C(Oc1ccc([S+]2CCCC2)c2ccccc12)C1CCCCC1]

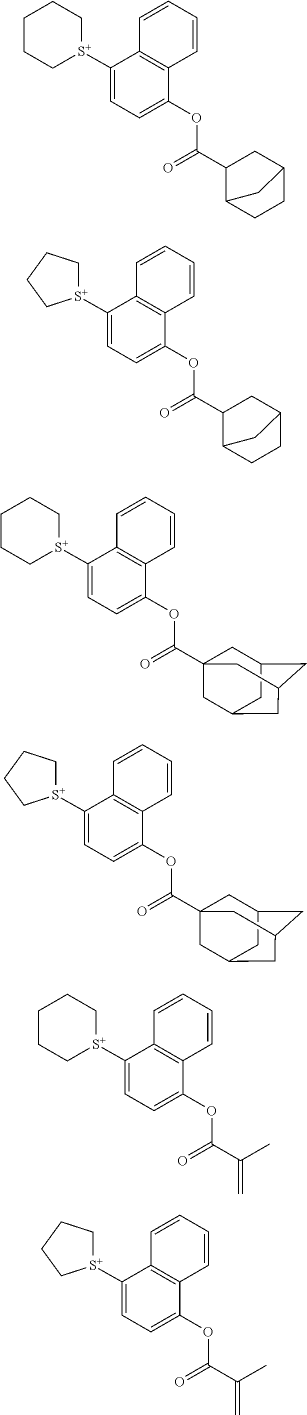 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

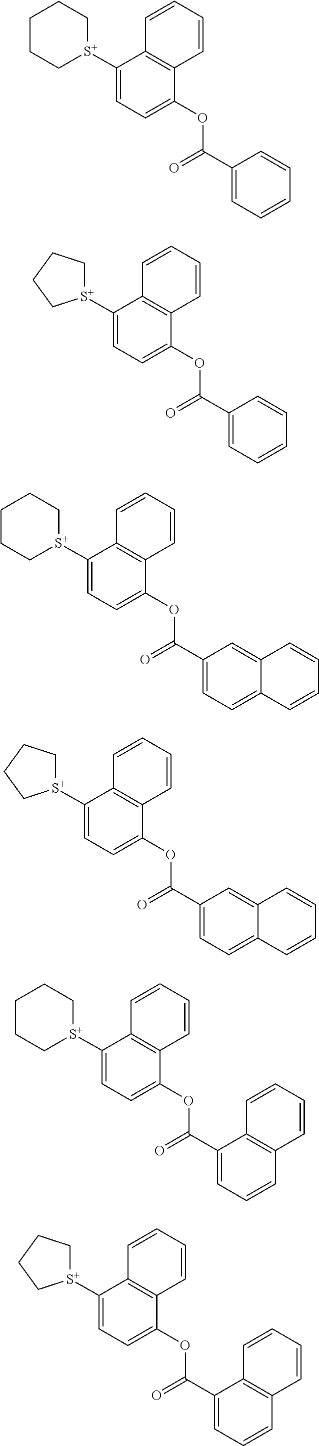 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

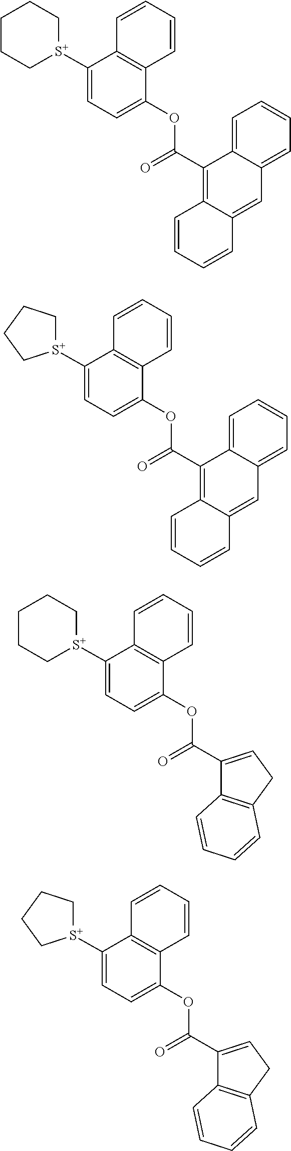 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21]

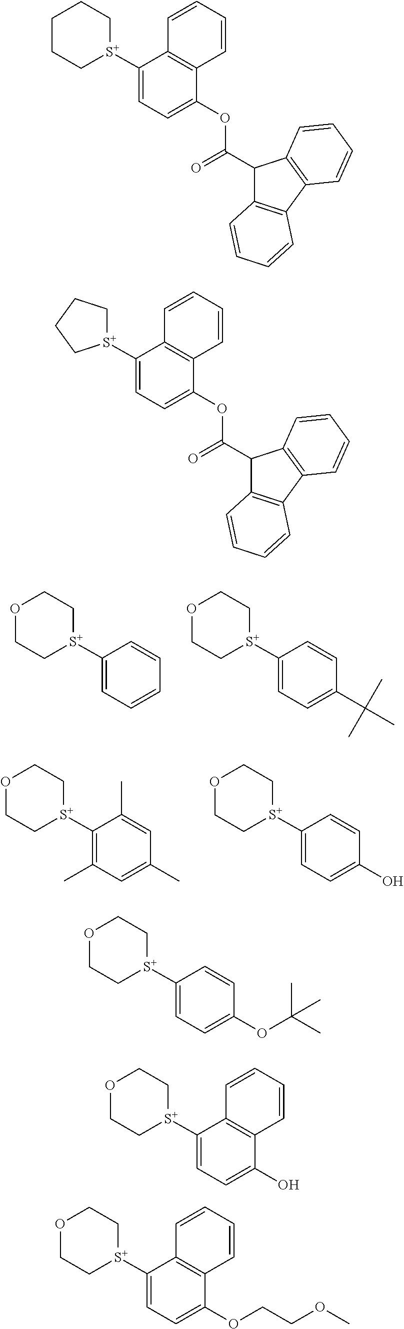

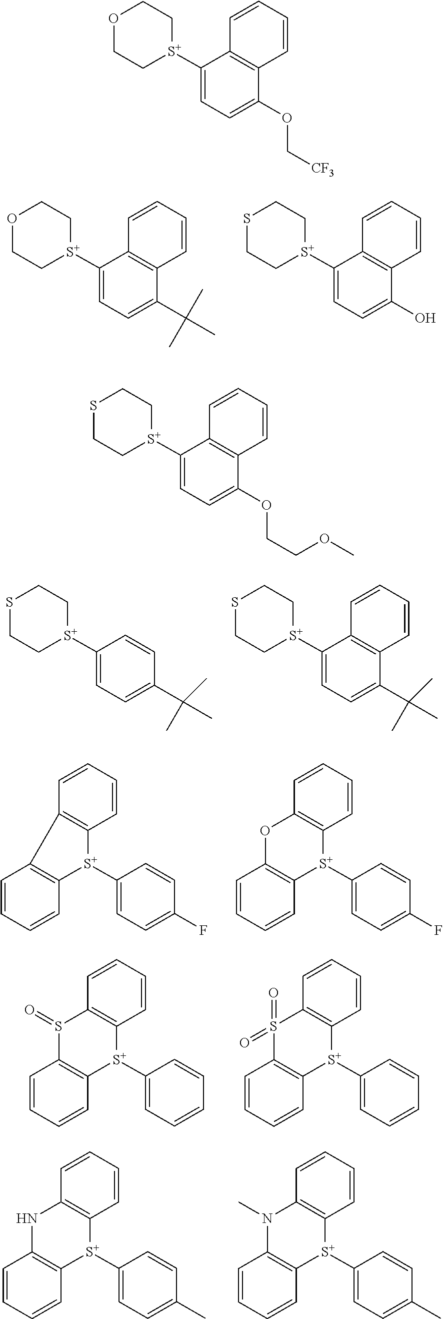

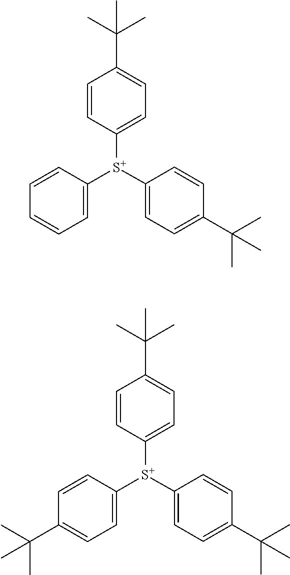

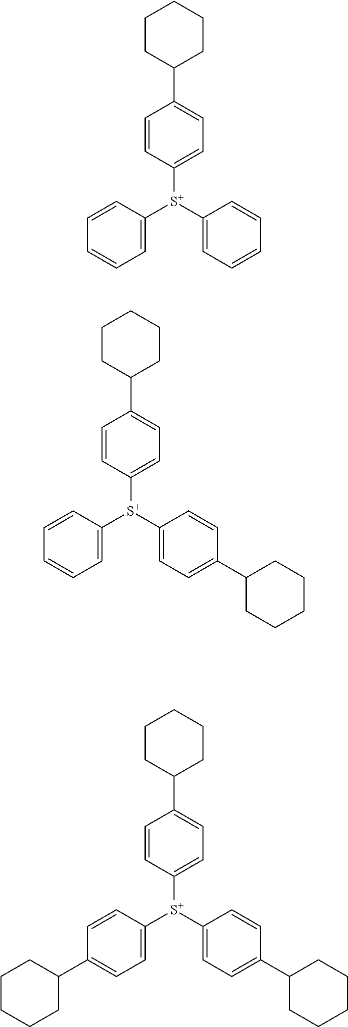 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

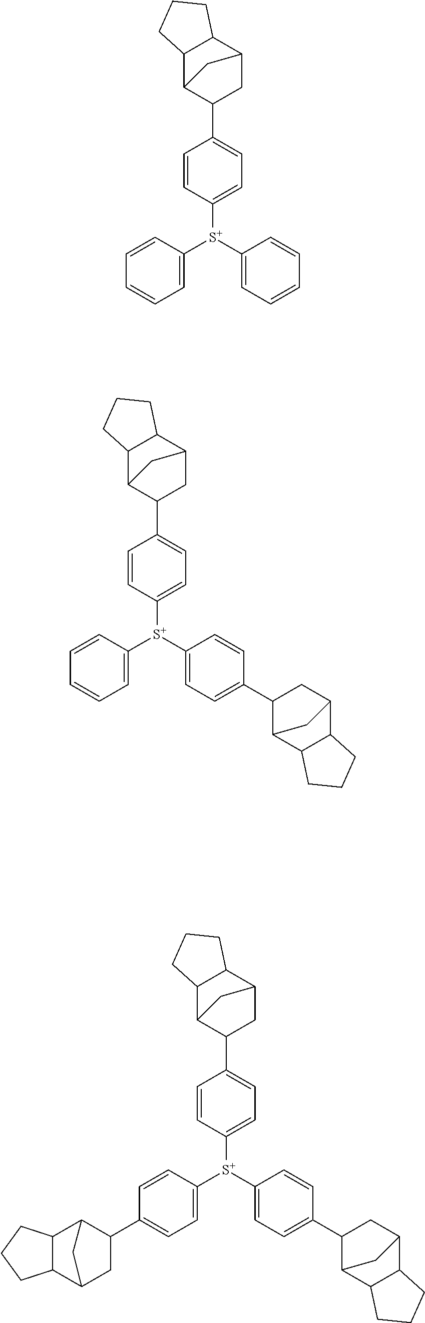 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

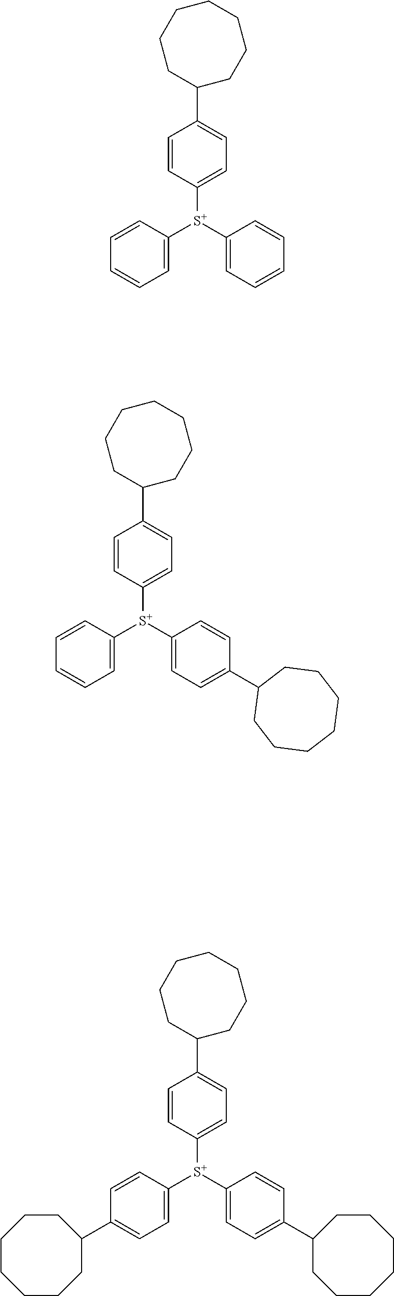 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

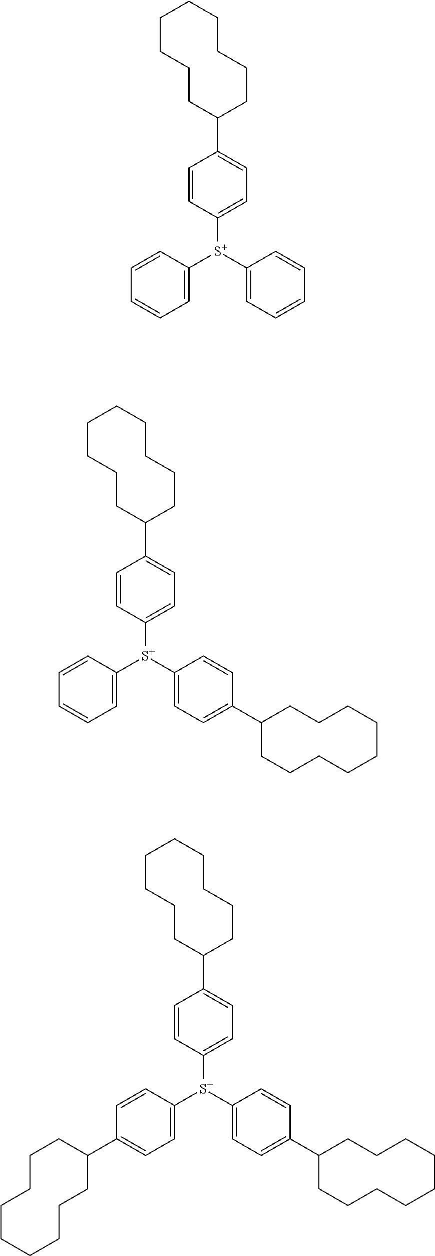 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

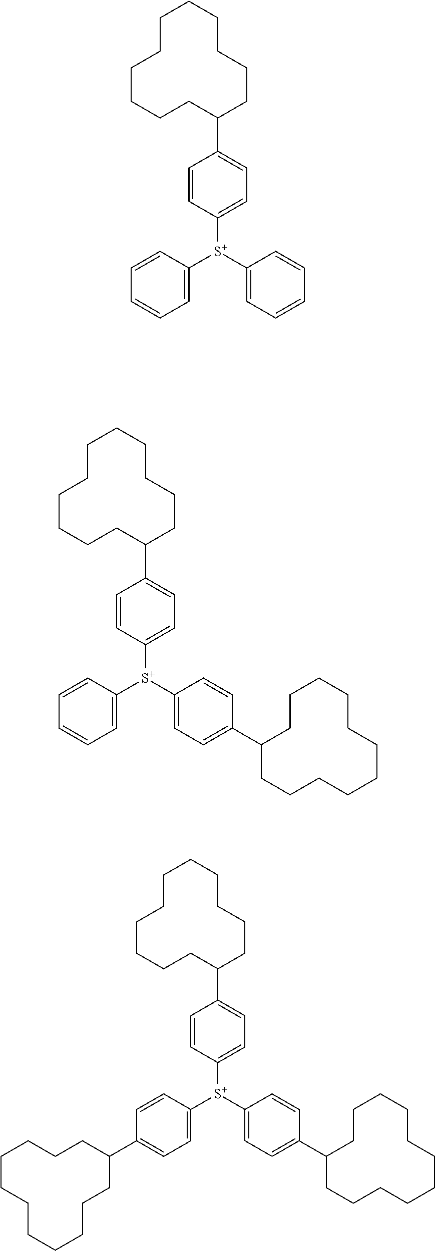 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

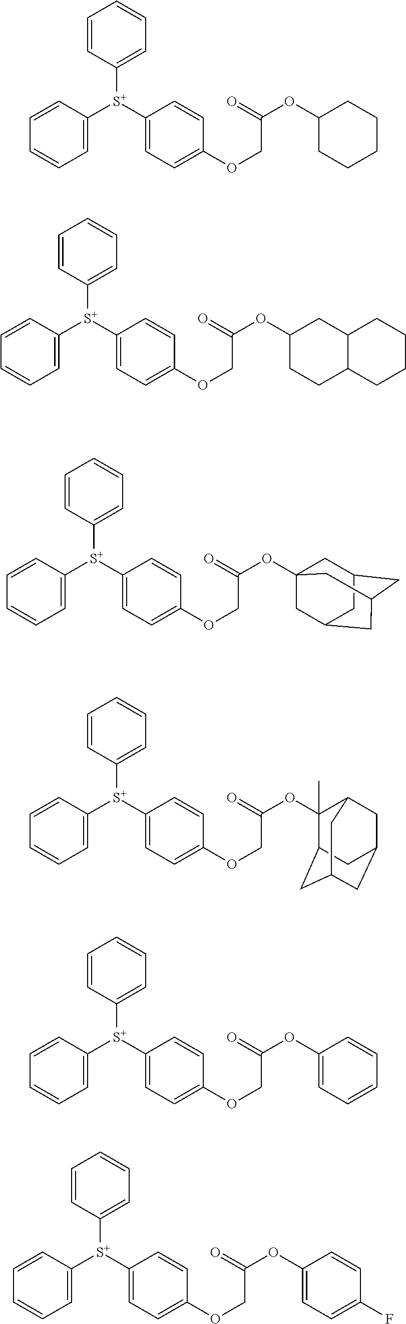 [SMILES: O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1 ; O=C(COc1ccc([S+](c2ccccc2)c2ccccc2)cc1)Oc1ccc(F)cc1]

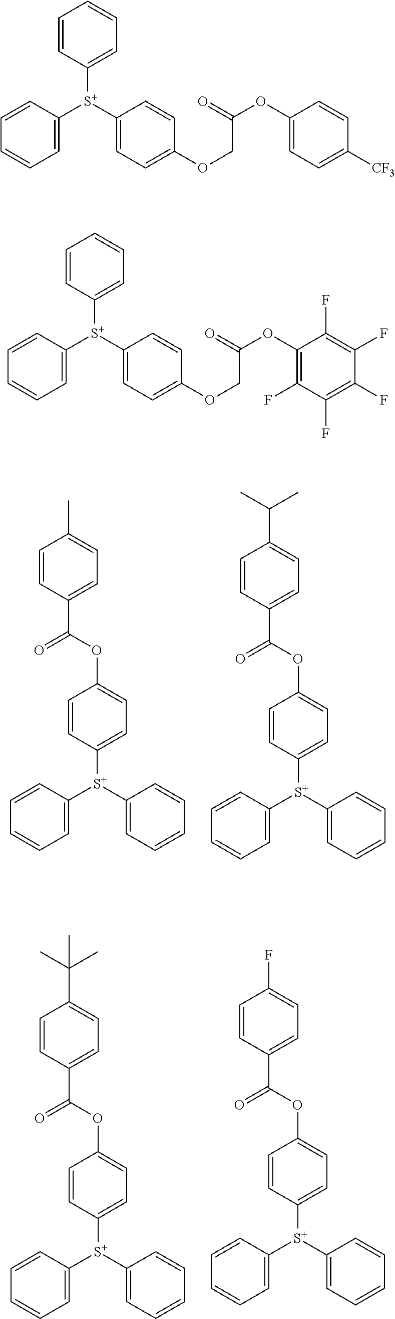 [SMILES: O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; Cc1ccc(C(=O)Oc2ccc([S+](c3ccccc3)c3ccccc3)cc2)cc1 ; CC(C)c1ccc(C(=O)Oc2ccc([S+](c3ccccc3)c3ccccc3)cc2)cc1 ; CC(C)(C)c1ccc(C(=O)Oc2ccc([S+](c3ccccc3)c3ccccc3)cc2)cc1 ; O=C(Oc1ccc([S+](c2ccccc2)c2ccccc2)cc1)c1ccc(F)cc1]

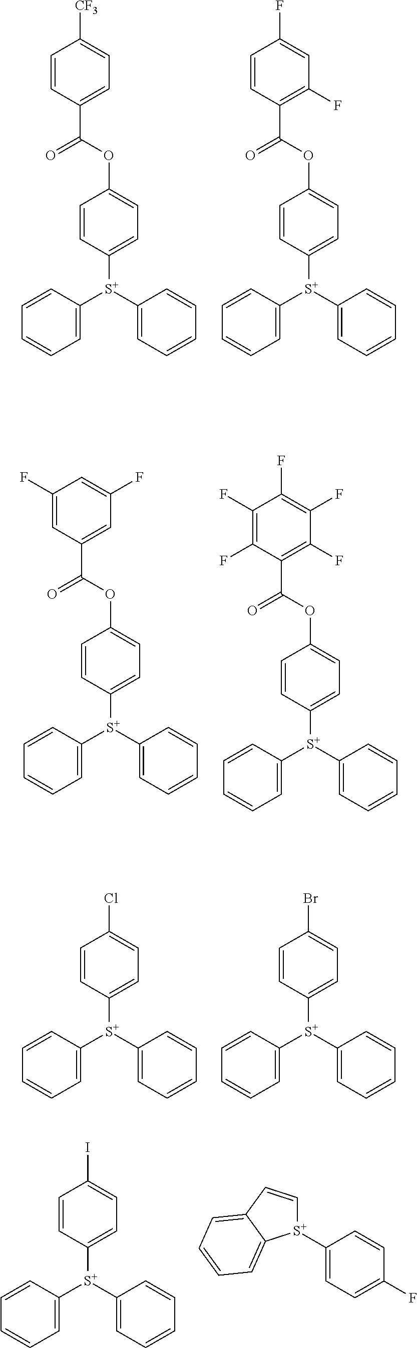

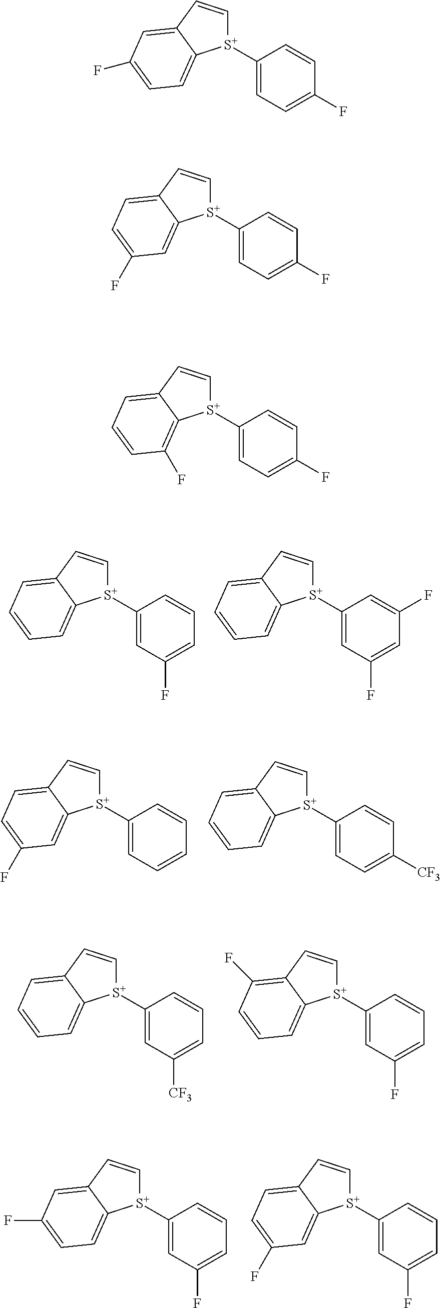

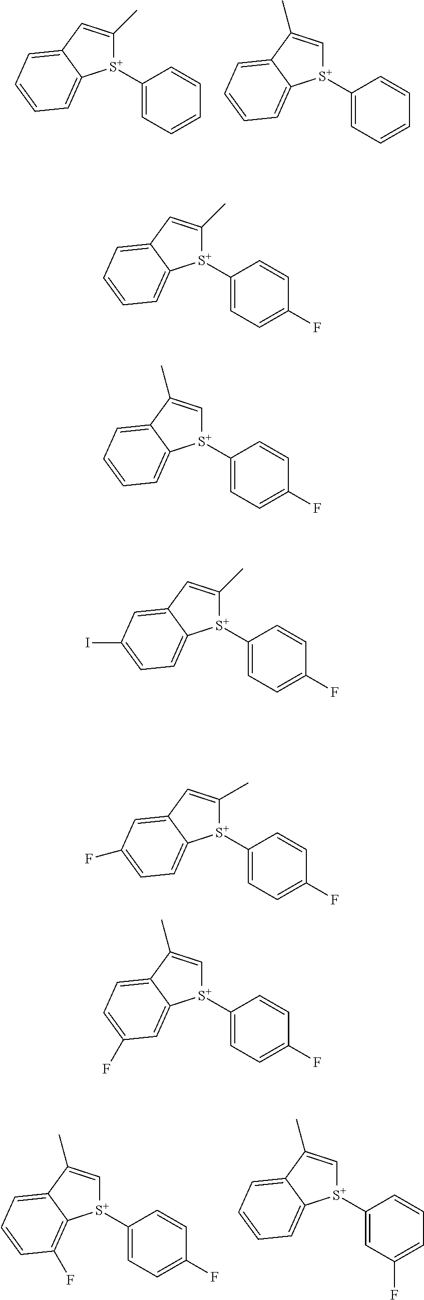

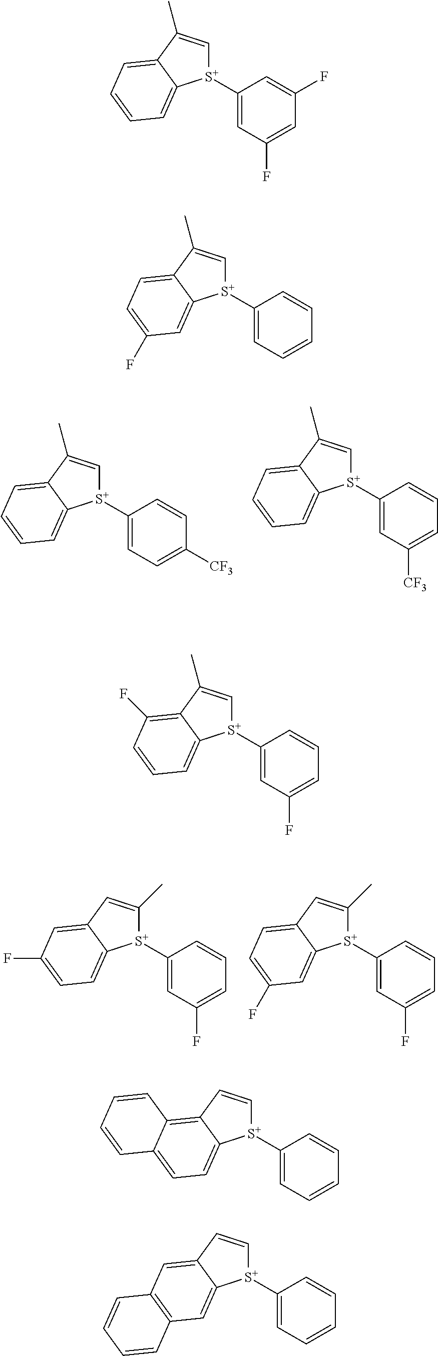

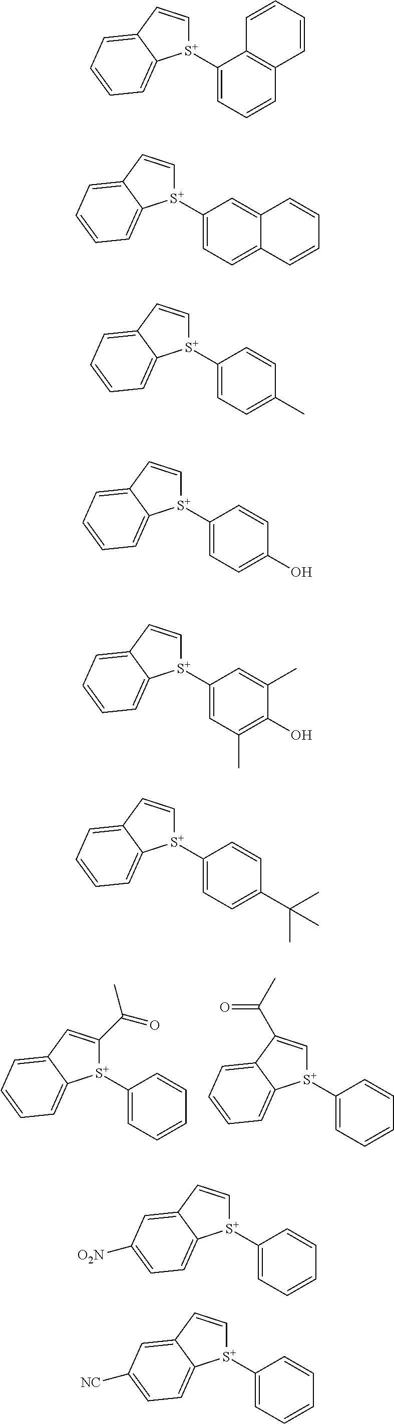

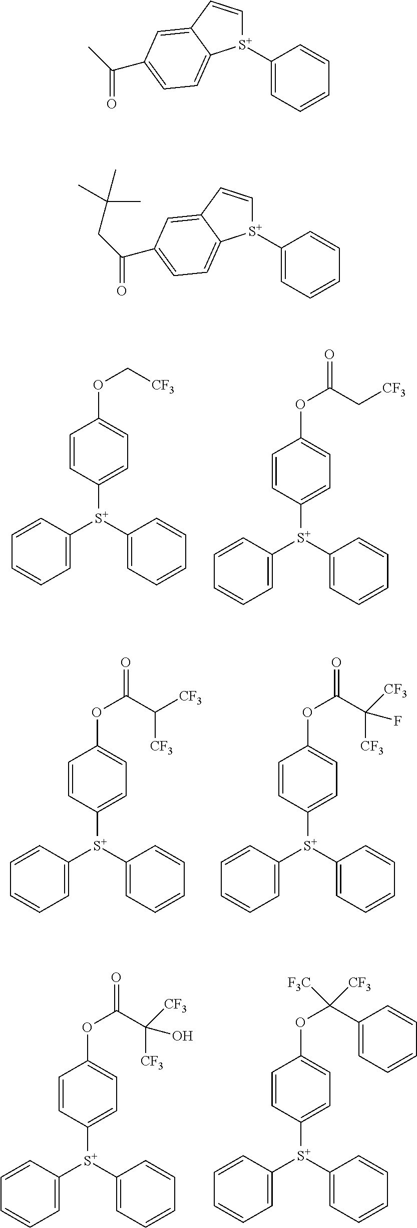

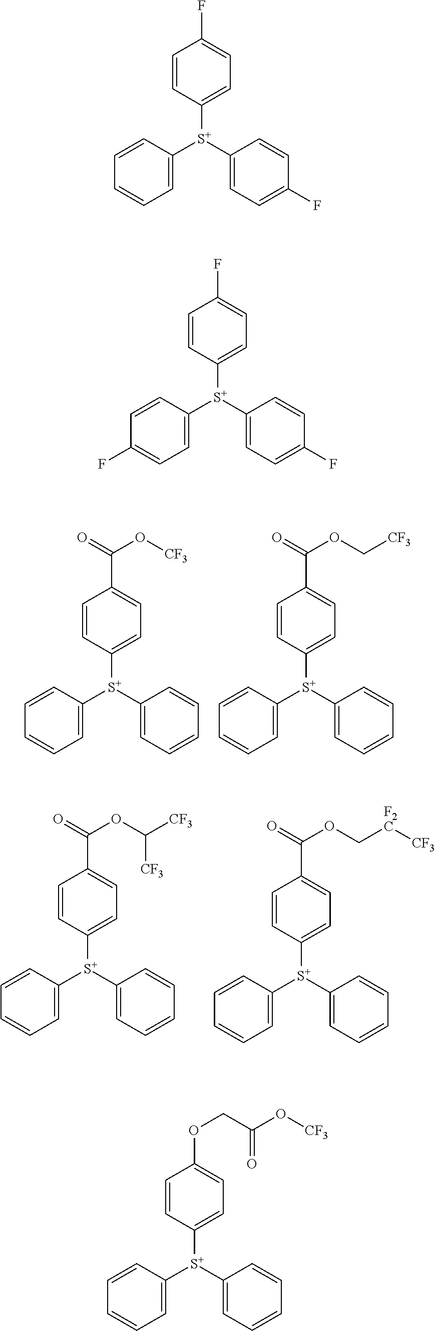

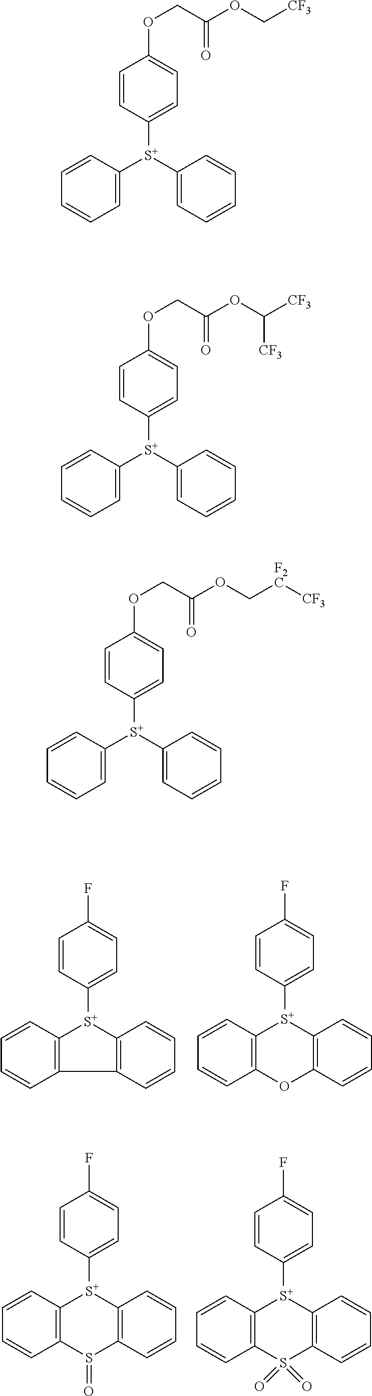

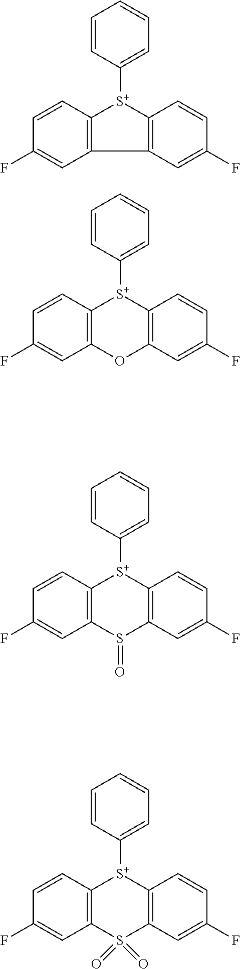 [SMILES: Fc1ccc2c(c1)c1cc(F)ccc1[s+]2-c1ccccc1 ; Fc1ccc2c(c1)Oc1cc(F)ccc1[S+]2c1ccccc1 ; O=S1c2cc(F)ccc2[S+](c2ccccc2)c2ccc(F)cc21 ; O=S1(=O)c2cc(F)ccc2[S+](c2ccccc2)c2ccc(F)cc21]

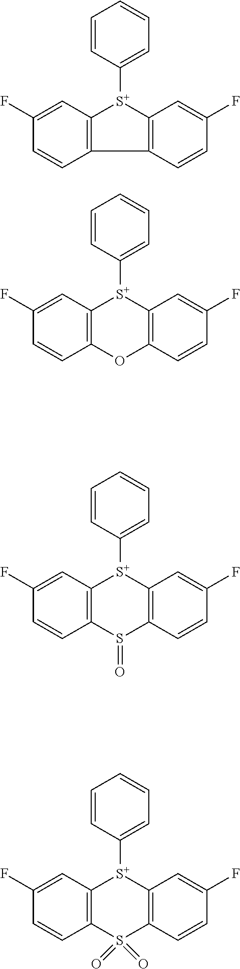 [SMILES: Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

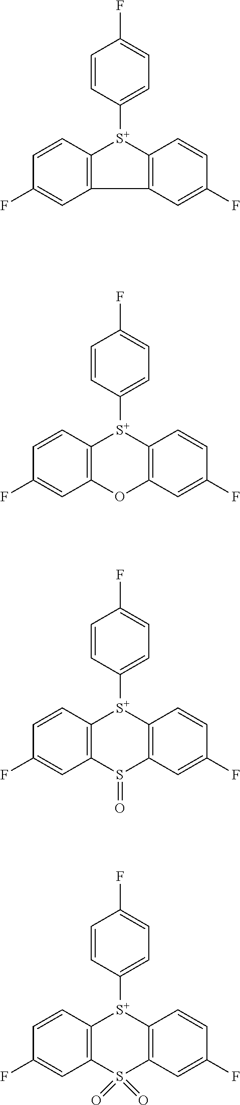 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21]

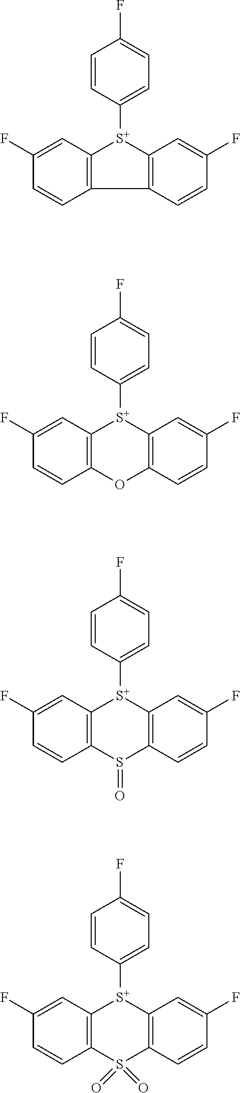 [SMILES: Fc1ccc(-[s+]2c3cc(F)ccc3c3ccc(F)cc32)cc1 ; Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

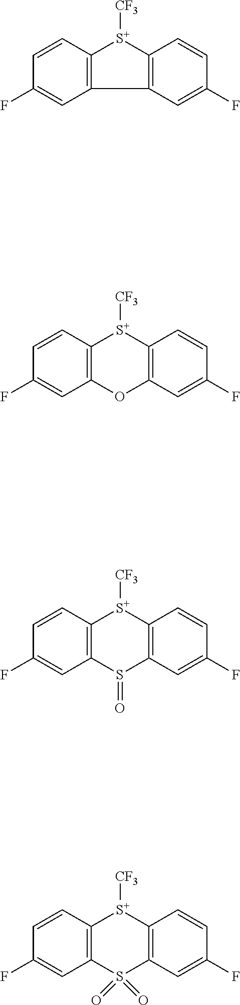 [SMILES: Fc1ccc2c(c1)c1cc(F)ccc1[s+]2C(F)(F)F ; Fc1ccc2c(c1)Oc1cc(F)ccc1[S+]2C(F)(F)F ; O=S1c2cc(F)ccc2[S+](C(F)(F)F)c2ccc(F)cc21 ; O=S1(=O)c2cc(F)ccc2[S+](C(F)(F)F)c2ccc(F)cc21]

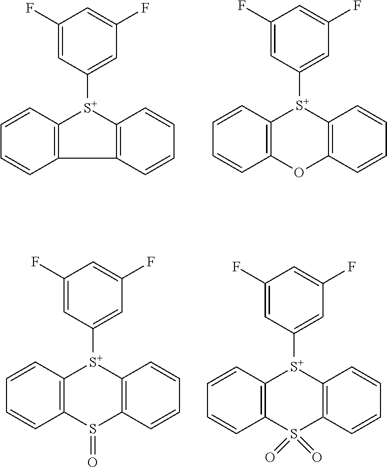 [SMILES: Fc1cc(F)cc(-[s+]2c3ccccc3c3ccccc32)c1 ; Fc1cc(F)cc([S+]2c3ccccc3Oc3ccccc32)c1 ; O=S1c2ccccc2[S+](c2cc(F)cc(F)c2)c2ccccc21 ; O=S1(=O)c2ccccc2[S+](c2cc(F)cc(F)c2)c2ccccc21]

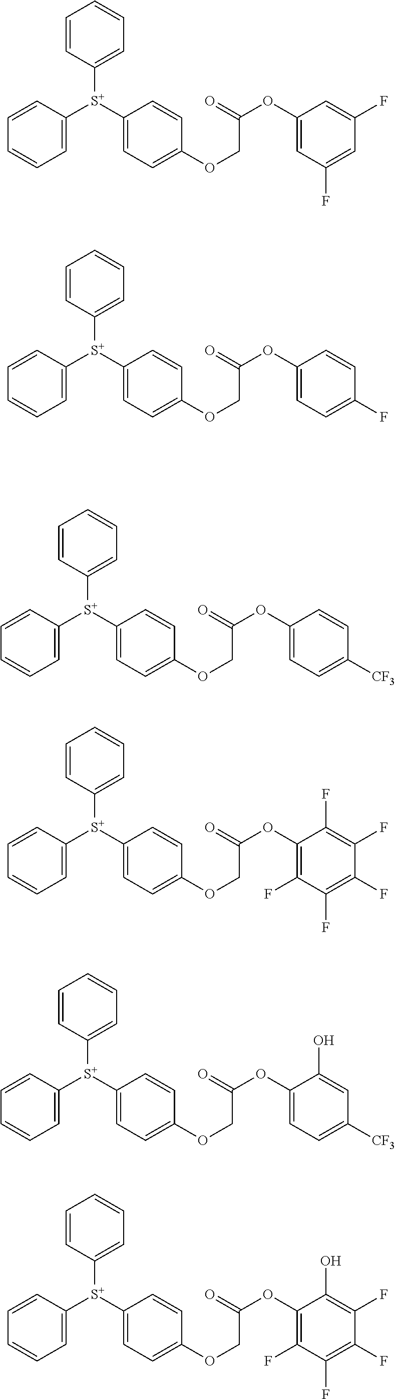 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

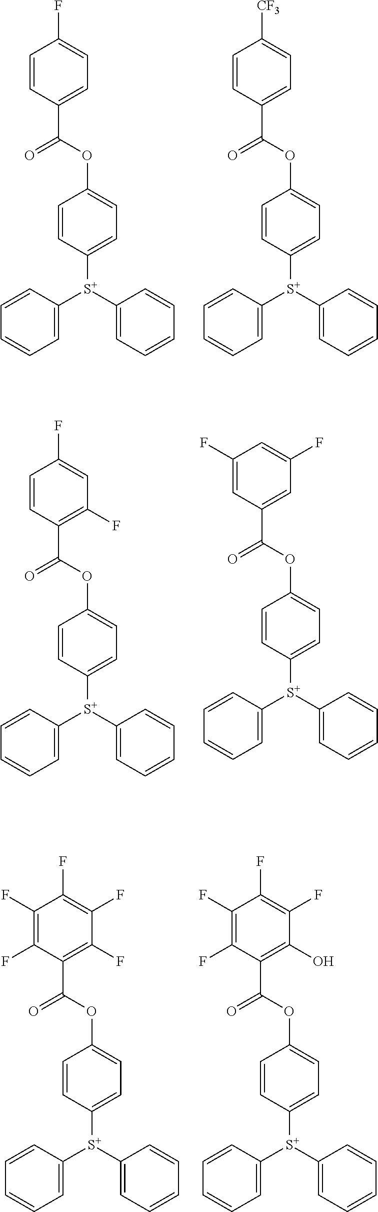 [SMILES: O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F]

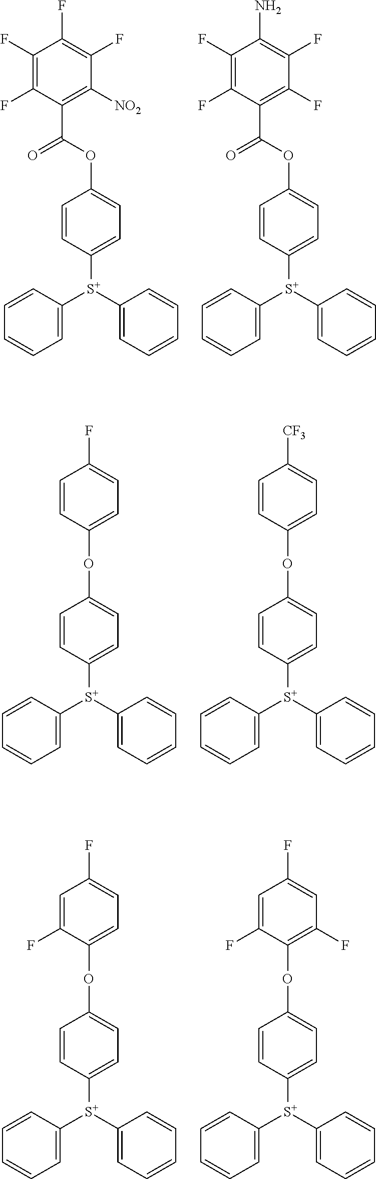 [SMILES: O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(F)c(Oc2ccc([S+](c3ccccc3)c3ccccc3)cc2)c(F)c1]

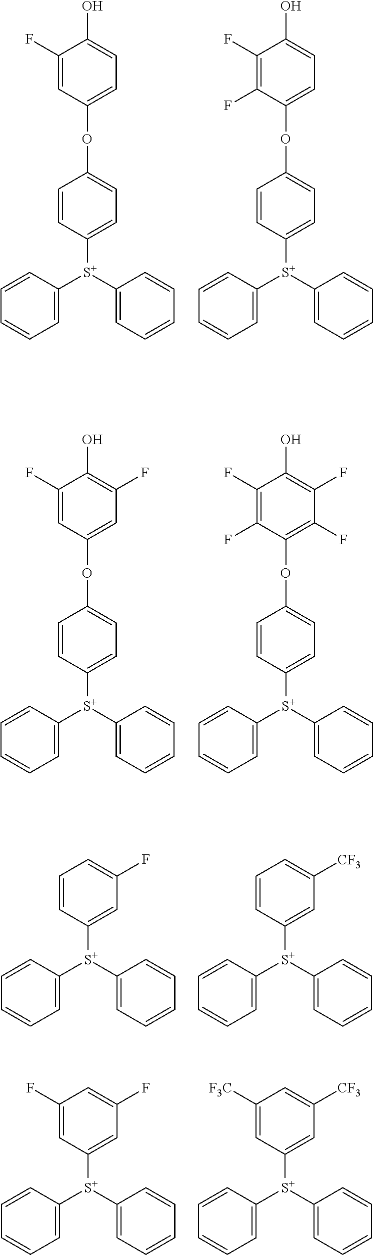

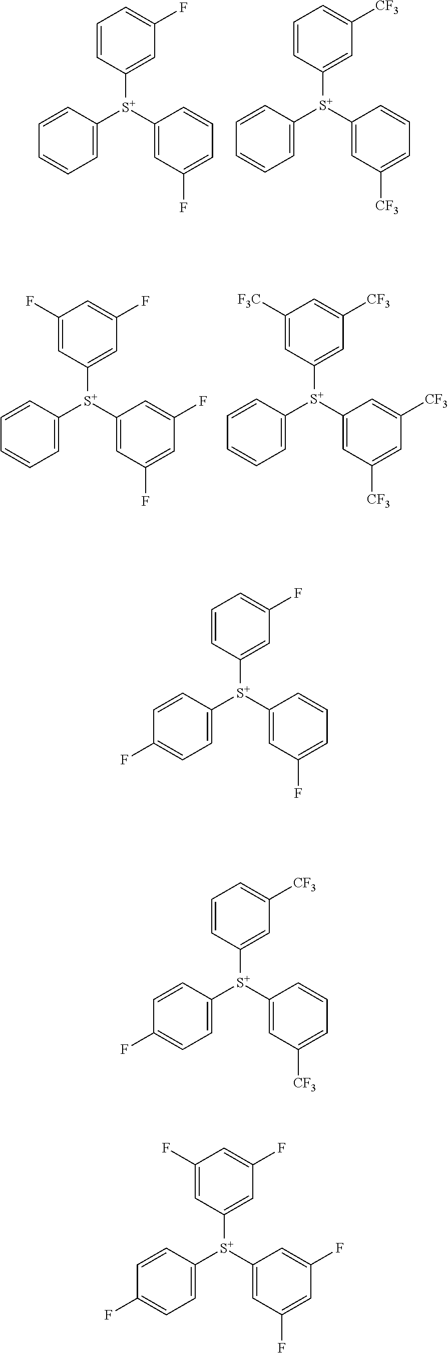

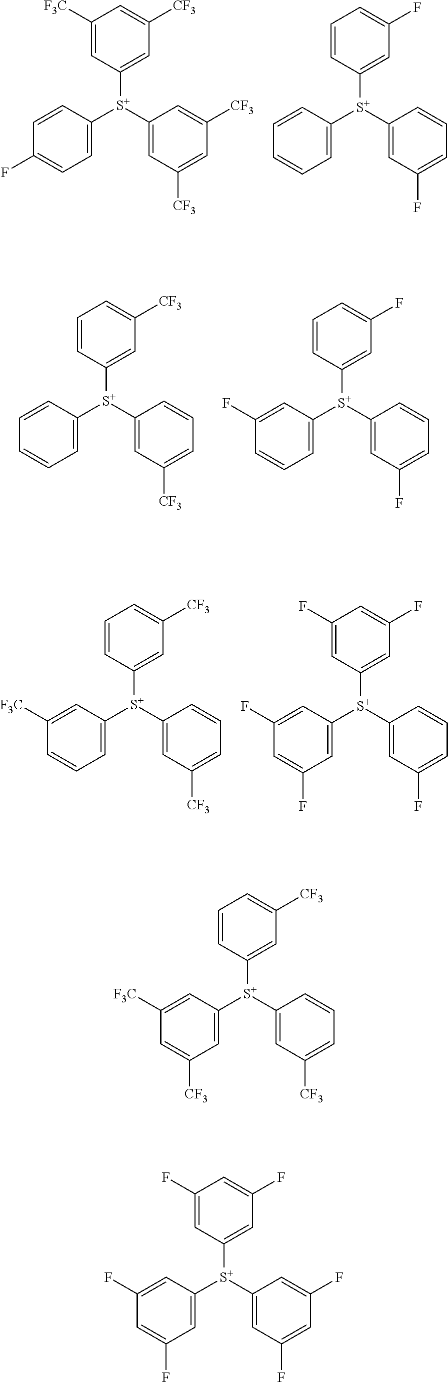

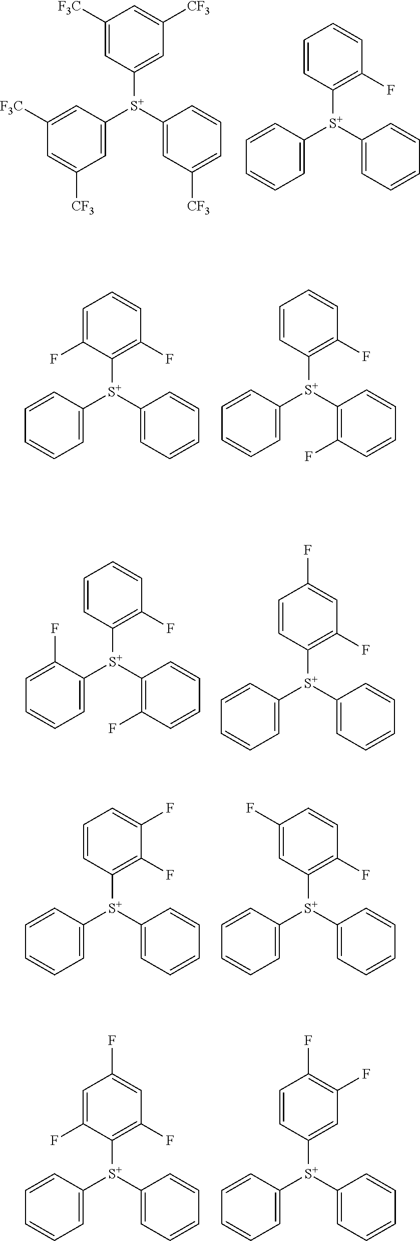

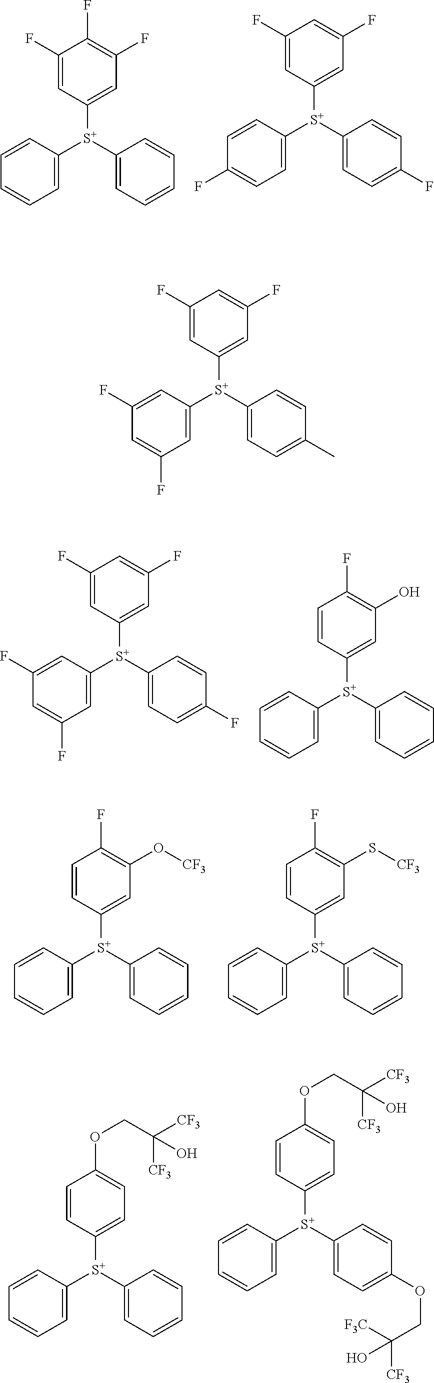

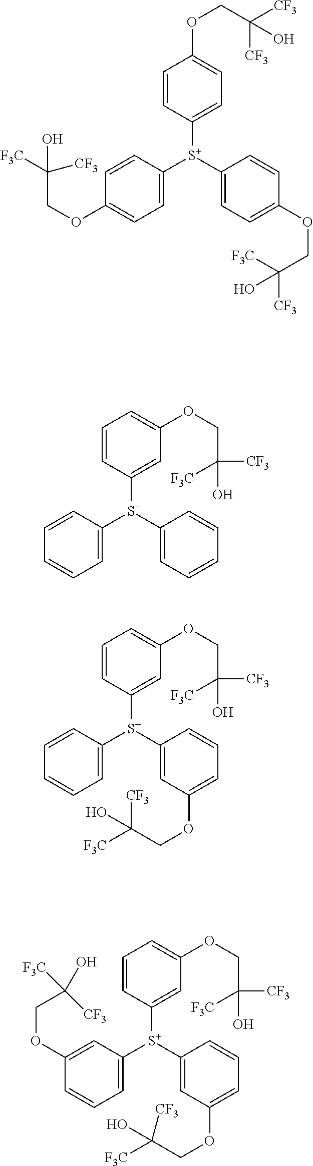 [SMILES: OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F ; OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F]

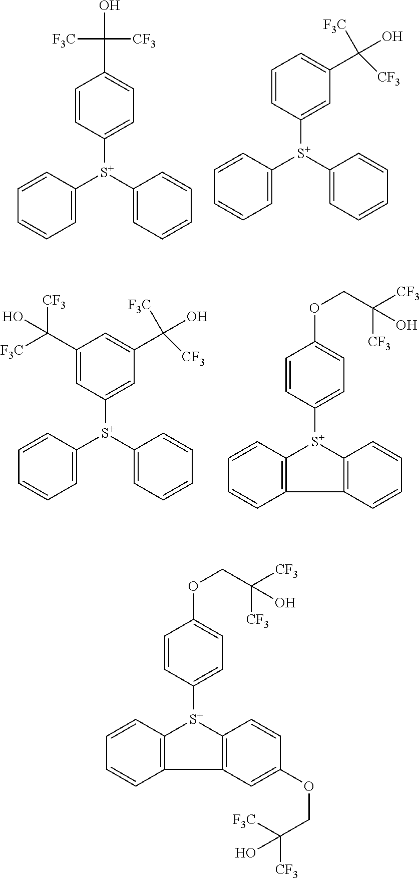 [SMILES: OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F]

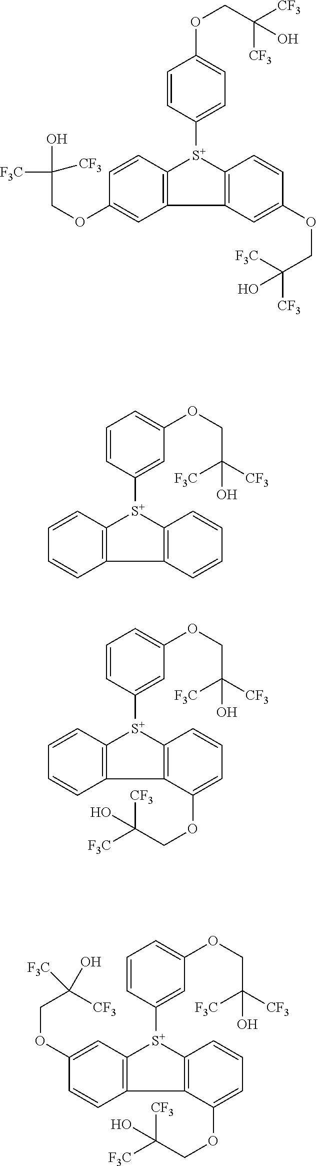 [SMILES: OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F]

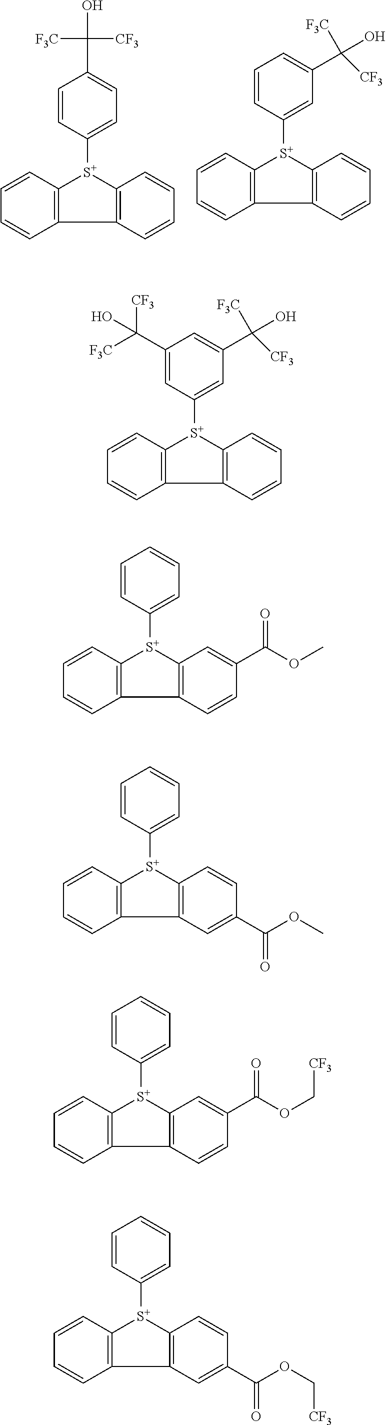

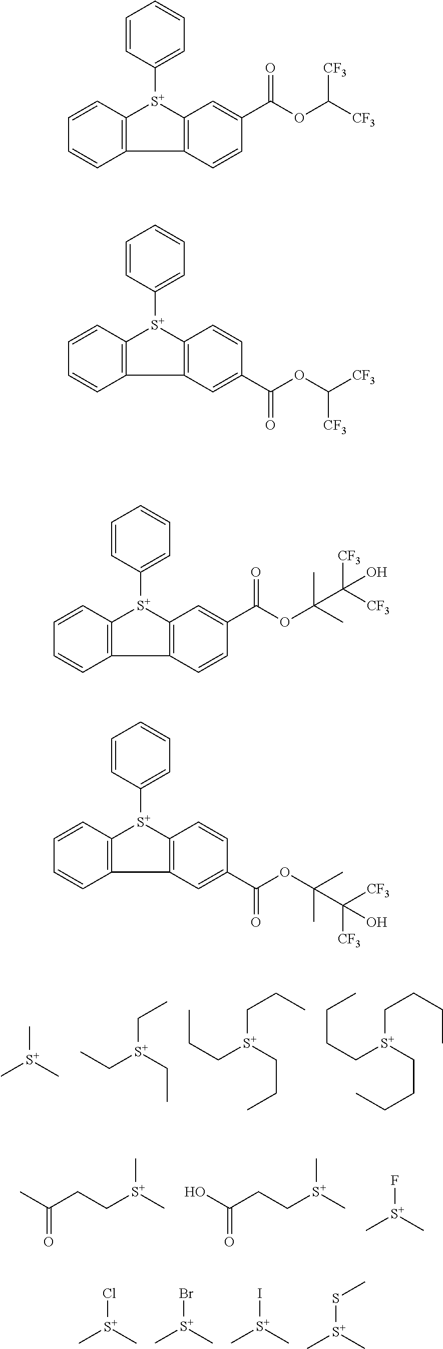

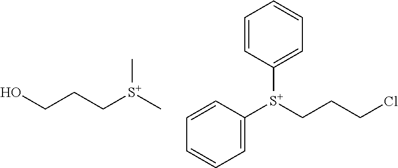

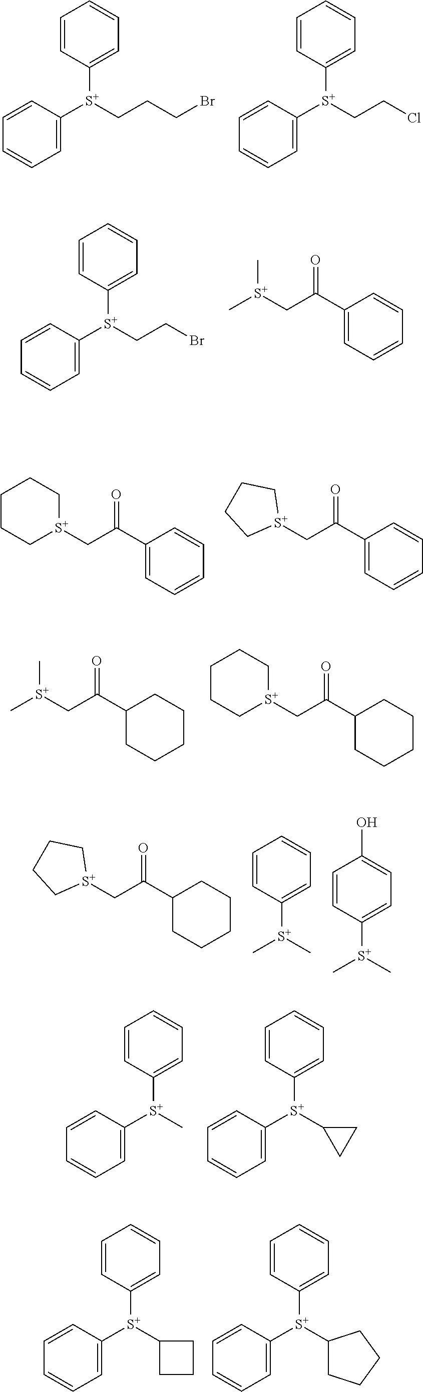

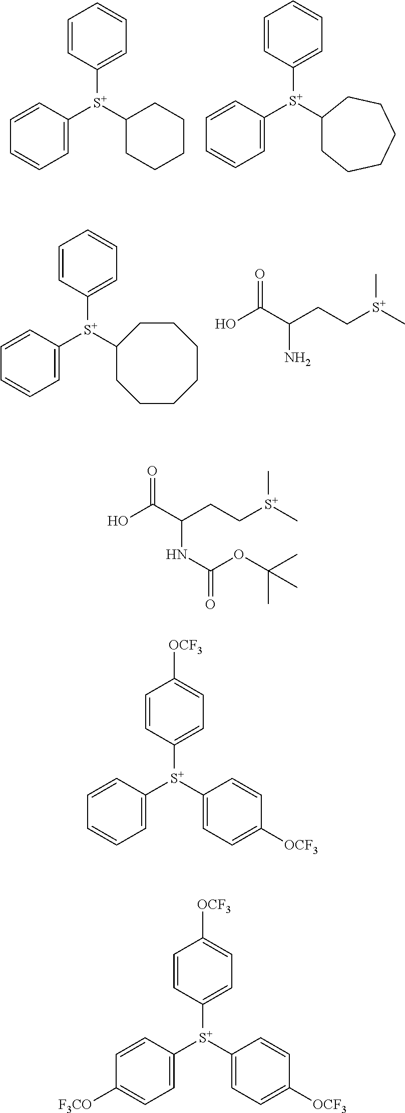

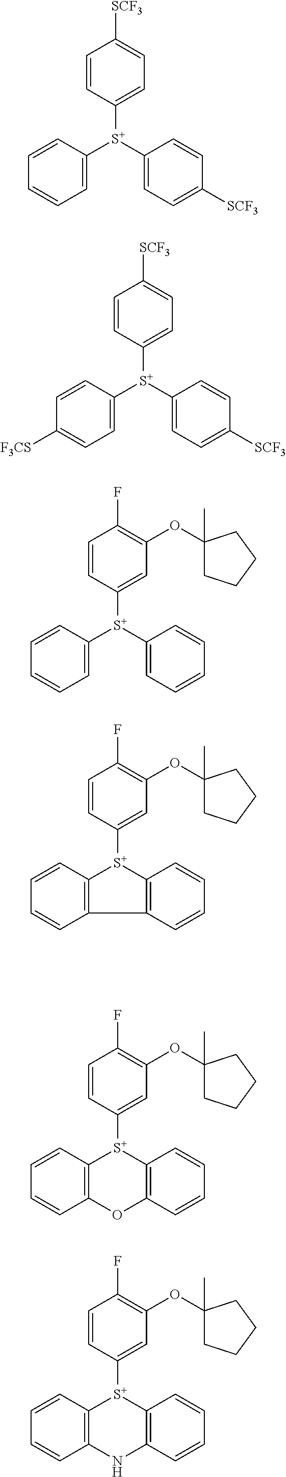

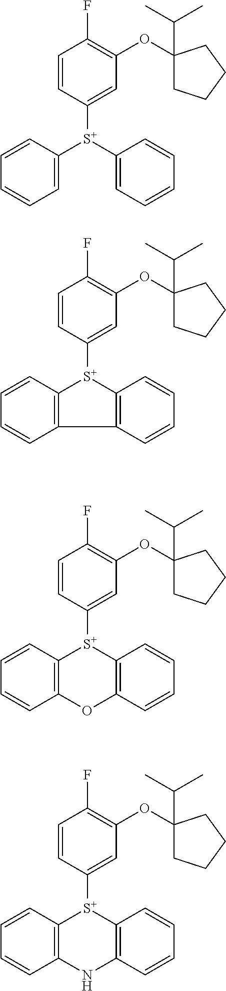 [SMILES: CC(C)C1(Oc2cc([S+](c3ccccc3)c3ccccc3)ccc2F)CCCC1 ; CC(C)C1(Oc2cc(-[s+]3c4ccccc4c4ccccc43)ccc2F)CCCC1 ; CC(C)C1(Oc2cc([S+]3c4ccccc4Oc4ccccc43)ccc2F)CCCC1 ; CC(C)C1(Oc2cc([S+]3c4ccccc4Nc4ccccc43)ccc2F)CCCC1]

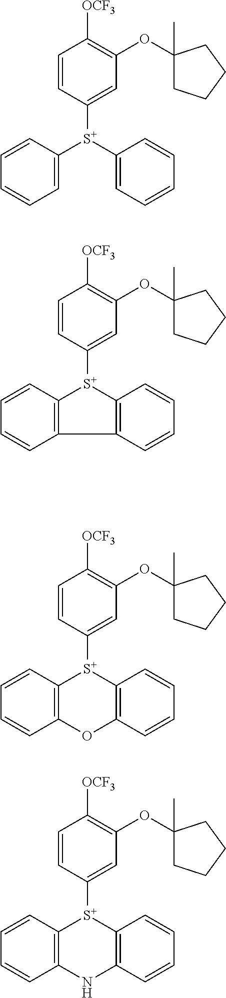 [SMILES: CC1(Oc2cc([S+](c3ccccc3)c3ccccc3)ccc2OC(F)(F)F)CCCC1 ; CC1(Oc2cc(-[s+]3c4ccccc4c4ccccc43)ccc2OC(F)(F)F)CCCC1 ; CC1(Oc2cc([S+]3c4ccccc4Oc4ccccc43)ccc2OC(F)(F)F)CCCC1 ; CC1(Oc2cc([S+]3c4ccccc4Nc4ccccc43)ccc2OC(F)(F)F)CCCC1]

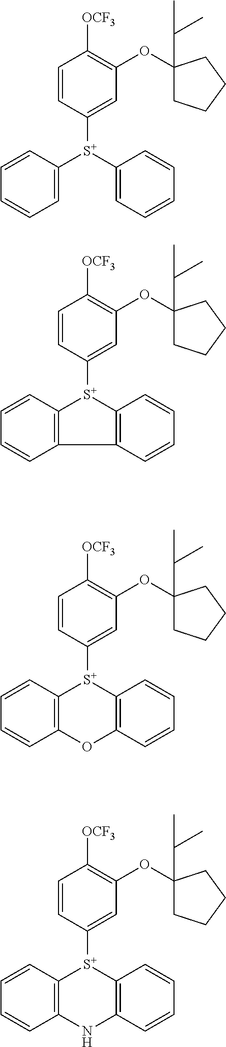 [SMILES: CC(C)C1(Oc2cc([S+](c3ccccc3)c3ccccc3)ccc2OC(F)(F)F)CCCC1 ; CC(C)C1(Oc2cc(-[s+]3c4ccccc4c4ccccc43)ccc2OC(F)(F)F)CCCC1 ; CC(C)C1(Oc2cc([S+]3c4ccccc4Oc4ccccc43)ccc2OC(F)(F)F)CCCC1 ; CC(C)C1(Oc2cc([S+]3c4ccccc4Nc4ccccc43)ccc2OC(F)(F)F)CCCC1]

Examples of the iodonium cation having formula (cation-2) are shown below, but not limited thereto.

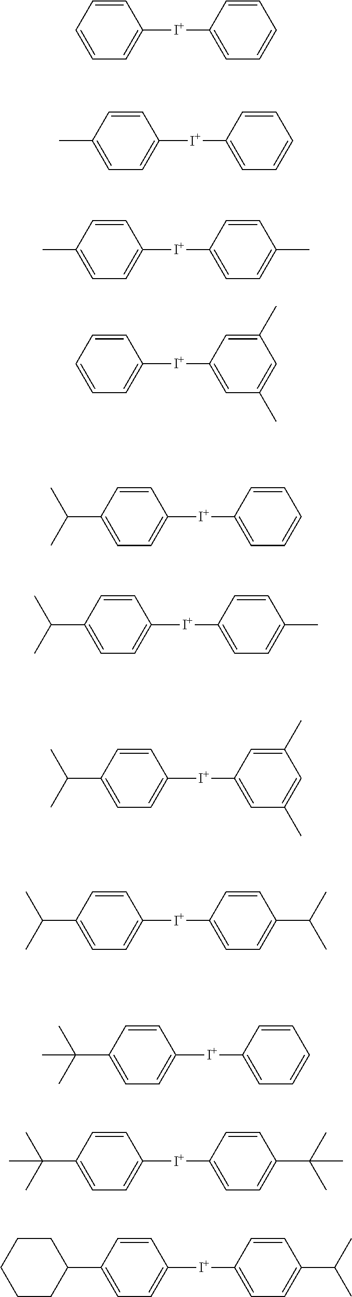

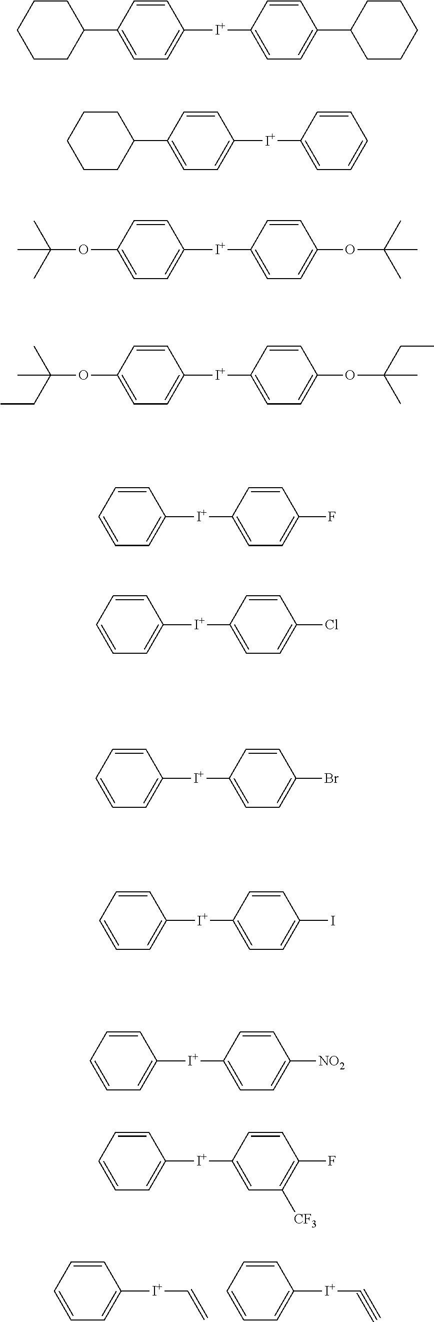

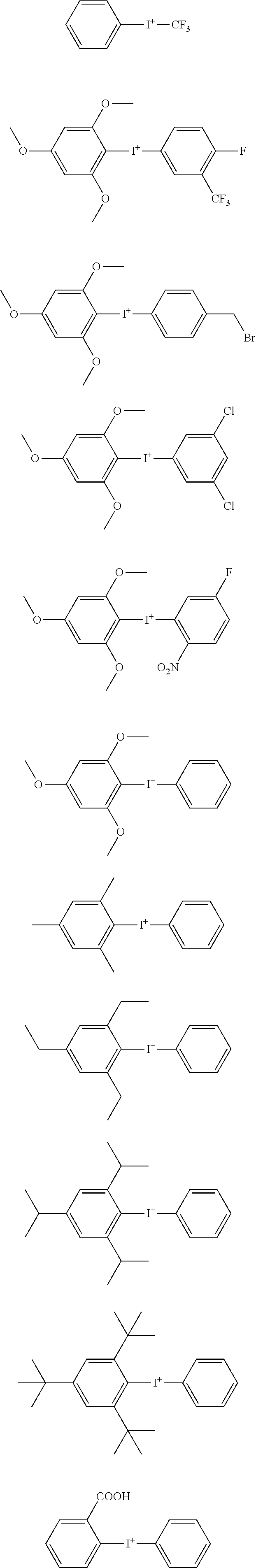

Examples of the repeat units (c1) to (c4) include arbitrary combinations of anions with cations, both as exemplified above.

Of the repeat units (c1) to (c4), repeat units (c2), (c3) and (c4) are preferred from the aspect of controlling acid diffusion, repeat units (c2) and (c4) are more preferred from the aspect of the acid strength of generated acid, and repeat units (c2) are most preferred from the aspect of solvent solubility.

The polymer may further comprise repeat units (d) of a structure having a hydroxy group protected with an acid labile group. The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

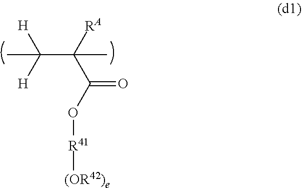

In formula (d1), R^{A} is as defined above. R⁴¹ is a C₁-C₃₀ (e+1)-valent hydrocarbon group which may contain a heteroatom. R⁴² is an acid labile group, and e is an integer of 1 to 4.

In formula (d1), the acid labile group R⁴² is deprotected under the action of acid so that a hydroxy group is generated. Although the structure of R⁴² is not particularly limited, an acetal structure, ketal structure, alkoxycarbonyl group and alkoxymethyl group having the following formula (d2) are preferred, with the alkoxymethyl group having formula (d2) being more preferred.

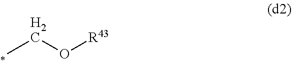

Herein R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the polymer may further comprise repeat units (e) derived from indene, benzofuran, benzothiophene, acenaphthylene, chromone, coumarin, norbornadiene and derivatives thereof. Examples of the monomer from which repeat units (e) are derived are shown below, but not limited thereto.

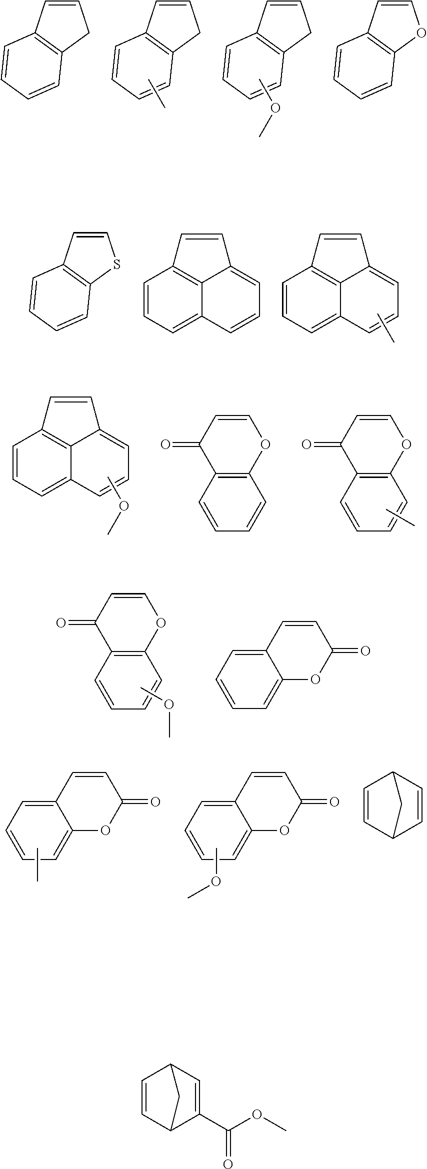

The polymer may further comprise repeat units (f) derived from indane, vinylpyridine or vinylcarbazole.

In the polymer, repeat units (A), (a1), (a2), (b1), (b2), (c1) to (c4), (d), (e), and (f) are incorporated in a ratio of preferably 0<A≤0.8, 0≤a1≤0.8, 0≤a2≤0.8, 0≤b1≤0.6, 0≤b2≤0.6, 0≤c1≤0.4, 0≤c2≤0.4, 0≤c3≤0.4, 0≤c4≤0.3, 0≤d≤0.3, 0≤e≤0.3, and 0≤f≤0.3; more preferably 0<A≤0.5, 0≤a1≤0.5, 0<a≤0.5, 0≤b1≤0.5, 0≤b2≤0.5, 0≤c1≤0.3 , 0≤c2≤0.3, 0≤c3≤0.3, 0≤c4≤0.3, 0≤d≤0.3, 0≤e≤0.3, and 0≤f≤0.3.

The polymer should preferably have a weight average molecular weight (Mw) in the range of 1,000 to 500,000, and more preferably 3,000 to 100,000, as measured by GPC versus polystyrene standards using tetrahydrofuran (THF) or N,N-dimethylformamide (DMF) solvent. A Mw in the range ensures that the resist film has etch resistance and eliminates the risk of resolution decline by a failure to provide a difference in dissolution rate before and after exposure.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours in view of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form a ultra-high-molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted by an acetal group which is susceptible to deprotection with acid, typically ethoxyethoxy, prior to polymerization, and the polymerization is followed by deprotection with weak acid and water. Alternatively, the hydroxy group is substituted by an acetyl, formyl or pivaloyl group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145](U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

### (A) Base Polymer

A further embodiment of the invention is a chemically amplified resist composition comprising (A) a base polymer containing the polymer defined above.

The base polymer (A) may be a single polymer or a blend of two or more polymers which differ in compositional ratio, Mw and/or Mw/Mn. Component (A) may also be a blend of the polymer defined above and a hydrogenated product of ring-opening metathesis polymerization (ROMP) polymer. For the ROMP, reference is made to JP-A 2003-066612.

### (B) Organic Solvent

The resist composition comprises an organic solvent as component (B). The organic solvent (B) is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, DAA and mixtures thereof because the base polymer (A) is most soluble therein.

The organic solvent (B) is preferably added in an amount of 200 to 5,000 parts by weight, and more preferably 400 to 3,500 parts by weight per 80 parts by weight of the base polymer (A). The organic solvent may be used alone or in admixture.

### (C) Quencher

The resist composition may further comprise a quencher as component (C). As used herein, the quencher refers to a compound capable of trapping the acid generated by the PAG in the resist composition to prevent the acid from diffusing to the unexposed region, for thereby forming the desired pattern.

Onium salts having the formulae (C₁) and (C₂) are useful as the quencher (C).

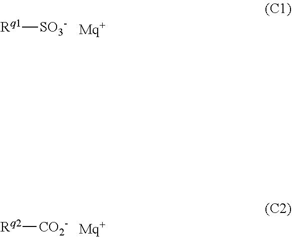

In formula (C₁), R^{q1} is hydrogen or a C₁-C₄₀ hydrocarbyl group which may contain a heteroatom, exclusive of the hydrocarbyl group in which the hydrogen atom bonded to the carbon atom at α-position of the sulfo group is substituted by fluorine or fluoroalkyl.

The hydrocarbyl group R^{q1} may be saturated or unsaturated and straight, branched or cyclic. Examples thereof include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; and C₆-C₄₀ aryl groups such as phenyl, naphthyl and anthracenyl. In these hydrocarbyl groups, some or all hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

In formula (C₂), R^{q2} is hydrogen or a C₁-C₄₀ hydrocarbyl group which may contain a heteroatom. Examples of the hydrocarbyl group R^{q2} include those exemplified above for R^{q}1 and fluorinated saturated hydrocarbyl groups such as trifluoromethyl and trifluoroethyl, and fluorinated aryl groups such as pentafluorophenyl and 4-trifluoromethylphenyl.

Examples of the anion in the onium salt having formula (C₁) are shown below, but not limited thereto.

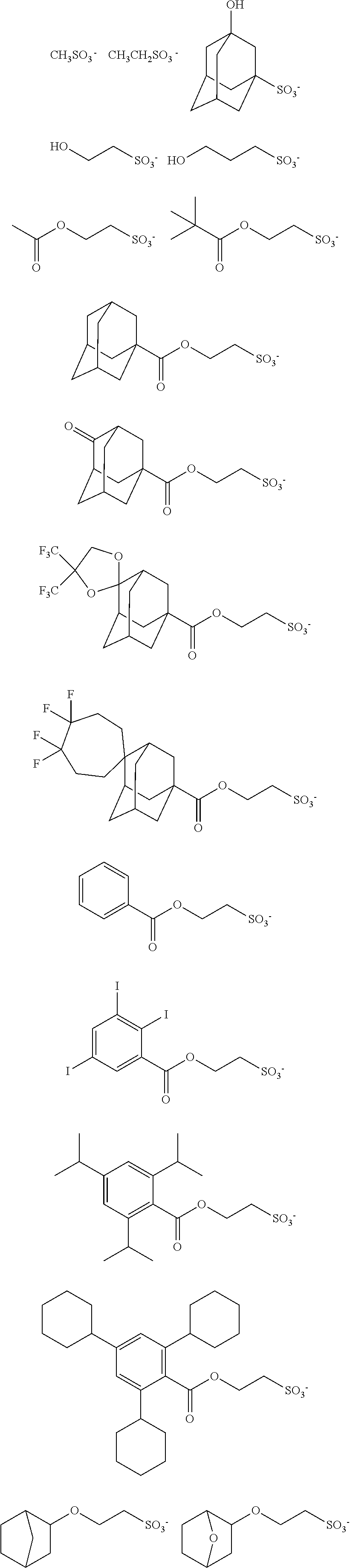

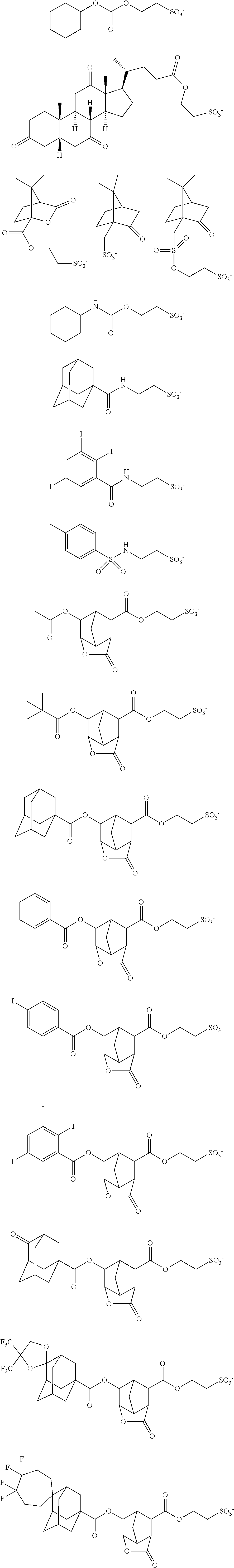

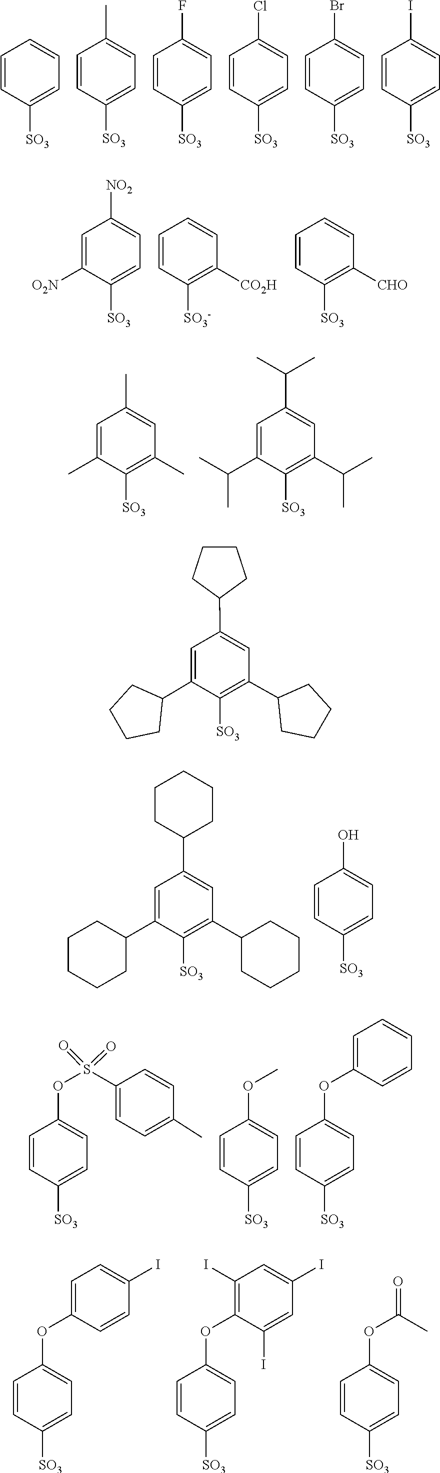

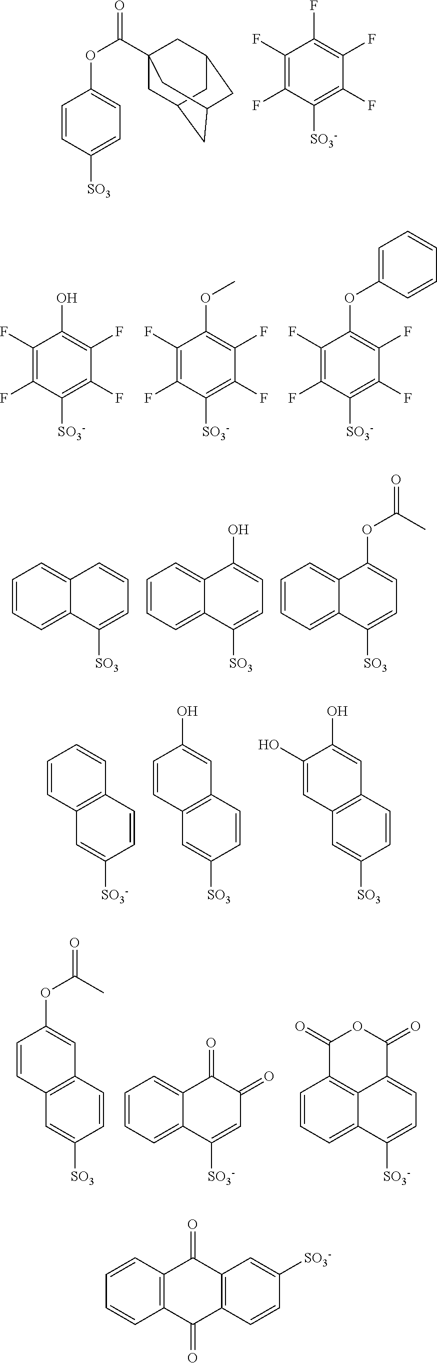

Examples of the anion in the onium salt having formula (C₂) are shown below, but not limited thereto.

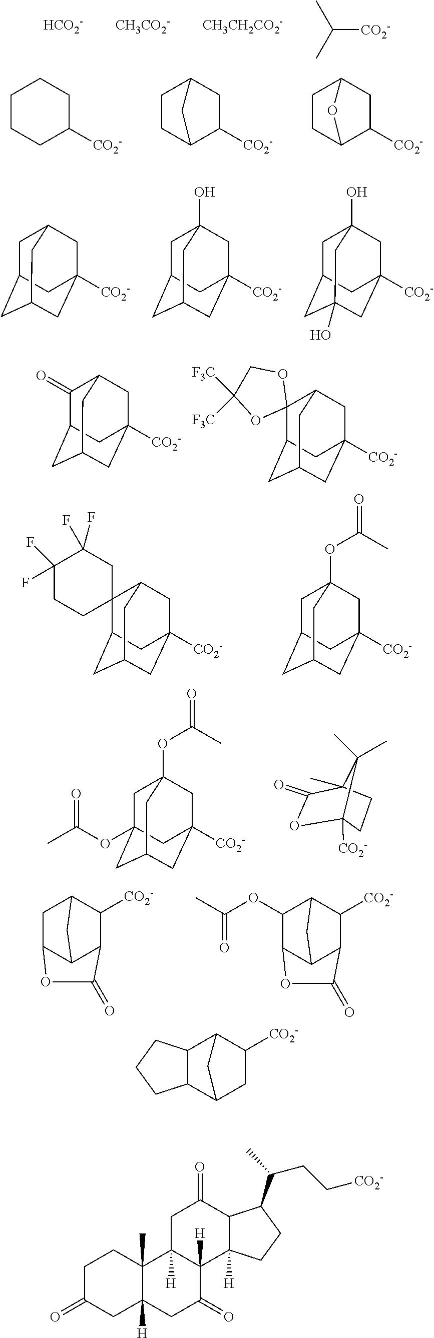

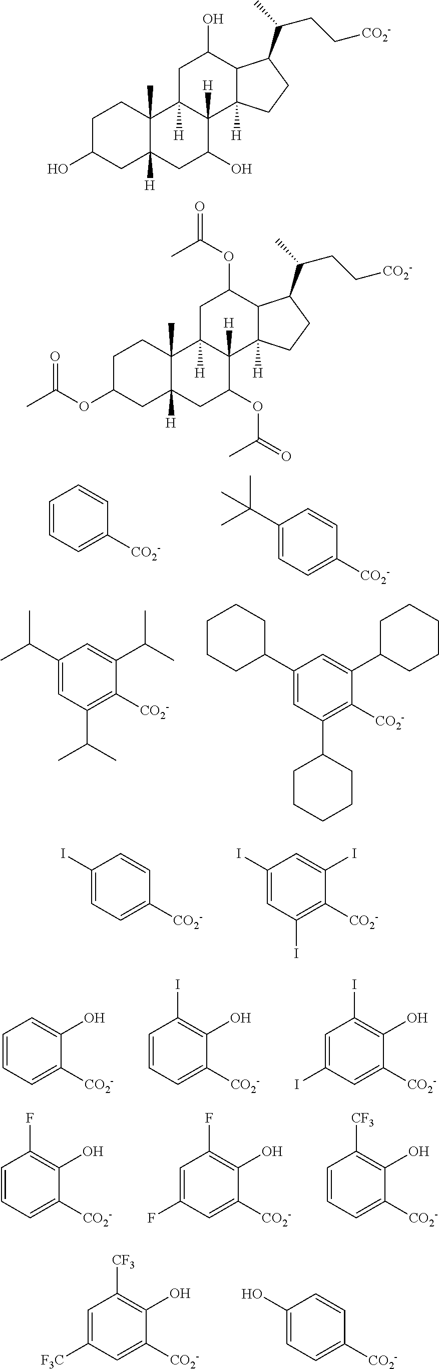

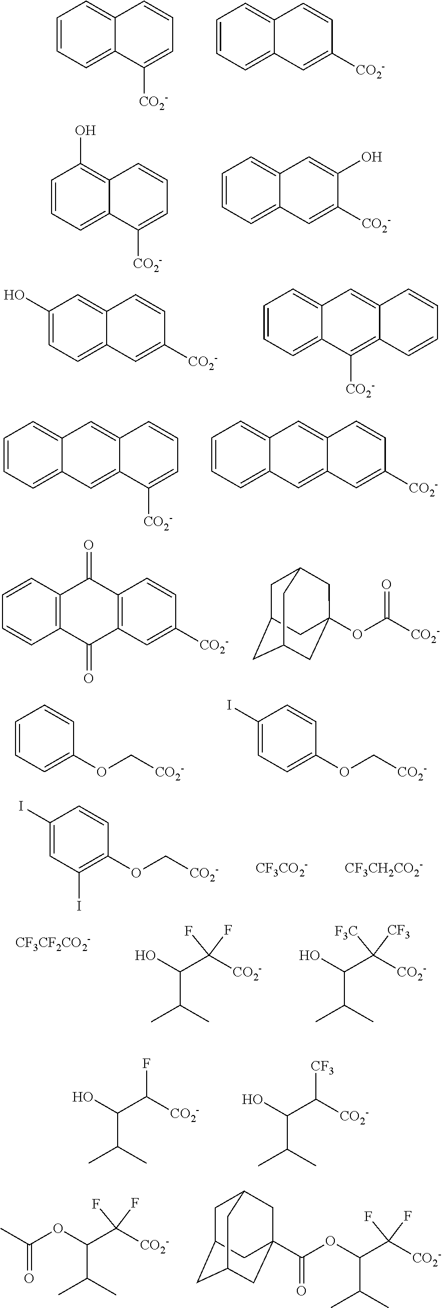

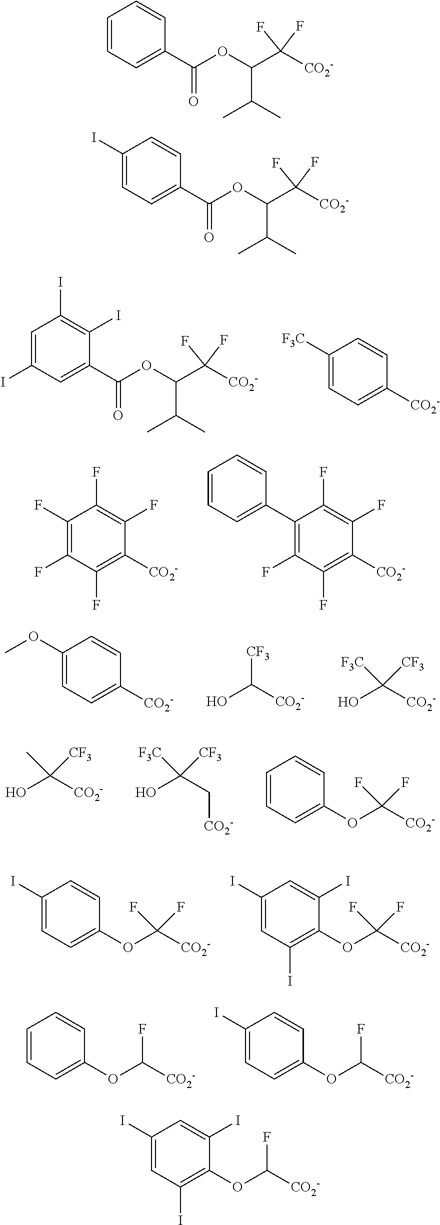

In formulae (C₁) and (C₂), Mq⁺ is an oniumn cation, which is preferably selected from sulfonium cations having formula (cation-1), iodonium cations having formula (cation-2), and ammonium cations having the following formula (cation-3).

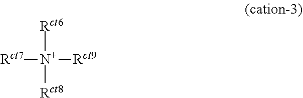

In formula (cation-3), R^{ct6} to R^{ct9} are each independently a C₁-C₄₀ hydrocarbyl group which may contain a heteroatom. A pair of R^{ct6} and R^{ct7} may bond together to form a ring with the nitrogen atom to which they are attached. Examples of the hydrocarbyl group are as exemplified above for R^{ct1} to R^{ct5} in formulae (cation-1) and (cation-2).

Examples of the ammonium cation having formula (cation-3) are shown below, but not limited thereto.

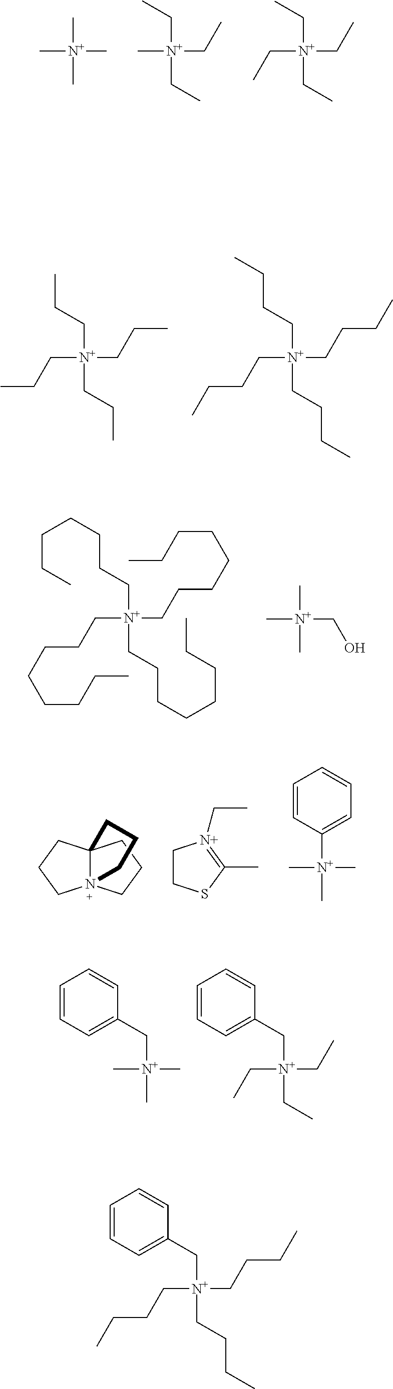

Examples of the onium salt having formula (C₁) or (C₂) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (C₁) or (C₂) functions as a quencher in the chemically amplified resist composition because the counter anion of the onium salt is a conjugated base of a weak acid. The onium salt having formula (C₁) or (C₂) functions as a quencher when used in combination with an onium salt type PAG having a conjugated base of a strong acid (typically a sulfonic acid which is fluorinated at α-position) as the counter anion. In a system using a mixture of an onium salt capable of generating a strong acid (e.g., α-position fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion. As used herein, the term “weak acid” indicates an acidity insufficient to deprotect an acid labile group from an acid labile group-containing unit in the polymer. The term “strong acid” refers to a compound having a sufficient acidity to induce deprotection reaction of an acid labile group.

Also useful as the quencher (C) are onium salts having a sulfonium cation and a phenoxide anion site in a common molecule as described in JP 6848776, onium salts having a sulfonium cation and a carboxylate anion site in a common molecule as described in JP 6583136 and JP-A 2020-200311, and onium salts having an iodonium cation and a carboxylate anion site in a common molecule as described in JP 6274755.

If a PAG capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the onium salt having formula (C₁) or (C₂) is used as the quencher (C), the amount of the onium salt used is preferably 0.1 to 20 parts by weight, more preferably 0.1 to 10 parts by weight per 80 parts by weight of the base polymer (A). As long as the amount of component (C) is in the range, a satisfactory resolution is available without a substantial lowering of sensitivity. The onium salt having formula (C₁) or (C₂) may be used alone or in admixture.

Also nitrogen-containing compounds may be used as the quencher (C). Suitable nitrogen-containing compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164](U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Photoacid Generator

The chemically amplified resist composition may comprise (D) a photoacid generator. The PAG is not particularly limited as long as it is capable of generating a strong acid upon exposure to high-energy radiation.

The preferred PAG is a salt having the formula (D1) or (D2).

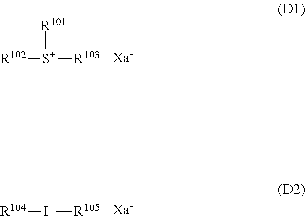

In formulae (D1) and (D2), R¹⁰¹ to R¹⁰⁵ are each independently a C₁-C₂₀ hydrocarbyl group which may contain a heteroatom. Any two of R¹⁰¹, R¹⁰² and R¹⁰³ may bond together to form a ring with the sulfur atom to which they are attached. Examples of the hydrocarbyl group are as exemplified above for R^{ct1} to R^{ct5} in formulae (cation-1) and (cation-2).

Examples of the sulfonium cation in the salt having formula (D1) are as exemplified above for the sulfonium cation having formula (cation-1). Examples of the iodonium cation in the salt having formula (D2) are as exemplified above for the iodonium cation having formula (cation-2).

In formulae (D1) and (D2), Xa⁻ is an anion of strong acid selected from formulae (c1-1) to (c1-5).

Compounds having the formula (D3) are also preferred as the PAG (D).

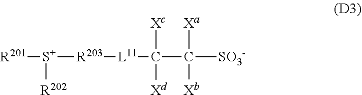

In formula (D3), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached.

The C₁-C₃₀ hydrocarbyl group represented by R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; and C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl, and combinations thereof. In these hydrocarbyl groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene. In these hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In formula (D3), L¹¹ is a single bond, ether bond or a C₁-C₂₀ hydrocarbylene group which may contain a heteroatom. The hydrocarbylene group may be saturated or unsaturated and straight, branched or cyclic. Examples thereof are as exemplified above for the hydrocarbylene group R²⁰³.

In formula (D3), X^{a}, X^{b}, X^{c} and X^{d} are each independently hydrogen, fluorine or trifluoromethyl, at least one of X^{a}, X^{b}, X^{c} and X^{d} being fluorine or trifluoromethyl.

Of the PAGs having formula (D3), those having formula (D3′) are preferred.

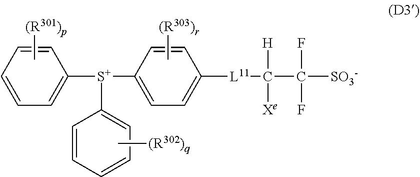

In formula (D3′), L¹¹ is as defined above. X^{e} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa} in formula (c1-1-1). The subscripts p and q are each independently an integer of 0 to 5, and r is an integer of 0 to 4.

Examples of the PAG having formula (D3) include those exemplified for the PAG having formula (2) in JP-A 2017-026980.

Of the foregoing PAGs, those having an anion of formula (c1-1-1) or (c1-4) are especially preferred because of reduced acid diffusion and high solubility in solvents. Also those having formula (D3′) are especially preferred because of extremely reduced acid diffusion.

When used, the PAG (D) is preferably added in an amount of 0.1 to 40 parts, and more preferably 0.5 to 20 parts by weight per 80 parts by weight of the base polymer (A). As long as the amount of the PAG is in the range, good resolution is achievable and the risk of foreign particles being formed after development or during stripping of resist film is avoided. The PAG may be used alone or in admixture. When the polymer contains any of repeat units (c1) to (c4), that is, in the case of polymer-bound photoacid generator, blending of PAG (D) may be omitted.

### (E) Surfactant

The resist composition may further include (E) a surfactant. Preferred are a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having the formula (surf-1) are also useful.

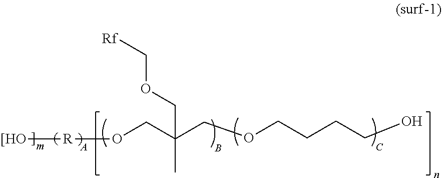

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

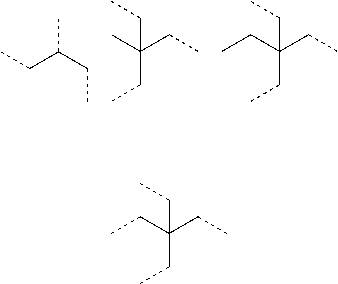

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The subscript m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (E1) to (E5).

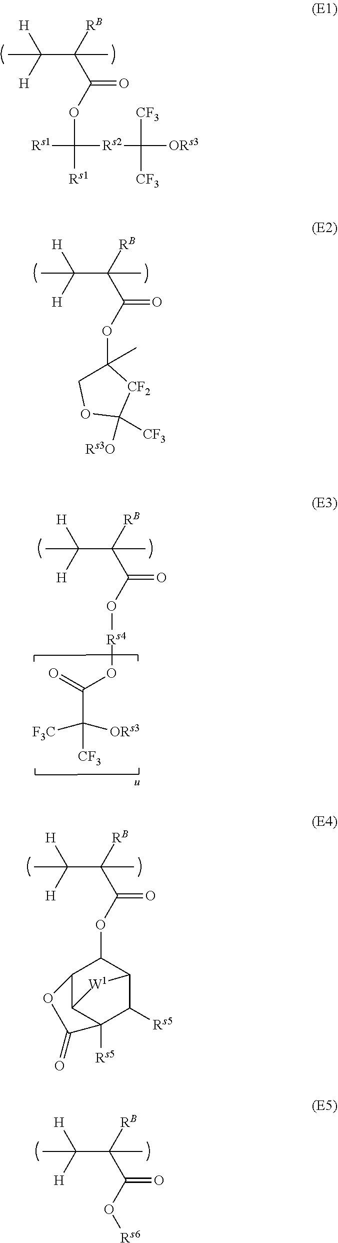

Herein, R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group represented by R^{s1} may be straight, branched or cyclic and is preferably saturated. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group represented by R^{s2} may be straight, branched or cyclic and is preferably saturated. Examples thereof include methylene, ethylene, propylene, butylene and pentylene.

The hydrocarbyl group represented by R^{S3} or R^{S6} may be saturated or unsaturated and straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-1) to (AL-3), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic and examples thereof include the foregoing hydrocarbyl or fluorinated hydrocarbyl groups from which u number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (E1) to (E5) are shown below, but not limited thereto. Herein R^{B} is as defined above.

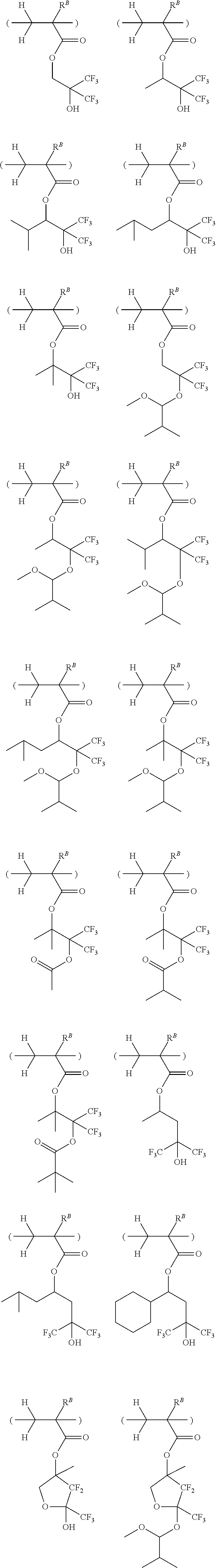

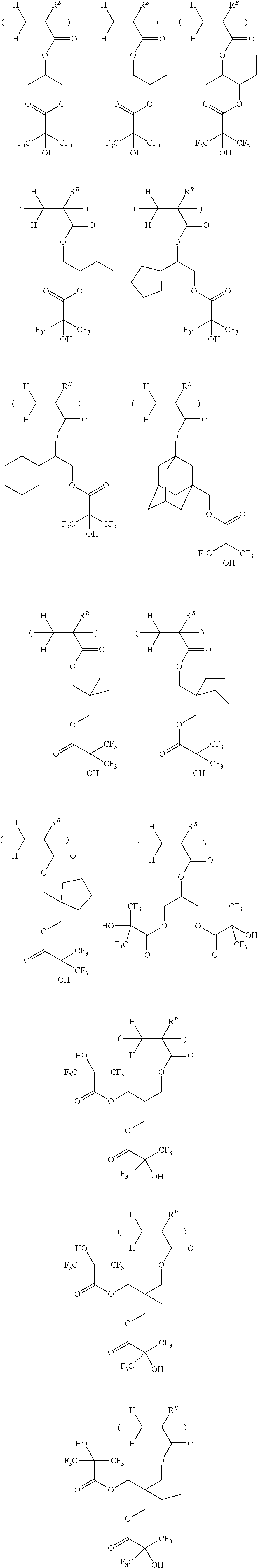

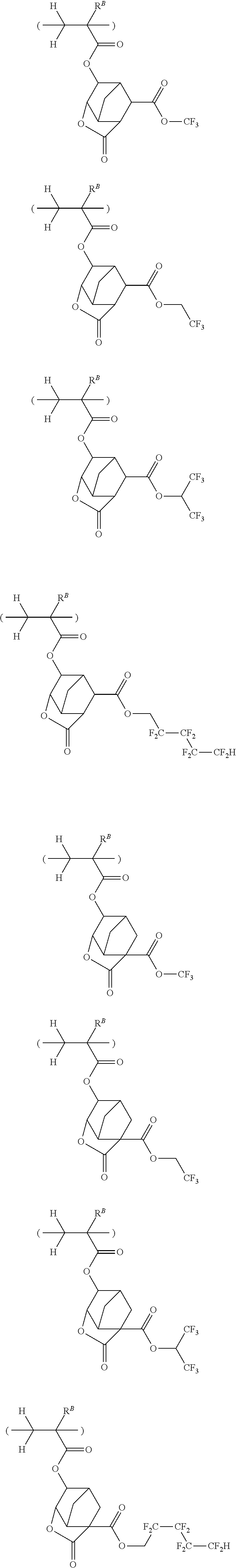

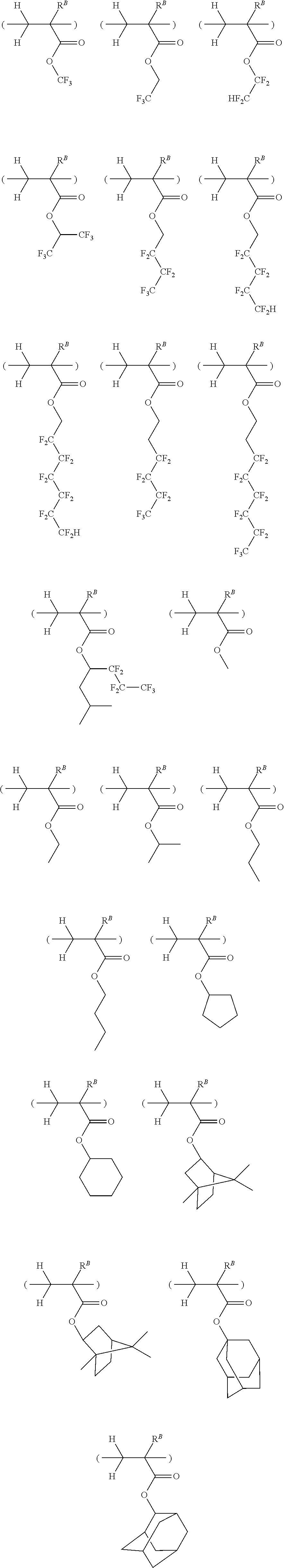

The polymeric surfactant may further contain repeat units other than the repeat units having formulae (E1) to (E5). Typical other repeat units are those derived from methacrylic acid and α-trifluoromethylacrylic acid derivatives. In the polymeric surfactant, the content of the repeat units having formulae (E1) to (E5) is preferably at least 20 mol %, more preferably at least 60 mol %, most preferably 100 mol % of the overall repeat units.

The polymeric surfactant preferably has a Mw of 1,000 to 500,000, more preferably 3,000 to 100,000 and a Mw/Mn of 1.0 to 2.0, more preferably 1.0 to 1.6.

The polymeric surfactant may be synthesized by any desired method, for example, by dissolving an unsaturated bond-containing monomer or monomers providing repeat units having formula (E1) to (E5) and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the resist composition contains a surfactant (E), the amount thereof is preferably 0.1 to 50 parts by weight, and more preferably 0.5 to 10 parts by weight per 80 parts by weight of the base polymer (A). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (E) may be used alone or in admixture.

(F) Other components

The resist composition may further comprise (F) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (B). An extra amount of the acid amplifier compound can make the acid diffusion control difficult and cause degradations to resolution and pattern profile. With respect to the remaining additives, reference should be made to JP-A 2009-269953 and JP-A 2010-215608.

### [Process]

A further embodiment of the invention is a process of forming a pattern from the resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture of two or more.

### EXAMPLES

Examples of the invention are given below by way of illustration and not by way of limitation. All parts (pbw) are by weight. The structure of a compound was determined by measuring molecular ion peaks by a mass analyzer (LC by 1100 series by Agilent and MASS by LC/MSD model by Agilent. The value of a molecular ion peak is expressed as MASS.

### [1] Synthesis of Monomers

### [Example 1-1] Synthesis of Monomer A1

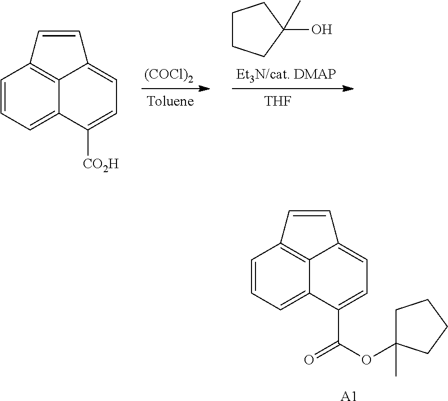

In nitrogen atmosphere, 19.6 g of acenaphthylenecarboxylic acid was suspended in 80 g of toluene. To the suspension, 15.2 g of oxalyl chloride was added dropwise. The solution was then heated at an interior temperature of 40° C. and aged for 4 hours. At the end of aging, the reaction solution was concentrated, obtaining 19.7 g of acid chloride. Then 12.0 g of 1-methylcyclopentanol, 1.2 g of 4-dimethylaminopyridine, and 80 g of THF were added to the concentrate, which was cooled in an ice bath. After cooling, a mixture of 13.2 g of triethylamine and 20 g of THF was added dropwise while maintaining the interior temperature below 20° C. At the end of addition, the interior temperature was raised to 50° C., at which the solution was aged for 12 hours. After aging, the reaction solution was cooled in an ice bath, to which 50 g of saturated sodium bicarbonate solution was added to quench the reaction. The target compound was extracted with a solvent consisting of 100 ml of toluene and 50 ml of ethyl acetate. This was followed by standard aqueous workup, solvent distillation, and purification by silica gel chromatography, obtaining Monomer A1 as colorless oily matter (amount 21.2 g, yield 76%).

The result of mass analysis of Monomer A1 is shown below.

MASS: 279.1 [M+H]⁺

### [Examples 1-2 to 1-8] Synthesis of Monomers A2 to A8

A variety of polymerizable monomers were synthesized by organic synthesis reactions using the corresponding reactants. The polymerizable monomers A2 to A8 having the structure shown below were used in the synthesis of polymers.

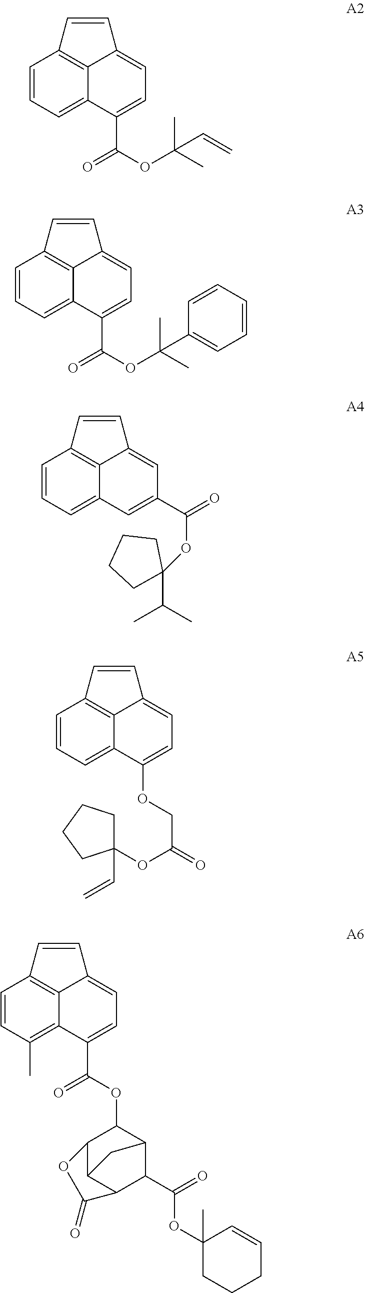

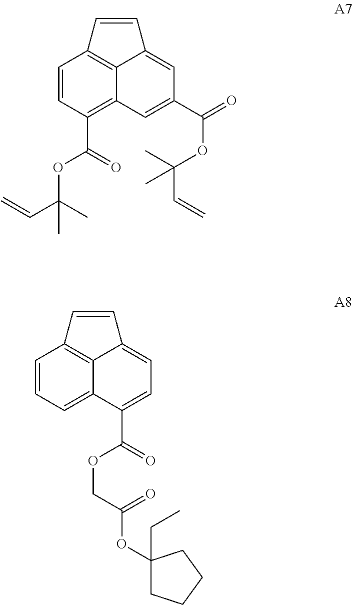 [SMILES: C=CC(C)(C)OC(=O)c1cc2c3c(ccc(C(=O)OC(C)(C)C=C)c3c1)C=C2 ; CCC1(OC(=O)COC(=O)c2ccc3c4c(cccc24)C=C3)CCCC1]

### [2] Synthesis of Polymers

A variety of monomers were used in the synthesis of polymers. The monomers other than Monomers A1 to A8 are shown below.

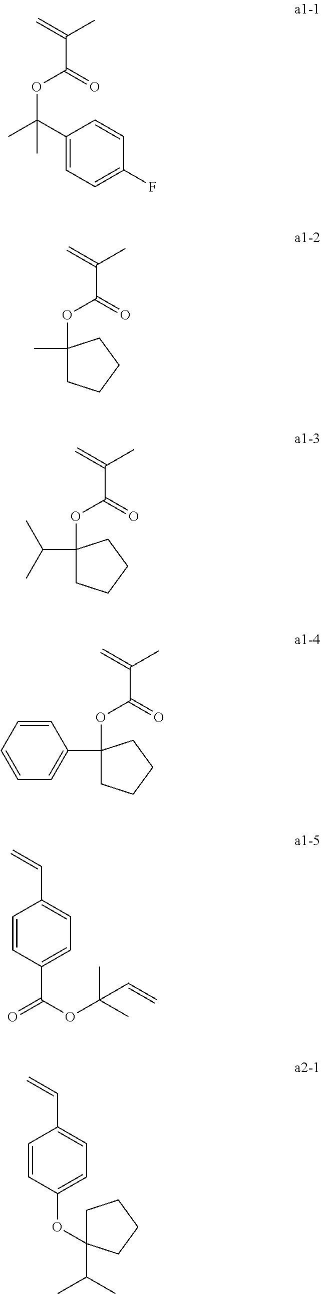 [SMILES: C=C(C)C(=O)OC(C)(C)c1ccc(F)cc1 ; C=C(C)C(=O)OC1(C)CCCC1 ; C=C(C)C(=O)OC1(C(C)C)CCCC1 ; C=C(C)C(=O)OC1(c2ccccc2)CCCC1 ; C=Cc1ccc(C(=O)OC(C)(C)C=C)cc1 ; C=Cc1ccc(OC2(C(C)C)CCCC2)cc1]

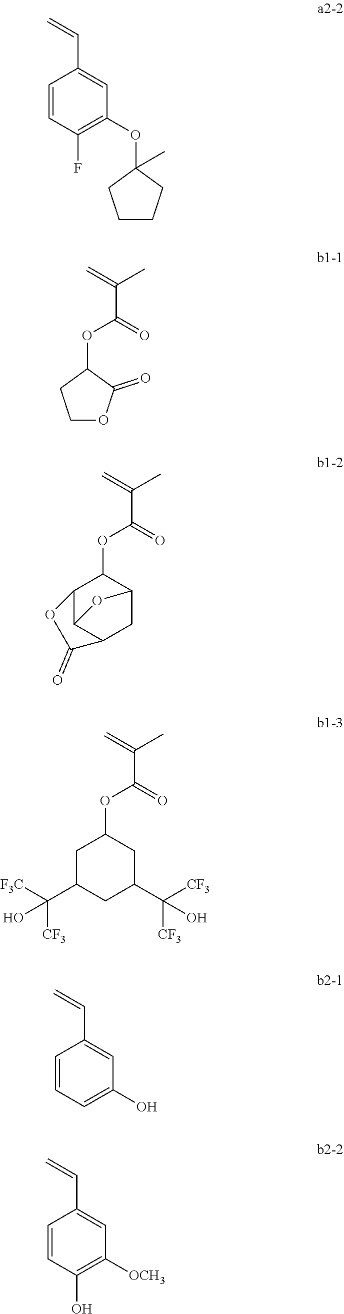

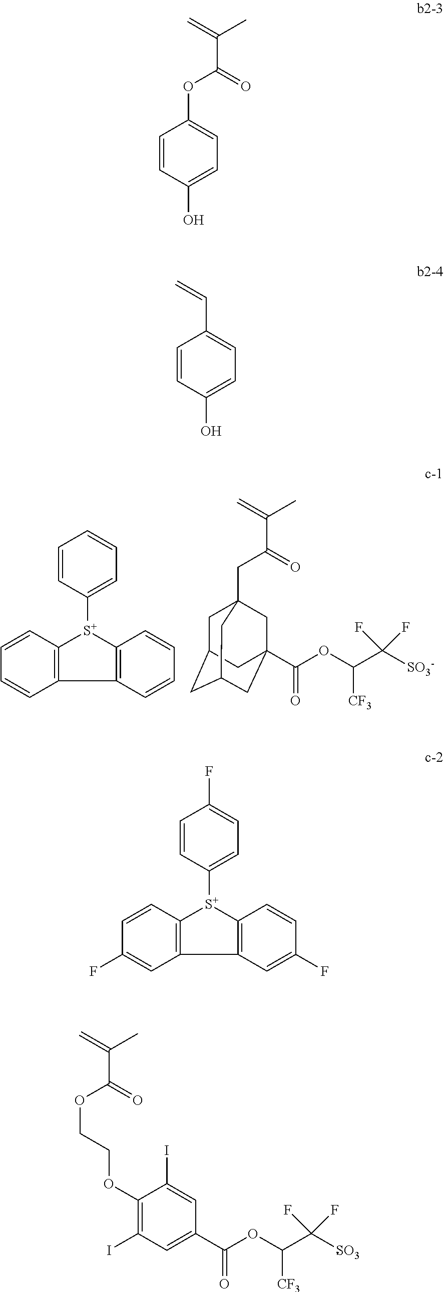

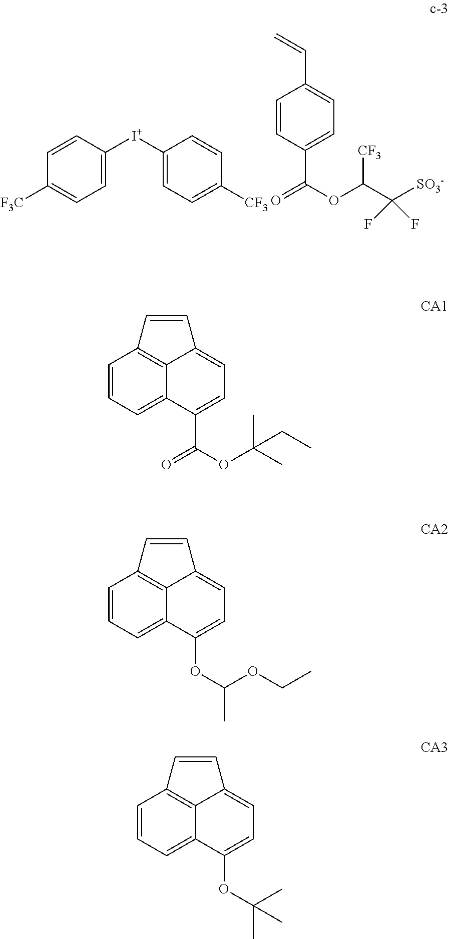

### [Example 2-1] Synthesis of Polymer P-1

A flask under nitrogen blanket was charged with 8.1 g of Monomer A1, 9.7 g of Monomer a1-1, 6.2 g of Monomer b2-1, 16.1 g of Monomer c-1, 1.68 g of dimethyl 2,2′-azobis(2-methylpropionate) (trade name V601, FUJIFILM Wako Pure Chemical Corp.), and 50 g of methyl ethyl ketone (MEK) to form a monomer/initiator solution. Another flask under nitrogen blanket was charged with 19 g of MEK, which was heated at 80° C. with stirring. While the temperature was maintained, the monomer/initiator solution was added dropwise to the flask over 4 hours. At the end of dropwise addition, the solution was continuously stirred for 2 hours while the temperature of 80° C. was maintained during polymerization. The polymerization solution was cooled to room temperature. With vigorous stirring, the solution was added dropwise to 1,200 g of hexane, whereupon a polymer precipitated. The solid precipitate was filtered, washed twice with 240 g of hexane, and vacuum dried at 50° C. for 20 hours, obtaining Polymer P-1 in white powder form (amount 39.2 g, yield 98%). Polymer P-1 had a Mw of 9,300 and a Mw/Mn of 1.79. It is noted that Mw is determined by GPC versus polystyrene standards using DMF solvent.

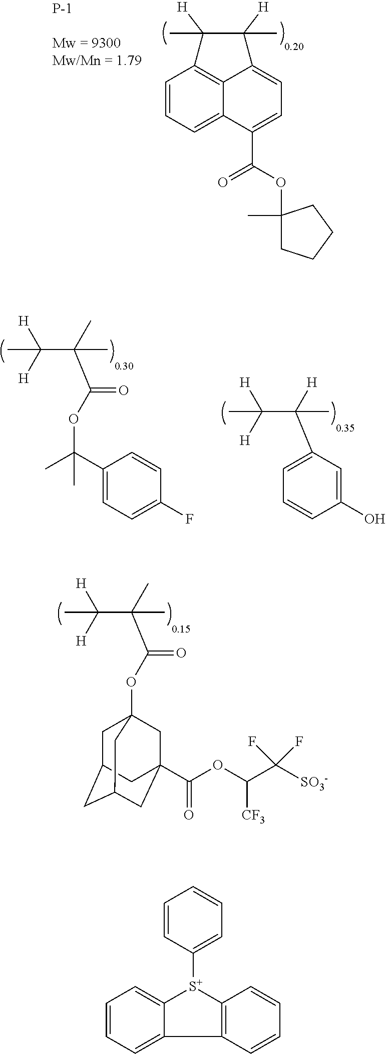

### [Examples 2-2 to 2-34 and Comparative Examples 1-1 to 1-16] Synthesis of Polymers P-2 to P-34 and Comparative Polymers CP-1 to CP-16

The polymers shown in Tables 1 and 2 were synthesized by the same procedure as in Example 2-1 except that the type and amount of monomers were changed.

TABLE 1

| | | | | Incor- | | Incor- | | Incor- | | Incor- | | Incor- | | |
|---|---|---|---|---|---|---|---|---|---|---|---|---|---|---|
| | | | | poration | | poration | | poration | | poration | | poration | | |
| | | | Unit | ratio | Unit | ratio | Unit | ratio | Unit | ratio | Unit | ratio | | |
| | | Polymer | A | (mol %) | a | (mol %) | b1 | (mol %) | b2 | (mol %) | c | (mol %) | Mw | Mw/Mn |
| Example | 2-1 | P-1 | A1 | 20 | a1-1 | 30 | — | — | b2-1 | 35 | c-1 | 15 | 9,300 | 1.79 |
| | 2-2 | P-2 | A2 | 20 | a1-1 | 30 | — | — | b2-1 | 35 | c-1 | 15 | 9,400 | 1.81 |
| | 2-3 | P-3 | A3 | 20 | a1-1 | 30 | — | — | b2-1 | 35 | c-1 | 15 | 9,200 | 1.80 |
| | 2-4 | P-4 | A4 | 20 | a1-1 | 30 | — | — | b2-1 | 35 | c-1 | 15 | 9,300 | 1.78 |
| | 2-5 | P-5 | A5 | 20 | a1-1 | 30 | — | — | b2-1 | 35 | c-1 | 15 | 9,300 | 1.82 |
| | 2-6 | P-6 | A6 | 15 | a1-1 | 35 | — | — | b2-1 | 35 | c-1 | 15 | 9,200 | 1.79 |
| | 2-7 | P-7 | A7 | 15 | a1-1 | 25 | — | — | b2-1 | 45 | c-1 | 15 | 9,400 | 1.78 |
| | 2-8 | P-8 | A1 | 20 | a1-2 | 30 | — | — | b2-2 | 30 | c-3 | 20 | 9,300 | 1.82 |
| | 2-9 | P-9 | A2 | 20 | a1-3 | 30 | — | — | b2-1 | 35 | c-2 | 15 | 9,400 | 1.81 |
| | 2-10 | P-10 | A3 | 20 | a1-4 | 30 | — | — | b2-1 | 35 | c-2 | 15 | 9,200 | 1.80 |
| | 2-11 | P-11 | A4 | 20 | a1-5 | 30 | — | — | b2-1 | 35 | c-1 | 15 | 9,300 | 1.78 |
| | 2-12 | P-12 | A5 | 20 | a2-1 | 30 | — | — | b2-1 | 35 | c-1 | 15 | 9,300 | 1.82 |
| | 2-13 | P-13 | A6 | 20 | a2-2 | 30 | — | — | b2-1 | 35 | c-2 | 15 | 9,200 | 1.79 |
| | 2-14 | P-14 | A7 | 15 | a1-1 | 30 | — | — | b2-3 | 40 | c-1 | 15 | 9,400 | 1.78 |
| | 2-15 | P-15 | A1 | 20 | a1-1 | 30 | b1-1 | 10 | b2-1 | 25 | c-2 | 15 | 9,400 | 1.81 |
| | 2-16 | P-16 | A2 | 20 | a1-3 | 30 | b1-2 | 10 | b2-1 | 25 | c-2 | 15 | 9,200 | 1.80 |
| | 2-17 | P-17 | A2 | 25 | a1-5 | 30 | b1-3 | 5 | b2-1 | 25 | c-1 | 15 | 9,300 | 1.78 |
| | 2-18 | P-18 | A1 | 50 | — | — | — | — | b2-1 | 35 | c-2 | 15 | 9,300 | 1.81 |
| | 2-19 | P-19 | A1 | 45 | — | — | — | — | b2-3 | 40 | c-2 | 15 | 9,200 | 1.79 |
| | 2-20 | P-20 | A2 | | — | — | b1-1 | 10 | b2-1 | 25 | c-2 | 20 | 9,400 | 1.78 |
| | 2-21 | P-21 | A1 | 20 | a1-1 | 35 | — | — | b2-1 | 45 | — | — | 6,700 | 1.67 |
| | 2-22 | P-22 | A2 | 20 | a1-3 | 30 | b1-1 | 10 | b2-3 | 40 | — | — | 6,800 | 1.66 |
| | 2-23 | P-23 | A3 | 25 | a1-5 | 30 | b1-2 | 10 | b2-2 | 35 | — | — | 6,500 | 1.65 |
| | 2-24 | P-24 | A5 | 20 | a2-1 | 35 | b1-3 | 5 | b2-1 | 40 | — | — | 6,800 | 1.66 |
| | 2-25 | P-25 | A6 | 15 | a1-4 | 35 | — | — | b2-3 | 50 | — | — | 6,800 | 1.68 |
| | 2-26 | P-26 | A7 | 20 | a2-2 | 30 | b1-2 | 10 | b2-2 | 40 | — | — | 6,600 | 1.68 |
| | 2-27 | P-27 | A4 | 35 | a1-3 | 25 | b1-2 | 10 | b2-1 | 30 | — | — | 6,500 | 1,67 |
| | 2-28 | P-28 | A2 | 35 | a1-4 | 15 | b1-3 | 10 | b2-3 | 40 | — | — | 6,800 | 1.64 |
| | 2-29 | P-29 | A2 | 50 | — | — | — | — | b2-1 | 50 | — | — | 6,400 | 1.63 |
| | 2-30 | P-30 | A1 | 40 | — | — | — | — | b2-1 | 60 | — | — | 6,500 | 1.65 |
| | 2-31 | P-31 | A8 | 20 | a1-1 | 30 | — | — | b2-1 | 35 | c-1 | 15 | 9,400 | 1.76 |
| | 2-32 | P-32 | A8 | 25 | a1-2 | 25 | — | — | b2-4 | 35 | c-2 | 15 | 9,100 | 1.75 |
| | 2-33 | P-33 | A8 | 25 | a2-2 | 30 | — | — | b2-4 | 30 | c-2 | 15 | 9,200 | 1.77 |
| | 2-34 | P-34 | A8 | 50 | — | — | — | — | b2-4 | 50 | — | — | 6,700 | 1.64 |

TABLE 2

| | | | | Incor- | | Incor- | | Incor- | | Incor- | | Incor- | | |
|---|---|---|---|---|---|---|---|---|---|---|---|---|---|---|
| | | | | poration | | poration | | poration | | poration | | poration | | |
| | | | Unit | ratio | Unit | ratio | Unit | ratio | Unit | ratio | Unit | ratio | | |
| | | Polymer | A | (mol %) | a | (mol %) | b1 | (mol %) | b2 | (mol %) | c | (mol %) | Mw | Mw/Mn |
| Compar- | 1-1 | CP-1 | CA1 | 20 | a1-1 | 30 | — | — | b2-1 | 35 | c-1 | 15 | 9,300 | 1.79 |
| ative | 1-2 | CP-2 | CA2 | 20 | a1-1 | 30 | — | — | b2-1 | 35 | c-1 | 15 | 9,500 | 1.82 |
| Example | 1-3 | CP-3 | CA3 | 20 | a1-1 | 30 | — | — | b2-1 | 35 | c-1 | 15 | 9,400 | 1.81 |
| | 1-4 | CP-4 | CA1 | 20 | a1-3 | 30 | — | — | b2-1 | 35 | c-2 | 15 | 9,600 | 1.81 |
| | 1-5 | CP-5 | CA2 | 20 | a2-1 | 30 | — | — | b2-1 | 35 | c-1 | 15 | 9,400 | 1.84 |
| | 1-6 | CP-6 | CA1 | 25 | a1-1 | 25 | b1-1 | 10 | b2-1 | 25 | c-2 | 15 | 9,400 | 1.82 |
| | 1-7 | CP-7 | CA2 | 20 | a1-3 | 30 | b1-2 | 10 | b2-1 | 25 | c-2 | 15 | 9,300 | 1.78 |
| | 1-8 | CP-8 | CA3 | 25 | a1-5 | 30 | b1-3 | 5 | b2-1 | 25 | c-1 | 15 | 9,500 | 1.84 |
| | 1-9 | CP-9 | CA1 | 50 | — | — | — | — | b2-1 | 35 | c-2 | 15 | 9,600 | 1.81 |
| | 1-10 | CP-10 | — | — | a1-1 | 55 | — | — | b2-1 | 35 | c-1 | 15 | 9,400 | 1.82 |
| | 1-11 | CP-11 | CA1 | 20 | a2-2 | 30 | — | — | b2-1 | 35 | c-3 | 15 | 9,500 | 1.78 |
| | 1-12 | CP-12 | CA1 | 20 | a1-1 | 35 | — | — | b2-2 | 45 | — | — | 6,600 | 1.65 |
| | 1-13 | CP-13 | CA2 | 20 | a1-3 | 30 | b1-1 | 10 | b2-3 | 40 | — | — | 6,700 | 1.68 |
| | 1-14 | CP-14 | CA3 | 35 | a1-4 | 15 | b1-3 | 10 | b2-3 | 40 | — | — | 6,500 | 1.64 |
| | 1-15 | CP-15 | CA1 | 40 | — | — | — | — | b2-1 | 60 | — | — | 6,600 | 1.66 |
| | 1-16 | CP-16 | — | — | a1-4 | 50 | b1-1 | 10 | b2-1 | 40 | — | — | 6,500 | 1.64 |

### [3] Preparation of Chemically Amplified Resist Compositions

### Examples 3-1 to 3-34 and Comparative Examples 2-1 to 2-16

A chemically amplified resist composition (R-1 to R-34, CR-1 to CR-16) in solution form was prepared by dissolving a polymer (P-1 to P-34) or comparative polymer (CP-1 to CP-16), photoacid generator (PAG-X or PAG-Y), and quencher (SQ-1 to SQ-3 and AQ-1) in an organic solvent in accordance with the recipe shown in Tables 3 and 4, and filtering the solution through a Teflon® filter with a pore size of 0.2 μm. The organic solvent contained 0.01% by weight of a surfactant A (Omnova Solutions, Inc.).

TABLE 3

| | | Resist | Base | Photoacid | | | |
|---|---|---|---|---|---|---|---|
| | | com- | polymer | generator | Quencher | Solvent 1 | Solvent 2 |
| | | position | (pbw) | (pbw) | (pbw) | (pbw) | (pbw) |
| Example | 3-1 | R-1 | P-1 (80) | — | SQ-1 (7.8) | PGMEA (2200) | DAA (900) |
| | 3-2 | R-2 | P-2 (80) | — | SQ-1 (7.6) | PGMEA (2200) | DAA (900) |
| | 3-3 | R-3 | P-3 (80) | — | SQ-1 (7.8) | PGMEA (2200) | DAA (900) |
| | 3-4 | R-4 | P-4 (80) | — | SQ-1 (7.8) | PGMEA (2200) | DAA (900) |
| | 3-5 | R-5 | P-5 (80) | — | SQ-1 (7.8) | PGMEA (2200) | DAA (900) |
| | 3-6 | R-6 | P-6 (80) | — | SQ-1 (7.4) | PGMEA (2200) | DAA (900) |
| | 3-7 | R-7 | P-7 (80) | — | SQ-1 (7.8) | PGMEA (2200) | DAA (900) |
| | 3-8 | R-8 | P-8 (80) | — | SQ-2 (7.8) | PGMEA (2200) | DAA (900) |
| | 3-9 | R-9 | P-9 (80) | — | SQ-3 (7.6) | PGMEA (2200) | DAA (900) |
| | 3-10 | R-10 | P-10 (80) | — | SQ-2 (7.8) | PGMEA (2200) | DAA (900) |
| | 3-11 | R-11 | P-11 (80) | — | SQ-1 (7.8) | PGMEA (2200) | DAA (900) |
| | 3-12 | R-12 | P-12 (80) | — | SQ-3 (8.8) | PGMEA (2200) | DAA (900) |
| | 3-13 | R-13 | P-13 (80) | — | SQ-2 (7.8) | PGMEA (2200) | DAA (900) |
| | 3-14 | R-14 | P-14 (80) | — | SQ-1 (7.8) | PGMEA (2200) | DAA (900) |
| | 3-15 | R-15 | P-15 (80) | PAG-1 (14.2) | SQ-1 (7.8) | PGMEA (2200) | DAA (900) |
| | 3-16 | R-16 | P-16 (80) | — | SQ-3 (7.8) | PGMEA (2200) | DAA (900) |
| | 3-17 | R-17 | P-17 (80) | PAG-2 (13.8) | SQ-2 (8.8) | PGMEA (2200) | DAA (900) |
| | 3-18 | R-18 | P-18 (80) | — | SQ-1 (3.6) | PGMEA (2200) | DAA (900) |
| | | | | | AQ-1 (3.6) | | |
| | 3-19 | R-19 | P-19 (80) | — | SQ-2 (7.8) | PGMEA (2200) | DAA (900) |
| | 3-20 | R-20 | P-20 (80) | — | SQ-1 (7.4) | PGMEA (2200) | DAA (900) |
| | 3-21 | R-21 | P-21 (80) | PAG-2 (25.8) | SQ-3 (7.3) | PGMEA (2200) | DAA (900) |
| | 3-22 | R-22 | P-22 (80) | PAG-1 (26.2) | SQ-1 (7.3) | PGMEA (2200) | DAA (900) |
| | 3-23 | R-23 | P-23 (80) | PAG-1 (25.4) | SQ-2 (7.3) | PGMEA (2200) | DAA (900) |
| | 3-24 | R-24 | P-24 (80) | PAG-1 (25.6) | SQ-1 (7.4) | PGMEA (2200) | DAA (900) |
| | 3-25 | R-25 | P-25 (80) | PAG-2 (26.2) | SQ-1 (6.8) | PGMEA (2200) | DAA (900) |
| | 3-26 | R-26 | P-26 (80) | PAG-1 (25.6) | SQ-1 (7.3) | PGMEA (2200) | DAA (900) |
| | 3-27 | R-27 | P-27 (80) | PAG-1 (24.6) | SQ-2 (3.6) | PGMEA (2200) | DAA (900) |
| | | | | | AQ-1 (3.6) | | |
| | 3-28 | R-28 | P-28 (80) | PAG-2 (25.6) | SQ-2 (7.3) | PGMEA (2200) | DAA (900) |
| | 3-29 | R-29 | P-29 (80) | PAG-1 (25.6) | SQ-1 (7.3) | PGMEA (2200) | DAA (900) |
| | 3-30 | R-30 | P-30 (80) | PAG-1 (25.6) | SQ-3 (7.5) | PGMEA (2200) | DAA (900) |
| | 3-31 | R-31 | P-31 (80) | — | SQ-1 (7.8) | PGMEA (2200) | DAA (900) |
| | 3-32 | R-32 | P-32 (80) | — | SQ-1 (7.9) | PGMEA (2200) | DAA (900) |
| | 3-33 | R-33 | P-33 (80) | — | SQ-1 (7.6) | PGMEA (2200) | DAA (900) |
| | 3-34 | R-34 | P-34 (80) | PAG-1 (25.6) | SQ-1 (7.8) | PGMEA (2200) | DAA (900) |

TABLE 4

| | | Resist | Base | Photoacid | | | |
|---|---|---|---|---|---|---|---|
| | | com- | polymer | generator | Quencher | Solvent 1 | Solvent 2 |
| | | position | (pbw) | (pbw) | (pbw) | (pbw) | (pbw) |
| Compar- | 2-1 | CR-1 | CP-1 (80) | — | SQ-1 (7.6) | PGMEA (2200) | DAA (900) |
| ative | 2-2 | CR-2 | CP-2 (80) | — | SQ-1 (7.6) | PGMEA (2200) | DAA (900) |
| Example | 2-3 | CR-3 | CP-3 (80) | — | SQ-1 (7.6) | PGMEA (2200) | DAA (900) |
| | 2-4 | CR-4 | CP-4 (80) | — | SQ-2 (7.2) | PGMEA (2200) | DAA (900) |
| | 2-5 | CR-5 | CP-5 (80) | — | SQ-3 (7.8) | PGMEA (2200) | DAA (900) |
| | 2-6 | CR-6 | CP-6 (80) | PAG-1 (14.2) | SQ-1 (7.8) | PGMEA (2200) | DAA (900) |
| | 2-7 | CR-7 | CP-7 (80) | — | SQ-3 (7.6) | PGMEA (2200) | DAA (900) |
| | 2-8 | CR-8 | CP-8 (80) | — | SQ-2 (8.4) | PGMEA (2200) | DAA (900) |
| | 2-9 | CR-9 | CP-9 (80) | — | SQ-3 (8.0) | PGMEA (2200) | DAA (900) |
| | 2-10 | CR-10 | CP-10 (80) | — | SQ-2 (7.8) | PGMEA (2200) | DAA (900) |
| | 2-11 | CR-11 | CP-11 (80) | — | SQ-1 (3.6) | PGMEA (2200) | DAA (900) |
| | | | | | AQ-1 (3.6) | | |
| | 2-12 | CR-12 | CP-12 (80) | PAG-1 (26.5) | SQ-2 (7.8) | PGMEA (2200) | DAA (900) |
| | 2-13 | CR-13 | CP-13 (80) | PAG-1 (25.6) | SQ-1 (7.3) | PGMEA (2200) | DAA (900) |
| | 2-14 | CR-14 | CP-14 (80) | PAG-2 (25.6) | SQ-2 (7.3) | PGMEA (2200) | DAA (900) |
| | 2-15 | CR-15 | CP-15 (80) | PAG-1 (25.6) | SQ-1 (7.3) | PGMEA (2200) | DAA (900) |
| | 2-16 | CR-16 | CP-16 (80) | PAG-2 (25.2) | SQ-3 (7.3) | PGMEA (2200) | DAA (900) |

The components in Tables 3 and 4 are identified below.
Organic solvent:
   PGMEA (propylene glycol monomethyl ether acetate)
   DAA (diacetone alcohol)
Photoacid generator: PAG-X and PAG-Y

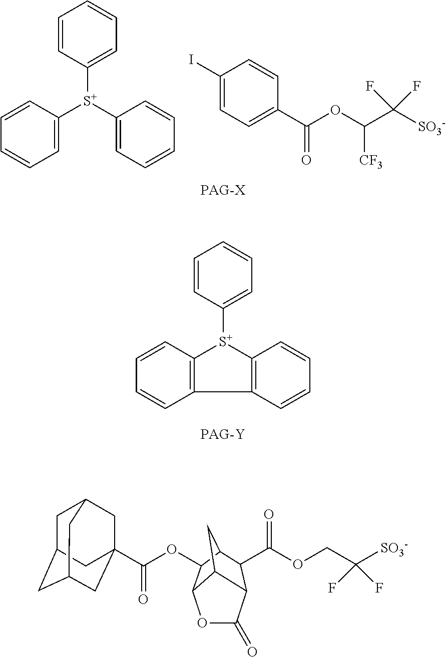

Quencher: SQ-1 to SQ-3 and AQ-1

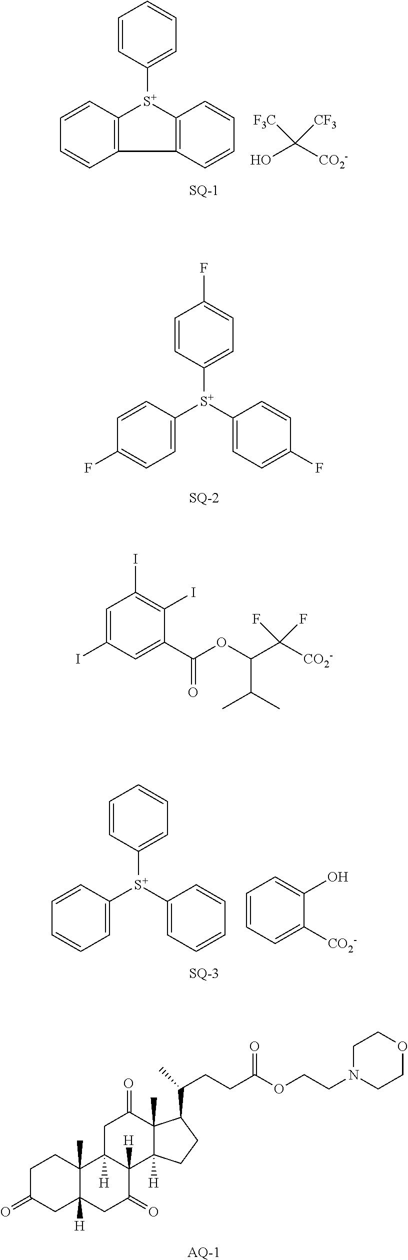

### Surfactant A: 3-methyl-3-(2,2,2-trifluoroethoxymethyl)oxetane/tetrahydrofuran/2,2-dimethyl-1,3-propanediol Copolymer (OMNOVA Solutions Inc.)

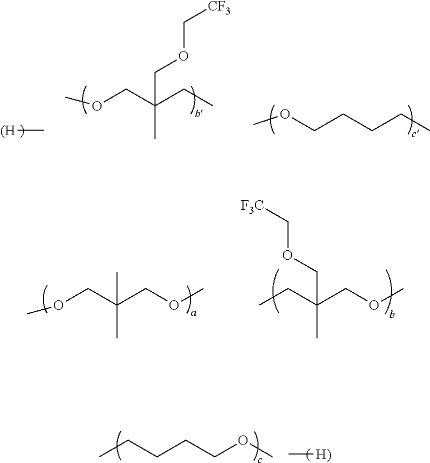

a:(b+b′):(c+c′)=1:4-7:0.01-1 (molar ratio)

### [4] EUV Lithography Test 1

### Examples 4-1 to 4-34 and Comparative Examples 3-1 to 3-16

Each of the chemically amplified resist compositions (R-1 to R-34, CR-1 to CR-16) in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, σ 0.9/0.6, dipole illumination), the resist film was exposed to EUV through a mask bearing a line-and-space (LS) pattern having a size of 18 nm and a pitch of 36 nm (on-wafer size) while varying the dose and focus (dose pitch: 1 mJ/cm², focus pitch: 0.020 μm). The resist film was baked (PEB) on a hotplate at the temperature shown in Tables 5 and 6 for 60 seconds and puddle developed in a 2.38 wt % TMAH aqueous solution for 30 seconds, rinsed with a rinse fluid containing surfactant, and spin dried to form a positive pattern.

The LS pattern as developed was observed under CD-SEM (CG6300, Hitachi High-Technologies Corp.) whereupon sensitivity, EL, LWR, DOF and collapse limit were evaluated by the following methods. The results are shown in Tables 5 and 6.

### [Evaluation of Sensitivity]

The optimum dose Eop (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation:

$$EL ( \% ) = ( \vert {E}_{{1}^{-}} {E}_{2} \vert / Eop ) \times 100$$

wherein E₁ is an optimum exposure dose which provides a LS pattern with a line width of 16.2 nm and a pitch of 36 nm, E₂ is an optimum exposure dose which provides a LS pattern with a line width of 19.8 nm and a pitch of 36 nm, and Eop is an optimum exposure dose which provides a LS pattern with a line width of 18 nm and a pitch of 36 nm. A greater value indicates better performance.

### [Evaluation of LWR]

For the LS pattern formed by exposure at the optimum dose Eop, the line width was measured at 10 longitudinally spaced apart points, from which a 3-fold value (36) of the standard deviation (6) was determined and reported as LWR. A smaller value of 36 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 5

| | | Resist | PEB | | | | | Collapse |
|---|---|---|---|---|---|---|---|---|
| | | com- | temp. | Eop | EL | LWR | DOF | limit |
| | | position | (° C.) | (mJ/cm²) | (%) | (nm) | (nm) | (nm) |
| Example | 4-1 | R-1 | 100 | 36 | 19 | 2.6 | 120 | 10.1 |
| | 4-2 | R-2 | 95 | 37 | 18 | 2.5 | 120 | 10.3 |
| | 4-3 | R-3 | 95 | 37 | 17 | 2.6 | 110 | 10.4 |
| | 4-4 | R-4 | 100 | 37 | 18 | 2.7 | 110 | 10.8 |
| | 4-5 | R-5 | 95 | 36 | 18 | 2.8 | 110 | 10.5 |
| | 4-6 | R-6 | 100 | 36 | 17 | 2.7 | 110 | 11.1 |
| | 4-7 | R-7 | 95 | 35 | 18 | 2.8 | 120 | 10.3 |
| | 4-8 | R-8 | 100 | 36 | 17 | 2.8 | 120 | 11.2 |
| | 4-9 | R-9 | 100 | 37 | 19 | 2.7 | 110 | 11.3 |
| | 4-10 | R-10 | 95 | 37 | 18 | 2.6 | 100 | 11.2 |
| | 4-11 | R-11 | 95 | 36 | 17 | 2.7 | 110 | 10.7 |
| | 4-12 | R-12 | 95 | 36 | 18 | 2.8 | 120 | 10.8 |
| | 4-13 | R-13 | 95 | 35 | 18 | 2.9 | 110 | 10.6 |
| | 4-14 | R-14 | 95 | 37 | 19 | 2.8 | 120 | 11.8 |
| | 4-15 | R-15 | 90 | 36 | 17 | 2.7 | 110 | 10.9 |
| | 4-16 | R-16 | 95 | 37 | 18 | 2.6 | 100 | 10.8 |
| | 4-17 | R-17 | 100 | 37 | 18 | 2.7 | 110 | 11.6 |
| | 4-18 | R-18 | 90 | 36 | 17 | 2.7 | 120 | 11.3 |
| | 4-19 | R-19 | 100 | 37 | 18 | 2.8 | 120 | 11.6 |
| | 4-20 | R-20 | 95 | 37 | 19 | 2.9 | 120 | 10.9 |
| | 4-21 | R-21 | 95 | 36 | 18 | 2.7 | 110 | 11.8 |
| | 4-22 | R-22 | 95 | 37 | 19 | 2.8 | 120 | 10.8 |
| | 4-23 | R-23 | 95 | 36 | 17 | 2.7 | 110 | 11.9 |
| | 4-24 | R-24 | 95 | 37 | 18 | 2.6 | 100 | 11.6 |
| | 4-25 | R-25 | 100 | 37 | 18 | 2.7 | 110 | 11.2 |
| | 4-26 | R-26 | 90 | 36 | 17 | 2.7 | 120 | 10.4 |
| | 4-27 | R-27 | 100 | 37 | 18 | 2.8 | 120 | 10.8 |
| | 4-28 | R-28 | 95 | 37 | 19 | 2.9 | 120 | 11.5 |
| | 4-29 | R-29 | 95 | 36 | 18 | 2.7 | 110 | 11.3 |
| | 4-30 | R-30 | 95 | 35 | 18 | 2.7 | 110 | 10.9 |
| | 4-31 | R-31 | 100 | 35 | 18 | 2.5 | 110 | 10.8 |
| | 4-32 | R-32 | 100 | 36 | 19 | 2.6 | 120 | 11.3 |
| | 4-33 | R-33 | 95 | 35 | 18 | 2.7 | 110 | 11.4 |
| | 4-34 | R-34 | 100 | 365 | 19 | 2.5 | 110 | 11.2 |

TABLE 6

| | | Resist | PEB | | | | | Collapse |
|---|---|---|---|---|---|---|---|---|
| | | com- | temp. | Eop | EL | LWR | DOF | limit |
| | | position | (° C.) | (mJ/cm²) | (%) | (nm) | (nm) | (nm) |
| Compar- | 3-1 | CR-1 | 100 | 40 | 16 | 3.2 | 70 | 12.1 |
| ative | 3-2 | CR-2 | 100 | 42 | 17 | 3.3 | 80 | 12.3 |
| Example | 3-3 | CR-3 | 95 | 41 | 16 | 3.2 | 90 | 12.4 |
| | 3-4 | CR-4 | 100 | 41 | 17 | 3.1 | 90 | 13.8 |
| | 3-5 | CR-5 | 100 | 41 | 15 | 3 | 70 | 12.6 |
| | 3-6 | CR-6 | 105 | 40 | 16 | 3.2 | 80 | 12.4 |
| | 3-7 | CR-7 | 100 | 39 | 16 | 3.2 | 70 | 12.7 |
| | 3-8 | CR-8 | 95 | 41 | 15 | 3.4 | 80 | 12.7 |
| | 3-9 | CR-9 | 95 | 40 | 15 | 3.1 | 90 | 12.6 |
| | 3-10 | CR-10 | 100 | 41 | 16 | 3.2 | 80 | 12.4 |
| | 3-11 | CR-11 | 95 | 40 | 15 | 3.3 | 90 | 12.6 |
| | 3-12 | CR-12 | 100 | 43 | 16 | 3.6 | 70 | 12.9 |
| | 3-13 | CR-13 | 100 | 42 | 15 | 3.7 | 90 | 12.7 |
| | 3-14 | CR-14 | 95 | 43 | 14 | 3.5 | 80 | 13.1 |
| | 3-15 | CR-15 | 100 | 42 | 16 | 3.5 | 70 | 12.9 |
| | 3-16 | CR-16 | 100 | 42 | 15 | 3.6 | 70 | 13.3 |

It is demonstrated in Tables 5 and 6 that chemically amplified resist compositions comprising PAGs within the scope of the invention exhibit a high sensitivity and improved values of EL, LWR and DOF. Small values of collapse limit attest that in forming a small-size pattern, the pattern is resistant to collapse. The resist compositions are useful in the EUV lithography process.

### [5] EUV Lithography Test 2

### Examples 5-1 to 5-34 and Comparative Examples 4-1 to 4-16

Each of the chemically amplified resist compositions (R-1 to R-34, CR-1 to CR-16) in Tables 3 and 4 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 7 and 8 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose (mJ/cm²) at which a pattern with a hole size of 23 nm was printed was determined as an index of sensitivity. The size of 50 holes was measured, from which a 3-fold value (3σ) of the standard deviation (σ) was determined as a dimensional variation (or CDU). The results are shown in Tables 7 and 8.

TABLE 7

| | | Resist | PEB temp. | Optimum dose | CDU |
|---|---|---|---|---|---|
| | | composition | (° C.) | (mJ/cm²) | (nm) |
| Example | 5-1 | R-1 | 95 | 24 | 2.2 |
| | 5-2 | R-2 | 90 | 25 | 2.4 |
| | 5-3 | R-3 | 95 | 24 | 2.4 |
| | 5-4 | R-4 | 100 | 25 | 2.5 |
| | 5-5 | R-5 | 95 | 25 | 2.3 |
| | 5-6 | R-6 | 95 | 25 | 2.4 |
| | 5-7 | R-7 | 90 | 24 | 2.4 |
| | 5-8 | R-8 | 95 | 24 | 2.5 |
| | 5-9 | R-9 | 100 | 25 | 2.5 |
| | 5-10 | R-10 | 95 | 26 | 2.4 |
| | 5-11 | R-11 | 100 | 25 | 2.6 |
| | 5-12 | R-12 | 90 | 24 | 2.6 |
| | 5-13 | R-13 | 95 | 26 | 2.5 |
| | 5-14 | R-14 | 100 | 26 | 2.4 |
| | 5-15 | R-15 | 95 | 25 | 2.4 |
| | 5-16 | R-16 | 95 | 24 | 2.5 |
| | 5-17 | R-17 | 100 | 25 | 2.5 |
| | 5-18 | R-18 | 90 | 25 | 2.4 |
| | 5-19 | R-19 | 100 | 26 | 2.6 |
| | 5-20 | R-20 | 95 | 24 | 2.5 |
| | 5-21 | R-21 | 100 | 23 | 2.4 |
| | 5-22 | R-22 | 95 | 24 | 2.6 |
| | 5-23 | R-23 | 90 | 24 | 2.6 |
| | 5-24 | R-24 | 95 | 25 | 2.5 |
| | 5-25 | R-25 | 90 | 24 | 2.5 |
| | 5-26 | R-26 | 95 | 25 | 2.4 |
| | 5-27 | R-27 | 95 | 25 | 2.6 |
| | 5-28 | R-28 | 95 | 24 | 2.4 |
| | 5-29 | R-29 | 95 | 25 | 2.3 |
| | 5-30 | R-30 | 100 | 24 | 2.4 |
| | 5-31 | R-31 | 100 | 25 | 2.5 |
| | 5-32 | R-32 | 100 | 24 | 2.3 |
| | 5-33 | R-33 | 95 | 26 | 2.2 |
| | 5-34 | R-34 | 100 | 25 | 2.4 |

TABLE 8

| | | Resist | PEB temp. | Optimum dose | CDU |
|---|---|---|---|---|---|
| | | composition | (° C.) | (mJ/cm²) | (nm) |
| Comparative | 4-1 | CR-1 | 95 | 29 | 2.8 |
| Example | 4-2 | CR-2 | 95 | 30 | 2.9 |
| | 4-3 | CR-3 | 95 | 31 | 2.9 |
| | 4-4 | CR-4 | 90 | 30 | 3.1 |
| | 4-5 | CR-5 | 95 | 30 | 2.9 |
| | 4-6 | CR-6 | 100 | 31 | 2.9 |
| | 4-7 | CR-7 | 95 | 31 | 3.2 |
| | 4-8 | CR-8 | 100 | 32 | 3.1 |
| | 4-9 | CR-9 | 90 | 39 | 3.0 |
| | 4-10 | CR-10 | 95 | 29 | 3.2 |
| | 4-11 | CR-11 | 90 | 31 | 2.9 |
| | 4-12 | CR-12 | 95 | 32 | 3.3 |
| | 4-13 | CR-13 | 95 | 31 | 3.2 |
| | 4-14 | CR-14 | 95 | 29 | 3.3 |
| | 4-15 | CR-15 | 95 | 30 | 3.2 |
| | 4-16 | CR-16 | 100 | 29 | 3.2 |

It is demonstrated in Tables 7 and 8 that chemically amplified resist compositions within the scope of the invention exhibit a high sensitivity and satisfactory CDU.

### [6] Dry Etching Test

### Examples 6-1 to 6-34 and Comparative Examples 5-1 to 5-16

Each of the polymers (Polymers P-1 to P-34 and CP-1 to CP-16 in Tables 1 and 2), 2 g, was dissolved in 10 g of cyclohexanone, and passed through a filter having a pore size of 0.2 μm, obtaining a polymer solution. The polymer solution was spin coated onto a mask blank of 152 mm squares having the outermost surface of Cr film and baked to form a polymer film of 300 nm thick.

### [Chlorine Gas Etching Test]

Using a mask dry etching instrument Gen-4 (Plasma Thermo Co., Ltd.), the polymer film was etched under the following conditions.
Chamber pressure: 6.0 mTorr
RF power: 700 V
Cl₂ flow rate: 185 sccm
O₂ flow rate: 55 sccm
He flow rate: 9 sccm
Time: 75 sec

The difference in film thickness before and after etching was determined. A smaller value of film thickness difference, i.e., a smaller loss indicates better etching resistance. The results of dry etching resistance are shown in Tables 9 and 10.

TABLE 9

| | | | Cl₂/O₂ gas etching |
|---|---|---|---|
| | | Polymer | rate (nm/min) |
| Example | 6-1 | P-1 | 95 |
| | 6-2 | P-2 | 98 |
| | 6-3 | P-3 | 97 |
| | 6-4 | P-4 | 97 |
| | 6-5 | P-5 | 96 |
| | 6-6 | P-6 | 95 |
| | 6-7 | P-7 | 97 |
| | 6-8 | P-8 | 100 |
| | 6-9 | P-9 | 98 |
| | 6-10 | P-10 | 98 |
| | 6-11 | P-11 | 97 |
| | 6-12 | P-12 | 99 |
| | 6-13 | P-13 | 97 |
| | 6-14 | P-14 | 96 |
| | 6-15 | P-15 | 101 |
| | 6-16 | P-16 | 96 |
| | 6-17 | P-17 | 98 |
| | 6-18 | P-18 | 102 |
| | 6-19 | P-19 | 96 |
| | 6-20 | P-20 | 99 |
| | 6-21 | P-21 | 97 |
| | 6-22 | P-22 | 98 |
| | 6-23 | P-23 | 102 |
| | 6-24 | P-24 | 100 |
| | 6-25 | P-25 | 101 |
| | 6-26 | P-26 | 97 |
| | 6-27 | P-27 | 96 |
| | 6-28 | P-28 | 98 |
| | 6-29 | P-29 | 100 |
| | 6-30 | P-30 | 98 |
| | 6-31 | P-31 | 97 |
| | 6-32 | P-32 | 96 |
| | 6-33 | P-33 | 96 |
| | 6-34 | P-34 | 97 |

TABLE 10

| | | | Cl₂/O₂ gas etching |
|---|---|---|---|
| | | Polymer | rate (nm/min) |
| Comparative | 5-1 | CP-1 | 109 |
| Example | 5-2 | CP-2 | 110 |
| | 5-3 | CP-3 | 112 |
| | 5-4 | CP-4 | 110 |
| | 5-5 | CP-5 | 109 |
| | 5-6 | CP-6 | 109 |
| | 5-7 | CP-7 | 108 |
| | 5-8 | CP-8 | 107 |
| | 5-9 | CP-9 | 106 |
| | 5-10 | CP-10 | 109 |
| | 5-11 | CP-11 | 110 |
| | 5-12 | CP-12 | 112 |
| | 5-13 | CP-13 | 113 |
| | 5-14 | CP-14 | 114 |
| | 5-15 | CP-15 | 110 |
| | 5-16 | CP-16 | 109 |

It has been demonstrated that the polymers within the scope of the invention display excellent resistance against dry etching with Cl₂/O₂ gas.

Japanese Patent Application No. 2023-169702 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A monomer having the formula (A): 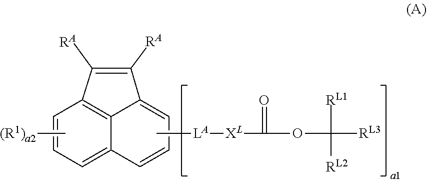
wherein a1 is 1 or 2, a2 is an integer of 0 to 4,
R^{A} is each independently hydrogen, fluorine, methyl or trifluoromethyl,
R¹ is halogen or a C₁-C₂₀ hydrocarbyl group which may contain a heteroatom, a plurality of R¹ may bond together to form a ring with the carbon atoms to which they are attached when a2 is 2 or more,
L^{A} is a single bond, ether bond, ester bond, amide bond, sulfonate ester bond, carbonate bond or carbamate bond,
X^{L} is a single bond or C₁-C₄₀ hydrocarbylene group which may contain a heteroatom,
R^{L1}, R^{L2} and R^{L3} are each independently a C₁-C₃₀ hydrocarbyl group which may contain a heteroatom, any two of R^{L1}, R^{L2} and R^{L3} may bond together to form a ring with the carbon atom to which they are attached, at least one of R^{L1}, R^{L2} and R^{L3} has at least one structure selected from a multiple bond, alicyclic and aromatic ring when they do not form a ring.

2. The monomer of claim 1, having the formula (A1): 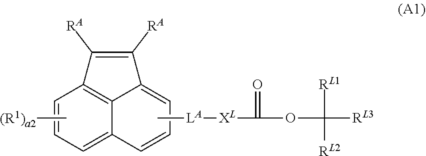
wherein a2, R^{A}, R¹, L^{A}, X^{L}, and R^{L1} to R^{L3} are as defined above.

3. The monomer of claim 2, having the formula (A2): 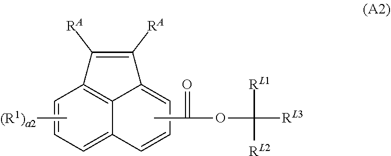
wherein a2, R^{A}, R¹, and R^{L1} to R^{L3} are as defined above.

4. A polymer comprising repeat units derived from the monomer of claim 1.

5. The polymer of claim 4, further comprising repeat units of at least one type selected from repeat units having the formula (a1) and repeat units having the formula (a2): 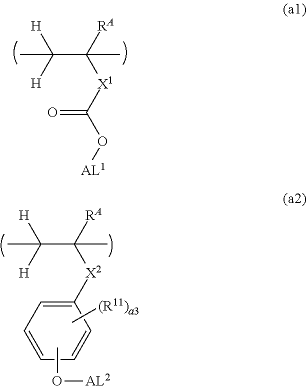
wherein R^{A} is each independently hydrogen, fluorine, methyl or trifluoromethyl,
X¹ is a single bond, phenylene, naphthylene, *—C(═O)—O—X¹¹—, or *—C(═O)—NH—X¹¹—, the phenylene or naphthylene group may be substituted with an optionally fluorinated C.C₁₀ alkoxy moiety or halogen, X¹¹ is a C₁-C₁₀ saturated hydrocarbylene group, phenylene, or naphthylene, the saturated hydrocarbylene group may contain a hydroxy, ether bond, ester bond or lactone ring,
X² is a single bond, *—C(═O)—O— or *—C(═O)—NH—,
* designates a point of attachment to the carbon atom in the backbone,
R¹¹ is halogen, cyano group, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL¹ and AL² are each independently an acid labile group, and
a3 is an integer of 0 to 4.

6. The polymer of claim 4, further comprising repeat units of at least one type selected from repeat units having the formula (b1) and repeat units having the formula (b2): 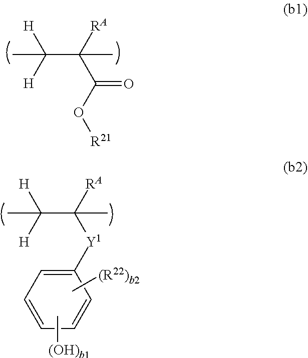
wherein R^{A} is each independently hydrogen, fluorine, methyl or trifluoromethyl,
Y¹ is a single bond or *—C(═O)—O—,*designates a point of attachment to the carbon atom in the backbone,
R²¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—),
R²² is halogen, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
b1 is an integer of 1 to 4, b2 is an integer of 0 to 4, and b1+b2 is from 1 to 5.

7. The polymer of claim 4, further comprising repeat units of at least one type selected from repeat units having the formulae (c1) to (c4): 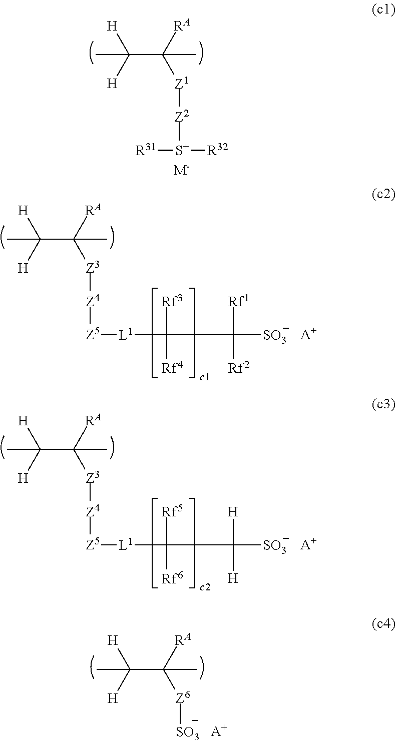
wherein R^{A} is each independently hydrogen, fluorine, methyl or trifluoromethyl,
Z¹ is a single bond or phenylene group,
Z² is **—C(═O)—O—Z²—, **—C(═O)—NH—Z²—, or **—O—Z²¹—, Z²¹ is a C₁-C₆ aliphatic hydrocarbylene group, phenylene group or a divalent group obtained by combining the foregoing, which may contain a carbonyl moiety, ester bond, ether bond or hydroxy moiety,
Z³ is each independently a single bond, phenylene, naphthylene, or *—C(═O)—O—Z—Z³¹ is a C₁-C₁₀ aliphatic hydrocarbylene group which may contain a hydroxy moiety, ether bond, ester bond or lactone ring, or phenylene or naphthylene group,
Z⁴ is each independently a single bond, ***—Z⁴¹—C(═O)—O—, ***—C(═O)—NH—Z⁴¹—, or ***—Z⁴¹—, Z⁴¹ is a C₁-C₂₀ hydrocarbylene group which may contain a heteroatom,
Z⁵ is each independently a single bond, ****—Z⁵—C(═O)—O—, ****—C(═O)—NH—Z^{5l}, or ****—O—Z⁵—, Z⁵¹ is a C₁-C₂₀ hydrocarbylene group which may contain a heteroatom,
Z⁶ is a single bond, methylene, ethylene, phenylene, fluorinated phenylene, trifluoromethyl-substituted phenylene, *—C(═O)—O—Z⁶¹—, *—C(═O)—N(H)—Z⁶¹—, or *—O—Z⁶¹, Z⁶¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone, ** designates a point of attachment to Z¹, *** designates a point of attachment to Z³, **** designates a point of attachment to Z⁴,
R³¹ and R³² are each independently a C₁-C₂₀ hydrocarbyl group which may contain a heteroatom, R³¹ and R³² may bond together to form a ring with the sulfur atom to which they are attached,
L¹ is a single bond, ether bond, ester bond, carbonyl group, sulfonate ester bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf^{V} and Rf⁶ are hydrogen at the same time,
M⁻ is a non-nucleophilic counter ion,
A⁺ is an onium cation, and
c1 and c2 are each independently an integer of 0 to 3.

8. A chemically amplified resist composition comprising a base polymer containing the polymer of claim 4 and an organic solvent.

9. The chemically amplified resist composition of claim 8, further comprising a quencher.

10. The chemically amplified resist composition of claim 8, further comprising a photoacid generator.

11. The chemically amplified resist composition of claim 8, further comprising a surfactant.

12. A pattern forming process comprising the steps of applying the chemically amplified resist composition of claim 8 onto a substrate to form a resist film thereon, exposing the resist film to high-energy radiation, and developing the exposed resist film in a developer.

13. The pattern forming process of claim 12 wherein the high-energy radiation is KrF excimer laser radiation, ArF excimer laser radiation, EB or EUV of wavelength 3 to 15 nm.
